SEQUENCE LISTING (494 sequences)

SEQ ID NO: 1 - protein, 20 aa, unidentified
MSQSGEENLQGSWVELHFSN

SEQ ID NO: 2 - protein, 8 aa, synthetic construct
WVELHFSN

SEQ ID NO: 3 - protein, 8 aa, synthetic construct
WVELHFFN

SEQ ID NO: 4 - protein, 5 aa, unidentified
PESTQ

SEQ ID NO: 5 - protein, 19 aa, synthetic construct
MSQGEENLQGSWVELHFSN

SEQ ID NO: 6 - protein, 20 aa, synthetic construct
LDAQHESGRSSSKSSHCDSP

SEQ ID NO: 7 - protein, 8 aa, synthetic construct
WVELAASN

SEQ ID NO: 8 - protein, 7 aa, synthetic construct
VELHFSN

SEQ ID NO: 9 - protein, 8 aa, synthetic construct
  SITE 8: note = Residue attached to a fluorophore
WVELHFFN

SEQ ID NO: 10 - protein, 8 aa, synthetic construct
  SITE 8: note = Residue attached to a Cy5.5 fluorophore
WVELHFFN

SEQ ID NO: 11 - protein, 8 aa, synthetic construct
WELVHFFN

SEQ ID NO: 12 - protein, 7 aa, synthetic construct
WELAASN

SEQ ID NO: 13 - protein, 12 aa, Human immunodeficiency virus 1
GRKKRRQRRRPQ

SEQ ID NO: 14 - protein, 7 aa, synthetic construct
WVELHFN

SEQ ID NO: 15 - protein, 9 aa, synthetic construct
  SITE 9: note = Residue attached to 5(6)-Carboxyfluorescein
WVELHFFNK

SEQ ID NO: 16 - protein, 32 aa, synthetic construct
GRKKRRQRRRPQMSQSGEENLQGSWVELHFSN

SEQ ID NO: 17 - protein, 32 aa, synthetic construct
GRKKRRQRRRPQLDAQHESGRSSSKSSHCDSP

SEQ ID NO: 18 - protein, 20 aa, synthetic construct
GRKKRRQRRRPQWVELHFFN

SEQ ID NO: 19 - protein, 20 aa, synthetic construct
GRKKRRQRRRPQWVELAASN

SEQ ID NO: 20 - protein, 20 aa, synthetic construct
MDGSGKQLGSGGPTSSKQIM

SEQ ID NO: 21 - protein, 20 aa, synthetic construct
SKQIMKTGAFLLQGFIQDRA

SEQ ID NO: 22 - protein, 20 aa, synthetic construct
AGPMAGKTPKLTLKQPPQDA

SEQ ID NO: 23 - protein, 20 aa, synthetic construct
DASTKKLSKCLRRIGDKLDS

SEQ ID NO: 24 - protein, 20 aa, synthetic construct
KLDSNMKLQRMIADVDTDSP

SEQ ID NO: 25 - protein, 20 aa, synthetic construct
TDSPRKVFFRVAADMFADGN

SEQ ID NO: 26 - protein, 20 aa, synthetic construct
WGRVVALFYFASKLVLKALC

SEQ ID NO: 27 - protein, 20 aa, synthetic construct
VPKLIRTIMGWTLDFLRKRL

SEQ ID NO: 28 - protein, 20 aa, synthetic construct
RLLVWIQDQGGWEGLLSYFG

SEQ ID NO: 29 - protein, 20 aa, synthetic construct
TWQTVTIFVAGVLTASLTIW

SEQ ID NO: 30 - protein, 20 aa, synthetic construct
MFADGNFNWGRVVALFYFAS

SEQ ID NO: 31 - protein, 20 aa, synthetic construct
LVWIQDQGGWEGLLSYFGTP

SEQ ID NO: 32 - protein, 20 aa, synthetic construct
ASTKKLSECLRRIGDKLDSN

SEQ ID NO: 33 - protein, 20 aa, synthetic construct
VPELIRTIMGWTLDFLRERL

SEQ ID NO: 34 - protein, 20 aa, synthetic construct
RLLVWIQDQGGWEGLLSYFG

SEQ ID NO: 35 - protein, 9 aa, synthetic construct
CLKRIGDEL

SEQ ID NO: 36 - protein, 20 aa, synthetic construct
MSQSCEENLQCSWVELHFEN

SEQ ID NO: 37 - protein, 10 aa, synthetic construct
MSQSCEENLQ

SEQ ID NO: 38 - protein, 187 aa, Mus musculus
MSQSGEENLQGSWVELHFSNGNGSSVPASVSIYNGDMEKILLDAQHESGRSSSKSSHCDSPPRSQTPQDTNRAEIDSHSFGEKNSTLSEEDYIERRREVESILKKNSDWIWDWSSRPENIPPKEFLFKHPKRTATLSMRNTSVMKKGGIFSADFLKVFLPSLLLSHLLAIGLGIYIGRRLTTSTSTF

SEQ ID NO: 39 - protein, 194 aa, Homo sapiens
MSQNGAPGMQEESLQGSWVELHFSNNGNGGSVPASVSIYNGDMEKILLDAQHESGRSSSKSSHCDSPPRSQTPQDTNRASETDTHSIGEKNSSQSEEDDIERRKEVESILKKNSDWIWDWSSRPENIPPKEFLFKHPKRTATLSMRNTSVMKKGGIFSAEFLKVFLPSLLLSHLLAIGLGIYIGRRLTTSTSTF

SEQ ID NO: 40 - protein, 19 aa, synthetic construct
SQSGEENLQGSWVELHFSN

SEQ ID NO: 41 - protein, 18 aa, synthetic construct
QSGEENLQGSWVELHFSN

SEQ ID NO: 42 - protein, 17 aa, synthetic construct
SGEENLQGSWVELHFSN

SEQ ID NO: 43 - protein, 16 aa, synthetic construct
GEENLQGSWVELHFSN

SEQ ID NO: 44 - protein, 15 aa, synthetic construct
EENLQGSWVELHFSN

SEQ ID NO: 45 - protein, 14 aa, synthetic construct
ENLQGSWVELHFSN

SEQ ID NO: 46 - protein, 13 aa, synthetic construct
NLQGSWVELHFSN

SEQ ID NO: 47 - protein, 12 aa, synthetic construct
LQGSWVELHFSN

SEQ ID NO: 48 - protein, 11 aa, synthetic construct
QGSWVELHFSN

SEQ ID NO: 49 - protein, 10 aa, synthetic construct
GSWVELHFSN

SEQ ID NO: 50 - protein, 9 aa, synthetic construct
SWVELHFSN

SEQ ID NO: 51 - protein, 6 aa, synthetic construct
ELHFSN

SEQ ID NO: 52 - protein, 5 aa, synthetic construct
LHFSN

SEQ ID NO: 53 - protein, 4 aa, synthetic construct
HFSN

SEQ ID NO: 54 - protein, 20 aa, synthetic construct
MSQSGEENLQGSWVELHAAA

SEQ ID NO: 55 - protein, 19 aa, synthetic construct
MSQSGEENLQGSWVEAAAN

SEQ ID NO: 56 - protein, 20 aa, synthetic construct
MSQSGEENLQGSWVEAAASN

SEQ ID NO: 57 - protein, 20 aa, synthetic construct
MSQSGEENLQGSWVAAAFSN

SEQ ID NO: 58 - protein, 20 aa, synthetic construct
MSQSGEENLQGSWAAAHFSN

SEQ ID NO: 59 - protein, 20 aa, synthetic construct
MSQSGEENLQAAAVELHFSN

SEQ ID NO: 60 - protein, 20 aa, synthetic construct
MSQSGEENLAAAWVELHFSN

SEQ ID NO: 61 - protein, 20 aa, synthetic construct
MSQSGEENAAASWVELHFSN

SEQ ID NO: 62 - protein, 20 aa, synthetic construct
MSQSGEEAAAGSWVELHFSN

SEQ ID NO: 63 - protein, 20 aa, synthetic construct
MSQSGEAAAQGSWVELHFSN

SEQ ID NO: 64 - protein, 20 aa, synthetic construct
MSQSGAAALQGSWVELHFSN

SEQ ID NO: 65 - protein, 20 aa, synthetic construct
MSQSAAANLQGSWVELHFSN

SEQ ID NO: 66 - protein, 20 aa, synthetic construct
MSQAAAENLQGSWVELHFSN

SEQ ID NO: 67 - protein, 20 aa, synthetic construct
MSAAAEENLQGSWVELHFSN

SEQ ID NO: 68 - protein, 19 aa, synthetic construct
MAAAEENLQGSWVELHFSN

SEQ ID NO: 69 - protein, 20 aa, synthetic construct
AAASGEENLQGSWVELHFSN

SEQ ID NO: 70 - protein, 20 aa, synthetic construct
MSQSGEENLQGSWVELHFAA

SEQ ID NO: 71 - protein, 20 aa, synthetic construct
MSQSGEENLQGSWVELHAAN

SEQ ID NO: 72 - protein, 20 aa, synthetic construct
MSQSGEENLQGSWVELAASN

SEQ ID NO: 73 - protein, 20 aa, synthetic construct
MSQSGEENLQGSWVEAAFSN

SEQ ID NO: 74 - protein, 20 aa, synthetic construct
MSQSGEENLQGSWVAAHFSN

SEQ ID NO: 75 - protein, 20 aa, synthetic construct
MSQSGEENLQGSWAALHFSN

SEQ ID NO: 76 - protein, 20 aa, synthetic construct
MSQSGEENLQGSAAELHFSN

SEQ ID NO: 77 - protein, 20 aa, synthetic construct
MSQSGEENLQGAAVELHFSN

SEQ ID NO: 78 - protein, 20 aa, synthetic construct
MSQSGEENLQAAWVELHFSN

SEQ ID NO: 79 - protein, 20 aa, synthetic construct
MSQSGEENLAASWVELHFSN

SEQ ID NO: 80 - protein, 20 aa, synthetic construct
MSQSGEAALQGSWVELHFSN

SEQ ID NO: 81 - protein, 20 aa, synthetic construct
MSQSGAANLQGSWVELHFSN

SEQ ID NO: 82 - protein, 20 aa, synthetic construct
MSQSAAENLQGSWVELHFSN

SEQ ID NO: 83 - protein, 20 aa, synthetic construct
MSQAAEENLQGSWVELHFSN

SEQ ID NO: 84 - protein, 20 aa, synthetic construct
MSAAGEENLQGSWVELHFSN

SEQ ID NO: 85 - protein, 20 aa, synthetic construct
MAASGEENLQGSWVELHFSN

SEQ ID NO: 86 - protein, 20 aa, synthetic construct
AAQSGEENLQGSWVELHFSN

SEQ ID NO: 87 - protein, 20 aa, synthetic construct
MDGSGEQLGSGGPTSSEQIM

SEQ ID NO: 88 - protein, 20 aa, synthetic construct
AGRMAGETPELTLEQPPQDA

SEQ ID NO: 89 - protein, 20 aa, synthetic construct
DASTKKLSECLRRIGDELDS

SEQ ID NO: 90 - protein, 20 aa, synthetic construct
ELDSNMELQRMIADVDTDSP

SEQ ID NO: 91 - protein, 20 aa, synthetic construct
TDSPREVFFRVAADMFADGN

SEQ ID NO: 92 - protein, 20 aa, synthetic construct
MFADGNFNWGRVVALFYFAS

SEQ ID NO: 93 - protein, 20 aa, synthetic construct
ASTKKLSECLRRIGDELDSN

SEQ ID NO: 94 - protein, 4 aa, synthetic construct
LQGS

SEQ ID NO: 95 - protein, 6 aa, synthetic construct
NLQGSW

SEQ ID NO: 96 - protein, 8 aa, synthetic construct
ENLQGSWV

SEQ ID NO: 97 - protein, 10 aa, synthetic construct
EENLQGSWVE

SEQ ID NO: 98 - protein, 12 aa, synthetic construct
GEENLQGSWVEL

SEQ ID NO: 99 - protein, 14 aa, synthetic construct
SGEENLQGSWVELH

SEQ ID NO: 100 - protein, 16 aa, synthetic construct
QSGEENLQGSWVELHF

SEQ ID NO: 101 - protein, 18 aa, synthetic construct
SQSGEENLQGSWVELHFS

SEQ ID NO: 102 - protein, 4 aa, synthetic construct
MSQS

SEQ ID NO: 103 - protein, 5 aa, synthetic construct
MSQSG

SEQ ID NO: 104 - protein, 6 aa, synthetic construct
MSQSGE

SEQ ID NO: 105 - protein, 7 aa, synthetic construct
MSQSGEE

SEQ ID NO: 106 - protein, 8 aa, synthetic construct
MSQSGEEN

SEQ ID NO: 107 - protein, 9 aa, synthetic construct
MSQSGEENL

SEQ ID NO: 108 - protein, 10 aa, synthetic construct
MSQSGEENLQ

SEQ ID NO: 109 - protein, 11 aa, synthetic construct
MSQSGEENLQG

SEQ ID NO: 110 - protein, 12 aa, synthetic construct
MSQSGEENLQGS

SEQ ID NO: 111 - protein, 13 aa, synthetic construct
MSQSGEENLQGSW

SEQ ID NO: 112 - protein, 14 aa, synthetic construct
MSQSGEENLQGSWV

SEQ ID NO: 113 - protein, 15 aa, synthetic construct
MSQSGEENLQGSWVE

SEQ ID NO: 114 - protein, 16 aa, synthetic construct
MSQSGEENLQGSWVEL

SEQ ID NO: 115 - protein, 17 aa, synthetic construct
MSQSGEENLQGSWVELH

SEQ ID NO: 116 - protein, 18 aa, synthetic construct
MSQSGEENLQGSWVELHF

SEQ ID NO: 117 - protein, 19 aa, synthetic construct
MSQSGEENLQGSWVELHFS

SEQ ID NO: 118 - protein, 20 aa, synthetic construct
MSQSGEENLQGSWVALHFSN

SEQ ID NO: 119 - protein, 20 aa, synthetic construct
MSQSGERNLQGSWVELHFSN

SEQ ID NO: 120 - protein, 20 aa, synthetic construct
MSQSKEENLQGSWVELHFSN

SEQ ID NO: 121 - protein, 20 aa, synthetic construct
MSQSGEENCQGSWVELHFSN

SEQ ID NO: 122 - protein, 20 aa, synthetic construct
MSQSGEENLQGSWVELHFSS

SEQ ID NO: 123 - protein, 20 aa, synthetic construct
MSQSGEENLQGSWVCLHFSN

SEQ ID NO: 124 - protein, 20 aa, synthetic construct
MSQSGESNLQGSWVELHFSN

SEQ ID NO: 125 - protein, 20 aa, synthetic construct
MSQSLEENLQGSWVELHFSN

SEQ ID NO: 126 - protein, 20 aa, synthetic construct
MSQSGEENDQGSWVELHFSN

SEQ ID NO: 127 - protein, 20 aa, synthetic construct
MSQSGEENLQGSWVELHFST

SEQ ID NO: 128 - protein, 20 aa, synthetic construct
MSQSGEENLQGSWVDLHFSN

SEQ ID NO: 129 - protein, 20 aa, synthetic construct
MSQSGETNLQGSWVELHFSN

SEQ ID NO: 130 - protein, 20 aa, synthetic construct
MSQSMEENLQGSWVELHFSN

SEQ ID NO: 131 - protein, 20 aa, synthetic construct
MSQSGEENEQGSWVELHFSN

SEQ ID NO: 132 - protein, 20 aa, synthetic construct
MSQSGEENLQGSWVELHFSV

SEQ ID NO: 133 - protein, 20 aa, synthetic construct
MSQSGEENLQGSWVFLHFSN

SEQ ID NO: 134 - protein, 20 aa, synthetic construct
MSQSGEVNLQGSWVELHFSN

SEQ ID NO: 135 - protein, 20 aa, synthetic construct
MSQSNEENLQGSWVELHFSN

SEQ ID NO: 136 - protein, 20 aa, synthetic construct
MSQSGEENFQGSWVELHFSN

SEQ ID NO: 137 - protein, 20 aa, synthetic construct
MSQSGEENLQGSWVELHFSW

SEQ ID NO: 138 - protein, 20 aa, synthetic construct
MSQSGEENLQGSWVGLHFSN

SEQ ID NO: 139 - protein, 20 aa, synthetic construct
MSQSGEWNLQGSWVELHFSN

SEQ ID NO: 140 - protein, 20 aa, synthetic construct
MSQSPEENLQGSWVELHFSN

SEQ ID NO: 141 - protein, 20 aa, synthetic construct
MSQSGEENGQGSWVELHFSN

SEQ ID NO: 142 - protein, 20 aa, synthetic construct
MSQSGEENLQGSWVELHFSY

SEQ ID NO: 143 - protein, 20 aa, synthetic construct
MSQSGEENLQGSWVHLHFSN

SEQ ID NO: 144 - protein, 20 aa, synthetic construct
MSQSGEYNLQGSWVELHFSN

SEQ ID NO: 145 - protein, 20 aa, synthetic construct
MSQSQEENLQGSWVELHFSN

SEQ ID NO: 146 - protein, 20 aa, synthetic construct
MSQSGEENHQGSWVELHFSN

SEQ ID NO: 147 - protein, 20 aa, synthetic construct
MSQSGEEALQGSWVELHFSN

SEQ ID NO: 148 - protein, 20 aa, synthetic construct
MSQSGEENLQGSWVILHFSN

SEQ ID NO: 149 - protein, 20 aa, synthetic construct
MSQSGEENLQGSWVELHASN

SEQ ID NO: 150 - protein, 20 aa, synthetic construct
MSQSREENLQGSWVELHFSN

SEQ ID NO: 151 - protein, 20 aa, synthetic construct
MSQSGEENIQGSWVELHFSN

SEQ ID NO: 152 - protein, 20 aa, synthetic construct
MSQSGEECLQGSWVELHFSN

SEQ ID NO: 153 - protein, 20 aa, synthetic construct
MSQSGEENLQGSWVKLHFSN

SEQ ID NO: 154 - protein, 20 aa, synthetic construct
MSQSGEENLQGSWVELHCSN

SEQ ID NO: 155 - protein, 20 aa, synthetic construct
MSQSSEENLQGSWVELHFSN

SEQ ID NO: 156 - protein, 20 aa, synthetic construct
MSQSGEENKQGSWVELHFSN

SEQ ID NO: 157 - protein, 20 aa, synthetic construct
MSQSGEEDLQGSWVELHFSN

SEQ ID NO: 158 - protein, 20 aa, synthetic construct
MSQSGEENLQGSWVLLHFSN

SEQ ID NO: 159 - protein, 20 aa, synthetic construct
MSQSGEENLQGSWVELHDSN

SEQ ID NO: 160 - protein, 20 aa, synthetic construct
MSQSTEENLQGSWVELHFSN

SEQ ID NO: 161 - protein, 20 aa, synthetic construct
MSQSGEENMQGSWVELHFSN

SEQ ID NO: 162 - protein, 20 aa, synthetic construct
MSQSGEEELQGSWVELHFSN

SEQ ID NO: 163 - protein, 20 aa, synthetic construct
MSQSGEENLQGSWVMLHFSN

SEQ ID NO: 164 - protein, 20 aa, synthetic construct
MSQSGEENLQGSWVELHESN

SEQ ID NO: 165 - protein, 20 aa, synthetic construct
MSQSVEENLQGSWVELHFSN

SEQ ID NO: 166 - protein, 20 aa, synthetic construct
MSQSGEENNQGSWVELHFSN

SEQ ID NO: 167 - protein, 20 aa, synthetic construct
MSQSGEEFLQGSWVELHFSN

SEQ ID NO: 168 - protein, 20 aa, synthetic construct
MSQSGEENLQGSWVNLHFSN

SEQ ID NO: 169 - protein, 20 aa, synthetic construct
MSQSGEENLQGSWVELHGSN

SEQ ID NO: 170 - protein, 20 aa, synthetic construct
MSQSWEENLQGSWVELHFSN

SEQ ID NO: 171 - protein, 20 aa, synthetic construct
MSQSGEENPQGSWVELHFSN

SEQ ID NO: 172 - protein, 20 aa, synthetic construct
MSQSGEEGLQGSWVELHFSN

SEQ ID NO: 173 - protein, 20 aa, synthetic construct
MSQSGEENLQGSWVPLHFSN

SEQ ID NO: 174 - protein, 20 aa, synthetic construct
MSQSGEENLQGSWVELHHSN

SEQ ID NO: 175 - protein, 20 aa, synthetic construct
MSQSYEENLQGSWVELHFSN

SEQ ID NO: 176 - protein, 20 aa, synthetic construct
MSQSGEENQQGSWVELHFSN

SEQ ID NO: 177 - protein, 20 aa, synthetic construct
MSQSGEEHLQGSWVELHFSN

SEQ ID NO: 178 - protein, 20 aa, synthetic construct
MSQSGEENLQGSWVQLHFSN

SEQ ID NO: 179 - protein, 20 aa, synthetic construct
MSQSGEENLQGSWVELHISN

SEQ ID NO: 180 - protein, 20 aa, synthetic construct
MSQSGEENLQGSWVELAFSN

SEQ ID NO: 181 - protein, 20 aa, synthetic construct
MSQSGEENRQGSWVELHFSN

SEQ ID NO: 182 - protein, 20 aa, synthetic construct
MSQSGEEILQGSWVELHFSN

SEQ ID NO: 183 - protein, 20 aa, synthetic construct
MSQSGEENLQGSWVRLHFSN

SEQ ID NO: 184 - protein, 20 aa, synthetic construct
MSQSGEENLQGSWVELHKSN

SEQ ID NO: 185 - protein, 20 aa, synthetic construct
MSQSGEENLQGSWVELCFSN

SEQ ID NO: 186 - protein, 20 aa, synthetic construct
MSQSGEENSQGSWVELHFSN

SEQ ID NO: 187 - protein, 20 aa, synthetic construct
MSQSGEEKLQGSWVELHFSN

SEQ ID NO: 188 - protein, 20 aa, synthetic construct
MSQSGEENLQGSWVSLHFSN

SEQ ID NO: 189 - protein, 20 aa, synthetic construct
MSQSGEENLQGSWVELHLSN

SEQ ID NO: 190 - protein, 20 aa, synthetic construct
MSQSGEENLQGSWVELDFSN

SEQ ID NO: 191 - protein, 20 aa, synthetic construct
MSQSGEENTQGSWVELHFSN

SEQ ID NO: 192 - protein, 20 aa, synthetic construct
MSQSGEELLQGSWVELHFSN

SEQ ID NO: 193 - protein, 20 aa, synthetic construct
MSQSGEENLQGSWVTLHFSN

SEQ ID NO: 194 - protein, 20 aa, synthetic construct
MSQSGEENLQGSWVELHMSN

SEQ ID NO: 195 - protein, 20 aa, synthetic construct
MSQSGEENLQGSWVELEFSN

SEQ ID NO: 196 - protein, 20 aa, synthetic construct
MSQSGEENVQGSWVELHFSN

SEQ ID NO: 197 - protein, 20 aa, synthetic construct
MSQSGEEMLQGSWVELHFSN

SEQ ID NO: 198 - protein, 20 aa, synthetic construct
MSQSGEENLQGSWVVLHFSN

SEQ ID NO: 199 - protein, 20 aa, synthetic construct
MSQSGEENLQGSWVELHNSN

SEQ ID NO: 200 - protein, 20 aa, synthetic construct
MSQSGEENLQGSWVELFFSN

SEQ ID NO: 201 - protein, 20 aa, synthetic construct
MSQSGEENWQGSWVELHFSN

SEQ ID NO: 202 - protein, 20 aa, synthetic construct
MSQSGEEPLQGSWVELHFSN

SEQ ID NO: 203 - protein, 20 aa, synthetic construct
MSQSGEENLQGSWVWLHFSN

SEQ ID NO: 204 - protein, 20 aa, synthetic construct
MSQSGEENLQGSWVELHPSN

SEQ ID NO: 205 - protein, 20 aa, synthetic construct
MSQSGEENLQGSWVELGFSN

SEQ ID NO: 206 - protein, 20 aa, synthetic construct
MSQSGEENYQGSWVELHFSN

SEQ ID NO: 207 - protein, 20 aa, synthetic construct
MSQSGEEQLQGSWVELHFSN

SEQ ID NO: 208 - protein, 20 aa, synthetic construct
MSQSGEENLQGSWVYLHFSN

SEQ ID NO: 209 - protein, 20 aa, synthetic construct
MSQSGEENLQGSWVELHQSN

SEQ ID NO: 210 - protein, 20 aa, synthetic construct
MSQSGEENLQGSWVELIFSN

SEQ ID NO: 211 - protein, 20 aa, synthetic construct
ASQSGEENLQGSWVELHFSN

SEQ ID NO: 212 - protein, 20 aa, synthetic construct
MSQSGEERLQGSWVELHFSN

SEQ ID NO: 213 - protein, 20 aa, synthetic construct
MSQSGAENLQGSWVELHFSN

SEQ ID NO: 214 - protein, 20 aa, synthetic construct
MSQSGEENLQGSWVELHRSN

SEQ ID NO: 215 - protein, 20 aa, synthetic construct
MSQSGEENLQGSWVELKFSN

SEQ ID NO: 216 - protein, 20 aa, synthetic construct
CSQSGEENLQGSWVELHFSN

SEQ ID NO: 217 - protein, 20 aa, synthetic construct
MSQSGEESLQGSWVELHFSN

SEQ ID NO: 218 - protein, 20 aa, synthetic construct
MSQSGCENLQGSWVELHFSN

SEQ ID NO: 219 - protein, 20 aa, synthetic construct
MSQSGEENLQGSWVELHSSN

SEQ ID NO: 220 - protein, 20 aa, synthetic construct
MSQSGEENLQGSWVELLFSN

SEQ ID NO: 221 - protein, 20 aa, synthetic construct
DSQSGEENLQGSWVELHFSN

SEQ ID NO: 222 - protein, 20 aa, synthetic construct
MSQSGEETLQGSWVELHFSN

SEQ ID NO: 223 - protein, 20 aa, synthetic construct
MSQSGDENLQGSWVELHFSN

SEQ ID NO: 224 - protein, 20 aa, synthetic construct
MSQSGEENLQGSWVELHTSN

SEQ ID NO: 225 - protein, 20 aa, synthetic construct
MSQSGEENLQGSWVELMFSN

SEQ ID NO: 226 - protein, 20 aa, synthetic construct
ESQSGEENLQGSWVELHFSN

SEQ ID NO: 227 - protein, 20 aa, synthetic construct
MSQSGEEVLQGSWVELHFSN

SEQ ID NO: 228 - protein, 20 aa, synthetic construct
MSQSGFENLQGSWVELHFSN

SEQ ID NO: 229 - protein, 20 aa, synthetic construct
MSQSGEENLQGSWVELHVSN

SEQ ID NO: 230 - protein, 20 aa, synthetic construct
MSQSGEENLQGSWVELNFSN

SEQ ID NO: 231 - protein, 20 aa, synthetic construct
FSQSGEENLQGSWVELHFSN

SEQ ID NO: 232 - protein, 20 aa, synthetic construct
MSQSGEEWLQGSWVELHFSN

SEQ ID NO: 233 - protein, 20 aa, synthetic construct
MSQSGGENLQGSWVELHFSN

SEQ ID NO: 234 - protein, 20 aa, synthetic construct
MSQSGEENLQGSWVELHWSN

SEQ ID NO: 235 - protein, 20 aa, synthetic construct
MSQSGEENLQGSWVELPFSN

SEQ ID NO: 236 - protein, 20 aa, synthetic construct
GSQSGEENLQGSWVELHFSN

SEQ ID NO: 237 - protein, 20 aa, synthetic construct
MSQSGEEYLQGSWVELHFSN

SEQ ID NO: 238 - protein, 20 aa, synthetic construct
MSQSGHENLQGSWVELHFSN

SEQ ID NO: 239 - protein, 20 aa, synthetic construct
MSQSGEENLQGSWVELHYSN

SEQ ID NO: 240 - protein, 20 aa, synthetic construct
MSQSGEENLQGSWVELQFSN

SEQ ID NO: 241 - protein, 20 aa, synthetic construct
HSQSGEENLQGSWVELHFSN

SEQ ID NO: 242 - protein, 20 aa, synthetic construct
MSQSGEENLAGSWVELHFSN

SEQ ID NO: 243 - protein, 20 aa, synthetic construct
MSQSGIENLQGSWVELHFSN

SEQ ID NO: 244 - protein, 20 aa, synthetic construct
MSQSGEENLQASWVELHFSN

SEQ ID NO: 245 - protein, 20 aa, synthetic construct
MSQSGEENLQGSWVELRFSN

SEQ ID NO: 246 - protein, 20 aa, synthetic construct
ISQSGEENLQGSWVELHFSN

SEQ ID NO: 247 - protein, 20 aa, synthetic construct
MSQSGEENLCGSWVELHFSN

SEQ ID NO: 248 - protein, 20 aa, synthetic construct
MSQSGKENLQGSWVELHFSN

SEQ ID NO: 249 - protein, 20 aa, synthetic construct
MSQSGEENLQCSWVELHFSN

SEQ ID NO: 250 - protein, 20 aa, synthetic construct
MSQSGEENLQGSWVELSFSN

SEQ ID NO: 251 - protein, 20 aa, synthetic construct
KSQSGEENLQGSWVELHFSN

SEQ ID NO: 252 - protein, 20 aa, synthetic construct
MSQSGEENLDGSWVELHFSN

SEQ ID NO: 253 - protein, 20 aa, synthetic construct
MSQSGLENLQGSWVELHFSN

SEQ ID NO: 254 - protein, 20 aa, synthetic construct
MSQSGEENLQDSWVELHFSN

SEQ ID NO: 255 - protein, 20 aa, synthetic construct
MSQSGEENLQGSWVELTFSN

SEQ ID NO: 256 - protein, 20 aa, synthetic construct
LSQSGEENLQGSWVELHFSN

SEQ ID NO: 257 - protein, 20 aa, synthetic construct
MSQSGEENLEGSWVELHFSN

SEQ ID NO: 258 - protein, 20 aa, synthetic construct
MSQSGMENLQGSWVELHFSN

SEQ ID NO: 259 - protein, 20 aa, synthetic construct
MSQSGEENLQESWVELHFSN

SEQ ID NO: 260 - protein, 20 aa, synthetic construct
MSQSGEENLQGSWVELVFSN

SEQ ID NO: 261 - protein, 20 aa, synthetic construct
NSQSGEENLQGSWVELHFSN

SEQ ID NO: 262 - protein, 20 aa, synthetic construct
MSQSGEENLFGSWVELHFSN

SEQ ID NO: 263 - protein, 20 aa, synthetic construct
MSQSGNENLQGSWVELHFSN

SEQ ID NO: 264 - protein, 20 aa, synthetic construct
MSQSGEENLQFSWVELHFSN

SEQ ID NO: 265 - protein, 20 aa, synthetic construct
MSQSGEENLQGSWVELWFSN

SEQ ID NO: 266 - protein, 20 aa, synthetic construct
PSQSGEENLQGSWVELHFSN

SEQ ID NO: 267 - protein, 20 aa, synthetic construct
MSQSGEENLGGSWVELHFSN

SEQ ID NO: 268 - protein, 20 aa, synthetic construct
MSQSGPENLQGSWVELHFSN

SEQ ID NO: 269 - protein, 20 aa, synthetic construct
MSQSGEENLQHSWVELHFSN

SEQ ID NO: 270 - protein, 20 aa, synthetic construct
MSQSGEENLQGSWVELYFSN

SEQ ID NO: 271 - protein, 20 aa, synthetic construct
QSQSGEENLQGSWVELHFSN

SEQ ID NO: 272 - protein, 20 aa, synthetic construct
MSQSGEENLHGSWVELHFSN

SEQ ID NO: 273 - protein, 20 aa, synthetic construct
MSQSGQENLQGSWVELHFSN

SEQ ID NO: 274 - protein, 20 aa, synthetic construct
MSQSGEENLQISWVELHFSN

SEQ ID NO: 275 - protein, 20 aa, synthetic construct
MSQSGEENLQGSWVEAHFSN

SEQ ID NO: 276 - protein, 20 aa, synthetic construct
RSQSGEENLQGSWVELHFSN

SEQ ID NO: 277 - protein, 20 aa, synthetic construct
MSQSGEENLIGSWVELHFSN

SEQ ID NO: 278 - protein, 20 aa, synthetic construct
MSQSGRENLQGSWVELHFSN

SEQ ID NO: 279 - protein, 20 aa, synthetic construct
MSQSGEENLQKSWVELHFSN

SEQ ID NO: 280 - protein, 20 aa, synthetic construct
MSQSGEENLQGSWVECHFSN

SEQ ID NO: 281 - protein, 20 aa, synthetic construct
SSQSGEENLQGSWVELHFSN

SEQ ID NO: 282 - protein, 20 aa, synthetic construct
MSQSGEENLKGSWVELHFSN

SEQ ID NO: 283 - protein, 20 aa, synthetic construct
MSQSGSENLQGSWVELHFSN

SEQ ID NO: 284 - protein, 20 aa, synthetic construct
MSQSGEENLQLSWVELHFSN

SEQ ID NO: 285 - protein, 20 aa, synthetic construct
MSQSGEENLQGSWVEDHFSN

SEQ ID NO: 286 - protein, 20 aa, synthetic construct
TSQSGEENLQGSWVELHFSN

SEQ ID NO: 287 - protein, 20 aa, synthetic construct
MSQSGEENLLGSWVELHFSN

SEQ ID NO: 288 - protein, 20 aa, synthetic construct
MSQSGTENLQGSWVELHFSN

SEQ ID NO: 289 - protein, 20 aa, synthetic construct
MSQSGEENLQMSWVELHFSN

SEQ ID NO: 290 - protein, 20 aa, synthetic construct
MSQSGEENLQGSWVEEHFSN

SEQ ID NO: 291 - protein, 20 aa, synthetic construct
VSQSGEENLQGSWVELHFSN

SEQ ID NO: 292 - protein, 20 aa, synthetic construct
MSQSGEENLMGSWVELHFSN

SEQ ID NO: 293 - protein, 20 aa, synthetic construct
MSQSGVENLQGSWVELHFSN

SEQ ID NO: 294 - protein, 20 aa, synthetic construct
MSQSGEENLQNSWVELHFSN

SEQ ID NO: 295 - protein, 20 aa, synthetic construct
MSQSGEENLQGSWVEFHFSN

SEQ ID NO: 296 - protein, 20 aa, synthetic construct
WSQSGEENLQGSWVELHFSN

SEQ ID NO: 297 - protein, 20 aa, synthetic construct
MSQSGEENLNGSWVELHFSN

SEQ ID NO: 298 - protein, 20 aa, synthetic construct
MSQSGWENLQGSWVELHFSN

SEQ ID NO: 299 - protein, 20 aa, synthetic construct
MSQSGEENLQPSWVELHFSN

SEQ ID NO: 300 - protein, 20 aa, synthetic construct
MSQSGEENLQGSWVEGHFSN

SEQ ID NO: 301 - protein, 20 aa, synthetic construct
YSQSGEENLQGSWVELHFSN

SEQ ID NO: 302 - protein, 20 aa, synthetic construct
MSQSGEENLPGSWVELHFSN

SEQ ID NO: 303 - protein, 20 aa, synthetic construct
MSQSGYENLQGSWVELHFSN

SEQ ID NO: 304 - protein, 20 aa, synthetic construct
MSQSGEENLQQSWVELHFSN

SEQ ID NO: 305 - protein, 20 aa, synthetic construct
MSQSGEENLQGSWVEHHFSN

SEQ ID NO: 306 - protein, 20 aa, synthetic construct
MSQSGEENLQGSWVELHFSA

SEQ ID NO: 307 - protein, 20 aa, synthetic construct
MSQSGEENLRGSWVELHFSN

SEQ ID NO: 308 - protein, 20 aa, synthetic construct
MSQSGEANLQGSWVELHFSN

SEQ ID NO: 309 - protein, 20 aa, synthetic construct
MSQSGEENLQRSWVELHFSN

SEQ ID NO: 310 - protein, 20 aa, synthetic construct
MSQSGEENLQGSWVEIHFSN

SEQ ID NO: 311 - protein, 20 aa, synthetic construct
MSQSGEENLQGSWVELHFSC

SEQ ID NO: 312 - protein, 20 aa, synthetic construct
MSQSGEENLSGSWVELHFSN

SEQ ID NO: 313 - protein, 20 aa, synthetic construct
MSQSGECNLQGSWVELHFSN

SEQ ID NO: 314 - protein, 20 aa, synthetic construct
MSQSGEENLQSSWVELHFSN

SEQ ID NO: 315 - protein, 20 aa, synthetic construct
MSQSGEENLQGSWVEKHFSN

SEQ ID NO: 316 - protein, 20 aa, synthetic construct
MSQSGEENLQGSWVELHFSD

SEQ ID NO: 317 - protein, 20 aa, synthetic construct
MSQSGEENLTGSWVELHFSN

SEQ ID NO: 318 - protein, 20 aa, synthetic construct
MSQSGEDNLQGSWVELHFSN

SEQ ID NO: 319 - protein, 20 aa, synthetic construct
MSQSGEENLQTSWVELHFSN

SEQ ID NO: 320 - protein, 20 aa, synthetic construct
MSQSGEENLQGSWVEMHFSN

SEQ ID NO: 321 - protein, 20 aa, synthetic construct
MSQSGEENLQGSWVELHFSE

SEQ ID NO: 322 - protein, 20 aa, synthetic construct
MSQSGEENLVGSWVELHFSN

SEQ ID NO: 323 - protein, 20 aa, synthetic construct
MSQSGEFNLQGSWVELHFSN

SEQ ID NO: 324 - protein, 20 aa, synthetic construct
MSQSGEENLQVSWVELHFSN

SEQ ID NO: 325 - protein, 20 aa, synthetic construct
MSQSGEENLQGSWVENHFSN

SEQ ID NO: 326 - protein, 20 aa, synthetic construct
MSQSGEENLQGSWVELHFSF

SEQ ID NO: 327 - protein, 20 aa, synthetic construct
MSQSGEENLWGSWVELHFSN

SEQ ID NO: 328 - protein, 20 aa, synthetic construct
MSQSGEGNLQGSWVELHFSN

SEQ ID NO: 329 - protein, 20 aa, synthetic construct
MSQSGEENLQWSWVELHFSN

SEQ ID NO: 330 - protein, 20 aa, synthetic construct
MSQSGEENLQGSWVEPHFSN

SEQ ID NO: 331 - protein, 20 aa, synthetic construct
MSQSGEENLQGSWVELHFSG

SEQ ID NO: 332 - protein, 20 aa, synthetic construct
MSQSGEENLYGSWVELHFSN

SEQ ID NO: 333 - protein, 20 aa, synthetic construct
MSQSGEHNLQGSWVELHFSN

SEQ ID NO: 334 - protein, 20 aa, synthetic construct
MSQSGEENLQYSWVELHFSN

SEQ ID NO: 335 - protein, 20 aa, synthetic construct
MSQSGEENLQGSWVEQHFSN

SEQ ID NO: 336 - protein, 20 aa, synthetic construct
MSQSGEENLQGSWVELHFSH

SEQ ID NO: 337 - protein, 20 aa, synthetic construct
MSASGEENLQGSWVELHFSN

SEQ ID NO: 338 - protein, 20 aa, synthetic construct
MSQSGEINLQGSWVELHFSN

SEQ ID NO: 339 - protein, 20 aa, synthetic construct
MSQSAEENLQGSWVELHFSN

SEQ ID NO: 340 - protein, 20 aa, synthetic construct
MSQSGEENLQGSWVERHFSN

SEQ ID NO: 341 - protein, 20 aa, synthetic construct
MSQSGEENLQGSWVELHFSI

SEQ ID NO: 342 - protein, 20 aa, synthetic construct
MSCSGEENLQGSWVELHFSN

SEQ ID NO: 343 - protein, 20 aa, synthetic construct
MSQSGEKNLQGSWVELHFSN

SEQ ID NO: 344 - protein, 20 aa, synthetic construct
MSQSCEENLQGSWVELHFSN

SEQ ID NO: 345 - protein, 20 aa, synthetic construct
MSQSGEENLQGSWVESHFSN

SEQ ID NO: 346 - protein, 20 aa, synthetic construct
MSQSGEENLQGSWVELHFSK

SEQ ID NO: 347 - protein, 20 aa, synthetic construct
MSDSGEENLQGSWVELHFSN

SEQ ID NO: 348 - protein, 20 aa, synthetic construct
MSQSGELNLQGSWVELHFSN

SEQ ID NO: 349 - protein, 20 aa, synthetic construct
MSQSDEENLQGSWVELHFSN

SEQ ID NO: 350 - protein, 20 aa, synthetic construct
MSQSGEENLQGSWVETHFSN

SEQ ID NO: 351 - protein, 20 aa, synthetic construct
MSQSGEENLQGSWVELHFSL

SEQ ID NO: 352 - protein, 20 aa, synthetic construct
MSESGEENLQGSWVELHFSN

SEQ ID NO: 353 - protein, 20 aa, synthetic construct
MSQSGEMNLQGSWVELHFSN

SEQ ID NO: 354 - protein, 20 aa, synthetic construct
MSQSEEENLQGSWVELHFSN

SEQ ID NO: 355 - protein, 20 aa, synthetic construct
MSQSGEENLQGSWVEVHFSN

SEQ ID NO: 356 - protein, 20 aa, synthetic construct
MSQSGEENLQGSWVELHFSM

SEQ ID NO: 357 - protein, 20 aa, synthetic construct
MSFSGEENLQGSWVELHFSN

SEQ ID NO: 358 - protein, 20 aa, synthetic construct
MSQSGENNLQGSWVELHFSN

SEQ ID NO: 359 - protein, 20 aa, synthetic construct
MSLSGEENLQGSWVELHFSN

SEQ ID NO: 360 - protein, 20 aa, synthetic construct
MSQSGEENLQGSWVELHFCN

SEQ ID NO: 361 - protein, 20 aa, synthetic construct
MLQSGEENLQGSWVELHFSN

SEQ ID NO: 362 - protein, 20 aa, synthetic construct
MSQYGEENLQGSWVELHFSN

SEQ ID NO: 363 - protein, 20 aa, synthetic construct
MSQSGEPNLQGSWVELHFSN

SEQ ID NO: 364 - protein, 20 aa, synthetic construct
MSMSGEENLQGSWVELHFSN

SEQ ID NO: 365 - protein, 20 aa, synthetic construct
MSQSGEENLQGSWVELHFDN

SEQ ID NO: 366 - protein, 20 aa, synthetic construct
MMQSGEENLQGSWVELHFSN

SEQ ID NO: 367 - protein, 20 aa, synthetic construct
MSQSGEENLQGSWAELHFSN

SEQ ID NO: 368 - protein, 20 aa, synthetic construct
MSQSGEQNLQGSWVELHFSN

SEQ ID NO: 369 - protein, 20 aa, synthetic construct
MSNSGEENLQGSWVELHFSN

SEQ ID NO: 370 - protein, 20 aa, synthetic construct
MSQSGEENLQGSWVELHFEN

SEQ ID NO: 371 - protein, 20 aa, synthetic construct
MNQSGEENLQGSWVELHFSN

SEQ ID NO: 372 - protein, 20 aa, synthetic construct
MSQSGEENLQGSWCELHFSN

SEQ ID NO: 373 - protein, 20 aa, synthetic construct
MSQSFEENLQGSWVELHFSN

SEQ ID NO: 374 - protein, 20 aa, synthetic construct
MSPSGEENLQGSWVELHFSN

SEQ ID NO: 375 - protein, 20 aa, synthetic construct
MSQSGEENLQGSWVELHFFN

SEQ ID NO: 376 - protein, 20 aa, synthetic construct
MPQSGEENLQGSWVELHFSN

SEQ ID NO: 377 - protein, 20 aa, synthetic construct
MSQSGEENLQGSWRELHFSN

SEQ ID NO: 378 - protein, 20 aa, synthetic construct
MSQSHEENLQGSWVELHFSN

SEQ ID NO: 379 - protein, 20 aa, synthetic construct
MSRSGEENLQGSWVELHFSN

SEQ ID NO: 380 - protein, 20 aa, synthetic construct
MSQSGEENLQGSWVELHFGN

SEQ ID NO: 381 - protein, 20 aa, synthetic construct
MQQSGEENLQGSWVELHFSN

SEQ ID NO: 382 - protein, 20 aa, synthetic construct
MSQSGEENLQGSWSELHFSN

SEQ ID NO: 383 - protein, 20 aa, synthetic construct
MSQSIEENLQGSWVELHFSN

SEQ ID NO: 384 - protein, 20 aa, synthetic construct
MSSSGEENLQGSWVELHFSN

SEQ ID NO: 385 - protein, 20 aa, synthetic construct
MSQSGEENLQGSWVELHFHN

SEQ ID NO: 386 - protein, 20 aa, synthetic construct
MRQSGEENLQGSWVELHFSN

SEQ ID NO: 387 - protein, 20 aa, synthetic construct
MSQSGEENLQGSWTELHFSN

SEQ ID NO: 388 - protein, 20 aa, synthetic construct
MSQSGEENLQGSWVEWHFSN

SEQ ID NO: 389 - protein, 20 aa, synthetic construct
MSVSGEENLQGSWVELHFSN

SEQ ID NO: 390 - protein, 20 aa, synthetic construct
MSQSGEENLQGSWVELHFIN

SEQ ID NO: 391 - protein, 20 aa, synthetic construct
MSQKGEENLQGSWVELHFSN

SEQ ID NO: 392 - protein, 20 aa, synthetic construct
MSQSGEENLQGSWWELHFSN

SEQ ID NO: 393 - protein, 20 aa, synthetic construct
MSQSGEENLQGSWVEYHFSN

SEQ ID NO: 394 - protein, 20 aa, synthetic construct
MSWSGEENLQGSWVELHFSN

SEQ ID NO: 395 - protein, 20 aa, synthetic construct
MSQSGEENLQGSWVELHFKN

SEQ ID NO: 396 - protein, 20 aa, synthetic construct
MSQLGEENLQGSWVELHFSN

SEQ ID NO: 397 - protein, 20 aa, synthetic construct
MSQSGEENLQGSWYELHFSN

SEQ ID NO: 398 - protein, 20 aa, synthetic construct
MSQSGEENAQGSWVELHFSN

SEQ ID NO: 399 - protein, 20 aa, synthetic construct
MSYSGEENLQGSWVELHFSN

SEQ ID NO: 400 - protein, 20 aa, synthetic construct
MSQSGEENLQGSWVELHFLN

SEQ ID NO: 401 - protein, 20 aa, synthetic construct
MSQMGEENLQGSWVELHFSN

SEQ ID NO: 402 - protein, 20 aa, synthetic construct
MSQSGEENLQGSAVELHFSN

SEQ ID NO: 403 - protein, 20 aa, synthetic construct
MSQSGEENLQGSWVELHFSP

SEQ ID NO: 404 - protein, 20 aa, synthetic construct
MSQSGEENLQGAWVELHFSN

SEQ ID NO: 405 - protein, 20 aa, synthetic construct
MDQSGEENLQGSWVELHFSN

SEQ ID NO: 406 - protein, 20 aa, synthetic construct
MSQNGEENLQGSWVELHFSN

SEQ ID NO: 407 - protein, 20 aa, synthetic construct
MSQSGEENLQGSCVELHFSN

SEQ ID NO: 408 - protein, 20 aa, synthetic construct
MSQSGEENLQGSWVELHFSQ

SEQ ID NO: 409 - protein, 20 aa, synthetic construct
MSQSGEENLQGCWVELHFSN

SEQ ID NO: 410 - protein, 20 aa, synthetic construct
MEQSGEENLQGSWVELHFSN

SEQ ID NO: 411 - protein, 20 aa, synthetic construct
MSQPGEENLQGSWVELHFSN

SEQ ID NO: 412 - protein, 20 aa, synthetic construct
MSQSGEENLQGSDVELHFSN

SEQ ID NO: 413 - protein, 20 aa, synthetic construct
MSQSGEENLQGSWVELHFSR

SEQ ID NO: 414 - protein, 20 aa, synthetic construct
MSQSGEENLQGDWVELHFSN

SEQ ID NO: 415 - protein, 20 aa, synthetic construct
MFQSGEENLQGSWVELHFSN

SEQ ID NO: 416 - protein, 20 aa, synthetic construct
MSQQGEENLQGSWVELHFSN

SEQ ID NO: 417 - protein, 20 aa, synthetic construct
MSQSGEENLQGSEVELHFSN

SEQ ID NO: 418 - protein, 20 aa, synthetic construct
MSGSGEENLQGSWVELHFSN

SEQ ID NO: 419 - protein, 20 aa, synthetic construct
MSQSGEENLQGTWVELHFSN

SEQ ID NO: 420 - protein, 20 aa, synthetic construct
MGQSGEENLQGSWVELHFSN

SEQ ID NO: 421 - protein, 20 aa, synthetic construct
MSQRGEENLQGSWVELHFSN

SEQ ID NO: 422 - protein, 20 aa, synthetic construct
MSQSGEENLQGSFVELHFSN

SEQ ID NO: 423 - protein, 20 aa, synthetic construct
MSHSGEENLQGSWVELHFSN

SEQ ID NO: 424 - protein, 20 aa, synthetic construct
MSQSGEENLQGVWVELHFSN

SEQ ID NO: 425 - protein, 20 aa, synthetic construct
MHQSGEENLQGSWVELHFSN

SEQ ID NO: 426 - protein, 20 aa, synthetic construct
MSQTGEENLQGSWVELHFSN

SEQ ID NO: 427 - protein, 20 aa, synthetic construct
MSQSGEENLQGSGVELHFSN

SEQ ID NO: 428 - protein, 20 aa, synthetic construct
MSISGEENLQGSWVELHFSN

SEQ ID NO: 429 - protein, 20 aa, synthetic construct
MSQSGEENLQGWWVELHFSN

SEQ ID NO: 430 - protein, 20 aa, synthetic construct
MIQSGEENLQGSWVELHFSN

SEQ ID NO: 431 - protein, 20 aa, synthetic construct
MSQVGEENLQGSWVELHFSN

SEQ ID NO: 432 - protein, 20 aa, synthetic construct
MSQSGEENLQGSHVELHFSN

SEQ ID NO: 433 - protein, 20 aa, synthetic construct
MSKSGEENLQGSWVELHFSN

SEQ ID NO: 434 - protein, 20 aa, synthetic construct
MSQSGEENLQGYWVELHFSN

SEQ ID NO: 435 - protein, 20 aa, synthetic construct
MKQSGEENLQGSWVELHFSN

SEQ ID NO: 436 - protein, 20 aa, synthetic construct
MSQWGEENLQGSWVELHFSN

SEQ ID NO: 437 - protein, 20 aa, synthetic construct
MSQSGEENLQGSIVELHFSN

SEQ ID NO: 438 - protein, 20 aa, synthetic construct
MSQSGEENLQGEWVELHFSN

SEQ ID NO: 439 - protein, 20 aa, synthetic construct
MSQSGEENLQGSWVELHFMN

SEQ ID NO: 440 - protein, 20 aa, synthetic construct
MTQSGEENLQGSWVELHFSN

SEQ ID NO: 441 - protein, 20 aa, synthetic construct
MSQSGEENLQGSWDELHFSN

SEQ ID NO: 442 - protein, 20 aa, synthetic construct
MSQSGEENLQGSKVELHFSN

SEQ ID NO: 443 - protein, 20 aa, synthetic construct
MSQSGEENLQGFWVELHFSN

SEQ ID NO: 444 - protein, 20 aa, synthetic construct
MSQSGEENLQGSWVELHFNN

SEQ ID NO: 445 - protein, 20 aa, synthetic construct
MVQSGEENLQGSWVELHFSN

SEQ ID NO: 446 - protein, 20 aa, synthetic construct
MSQSGEENLQGSWEELHFSN

SEQ ID NO: 447 - protein, 20 aa, synthetic construct
MSQSGEENLQGSLVELHFSN

SEQ ID NO: 448 - protein, 20 aa, synthetic construct
MSQSGEENLQGGWVELHFSN

SEQ ID NO: 449 - protein, 20 aa, synthetic construct
MSQSGEENLQGSWVELHFPN

SEQ ID NO: 450 - protein, 20 aa, synthetic construct
MWQSGEENLQGSWVELHFSN

SEQ ID NO: 451 - protein, 20 aa, synthetic construct
MSQSGEENLQGSWFELHFSN

SEQ ID NO: 452 - protein, 20 aa, synthetic construct
MSQSGEENLQGSMVELHFSN

SEQ ID NO: 453 - protein, 20 aa, synthetic construct
MSQSGEENLQGHWVELHFSN

SEQ ID NO: 454 - protein, 20 aa, synthetic construct
MSQSGEENLQGSWVELHFQN

SEQ ID NO: 455 - protein, 20 aa, synthetic construct
MYQSGEENLQGSWVELHFSN

SEQ ID NO: 456 - protein, 20 aa, synthetic construct
MSQSGEENLQGSWGELHFSN

SEQ ID NO: 457 - protein, 20 aa, synthetic construct
MSQSGEENLQGSNVELHFSN

SEQ ID NO: 458 - protein, 20 aa, synthetic construct
MSQSGEENLQGIWVELHFSN

SEQ ID NO: 459 - protein, 20 aa, synthetic construct
MSQSGEENLQGSWVELHFRN

SEQ ID NO: 460 - protein, 20 aa, synthetic construct
MSQAGEENLQGSWVELHFSN

SEQ ID NO: 461 - protein, 20 aa, synthetic construct
MSQSGEENLQGSWHELHFSN

SEQ ID NO: 462 - protein, 20 aa, synthetic construct
MSQSGEENLQGSPVELHFSN

SEQ ID NO: 463 - protein, 20 aa, synthetic construct
MSQSGEENLQGKWVELHFSN

SEQ ID NO: 464 - protein, 20 aa, synthetic construct
MSQSGEENLQGSWVELHFTN

SEQ ID NO: 465 - protein, 20 aa, synthetic construct
MSQCGEENLQGSWVELHFSN

SEQ ID NO: 466 - protein, 20 aa, synthetic construct
MSQSGEENLQGSWIELHFSN

SEQ ID NO: 467 - protein, 20 aa, synthetic construct
MSQSGEENLQGSQVELHFSN

SEQ ID NO: 468 - protein, 20 aa, synthetic construct
MSQSGEENLQGLWVELHFSN

SEQ ID NO: 469 - protein, 20 aa, synthetic construct
MSQSGEENLQGSWVELHFVN

SEQ ID NO: 470 - protein, 20 aa, synthetic construct
MSQDGEENLQGSWVELHFSN

SEQ ID NO: 471 - protein, 20 aa, synthetic construct
MSQSGEENLQGSWKELHFSN

SEQ ID NO: 472 - protein, 20 aa, synthetic construct
MSQSGEENLQGSRVELHFSN

SEQ ID NO: 473 - protein, 20 aa, synthetic construct
MSQSGEENLQGMWVELHFSN

SEQ ID NO: 474 - protein, 20 aa, synthetic construct
MSQSGEENLQGSWVELHFWN

SEQ ID NO: 475 - protein, 20 aa, synthetic construct
MSQEGEENLQGSWVELHFSN

SEQ ID NO: 476 - protein, 20 aa, synthetic construct
MSQSGEENLQGSWLELHFSN

SEQ ID NO: 477 - protein, 20 aa, synthetic construct
MSQSGEENLQGSSVELHFSN

SEQ ID NO: 478 - protein, 20 aa, synthetic construct
MSQSGEENLQGNWVELHFSN

SEQ ID NO: 479 - protein, 20 aa, synthetic construct
MSQSGEENLQGSWVELHFYN

SEQ ID NO: 480 - protein, 20 aa, synthetic construct
MSQFGEENLQGSWVELHFSN

SEQ ID NO: 481 - protein, 20 aa, synthetic construct
MSQSGEENLQGSWMELHFSN

SEQ ID NO: 482 - protein, 20 aa, synthetic construct
MSQSGEENLQGPWVELHFSN

SEQ ID NO: 483 - protein, 20 aa, synthetic construct
MSQSGEENLQGSWVELHFAN

SEQ ID NO: 484 - protein, 20 aa, synthetic construct
MSQGGEENLQGSWVELHFSN

SEQ ID NO: 485 - protein, 20 aa, synthetic construct
MSQSGEENLQGSWNELHFSN

SEQ ID NO: 486 - protein, 20 aa, synthetic construct
MSQSGEENLQGQWVELHFSN

SEQ ID NO: 487 - protein, 20 aa, synthetic construct
MAQSGEENLQGSWVELHFSN

SEQ ID NO: 488 - protein, 20 aa, synthetic construct
MSQHGEENLQGSWVELHFSN

SEQ ID NO: 489 - protein, 20 aa, synthetic construct
MSQSGEENLQGSWPELHFSN

SEQ ID NO: 490 - protein, 20 aa, synthetic construct
MSQSGEENLQGRWVELHFSN

SEQ ID NO: 491 - protein, 20 aa, synthetic construct
MCQSGEENLQGSWVELHFSN

SEQ ID NO: 492 - protein, 20 aa, synthetic construct
MSQIGEENLQGSWVELHFSN

SEQ ID NO: 493 - protein, 20 aa, synthetic construct
MSQSGEENLQGSWQELHFSN

SEQ ID NO: 494 - protein, 20 aa, synthetic construct
SEQIMKTGAFLLQGFIQDRA

(19) United States
(12) Patent Application Publication
(10) Pub. No.: US 2025/0270268 A1
(43) Pub. Date: Aug. 28, 2025

(54) AMELIORATION AND TREATMENT OF INFARCTION DAMAGE

(71) Applicant: Bimyo GmbH, Monheim (DE)

(72) Inventors: Tienush RASSAF, Essen (DE); Ulrike HENDGEN-COTTA, Dinslaken (DE)

(21) Appl. No.: 19/199,756

(22) Filed: May 6, 2025

Related U.S. Application Data
(63) Continuation of application No. 18/413,753, filed on Jan. 16, 2024, now Pat. No. 12,344,644.
Application No. 18/413,753 is a continuation of application No. PCT/EP2022/080979, filed on Nov. 7, 2022.
(60) Provisional application No. 63/276,028, filed on Nov. 5, 2021.

(51) Int. Cl. C07K 14/47; A61K 38/17; A61P 9/10; C07K 19/00

(52) U.S. Cl. CPC C07K 14/4747; A61K 38/1761; A61P 9/10; C07K 19/00

(57) ABSTRACT

Infarction damage is ameliorated or treated through the administration of a mitochondrial degradation inhibitor, such as one that acts by inhibiting translocation of one or more molecules across a mitochondrial membrane, for example BAX/BNIP3 complexes involved in mitochondrial degradation. By preventing mitochondrial degradation, one allows more efficient oxygenation of an infarcted region, allowing for a greater degree of cell survival and a more successful recovery.

### CROSS-REFERENCE TO RELATED APPLICATIONS

This document claims the benefit of priority to U.S. Provisional Application Ser. No. 63/276,028, filed Nov. 5, 2021, the contents of which are hereby incorporated by reference in their entirety, this document is a continuation of PCT Application Number PCT/EP2022/080979, filed Nov. 7, 2022, the contents of which are hereby incorporated by reference in their entirety, and this document is a continuation of U.S. application Ser. No. 18/413,753, filed Jan. 16, 2024, the contents of which are hereby incorporated by reference in their entirety.

### SEQUENCE LISTING

The instant application contains a Sequence Listing which has been submitted electronically in XML file format and is hereby incorporated by reference in its entirety. Said XML copy, created on Jan. 12, 2024, is named HNM_001US2_SL.xml and is 435,340 bytes in size.

### BACKGROUND

Side effects are an ongoing concern in the broad field of patient intervention. A recurrent concern is that an intervention may inadvertently or secondarily harm a patient by triggering secondary responses in a patient that are contradictory to the intended effect.

These secondary responses are particularly problematic if they result in cell signaling that may lead to initiation of cell death. A number of interventions independently pose this risk. These range from chemotherapy intended to target proliferating cancer cells, incidental trauma associated with surgery, peripheral nerve injury, compression of parts of the body that causes muscle breakdown, burn injury, and wasting illness such as or associated with, for example, cancer, pulmonary, neurological and hematological diseases or re-oxygenation of the heart muscle or brain tissue of an individual suffering from a circulatory disruption such as a heart attack or stroke, among others.

A common theme among these interventions is that they risk initiating any number of programmed cell death pathways in cells of the individual intended to be treated. These cell death pathways are often mitochondrially triggered or comprise a mitochondrial component, such as aberrations in calcium ion or reactive oxygen species accumulation, changes in inner membrane polarization, pore formation in the outer membrane, release of cytochrome c, mitochondrial swelling or even lysis.

Treating acute circulatory disruptions, for example, presents a number of challenges. During circulatory disruption, cells, cell populations, tissue or organs (collectively, impacted areas') are starved of oxygen. This has a great negative impact upon cellular respiration at the mitochondria. However, rapid reintroduction of oxygen into an impacted area triggers a number of responses in the mitochondria, such as disintegration of mitochondrial membranes, which counterintuitively leads to initiation of cell death pathway signaling that manifests cardiac dysfunction.

Similarly, chemotherapy targeted towards selective killing of malignancy-driving cells or of tumors generally is similarly problematic. Either the targeted cells themselves or other cells in the patient may respond to the treatment by initiating a cell death pathway that involves changes in mitochondrial status, such as mitochondrial disintegration. This may often signal a similar response in related cells, which may exacerbate the detrimental side effects of a chemotherapy or even limit its efficacy by preventing the use of chemotherapeutic at a level sufficient to target the malignancy or harmful cells.

Disintegration of mitochondrial membranes is implicated as a final cell death pathway (Karch and Molkentin, 2015; Murphy et al., 2016; Hendgen-Cotta et al. 2019). Loss of the impermeability of the mitochondrial inner membrane (MIM) to small solutes causes depolarization (Δψm) with cessation of ATP synthesis, mitochondrial swelling followed by membrane rupture and necrotic cell death (Bernardi et al., 2015; Kwong and Molkentin, 2015; Galluzzi et al., 2018). Pore formation in the outer mitochondrial membrane (MOMP) facilitates cytosolic release of apoptogens like cytochrome c, apoptosis inducing factor (AIF) and endonuclease G leading to apoptosis (Ong and Gustafsson, 2012). Accordingly, damaged mitochondria contribute to various pathologies including neurological and cardiovascular disorders (Hendgen-Cotta et al., 2008; Kloner et al., 2017; Nunnari and Suomalainen, 2012; Fuchs and Steller, 2011). Prior research on reducing mitochondrial-driven cell death in humans has largely focused on mitochondrial proteins or phospholipids, e.g., cyclophilin D and cardiolipin. However, this requires a direct interaction of therapeutic agents with mitochondria (Atar et al., 2015; Cung et al., 2015; Gibson et al., 2016; Schaller et al., 2010). Despite generally promising experimental evidence, such agents have failed to reduce cell death (Atar et al., 2015; Cung et al., 2015; Gibson et al., 2016; Hausenloy et al., 2017; Schaller et al., 2010). Deathly mitochondrial pathways are proposed to be, inter alia, regulated by BCL-2 family members (Galluzzi et al., 2018), with the functional importance of BAX being confirmed as the paramount effector (Garner et al., 2019; Kalkavan and Green, 2018; Karch and Molkentin, 2015; Wei et al., 2001, Whelan et al., 2012). The precise molecular activator and the mechanisms governing BAX translocation to mitochondria, its activation, mitochondrial interaction and pore formation in vivo are largely unknown (Luna-Vargas and Chipuk, 2016).

Evidence from in vitro and ex vivo experiments suggests a pathogenic role for the BH3-only protein BNIP3, being primarily localized to mitochondria, in mitochondrial perturbation by homodimerization (Diwan et al., 2007; Gustafsson, 2011; Hamacher-Brady et al., 2007; Karch and Molkentin, 2015; Kubasiak et al., 2002; Kubli et al., 2007, Regula et al., 2002; Vande Velde et al., 2000).

Recent findings that BNIP3 is able to interact with recombinant and mitochondrial BAX in vitro, whose binding seems to be important for their insertion into the MOM (Hendgen-Cotta et al., 2017), suggest the possibility of BNIP3 as a regulator of BAX. That may signify BNIP3/BAX an important potential cytosolic therapeutic target for mitochondria and cell protection. Elucidating the BNIP3 activity on cell death remains challenging because the structural basis of BNIP3 function is incomplete. Accordingly, the precise mechanisms of the detrimental functionality of BNIP3 on BAX and mitochondrial-driven cell death and to develop a therapy option was investigated, with the goal of understanding mitochondrial signaling as well as understanding approaches to modulating the impact of treatments on mitochondrial-induced cell death signaling to prevent mitochondrial damage with down-stream signaling that leads to both cell dysfunction and cell death and may manifest cardiac dysfunction, cardiotoxicity and cardiac wasting.

### SUMMARY

Disclosed herein are methods, compositions, compositions for use and treatment regimens for ameliorating a side effect of an intervention. Some such are methods, compositions, compositions for use and treatment regimens comprise administering a mitochondrial protecting or stabilizing agent such as a mitochondrial interaction and import inhibitor. Without being bound by theory, such a treatment may decrease cell death signaling arising from destabilized mitochondria, thereby reducing cell death signaling that may otherwise accompany an intervention. Through practice of the disclosure herein, one is able to reduce the harm resulting from the methods, compositions, compositions for use and treatment regimens of an intervention.

A number of exemplary intervention events are consistent with the disclosure herein. Some intervention events comprise reoxygenation in response to a circulatory disruption so as to ameliorate infarction damage or risk of infarction damage, such as may occur pursuant to reoxygenation of oxygen starved tissue, such as may occur through application of a stent or other circulatory restorative procedure.

Additional intervention events comprise administration of a radiotherapeutic or a chemotherapeutic, such as a chemotherapeutic that targets a cancerous cell, cell population, or tumor. Some such chemotherapeutics cause off-target or unintended cell death, such as cardiomyocyte death or dysfunction, which may lead to e.g. cardiac dysfunction or cardiac wasting (with or without chemotherapy or cancer therapy). Exemplary chemotherapeutics used to target cancer cells or tumors comprise, for example, a compound selected from the list consisting of an Alkylating agent, such as altretamine, busulfan, carboplatin, carmustine, cisplatin, cyclophosphamide, dacarbazine, ifosfamide, lomustine, melphalan, temozolomide, trabectedin; Antimetabolite, such as 5-fluorouracil, 6-mercaptopurine, azacitidine, capecitabine, clofarabine, cytarabine, floxuridine, fludarabine, gemcitabine, methotrexate, pemetrexed, pentostatin, pralatrexate, trifluridine and tipiracil; a Plant alkyloid, such as, vincristine, vinblastine, vinorelbine, paclitaxel, docetaxel, etoposide, teniposide, irinotecan, topotecan; antibodies, such as HER2 inhibitors e.g. trastuzumab, pertuzumab, margetuximab, immune checkpoint inhibitors e.g. nivolumab (anti-PD-1), avelumab (anti-PD-L1), ipilimumab (anti-CTLA-4), relatlimab (anti-LAG-3); an anti-tumor agent, such as, daunorubicin, doxorubicin, doxorubicin liposomal, epirubicin, idarubicin, and valrubicin. Exemplary chemotherapeutics include anthracyclines, such as daunorubicin, doxorubicin, doxorubicin liposomal, epirubicin, idarubicin, and valrubicin, for example doxorubicin.

Some mitochondrial stabilization factors, such as mitochondrial interaction and import inhibitors consistent with the disclosure herein inhibit or block BNIP3 activity. Mitochondrial indications beneficially impacted by inhibitors disclosed herein variously comprise at least one indication selected from the list comprising DOX cell death induction, mitochondrial membrane potential destabilization, mitochondrial pore opening prevention, mitochondrial calcium ion overload prevention, mitochondrial ROS or accumulation prevention. Administration of a mitochondrial stabilizer such as an interaction and import inhibitor may in various embodiments attenuate cardiomyocyte dysfunction, death or cardiotoxicity, inflammatory response, cardiac dysfunction or cardiac wasting, preserve cardiac function, increase mitochondrial fitness, preserve autophagic flux, or otherwise stabilize the mitochondrion so as to inhibit mitochondrion-mediated cell dysfunction and death signaling, and inflammatory response.

Compositions consistent with the disclosure herein may be contacted to an individual through a broad range of routes, such as ingestion, transdermal administration, or injection, among others.

Exemplary mitochondrial protection and/or stabilizing agents include mitochondrial interaction and import inhibitors such as BNIP3 fragments as disclosed herein or BAX fragments and other agents in the art.

Consistent with the above and elsewhere in the present disclosure, also disclosed herein are compositions for use in ameliorating a negative impact of a treatment. Some such compositions comprise oxygen or a chemotherapeutic, for example, and a mitochondrial protecting and/or stabilizing agent such as a mitochondrial import inhibitor.

Some such compositions and methods disclosed herein further comprise identifying a patient to which a mitochondrial protecting and/or stabilizing agent such as a mitochondrial interaction and import inhibitor is to be co-administered. A patient may be identified, most directly, as suffering from a circulatory disruption such as a heart attack or stroke. Such a patient may be subjected to a treatment to restore circulation and oxygen exposure to one or more tissues, and may be co-administered a mitochondrial protecting and/or stabilizing agent such as a mitochondrial interaction and import inhibitor pursuant to addressing the circulatory interruption. Such a treatment may comprise administration of a pharmaceutical, or subjecting the patient to a circulatory deblocking process such as application of a stent, or both.

A patient may further be identified by being a candidate for a cancer therapy such as chemotherapy or radiotherapy, for example, or may be an individual scheduled or expecting a surgical intervention, or a patient undergoing or having undergone such an intervention, such as an intervention that may result in patient bruising or blood clot formation. Additional patient identification criteria include status as a human patient having an elevated or substantial chronic infarction risk, such as a risk associated with a particular condition. Examples include, among others, Diabetes, Hypertension, Hyperlipidemia, Obesity, Genetic disorders, Stress, Smoking or smoking related lung or other tissue harm, heart disease, stroke, burn injury, crush injuries, peripheral nerve injuries, cardiac wasting, cachexia, sarcopenia and muscle wasting in e.g. cancer, pulmonary, neurological, viral, hematological, renal and hepatic disease or (auto)Inflammatory diseases. Additional patient identification criteria are contemplated and consistent with the disclosure herein.

Consistent with the above, disclosed herein are compositions and methods for the treatment and amelioration of intervention related cell death such as infarction damage, for example as occurs upon disruption of oxygenation of a cell population in a mammal. Through practice of the disclosure herein, one is able to reduce the harm resulting from infarction, through administration subsequent to, concurrent with, or prior to an infarction event or infarction intervention, of a medicament to regulate mitochondrial activity or viability, such as a composition comprising a mitochondrial interaction inhibitor or a mitochondrial translocation inhibitor, or other mitochondrial integrity-preserving agent.

A number of compositions are consistent with the disclosure herein. Exemplary compositions comprise a regulator of mitochondrial activity or viability. Some such regulators act by inhibiting mitochondrial membrane potential degradation, inhibiting cytochrome c release, or otherwise inhibiting mitochondrial degradation, such as mitochondrial degradation involved in or signaling cellular apoptosis, necrosis or other cell death signaling pathway. Some such regulators stabilize mitochondria, maintain mitochondria homeostasis, or inhibit mitochondrial interaction, or mitochondrial import or signaling.

Some such compositions effect this inhibition by blocking signaling to the mitochondria, for example, by inhibiting some or all interactions with or translocation across at least one mitochondrial membrane, or by inhibiting both interactions with and translocation across mitochondrial membranes. In some cases, the block is general, while in alternate cases a particular molecule, complex or signaling moiety is prevented from translocation across the membrane. For example, some compositions act by inhibiting protein complexes, such as BAX/BNIP3 complexes from protein-protein interactions, insertion into the membrane or translocation across the membrane.

Some such compositions effect this inhibition by blocking signaling to the mitochondria, for example, by inhibiting formation or configurational changes in signaling or catalytic molecule complexes in the cytoplasm. In some cases, the block is general, while in alternate cases a particular molecule, complex or signaling moiety is prevented from forming or undergoing a conformational change. For example, some compositions act by inhibiting protein complexes, such as BAX/BNIP3 complexes from forming or from undergoing conformational changes, such as changes pursuant to or prerequisite for translocation across a membrane.

Exemplary compositions inhibit proteins or protein complexes through a number of mechanisms, for example by presenting a portion of one of the protein constituents, so as to interfere with a binding partner's ability to interact with a corresponding region of the native protein of which the inhibitor comprises a portion. The portion of the protein constituent may be wild-type or unaltered. Alternately, some compositions comprise a protein or polypeptide or peptide having a region that exhibits some identity to one of the protein constituents of a complex, but that has been modified at one or more regions, such as one or more amino acid residue positions, such as to improve stability, binding, complex inhibition, or other activity.

Some such compositions inhibit or influence BAX/BNIP3 complexes so as to impact mitochondrial interaction and translocation and comprise a segment of a complex constituent. Exemplary segments include BNIP3 segments, such as N-terminal segments of BNIP3, for example regions drawn from MSQSGEENLQGSWVELHFSN (SEQ ID NO: 1) of BNIP3, in particular WVELHFSN (SEQ ID NO: 2) of BNIP3. Some such composition present unmodified segments of BNIP3, while alternate compositions present modified BNIP3 segments, such as polypeptides or peptides comprising the sequence WVELHFFN (SEQ ID NO: 3), sometimes referred to as a PepB sequence herein.

Accordingly, disclosed herein are compositions and methods relating to reduction of infarction harm. Some such methods relate to ameliorating infarction or infarction damage to a subject at risk of infarction or an infarction damage risk event. Alternately or in combination, some such methods relate to ameliorating infarction damage to a subject suffering from or having experienced an infarction event such as an acute infarction event.

Similarly, disclosed herein are compositions and methods relating to reduction of harm arising from chemotherapy or radiotherapy. Some such methods relate to ameliorating secondary chemotherapy effects to a subject undergoing chemotherapy or radiotherapy, wherein the subject is at risk of infarction or an infarction damage risk event. Alternately or in combination, some such methods relate to ameliorating infarction damage to a subject suffering from or having experienced an infarction event such as an acute infarction event.

Said methods variously comprise administering a mitochondrial membrane interaction and translocation inhibitor, or other mitochondrial integrity-preserving agent. Some such mitochondrial membrane interaction and translocation inhibitors inhibit BAX translocation into or through a mitochondrial membrane. Some such mitochondrial membrane interaction and translocation inhibitors inhibit BNIP3 interaction with, translocation into or through a mitochondrial membrane, or both BAX and BNIP3 interaction with, translocation into or through a mitochondrial membrane.

The infarction damage risk event variously comprises a heart attack, stroke, kidney failure, hepatic failure, organ transplantation, chemotherapy or other cancer treatment, surgery, burn injury, crush injuries, peripheral nerve injury, cardiac wasting, cachexia, sarcopenia and wasting illness such as, for example, pulmonary, neurological viral, and hematological diseases or any disorder associated with or arising from a failure to oxygenate an organ, tissue or cell population, in particular an acute circulation failure, or any disorder associated with disruption on mitochondrial activity, stability or homeostasis, such as may result in cell dysfunction and death signaling.

Interaction and translocation inhibitors, or other mitochondrial integrity-preserving agents, are often administered intravenously or via catheter, such as at an injection or introduction site proximal to a site of likely infarction damage, either through continuous administration or as a single bolus or plurality of bolus dosages. Alternate routes of administration comprise injection, via catheter transdermal administration or ingestion.

The interaction inhibitor, translocation inhibitor, or other mitochondrial integrity-preserving agent, is administered prior to an infarction damage risk event, during or even subsequent to or over a certain period of time post an infarction damage risk event, such as surgery organ transplant, bruising or other harm.

In particular, in some cases the interaction and translocation inhibitor or other mitochondrial integrity-preserving agent is administered prior to or concurrent with an acute event such as a surgery, such as a surgery for which an infarction risk is associated. Similarly, the interaction and translocation inhibitor is in some cases administered to a patient having suffered an acute event such as a trauma which is associated with a higher subsequent risk of an infarction event. Alternately, as above, in some cases the interaction and translocation inhibitor is administered subsequent to an infarction event such as an acute infarction event. In some cases the interaction and translocation inhibitor or other mitochondrial integrity-preserving agent is administered prior to or concurrent with a cancer therapy intervention such as a chemotherapy treatment regimen. The interaction and translocation inhibitor administered subsequent to an infarction event such as an acute infarction event is optionally administered prior to, concurrent with or in addition over a certain period of time post reoxygenation of an infarcted tissue or cell population.

Interaction and translocation inhibitors are in these cases variously administered in carriers that lack available oxygen molecules, or possess oxygen molecules at a concentration insufficient to lead to reoxygenation. Alternatively, in some cases interaction and translocation inhibitors are administered concurrently with or in a carrier comprising oxygen molecules, such as at a concentration sufficient to effect reoxygenation of infarcted tissues or cell populations, or at a level consistent with preparing infarcted tissues or cell populations for subsequent oxygenation.

The interaction and translocation inhibitor or other mitochondrial integrity-preserving agent is variously administered continuously, through multiple regular or irregular doses or in a single dose.

Alternately, some methods herein relate to treatment of chronic infarction risk conditions. In these cases, the interaction and translocation inhibitor or other medicament is administered alone or as part of a treatment regimen administered to a mammal such as a human patient having an elevated or substantial chronic infarction risk, such as a risk associated with a particular condition. Examples include, among others, Diabetes, Hypertension, Hyperlipidemia, renal failure, hepatic failure, Obesity, Genetic disorders, Stress, Smoking or smoking related lung or other tissue harm, heart disease, stroke, burn injury, crush injuries, peripheral nerve injuries, cachexia, sarcopenia and muscle wasting, wasting illness such as pulmonary, neurological, viral, hematological disease, cardiac wasting or (auto)Inflammatory diseases. Often, these embodiments of the disclosure comprise repeated or regular administration of an inhibitor, such as through injection, in a manner somewhat analogous to regular insulin administration for blood sugar level management or via a port.

Some preferred interaction and translocation inhibitors or other mitochondrial integrity-preserving agents comprise a chimeric protein, polypeptide or peptide, such as segment having at least 75% identity to at least 8 consecutive residues of BNIP3, such as a segment wherein the at least 8 consecutive residues of BNIP3 comprise a phenylalanine residue at a 7^{th} of the at least 8 consecutive residues of BNIP3. In some cases, these 8 consecutive residues comprise residues having at least 75% identity to residues corresponding to residues 13-20 of BNIP3. The 8 consecutive residues in some cases comprise residues having at least 87.5% identity to residues corresponding to residues 13-20 of BNIP3.

Larger protein complex fragments are also contemplated as constituents of compositions to be used in the compositions and methods disclosed herein. For example, again looking to the BAX/BNIP3 complex targeting approach, compositions variously comprise BAX or a BNIP a segment having at least 87.5% identity or at least 75% identity to no more than 50 residues of a complex constituent such as BNIP3. In representative embodiments, the BNIP3 fragment does not comprise a BH3 motif, and or does not comprise a PESTQ motif (SEQ ID NO: 4), and or does not comprise a transmembrane domain or C-terminal BNIP3 domain. Often, the protein segment comprises a BAX binding motif, a BNIP3 binding motif or a segment that binds at least one of BAX and BNIP3.

Protein or peptide segments for use in methods herein often comprise constituent segments from multiple sources; that is, protein or peptide segments as contemplated for use herein are often part of chimeric proteins, for example in combination with a cellular import signal or cellular localization signal, such as the HIV TAT cellular import signal motif.

Methods contemplated herein in some cases comprise inhibiting mitochondrial interaction and translocation, for example using protein segments or peptide segments discussed above or other compositions consistent with the disclosure herein to inhibit mitochondrial translocation, exhibit efficacy in reducing damage from subsequent, concurrent or prior infarction events. In various cases, herein the translocation inhibitor or other mitochondrial integrity-preserving agent reduces damage from a subsequent infarction event by at least 10%, at least 20%, at least 30%, at least 40%, at least 50% or at least 75%. Similarly, in various cases herein the interaction and translocation inhibitor reduces recovery time from a subsequent infarction event by at least 10%, at least 20%, at least 30%, at least 40%, at least 50% or at least 75%. Various methods herein reduce BAX mitochondrial concentration by at least 30% or for example by at least 10%, at least 20%, at least 30%, at least 40%, at least 50% or at least 75% relative to an untreated reference relative to an untreated reference. Various methods herein reduce BNIP mitochondrial concentration by at least 30% or for example by at least 10%, at least 20%, at least 30%, at least 40%, at least 50% or at least 75% relative to an untreated reference relative to an untreated reference. Various methods herein reduce mitochondrial swelling by at least 30% or for example by at least 10%, at least 20%, at least 30%, at least 40%, at least 50% or at least 75% relative to an untreated reference relative to an untreated reference. Various methods herein reduce BAX active concentration by at least 30% or for example by at least 10%, at least 20%, at least 30%, at least 40%, at least 50% or at least 75% relative to an untreated reference relative to an untreated reference. Various methods herein reduce cytochrome c release by at least 30% or for example by at least 10%, at least 20%, at least 30%, at least 40%, at least 50% or at least 75% relative to an untreated reference relative to an untreated reference. Various methods herein improve Inf/AAR by at least 50% or for example by at least 10%, at least 20%, at least 30%, at least 40%, at least 50%, at least 60%, at least 70%, at least 80% or at least 90% relative to an untreated reference relative to an untreated reference. Various methods herein reduce caspase activity by at least 30% or for example by at least 10%, at least 20%, at least 30%, at least 40%, at least 50% or at least 75% relative to an untreated reference relative to an untreated reference. Various methods herein reduce membrane depolarization by at least 30% or for example by at least 10%, at least 20%, at least 30%, at least 40%, at least 50% or at least 75% relative to an untreated reference.

Some methods disclose herein further comprise monitoring infarction damage recovery, monitoring mitochondrial status, or monitoring both infarction damage recovery and monitoring mitochondrial status, such that often one is able to determine an impact of treatment upon a mammal such as a human patient's recovery.

Also disclosed herein are compositions and methods for the identification of a molecule suitable for amelioration, reduction or treatment of infarction or infarction associated cell or tissue damage. Some such methods relate to binding of BAX/BNIP3 oligomeric complexes as indicators of a role for the binding molecule in the inhibition of BAX/BNIP3 mediated necrosis and or apoptosis.

Some such methods for the identification of a molecule suitable for amelioration, reduction or treatment of infarction or infarction associated cell or tissue damage comprise assaying for binding of the molecule to BAX/BNIP3 oligomeric complexes, in particular, wherein binding of the molecule to the BAX/BNIP3 oligomeric complex indicates efficacy in infarction amelioration. Assaying for binding variously comprises assaying for colocalization of a molecule and BAX/BNIP3 oligomeric complexes, for example as determined by fluorescence microscopy. Alternately or in combination, said assaying comprises assaying for comigration of the molecule and BAX/BNIP3 oligomeric complexes, often wherein said comigration is performed under conditions to maintain oligomeric BAX/BNIP3 oligomeric complex integrity, such as nondenaturing conditions. Comigration is determined, for example using any of gel electrophoresis, SDS-PAGE or Western Blot analysis. Assaying for binding may additionally or alternately comprise assaying for coprecipitation of the molecule and BAX/BNIP3 oligomeric complexes, for example using co-immunoprecipitation. Any of the assays above are performed on cells or cell extracts, for example cells or cell extracts subjected to oxygen starvation, and are in some cases performed shortly after oxygen starvation, such as within 10 minutes of oxygen starvation, or alternately no more than any of 1, 2, 3, 4, 5, 6, 7, 8, 9, 10, 11, 12, 13, 14, 15, 20, 25, 30 or more than 30 minutes after oxygen starvation. Any of these assays may be performed at baseline, or after ischemia at 5 minutes, 10 minutes, 30 minutes, 1 hour, 2 hours, 4 hours, 8 hours 16 hours 24 hours or longer after reperfusion.

Some such methods for the identification of a molecule suitable for amelioration, reduction or treatment of infarction or infarction associated cell or tissue damage comprise assaying for BAX/BNIP3 complex localization upon contacting to the molecule. Said contacting may occur, for example, in a cell subjected to oxygen starvation or an extract of a cell subjected to oxygen starvation, in particular an extract that comprises functional mitochondrial membranes or functional mitochondria. In some such assays, retention of BAX/BNIP3 outside of a mitochondrion of the cell indicates efficacy in infarction amelioration. Similarly, inhibition of BAX/BNIP3 translocation into a mitochondrion of the cell in some cases indicates efficacy in infarction amelioration. Inhibition of BAX translocation into a mitochondrion of the cell in some cases indicates efficacy in infarction amelioration. In some cases inhibition of BNIP3 translocation into a mitochondrion of the cell indicates efficacy in infarction amelioration. Assaying for BAX, BNIP3 or BAX/BNIP3 localization may comprise fluorescence assays, such as may be effected using fluorescently labeled antibodies or fluorescent-protein or epitope labeled BAX, BNIP3 or BAX and BNIP3 proteins. Alternately, or in combination, molecule localization may be determined using immunofluorescence or detection or a radiolabel and overlaying such signal against an image of the cell or mitochondrion in question. Alternately or in combination, complex localization comprises one or more of assaying for mitochondrial integrity, assaying for mitochondrial swelling, assaying for cytochrome c release, or assaying for caspase-3 activity. Any of the assays above are performed on cells or cell extracts, for example cells or cell extracts subjected to oxygen starvation, and are in some cases performed shortly after oxygen starvation, such as within 10 minutes of oxygen starvation, or alternately no more than any of 1, 2, 3, 4, 5, 6, 7, 8, 9, 10, 11, 12, 13, 14, 15, 20, 25, 30 or more than 30 minutes after oxygen starvation. Any of these assays may be performed at baseline, or after ischemia at 5 minutes, 10 minutes, 30 minutes, 1 hour, 2 hours, 4 hours, 8 hours 16 hours 24 hours or longer after reperfusion.

### INCORPORATION BY REFERENCE

All publications, patents, and patent applications mentioned in this specification are herein incorporated by reference to the same extent as if each individual publication, patent, or patent application was specifically and individually indicated to be incorporated by reference. In particular, WO 02/02743, published Jan. 10, 2002 is hereby incorporated by reference in its entirety. Similarly, WO 2020/229632, published Nov. 19, 2020 is hereby incorporated by reference in its entirety.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

1A. BNIP3 oligomeric expression.

FIG. 1B. BNIP3 oligomeric expression.

FIG. 1C. Schematic of assay progression.

FIG. 1D. TAT-BNIP3 restores wild-type infarction ratios.

FIG. 1E. delta-TM administration returns Troponin I toward sham treatment levels.

FIG. 1F. delta-TM reduces or delays ApopTag positive events relative to vehicle treated infarction tissue.

FIG. 1G. delta-TM reduces mitochondrial depolarization proportion.

FIG. 1H. delta-TM reduces mitochondrial swelling to sham treatment levels, as indicated by increased OD540 values.

FIG. 11. Images indicating that delta-TM reduces mitochondrial swelling to sham treatment levels.

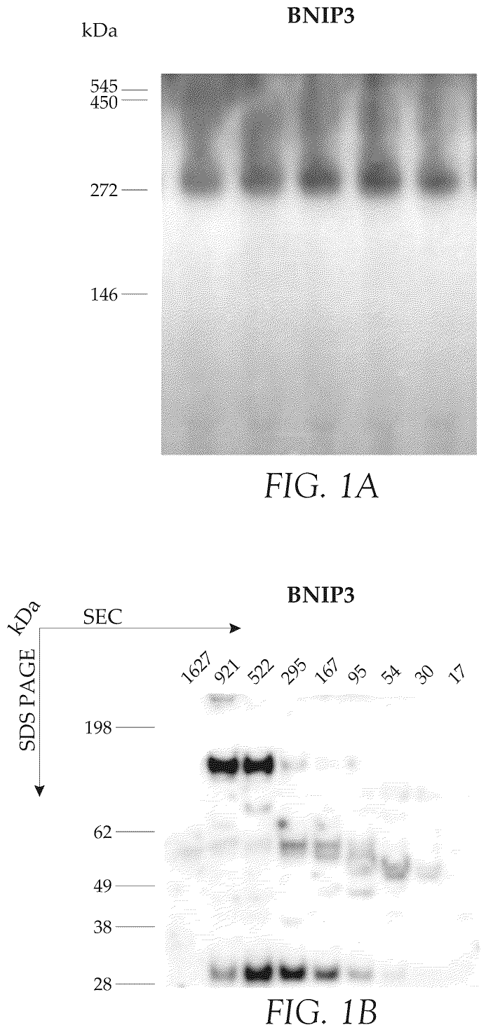

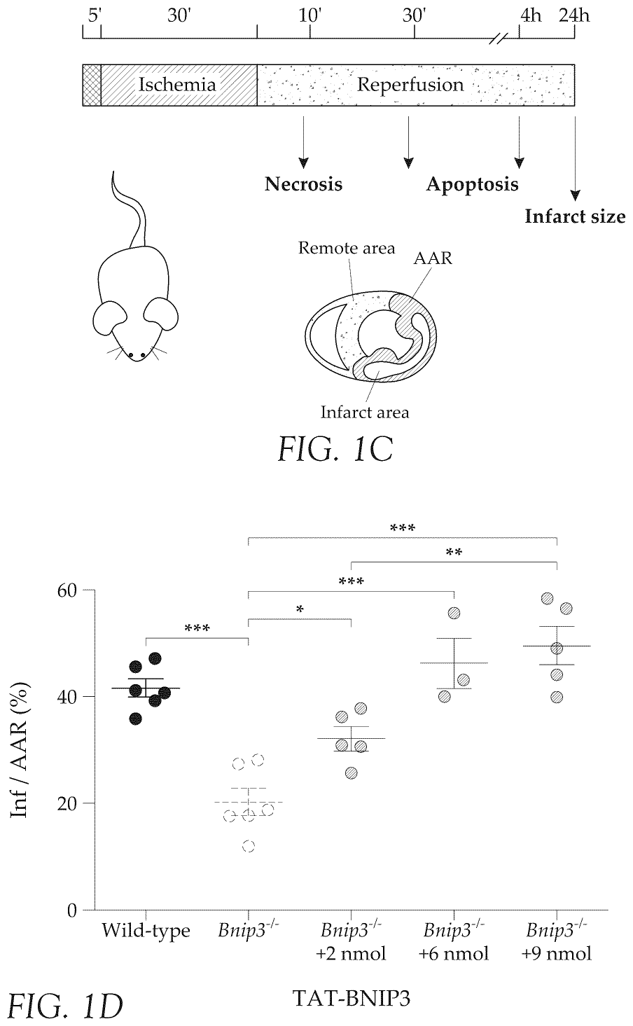

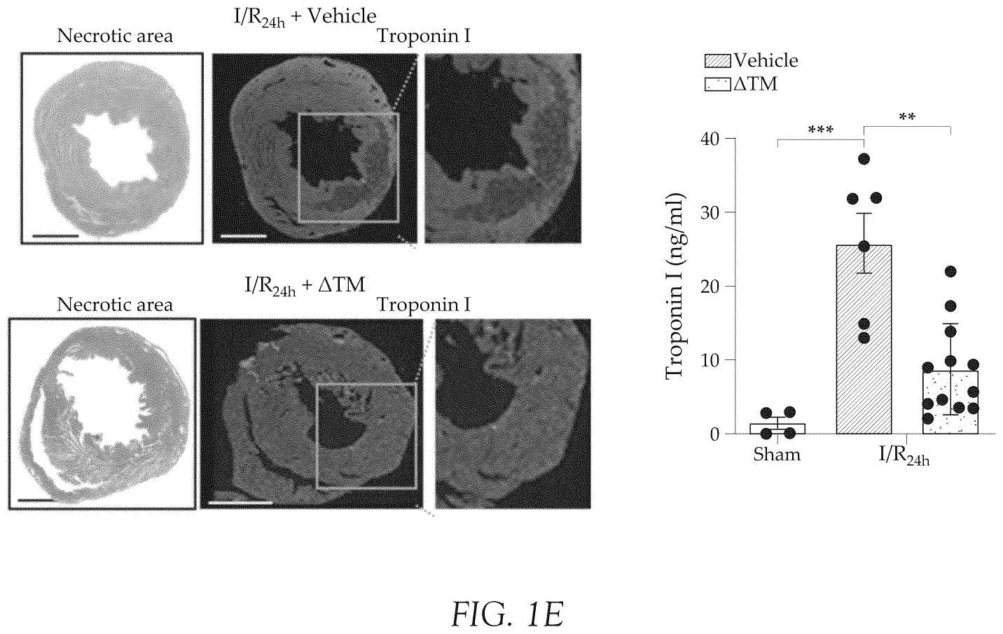

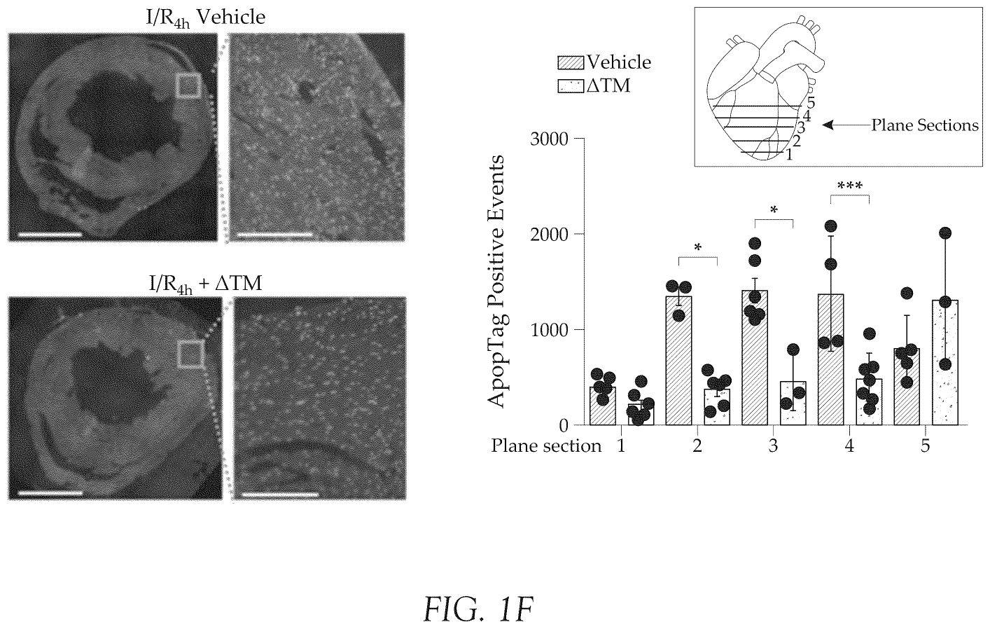

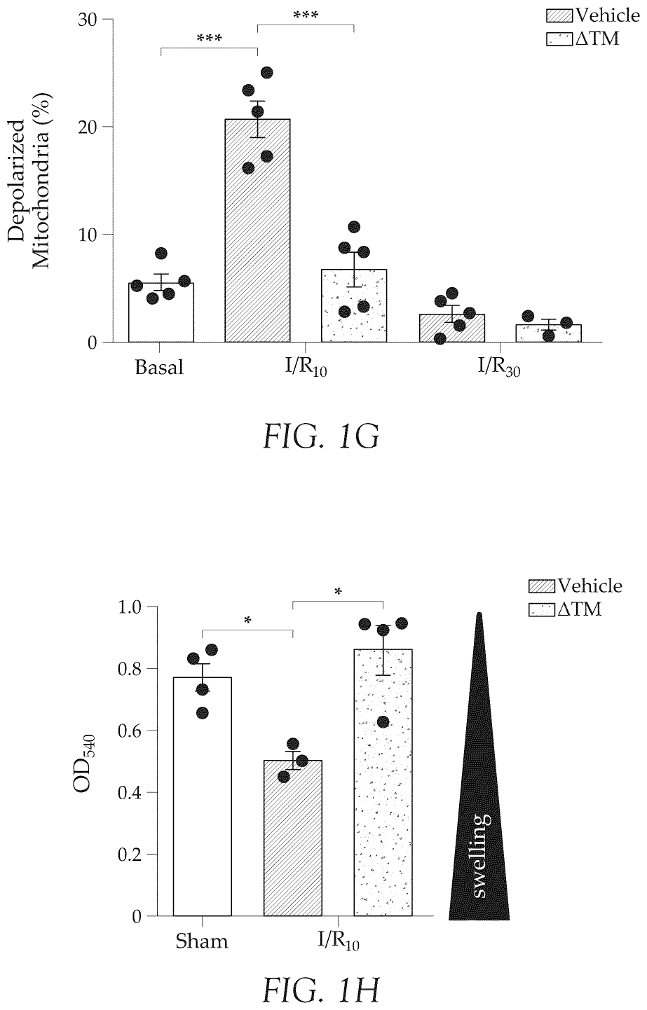

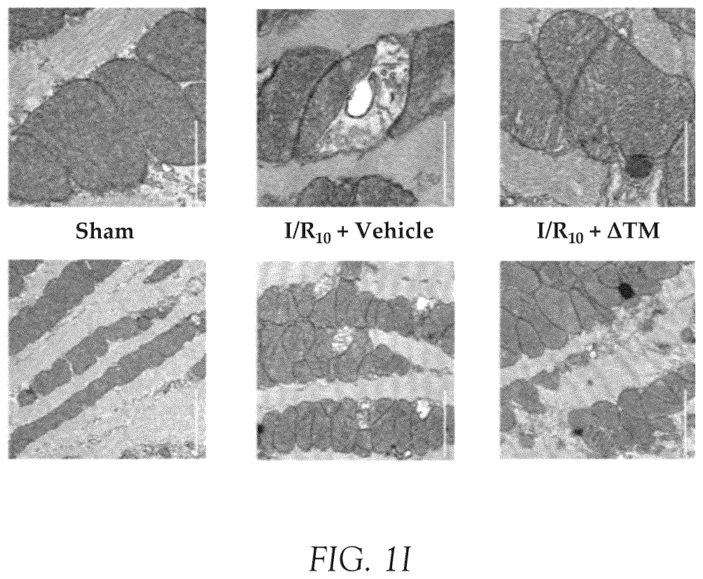

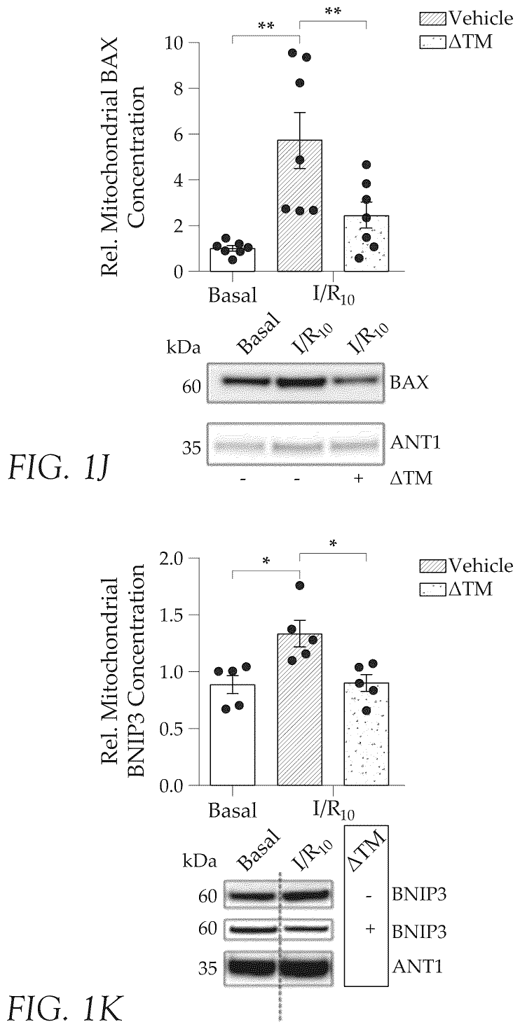

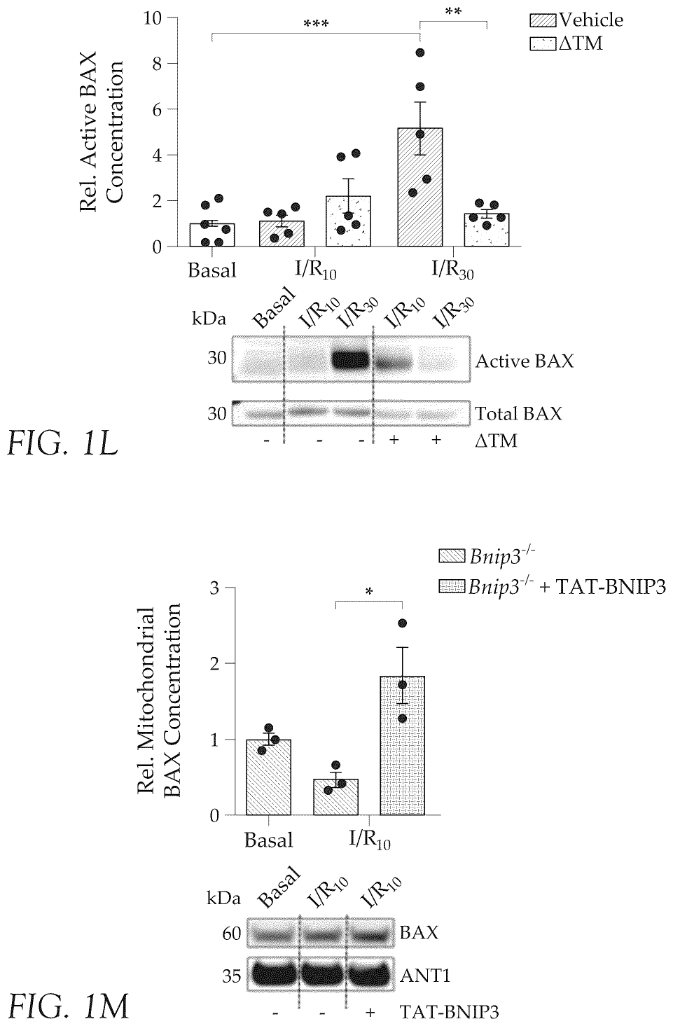

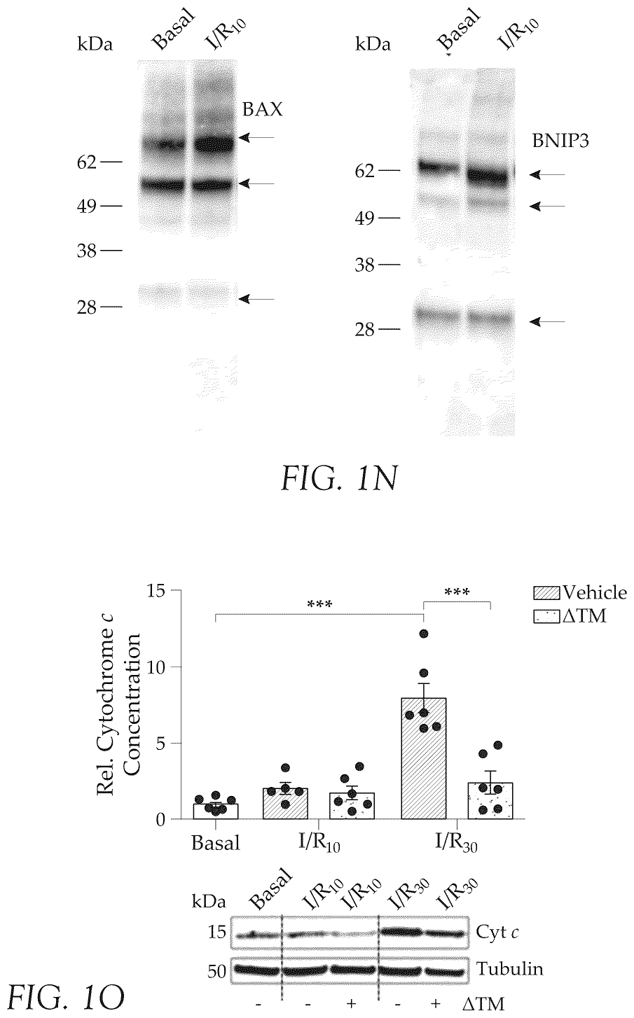

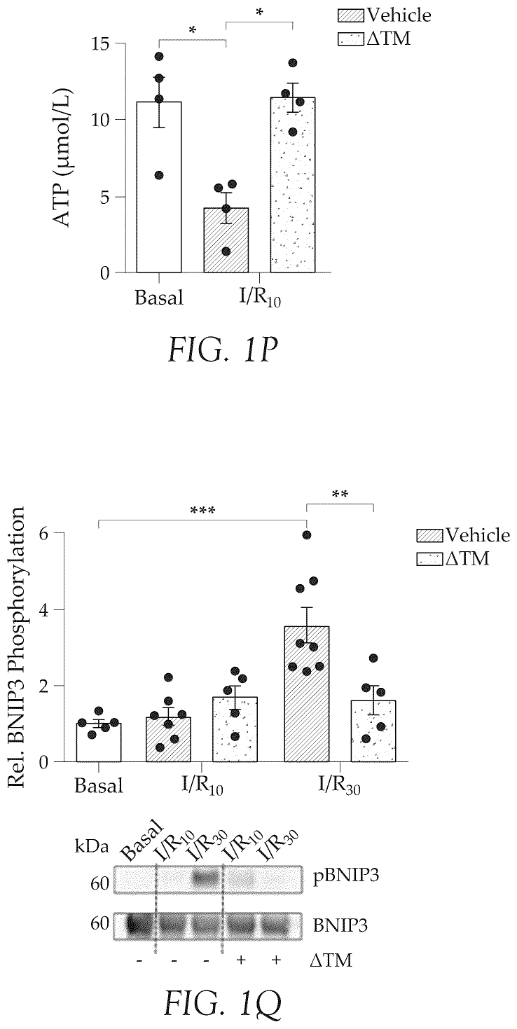

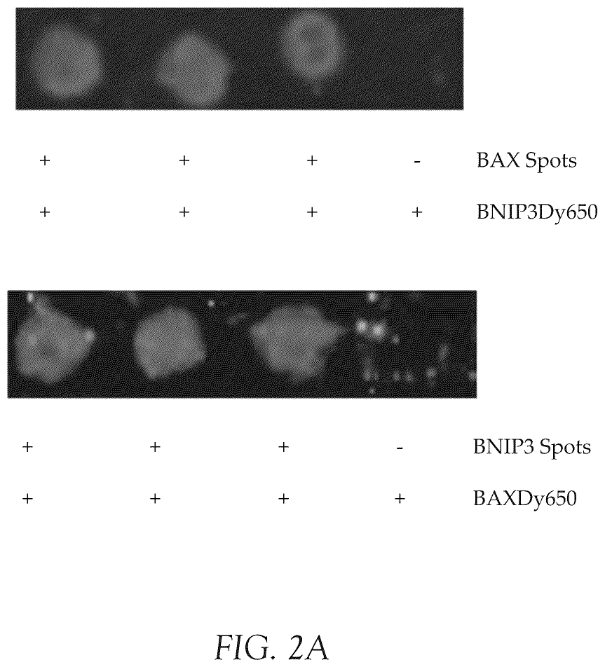

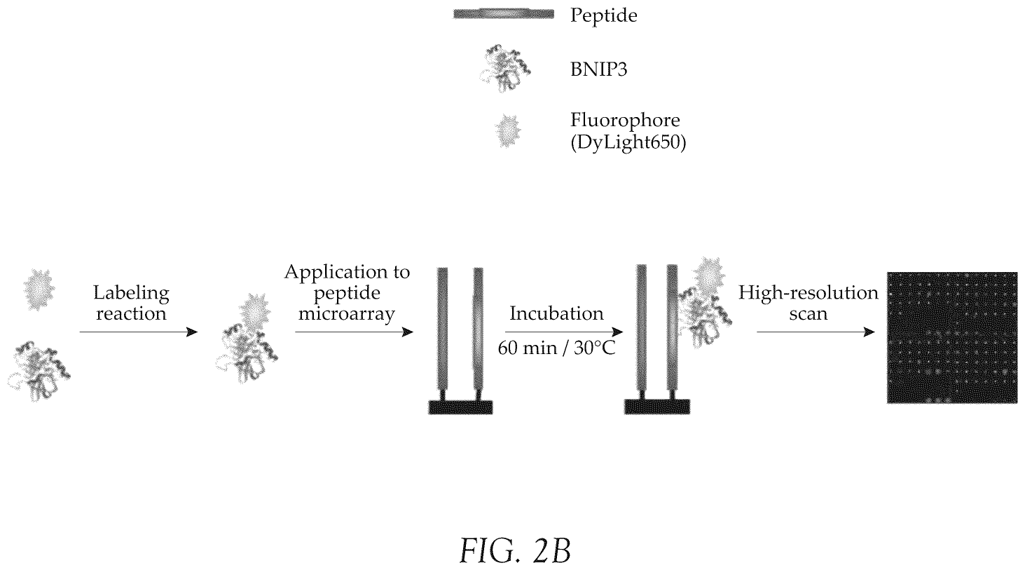

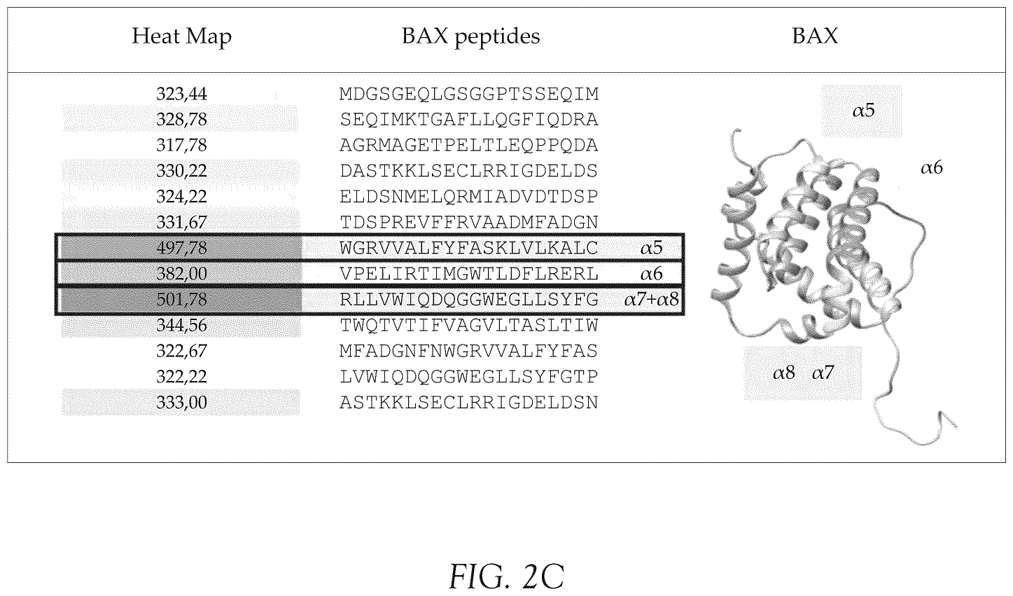

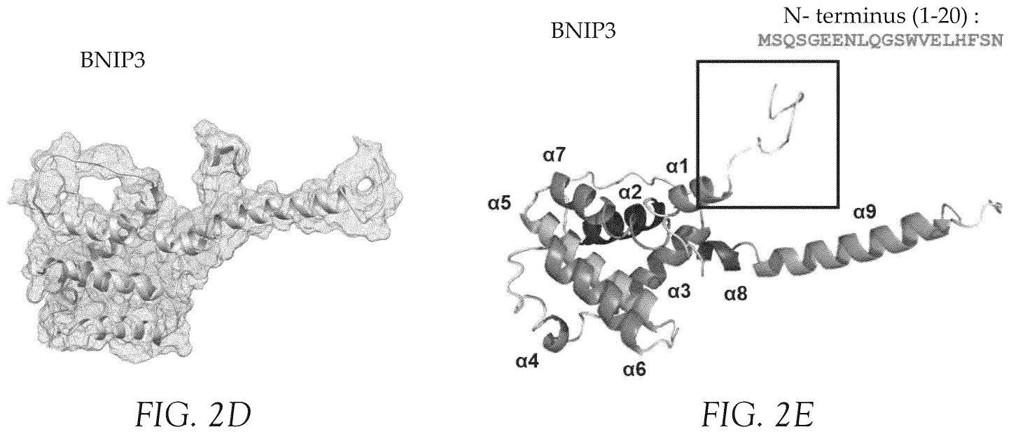

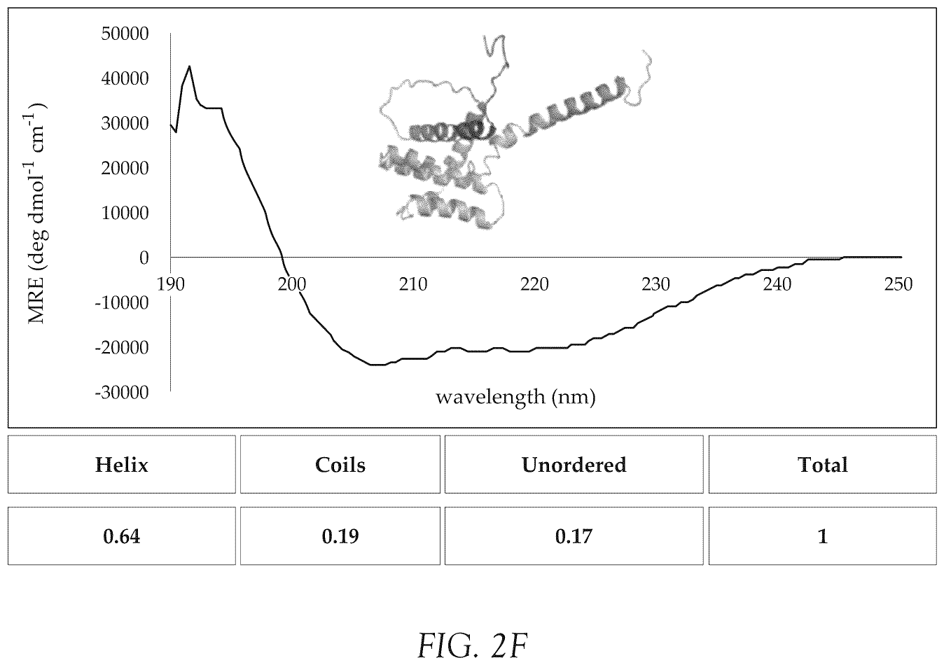

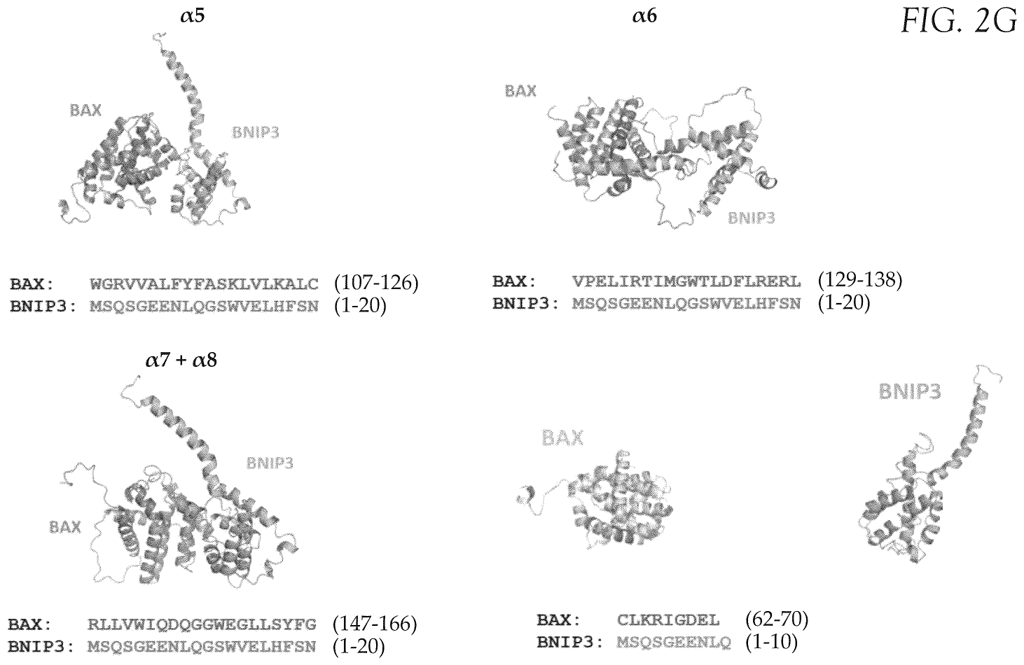

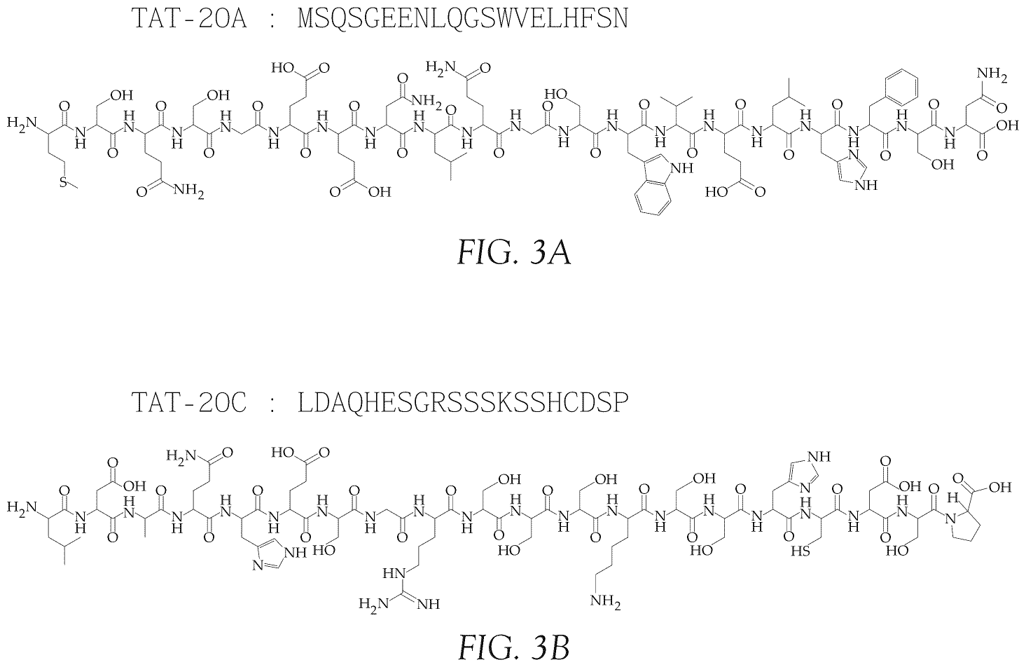

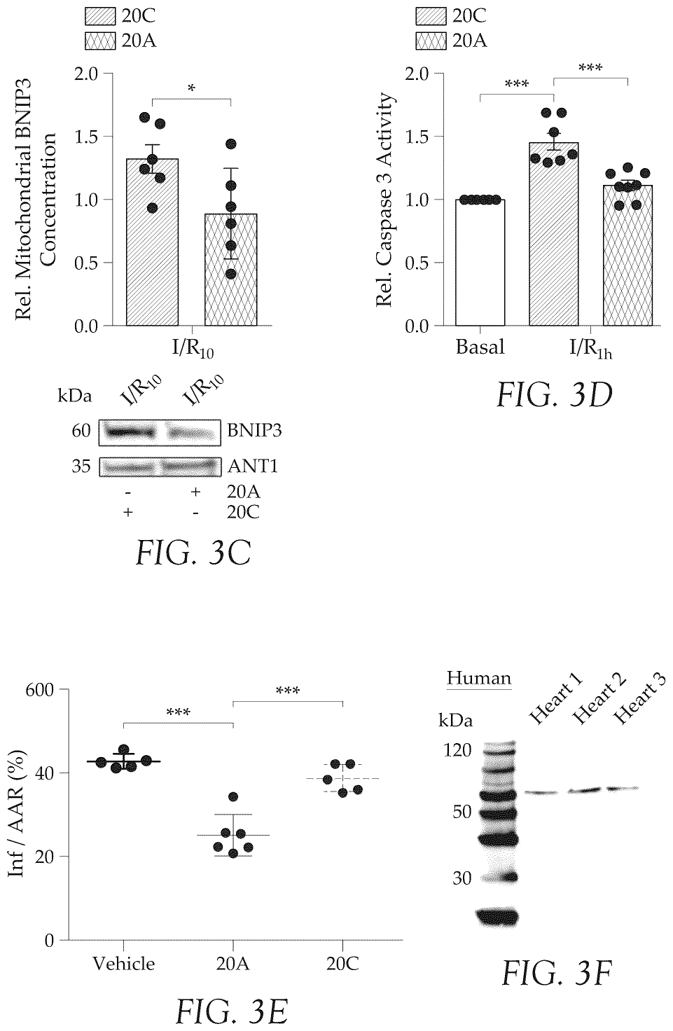

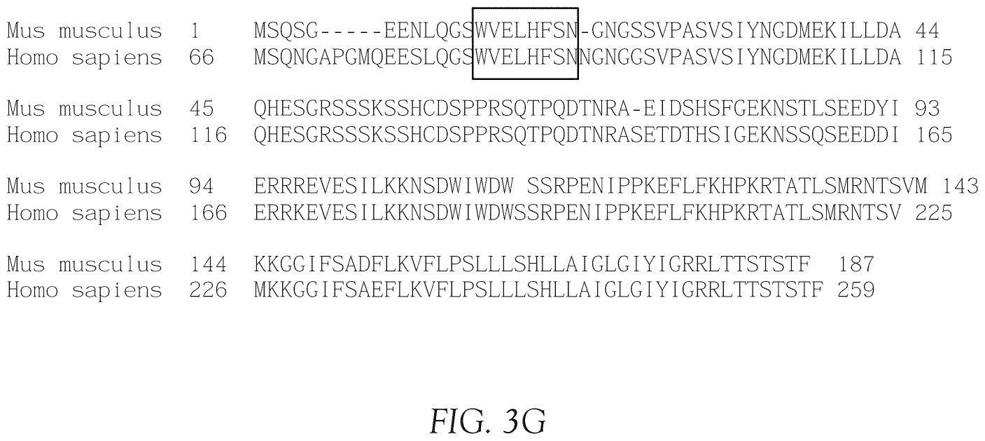

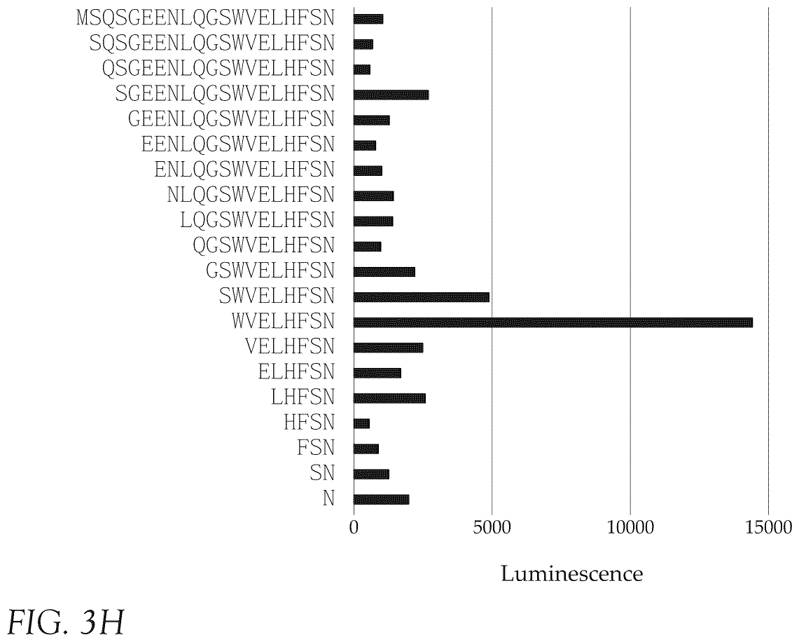

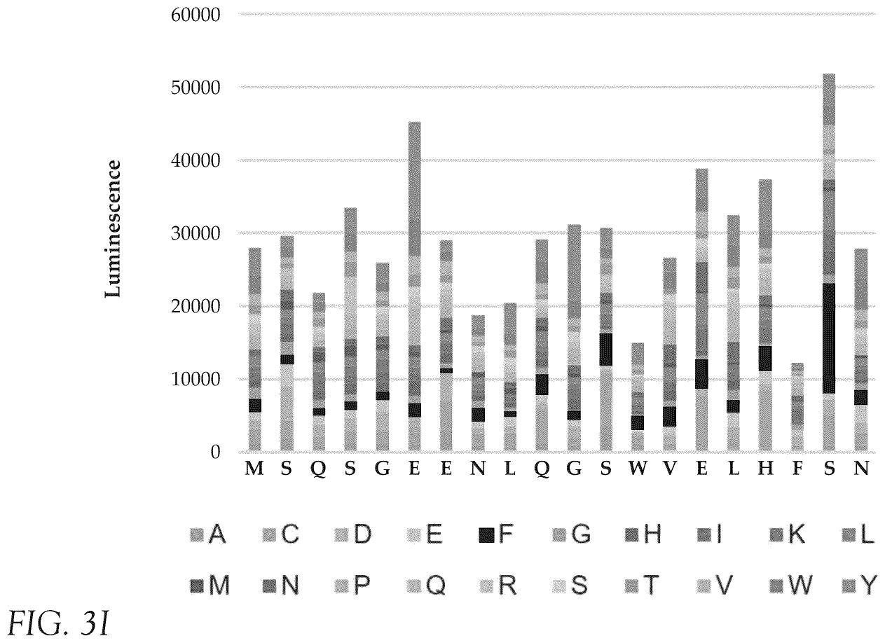

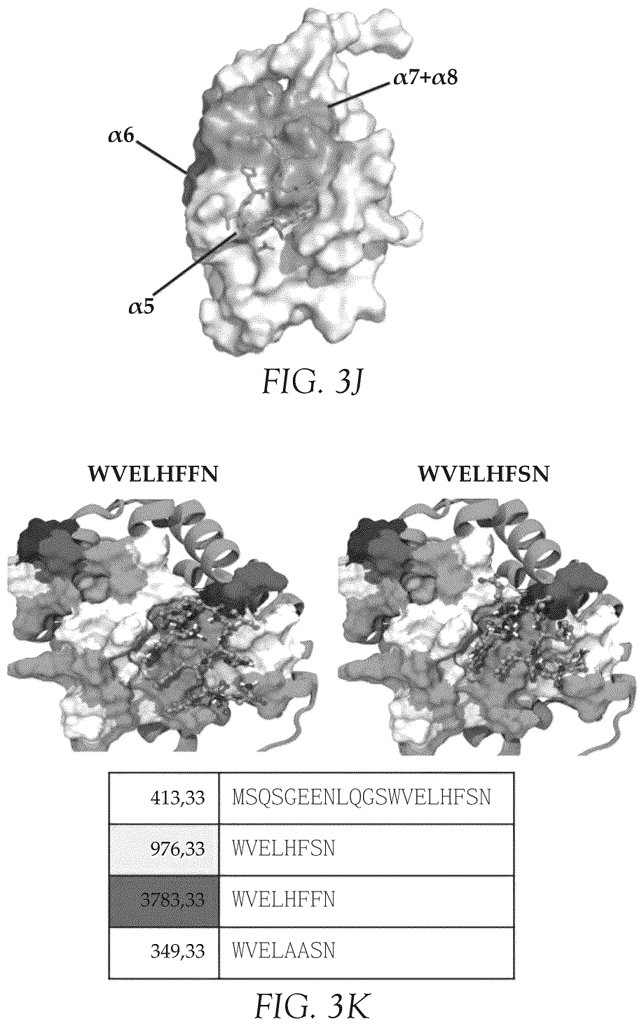

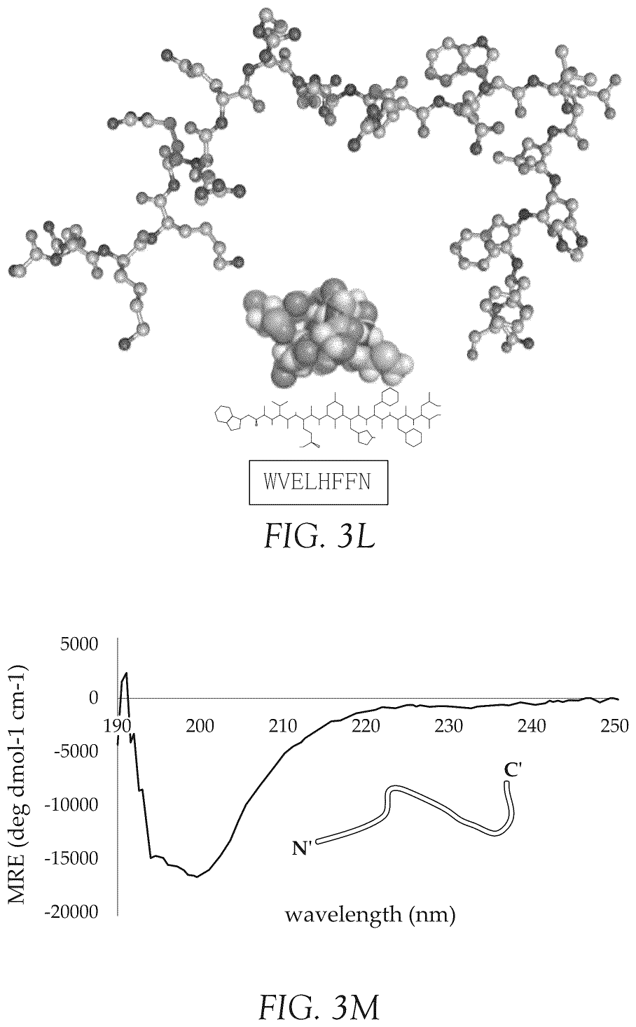

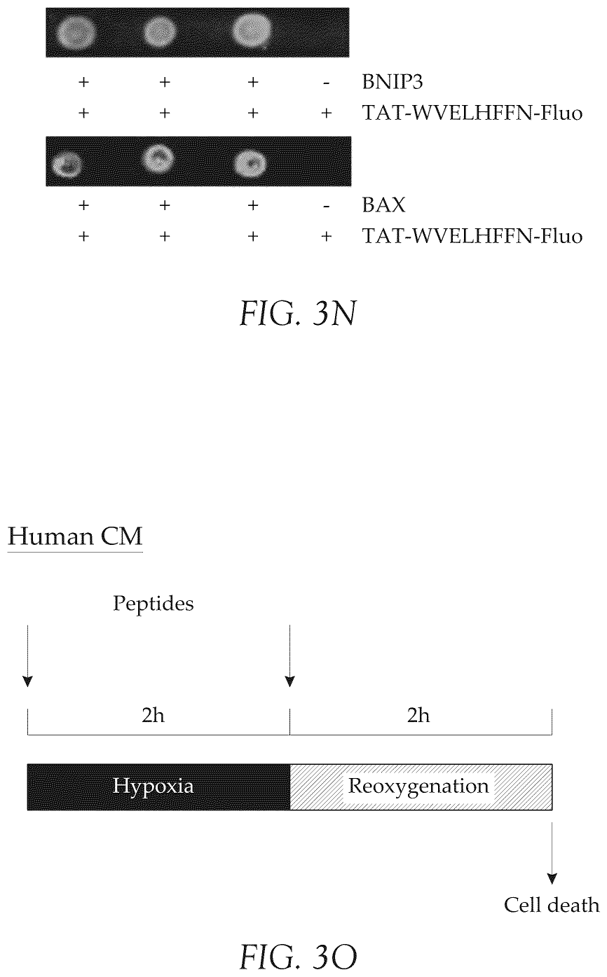

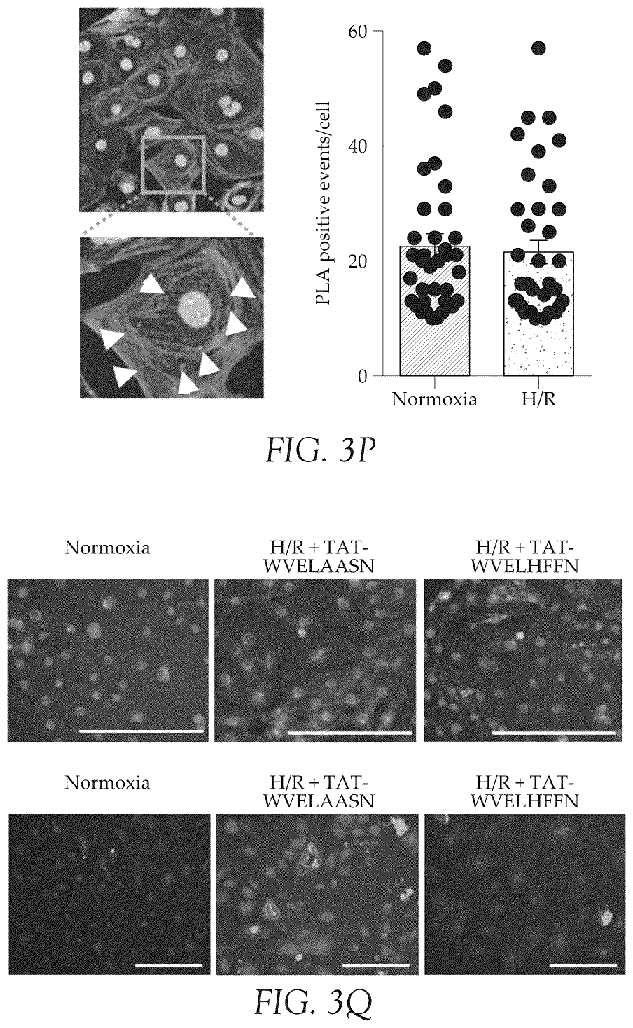

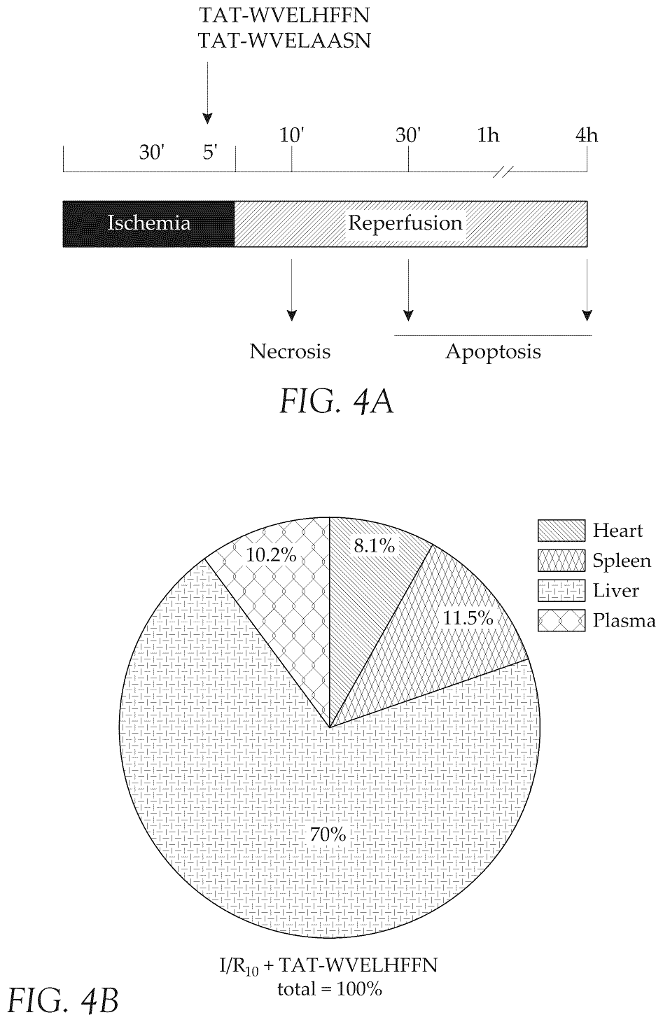

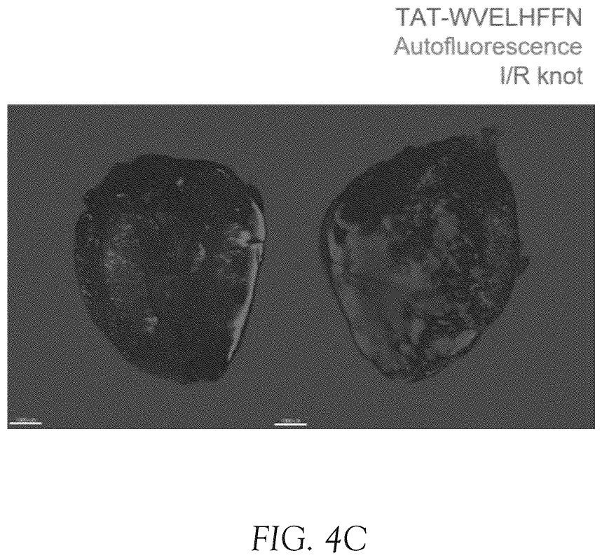

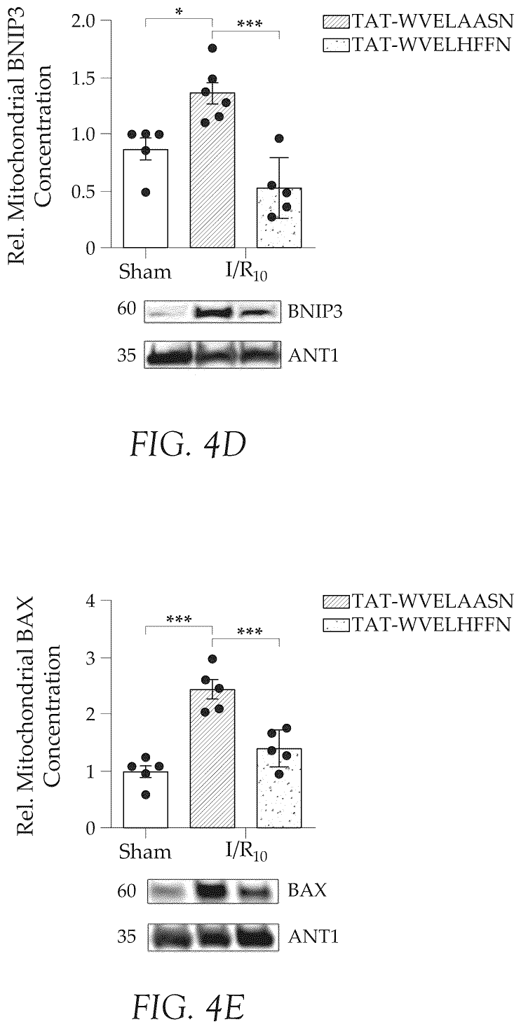

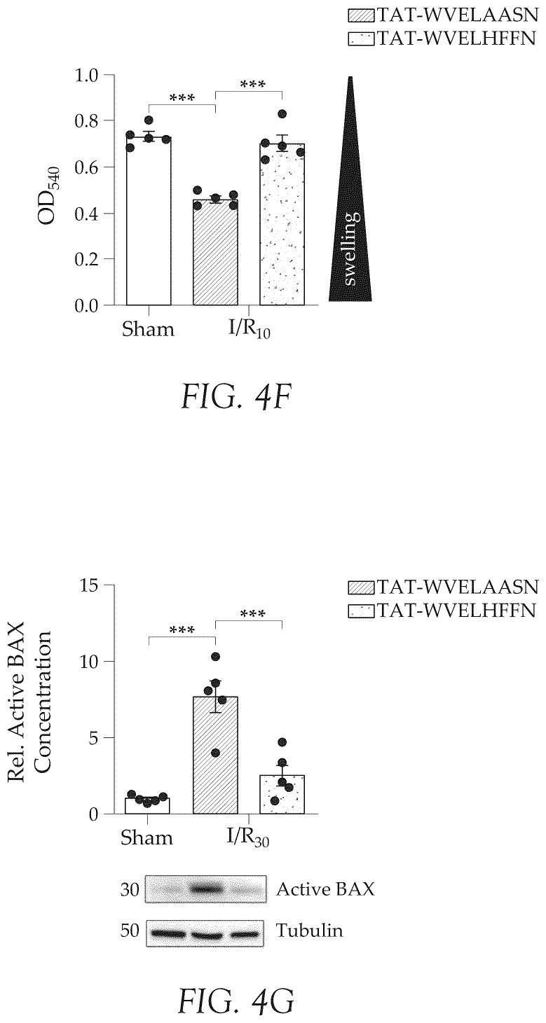

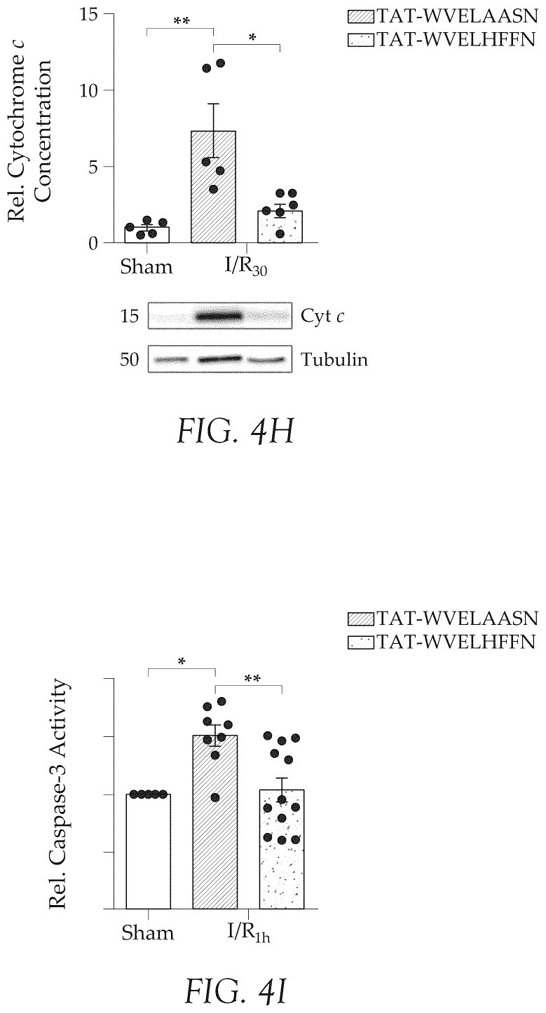

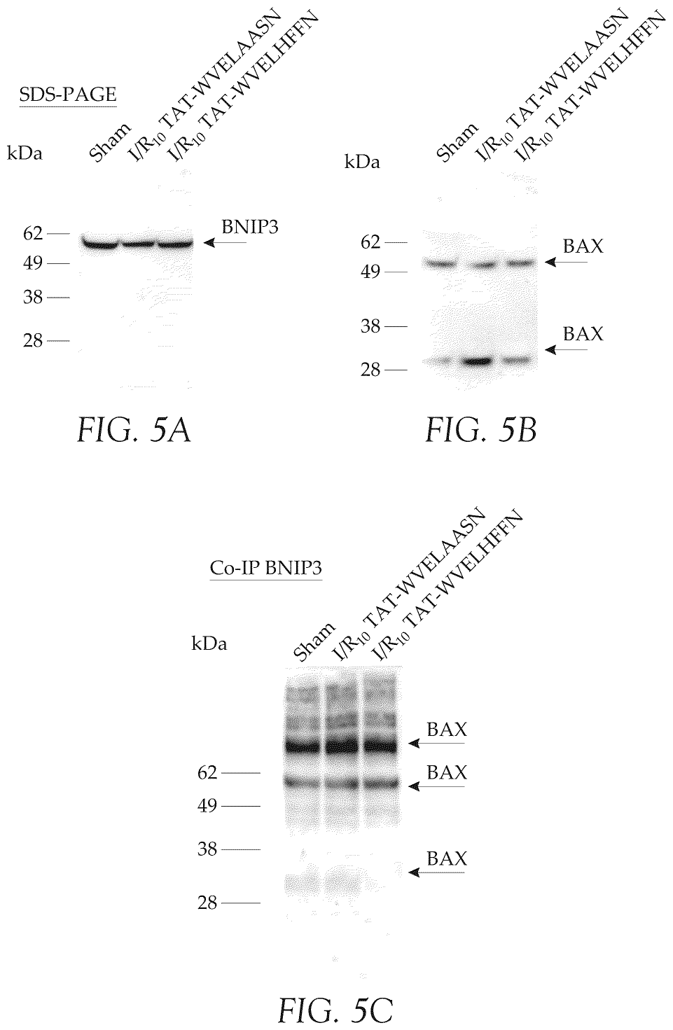

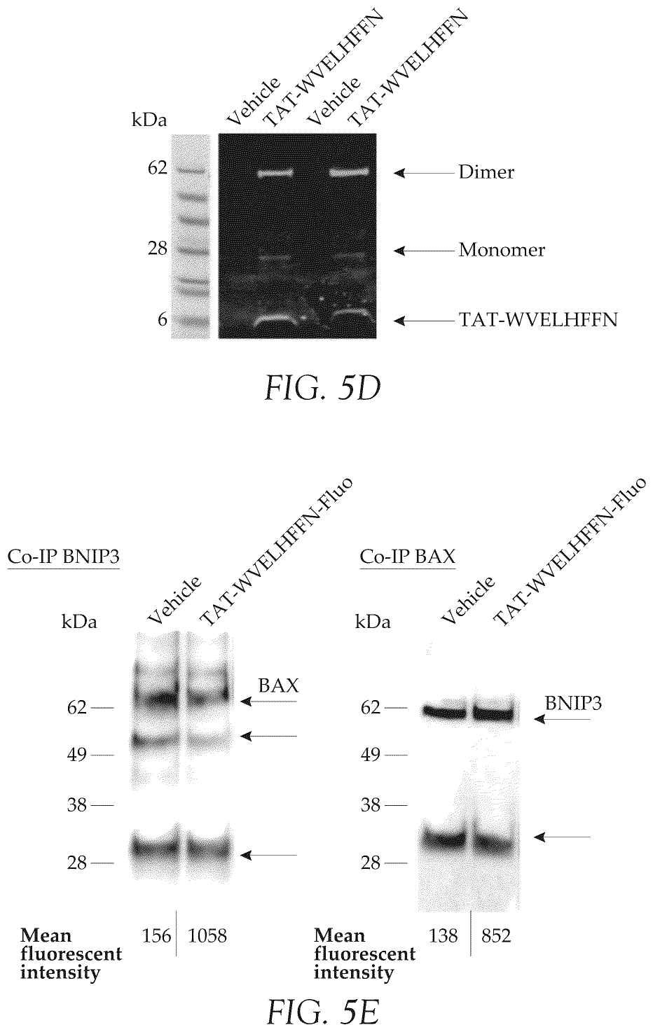

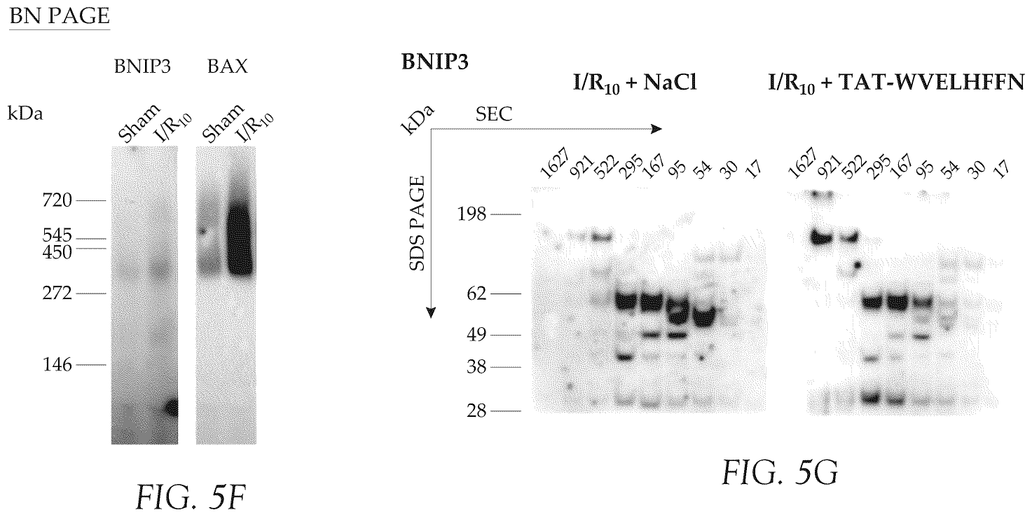

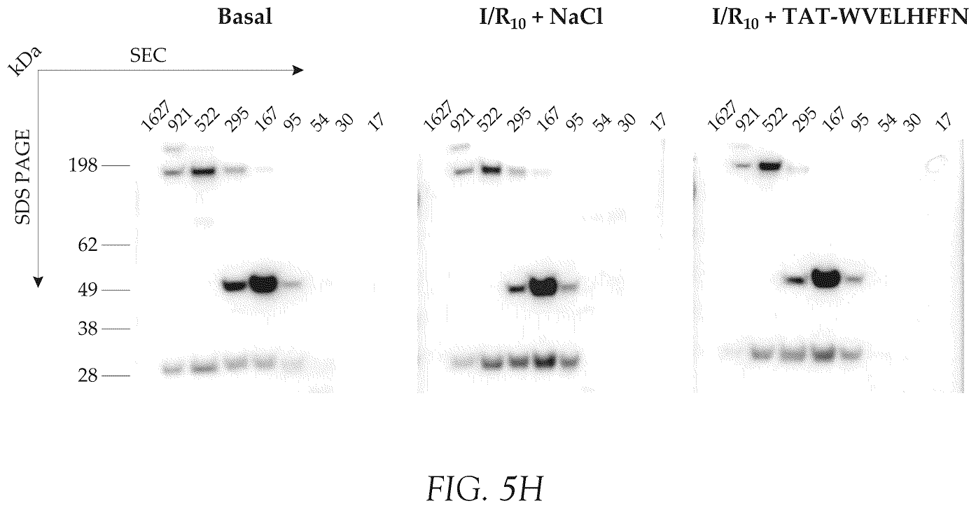

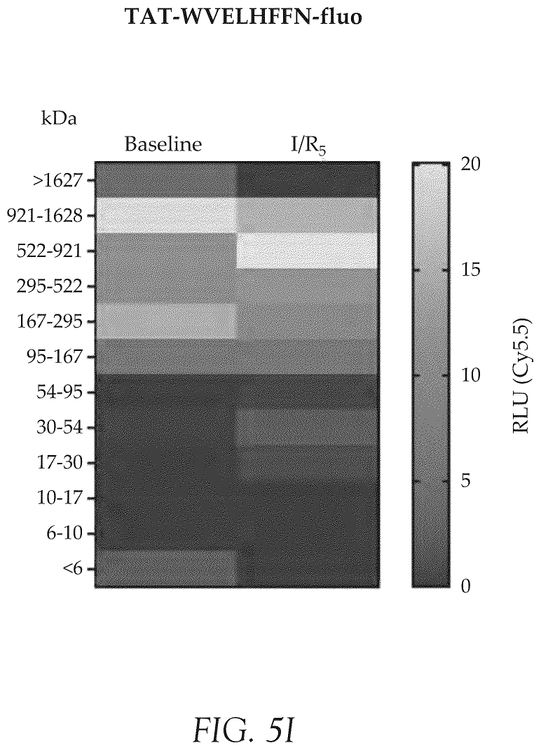

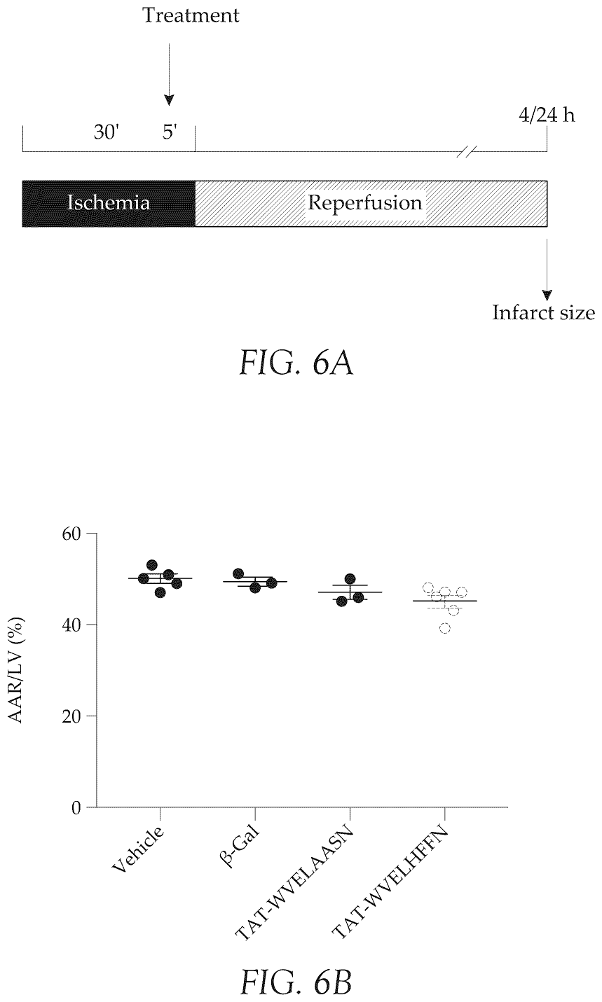

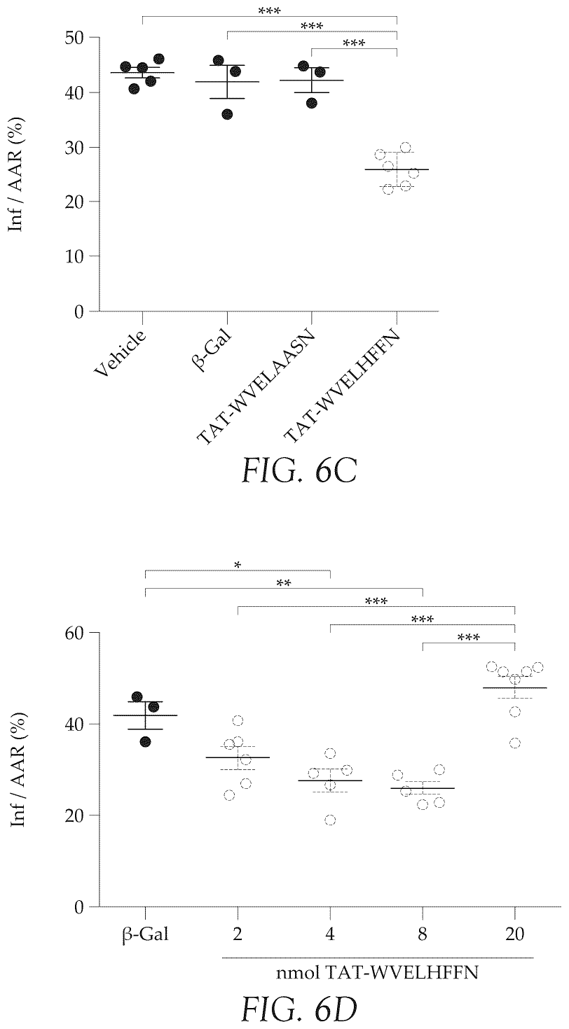

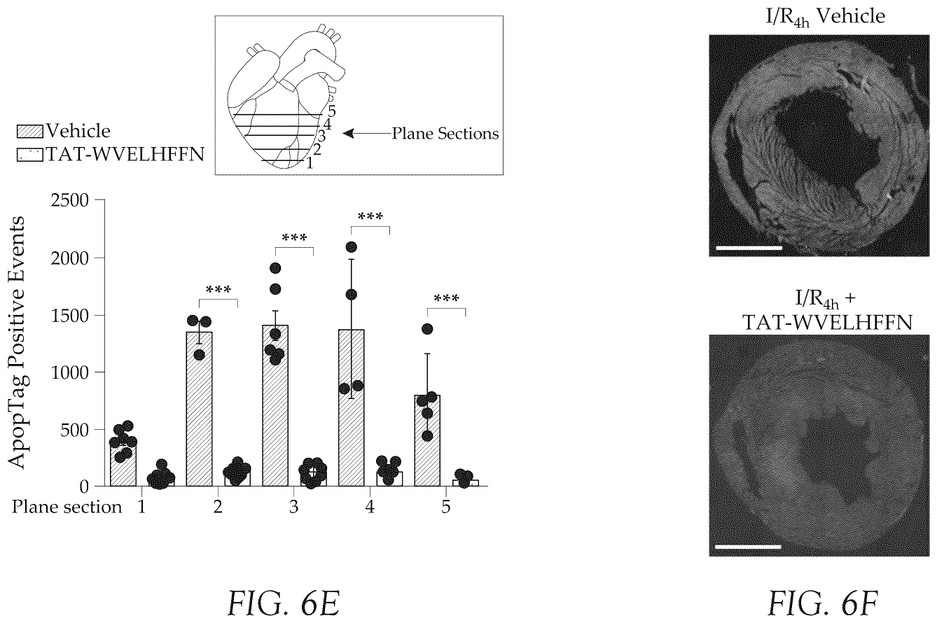

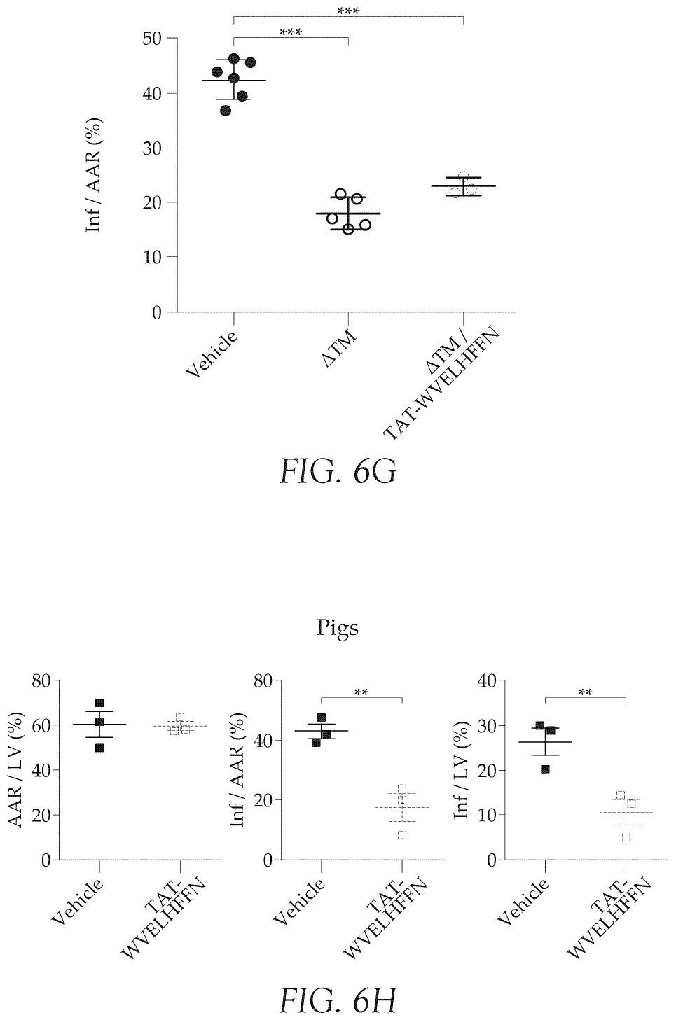

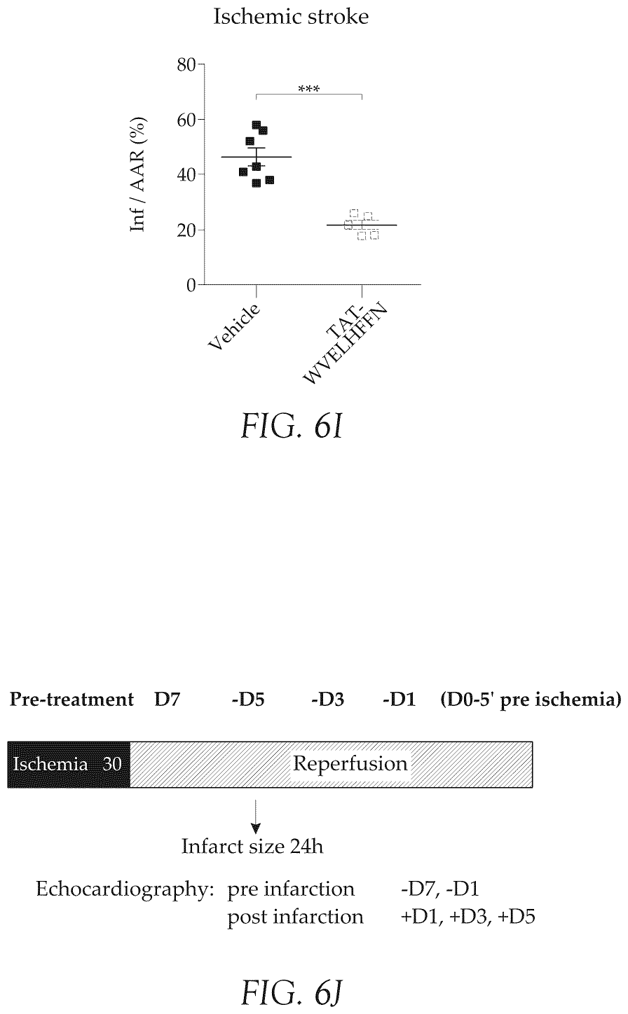

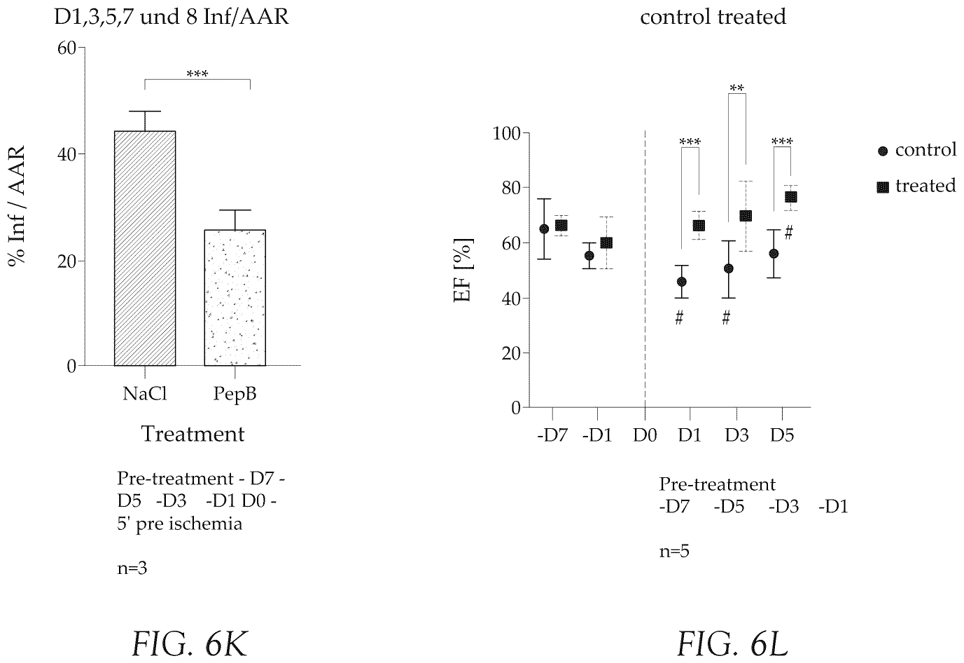

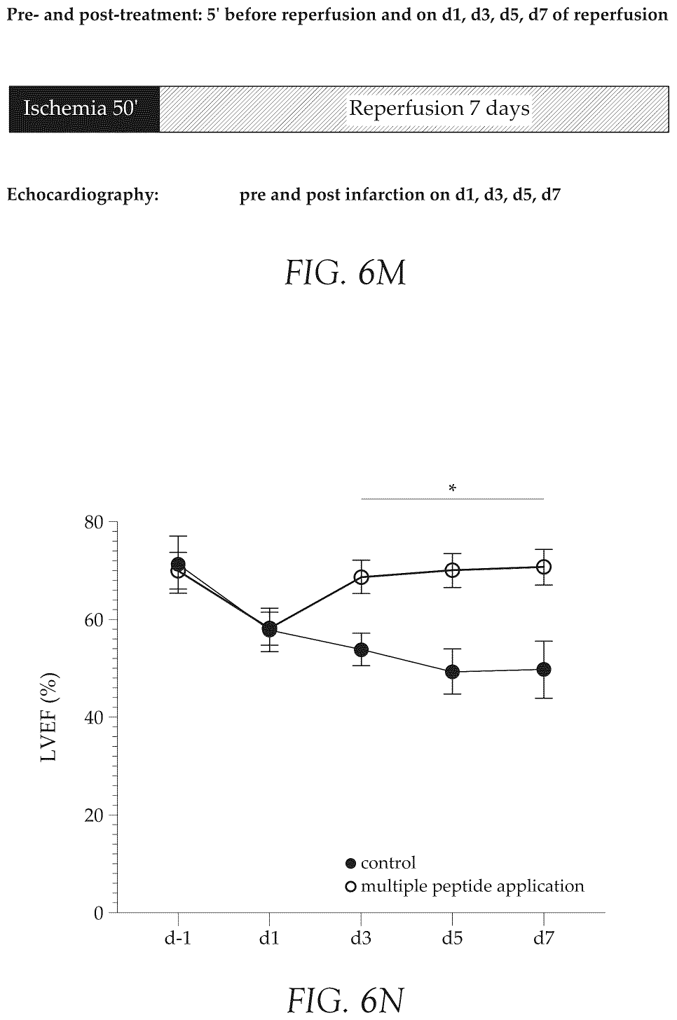

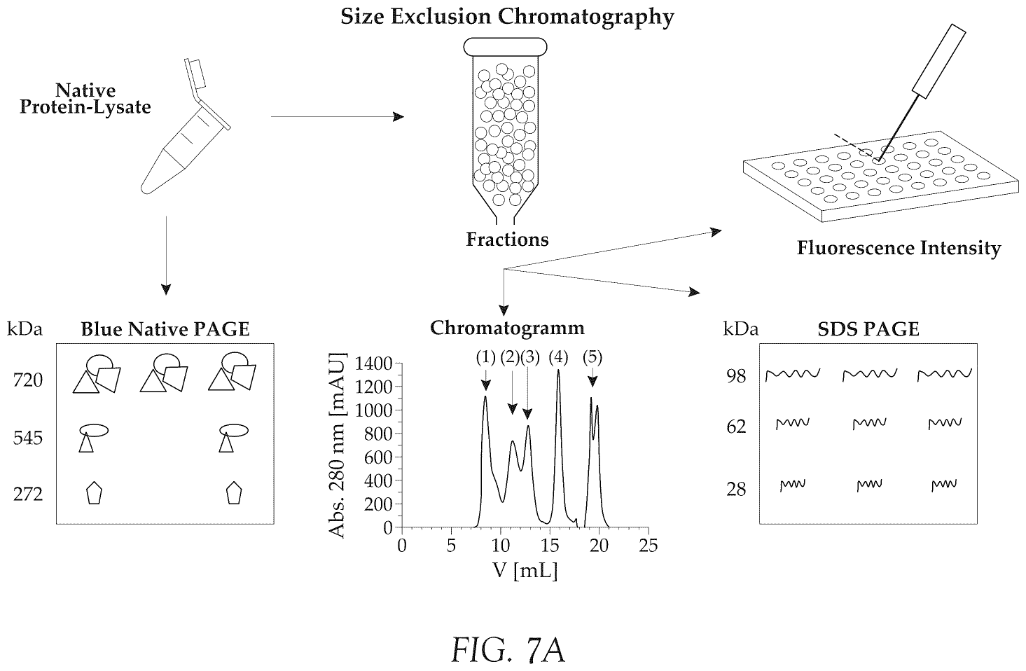

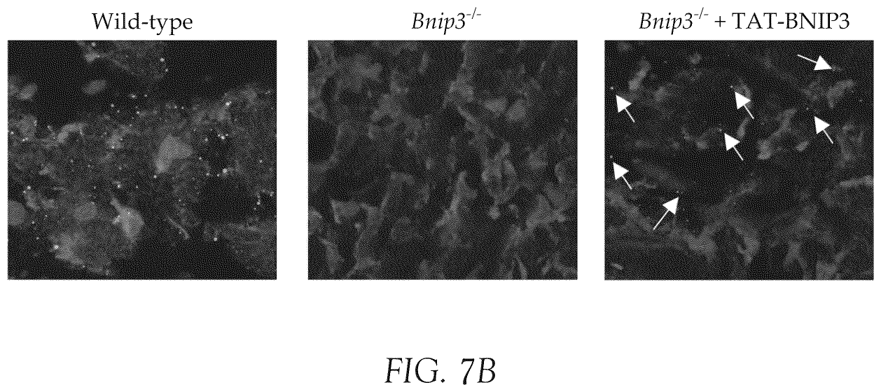

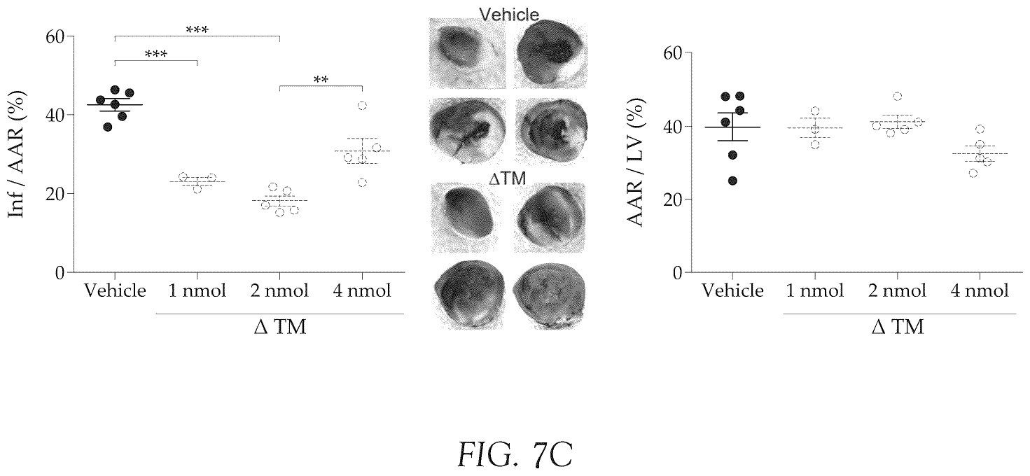

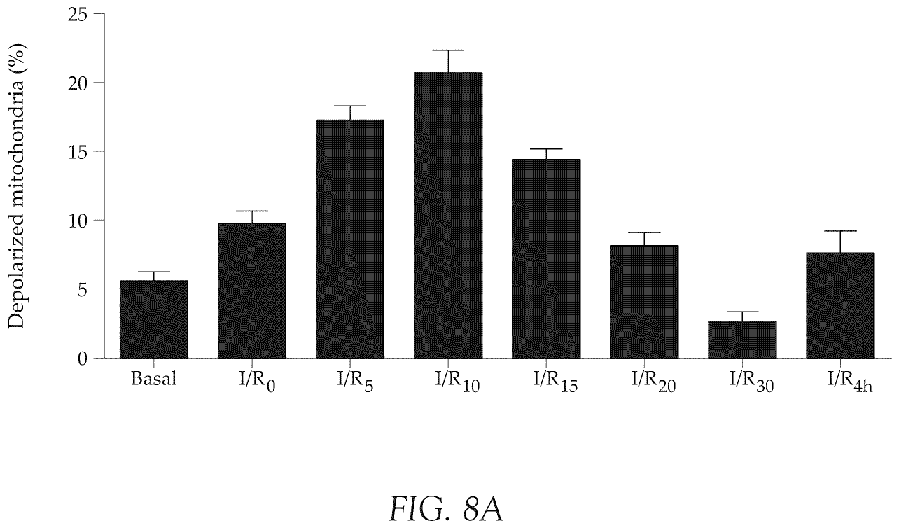

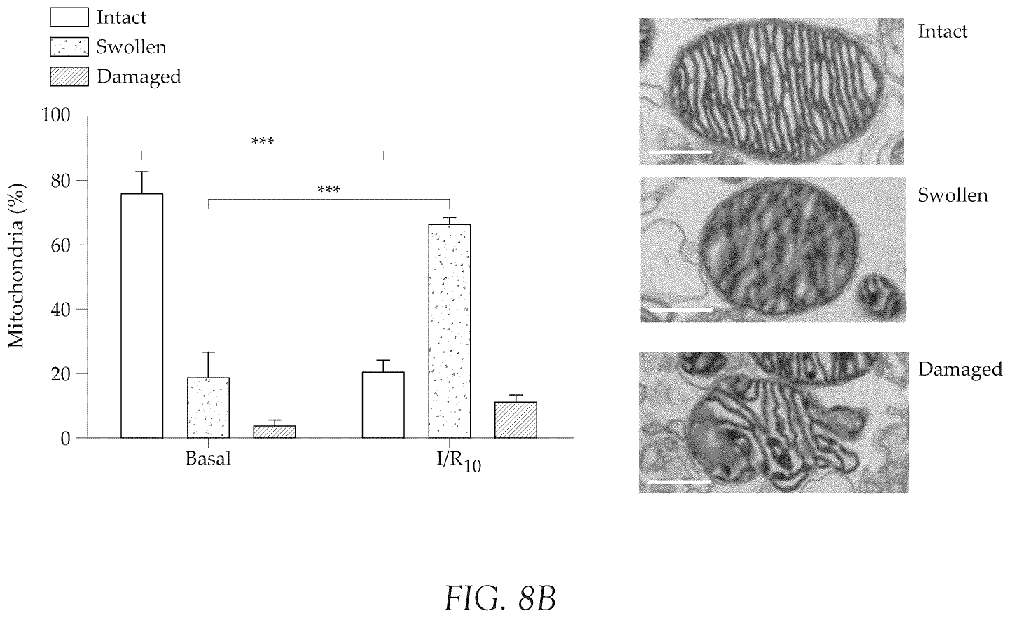

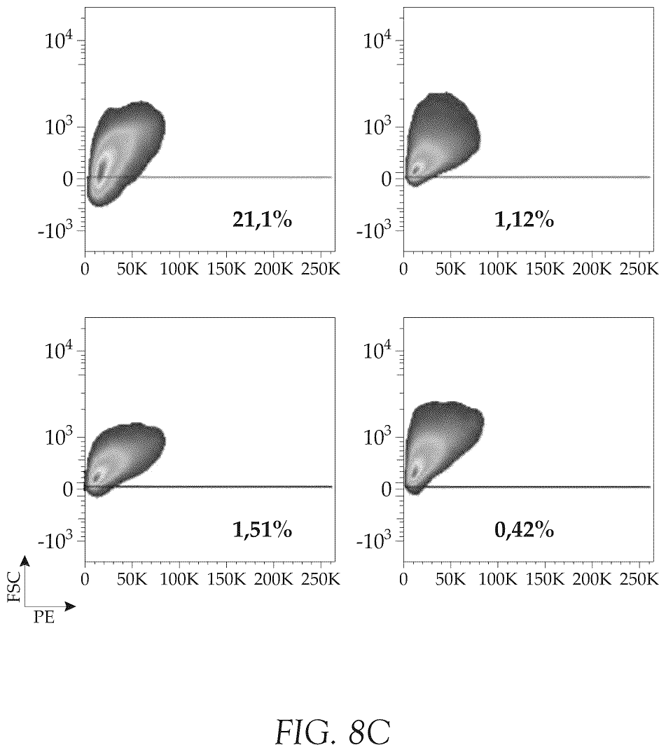

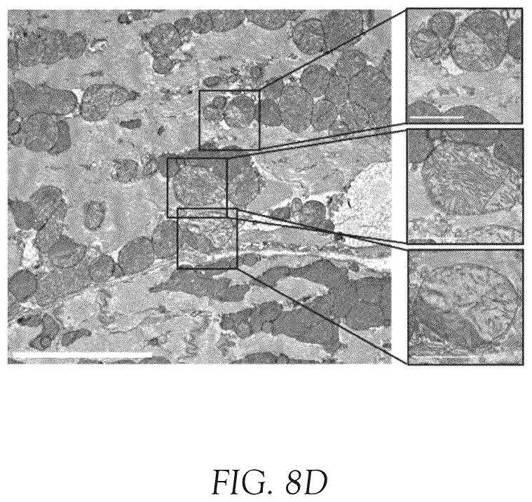

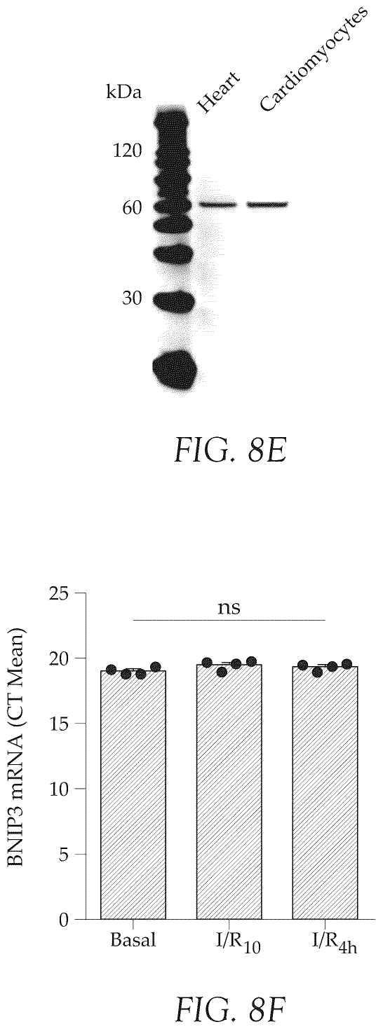

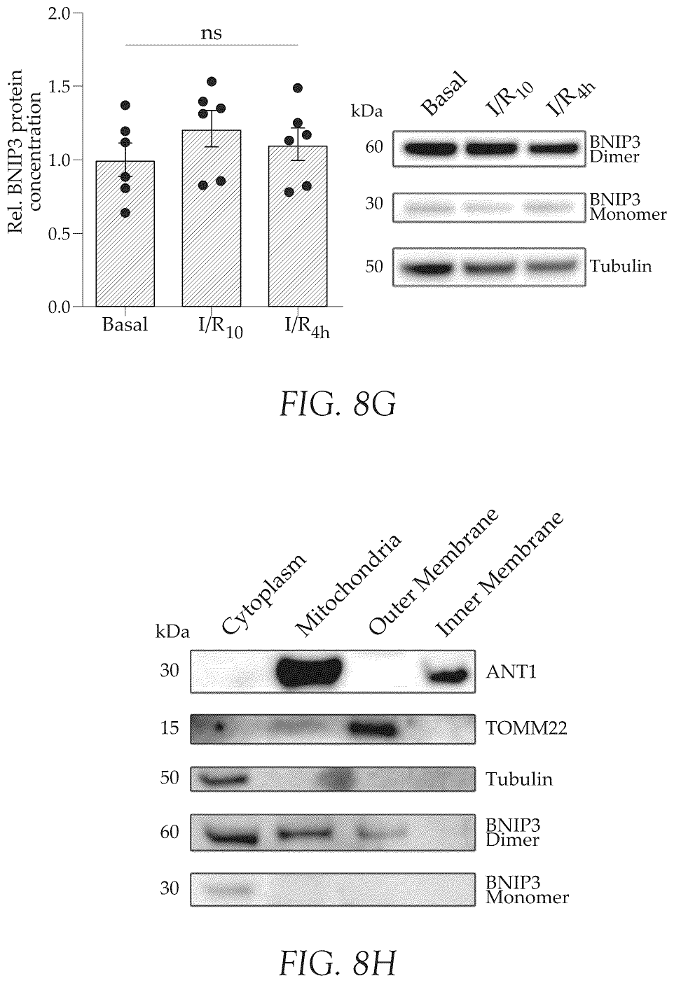

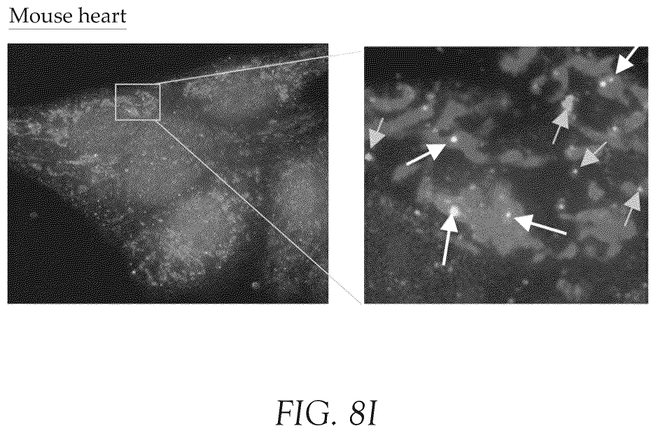

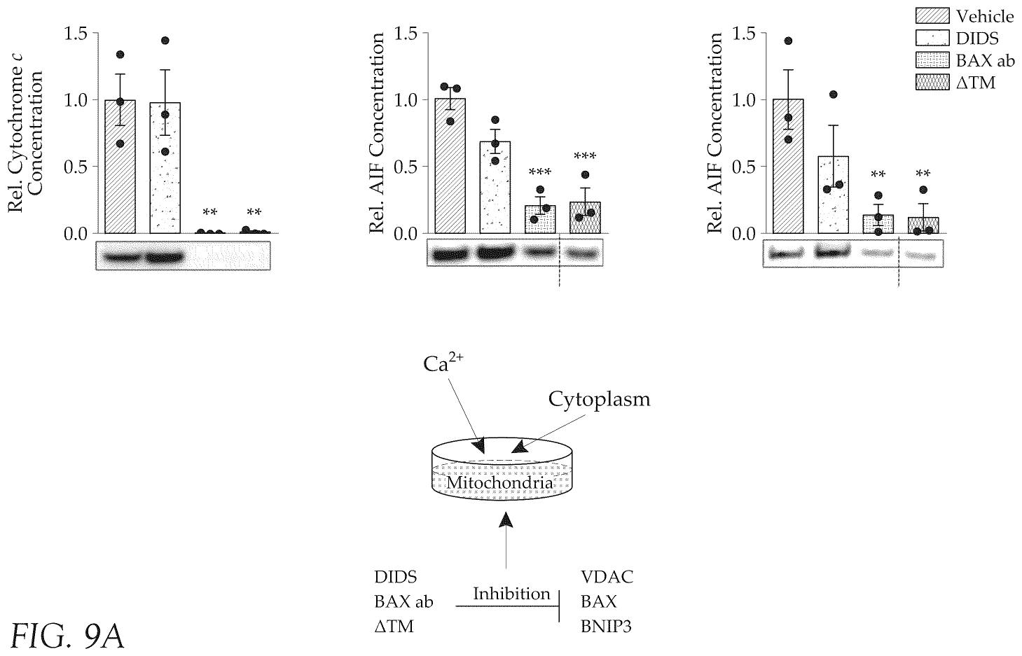

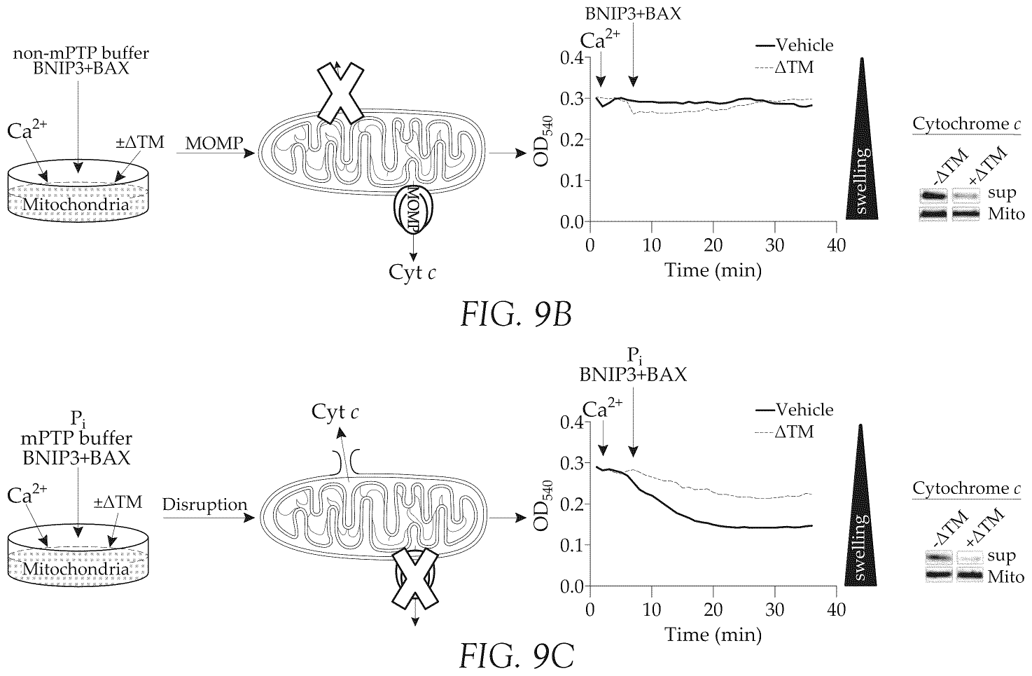

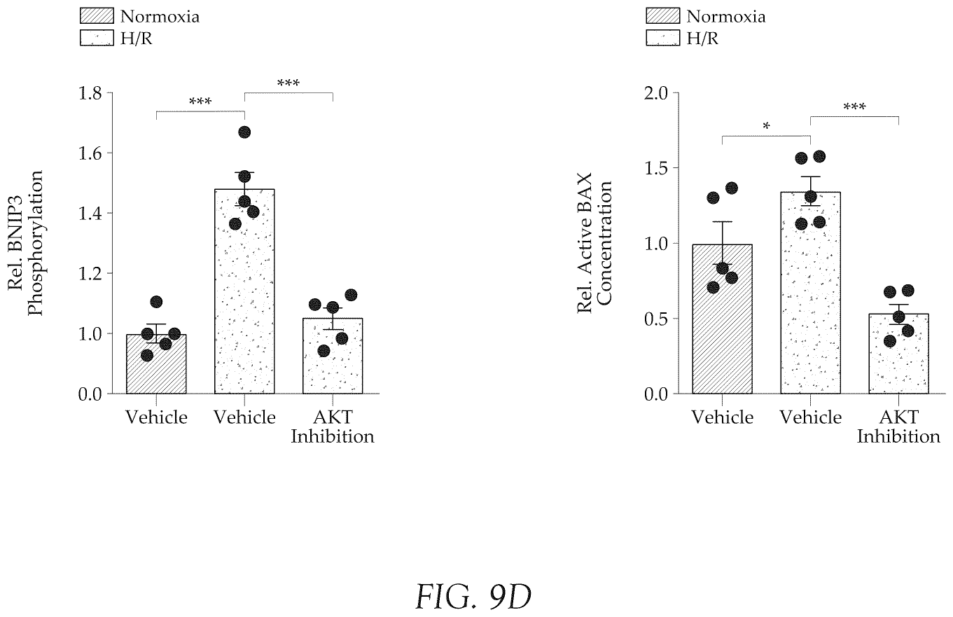

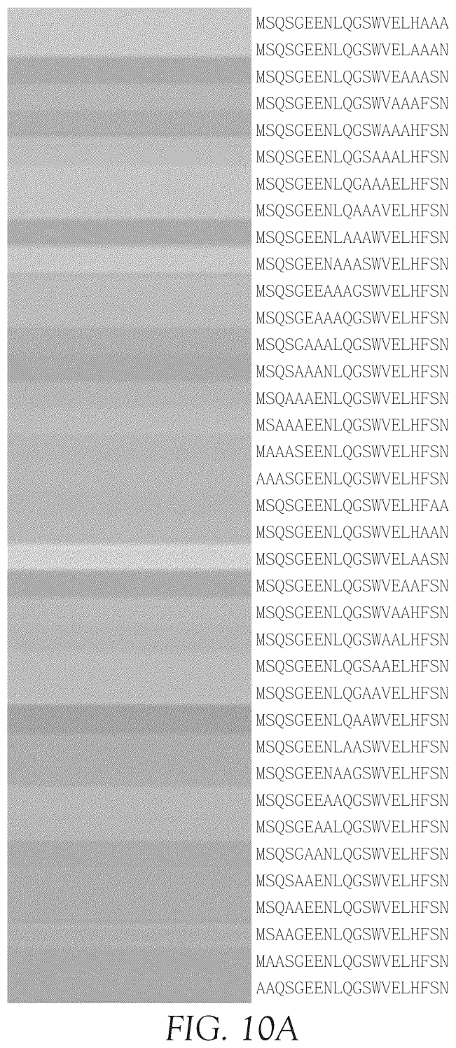

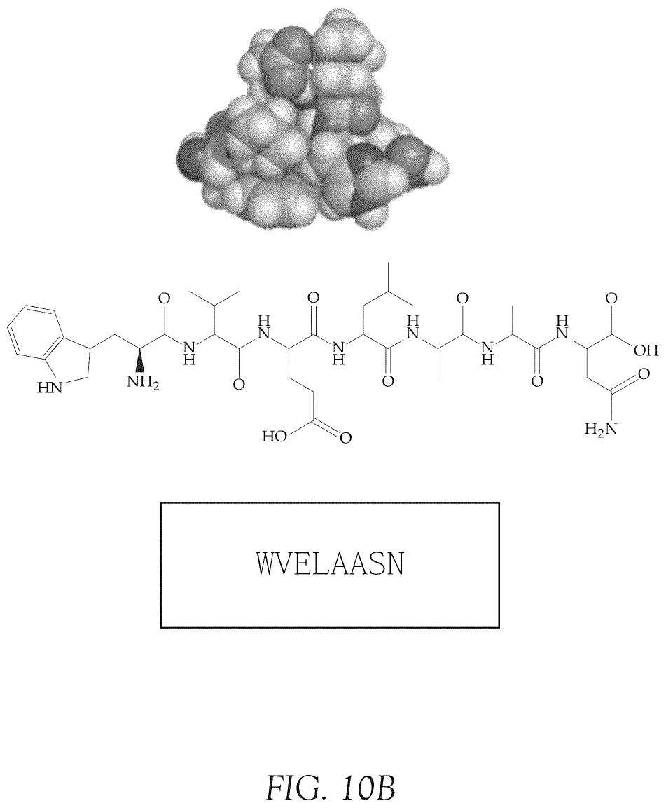

FIG. 1J. delta-TM reduces relative mitochondrial BAX levels to near sham levels.

FIG. 1K. delta-TM reduces relative mitochondrial BNIP3 levels to sham levels.

FIG. 1L. delta-TM reduces relative active BAX concentration to near basal levels at I/R30.

FIG. 1M. TAT-BNIP3 induces mitochondrial BAX accumulation in a Bnip3^{-/-} genetic background.

FIG. 1N. BAX and BNIP3 blots.

FIG. 1O. delta-TM maintains relative cytochrome c concentration at near basal levels at I/R30.

FIG. 1P. delta-TM restores ATP to basal levels.

FIG. 1Q. delta-TM maintains relative BNIP3 phosphorylation at near basal levels.

FIG. 2A. BAX and BNIP3 bind in a spot assay.

FIG. 2B. Treatment schematic.

FIG. 2C. BAX peptide binding heat map. Figure discloses SEQ ID NOS 20-32, respectively, in order of appearance.

FIG. 2D. BNIP3 folding model.

FIG. 2E. BNIP3 N-terminus. Figure discloses SEQ ID NO: 1.

FIG. 2F. BNIP3 Secondary structure prediction.

FIG. 2G. BNIP3-BAX binding prediction. Figure discloses SEQ ID NOS 26, 33, 1, 1, and 34-37, respectively, in order of appearance.

FIG. 3A. TAT-20A MSQGEENLQGSWVELHFSN (SEQ ID NO: 5). Figure discloses

“MSQSGEENLQGSWVELHFSN” as SEQ ID NO: 1.

FIG. 3B. TAT-20C LDAQHESGRSSSKSSHCDSP (SEQ HD NO. 6). Figure discloses

“LDAQHESGRSSSKSSHCDSP” as SEQ ID NO: 6.

FIG. 3C. 20A shows reduced relative BNIP3 mitochondrial concentration.

FIG. 3D. 20A restores basal relative caspase 3 activity.

FIG. 3E. 20A improves Inf/AAR percent relative to vehicle or 20C.

FIG. 3F. Human protein blot.

FIG. 3G. Mouse/human alignment. Figure discloses SEQ ID NOS 38-39, respectively, in order of appearance.

FIG. 3H. Truncation fluorescence assay for 20A N-terminal truncations. WVELHFSN (SEQ ID NO: 2) shows elevated levels. Figure discloses SEQ ID NOS 1, 40-50, 2, 8, and 51-53, respectively, in order of appearance.

FIG. 3I. Substitution fluorescence assay.

FIG. 3J. Binding pocket prediction.

FIG. 3K. Interaction heat map. Figure discloses SEQ ID NOS 3, 2, 1, 2, 3, and 7, respectively, in order of appearance.

FIG. 3L. Interaction model. Figure discloses “WVELHFFN” as SEQ ID NO: 3.

FIG. 3M. Folding prediction.

FIG. 3N. TAT-WVELHFFN (“WVELHFFN” disclosed as SEQ ID NO: 3) binds BNIP3 and BAX. Figure discloses “WVELHFFN-Fluo” as SEQ ID NO: 9.

FIG. 3O. Time course model.

FIG. 3P. PLA positive events per cell.

FIG. 3Q. Comparison of TAT-peptide variants. Figure discloses “WVELHFFN” as SEQ ID NO: 3 and “WVELAASN” as SEQ ID NO: 7.

FIG. 4A. Treatment regimen. Figure discloses SEQ ID NOS 3 and 7, respectively, in order of appearance.

FIG. 4B. Peptide localizations. Figure discloses SEQ ID NO: 3.

FIG. 4C. Peptide localization image. Figure discloses SEQ ID NO: 3.

FIG. 4D. TAT-WVELHFFN (“WVELHFFN” disclosed as SEQ ID NO: 3) counteracts TAT-WVELAASN (“WVELAASN” disclosed as SEQ ID NO: 7) increase in relative mitochondrial BNIP3 concentration. Figure discloses SEQ ID NOS 7 and 3, respectively, in order of appearance.

FIG. 4E. TAT-WVELHFFN (“WVELHFFN” disclosed as SEQ ID NO: 3) counteracts TAT-WVELAASN (“WVELAASN” disclosed as SEQ ID NO: 7) increase in relative mitochondrial BAX concentration. Figure discloses SEQ ID NOS 7 and 3, respectively, in order of appearance.

FIG. 4F. TAT-WVELHFFN (“WVELHFFN” disclosed as SEQ ID NO: 3) counteracts TAT-WVELAASN (“WVELAASN” disclosed as SEQ ID NO: 7) increase in relative mitochondrial swelling. Figure discloses SEQ ID NOS 7 and 3, respectively, in order of appearance.

FIG. 4G. TAT-WVELHFFN (“WVELHFFN” disclosed as SEQ ID NO: 3) counteracts TAT-WVELAASN (“WVELAASN” disclosed as SEQ ID NO: 7) increase in relative active BAX concentration. Figure discloses SEQ ID NOS 7 and 3, respectively, in order of appearance.

FIG. 4H. TAT-WVELHFFN (“WVELHFFN” disclosed as SEQ ID NO: 3) counteracts TAT-WVELAASN (“WVELAASN” disclosed as SEQ ID NO: 7) increase in relative cytochrome c concentration. Figure discloses SEQ ID NOS 7 and 3, respectively, in order of appearance.

FIG. 41. TAT-WVELHFFN (“WVELHFFN” disclosed as SEQ ID NO: 3) counteracts TAT-WVELAASN (“WVELAASN” disclosed as SEQ ID NO: 7) increase in relative Caspase 3 activity. Figure discloses SEQ ID NOS 7 and 3, respectively, in order of appearance.

FIG. 5A. BNIP3 accumulation. Figure discloses SEQ ID NOS 7 and 3, respectively, in order of appearance.

FIG. 5B. BAX accumulation. Figure discloses SEQ ID NOS 7 and 3, respectively, in order of appearance.

FIG. 5C. BAX accumulation. Figure discloses SEQ ID NOS 7 and 3, respectively, in order of appearance.

FIG. 5D. BAX accumulation. Figure discloses SEQ ID NO: 3.

FIG. 5E. BAX and BNIP3 coimmunoprecipitation results. Figure discloses “WVELHFFN-Fluo” as SEQ ID NO: 9.

FIG. 5F. BAX and BNIP3 accumulation results.

FIG. 5G. BNIP3 accumulation impacted by TAT-WVELHFFN (“WVELHFFN” disclosed as SEQ ID NO: 3). Figure discloses SEQ ID NO: 3.

FIG. 5H. Accumulation results. Figure discloses SEQ ID NO: 3.

FIG. 5I. TAT-WVELHFFN (“WVELHFFN” disclosed as SEQ ID NO: 3) migration in baseline and I/R tissues. Figure discloses SEQ ID NO: 9.

FIG. 6A. Assay time course schematic.

FIG. 6B. AAR/LV % for various treatments. Figure discloses SEQ ID NOS 7 and 3, respectively, in order of appearance.

FIG. 6C. TAT-WVELHFFN (“WVELHFFN” disclosed as SEQ ID NO: 3) improves Inf/AAR %. Figure discloses SEQ ID NOS 7 and 3, respectively, in order of appearance.

FIG. 6D. TAT-WVELHFFN (“WVELHFFN” disclosed as SEQ ID NO: 3) improves Inf/AAR % at concentrations below 20 nmol. Figure discloses SEQ ID NO: 3.

FIG. 6E. TAT-WVELHFFN (“WVELHFFN” disclosed as SEQ ID NO: 3) reduces ApopTag events. Figure discloses SEQ ID NO: 3.

FIG. 6F. Treatment image. Figure discloses SEQ ID NO: 3.

FIG. 6G. deltaTM and deltaTM/TAT-WVELHFFN (“WVELHFFN” disclosed as SEQ ID NO: 3) reduce Inf/AAR %. Figure discloses SEQ ID NO: 3.

FIG. 6H. TAT-WVELHFFN (“WVELHFFN” disclosed as SEQ ID NO: 3) reduces Inf/AAR and Inf/LV in pig infarction events. Figure discloses “WVELHFFN” as SEQ ID NO: 3.

FIG. 6I. TAT-WVELHFFN (“WVELHFFN” disclosed as SEQ ID NO: 3) reduces Inf/AAR in pig ischemic stroke events. Figure discloses SEQ ID NO: 3.

FIG. 6J. Treatment course.

FIG. 6K PepB treatment reduces Inf/AAR.

FIG. 6L. Pre-treatment preserves cardiac function over control.

FIG. 6M. Treatment course.

FIG. 6N. Additional post-treatment improves cardiac function over control.

FIG. 7A. Assay flow diagram related to FIG. 1B.

FIG. 7B. TAT-BNIP3 reduces signal in BnipY background related to FIG. 1D.

FIG. 7C. deltaTM reduces Inf/AAR relative to vehicle related to FIG. 1E.

FIG. 8A. Mitochondrial depolarization assays related to FIG. 1G.

FIG. 8B. Mitochondrial swelling and damage assays related to FIG. 1H and FIG. 1I.

FIG. 8C. Flow cytometry results related to FIG. 1G.

FIG. 8D. Mitochondrial images related to FIG. 1H and FIG. 1I.

FIG. 8E. Heart and cardiomyocyte protein blot related to FIG. 1J and FIG. 1K.

FIG. 8F. BNIP3 mRNA accumulation levels related to FIG. 1J and FIG. 1K.

FIG. 8G. Relative BNIP3 protein concentrations related to FIG. 1J and FIG. 1K.

FIG. 8H. Subcellular fractionation studies for BNIP3 monomer and dimer. Dimers indicate increased mitochondrial localization related to FIG. 1J and FIG. 1K.

FIG. 8I. Mouse heart localization images related to FIG. 1J and FIG. 1K.

FIG. 9A. Accumulation levels for relative cytochrome c, AF, Active BAX, for Vehicle, DIDS, BAX and deltaTM, and assay schematic related to FIG. 1L.

FIG. 9B. Assay schematic related to FIG. 1L.

FIG. 9C. Assay schematic related to FIG. 1L.

FIG. 9D. Relative BNIP3 phosphorylation and Active BAX concentration are impacted by AKT inhibition related to FIG. 1Q.

FIG. 10A. Mutation alanine scan heat map related to FIG. 3H. Figure discloses SEQ ID NOS 54-86, respectively, in order of appearance.

FIG. 10B. WVELAASN (SEQ ID NO: 7) folding prediction related to FIG. 3L.

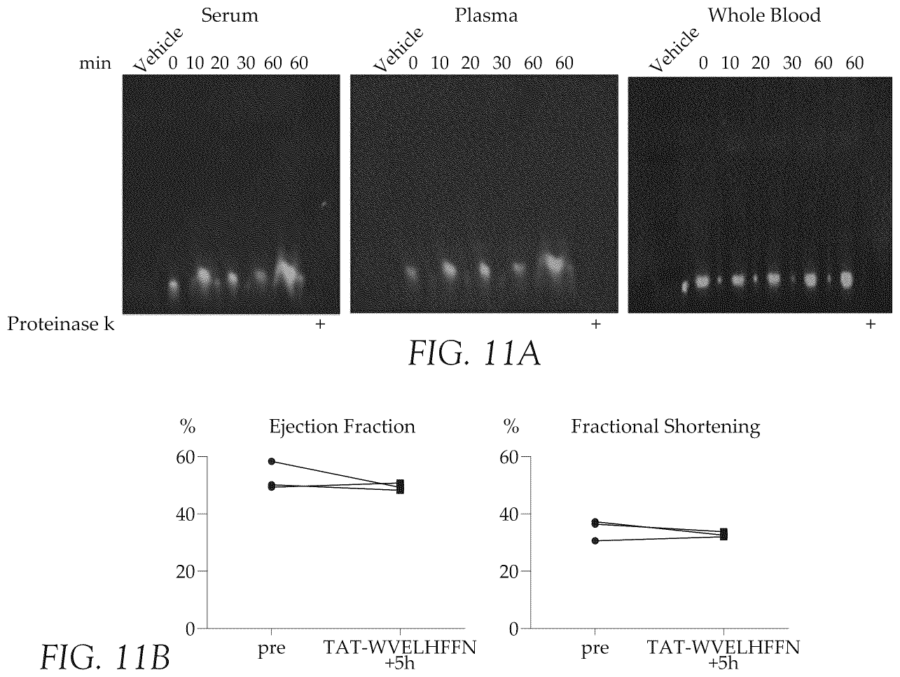

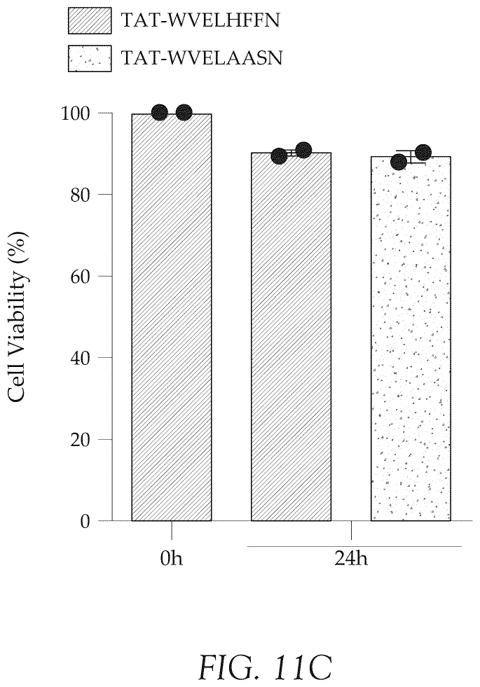

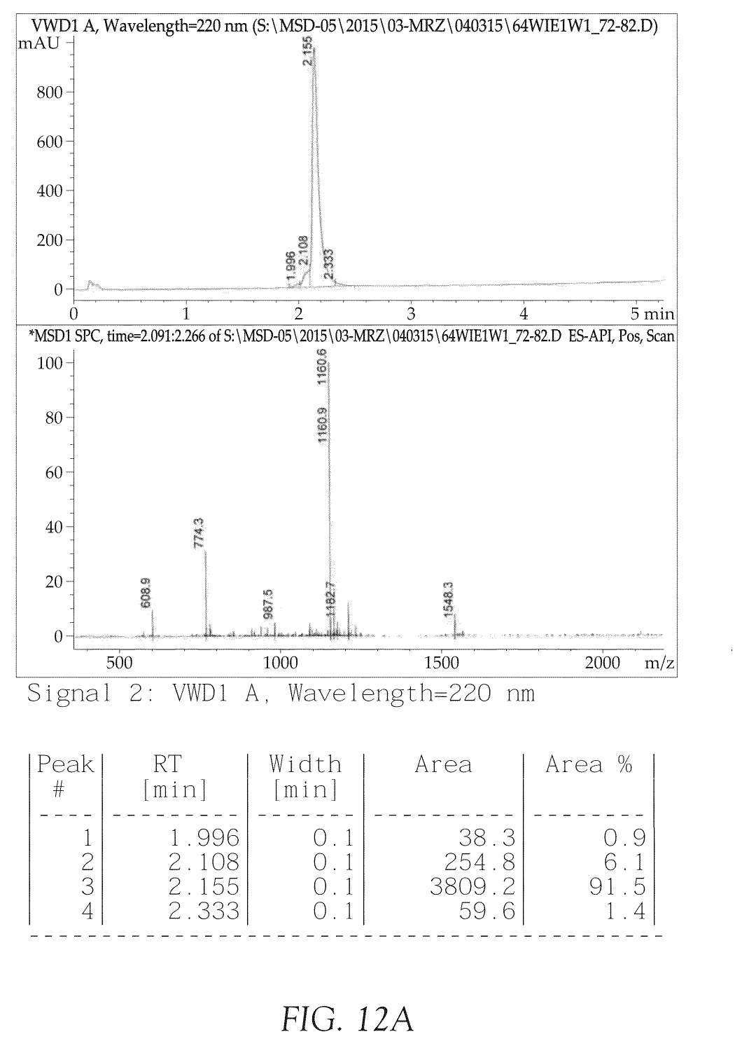

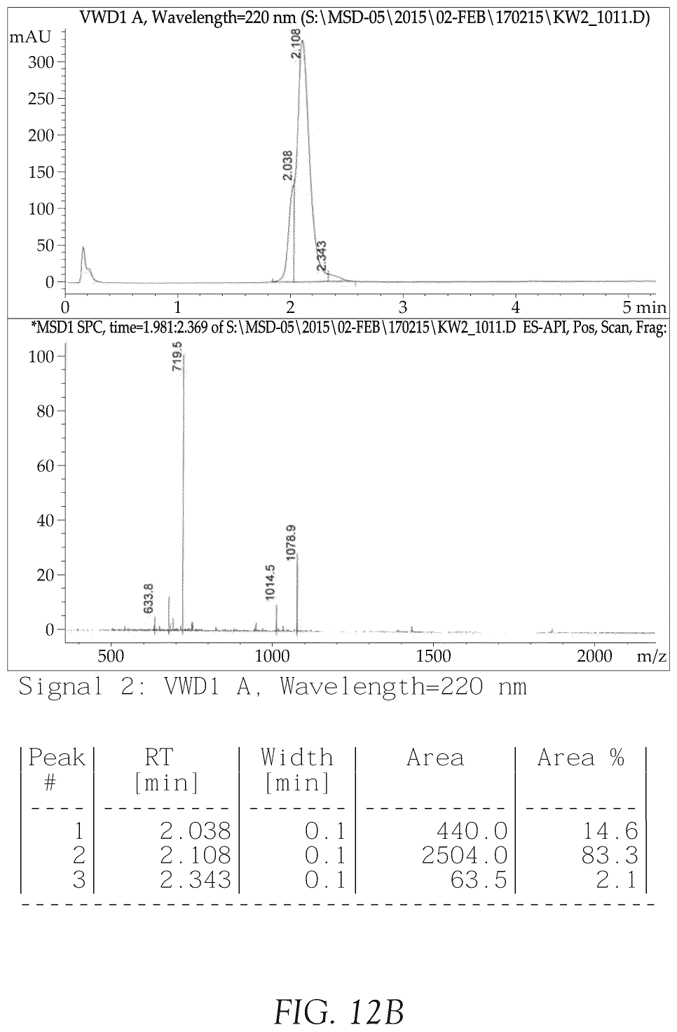

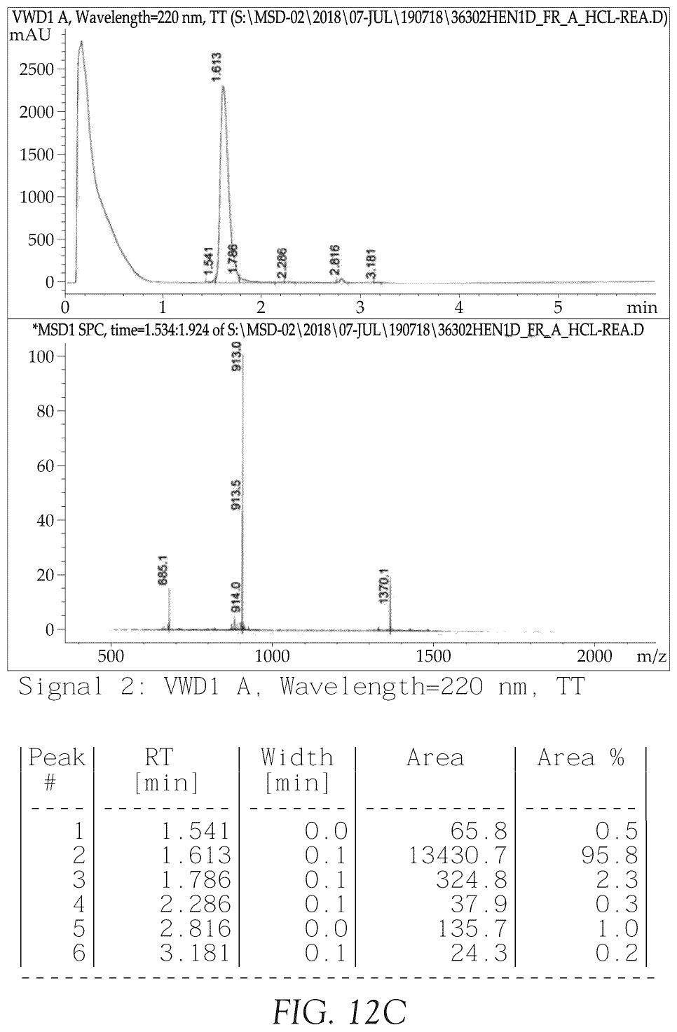

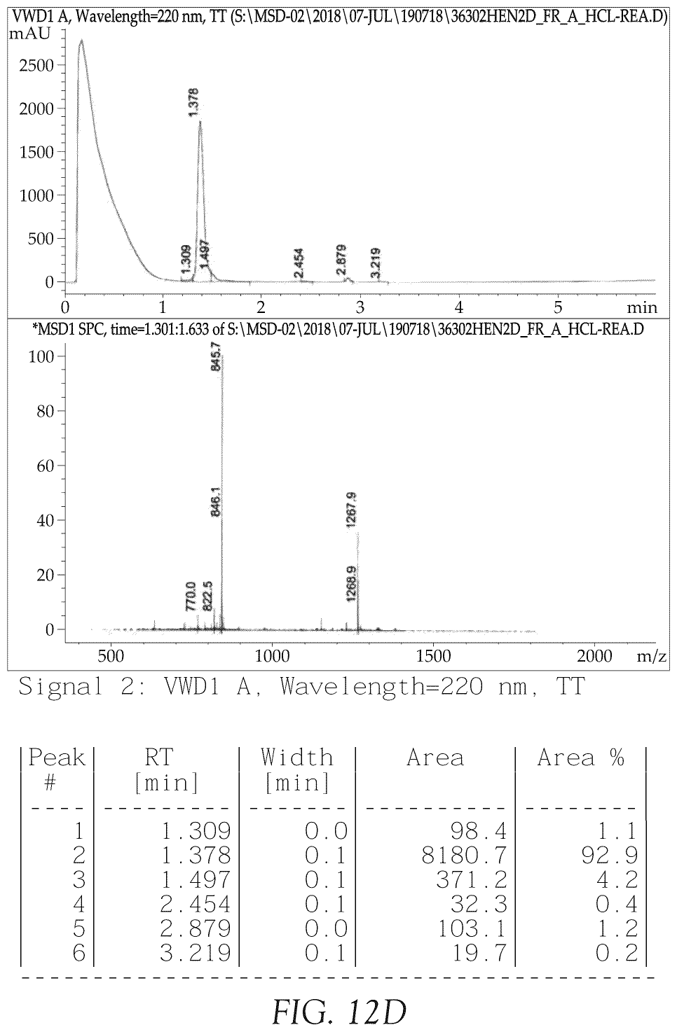

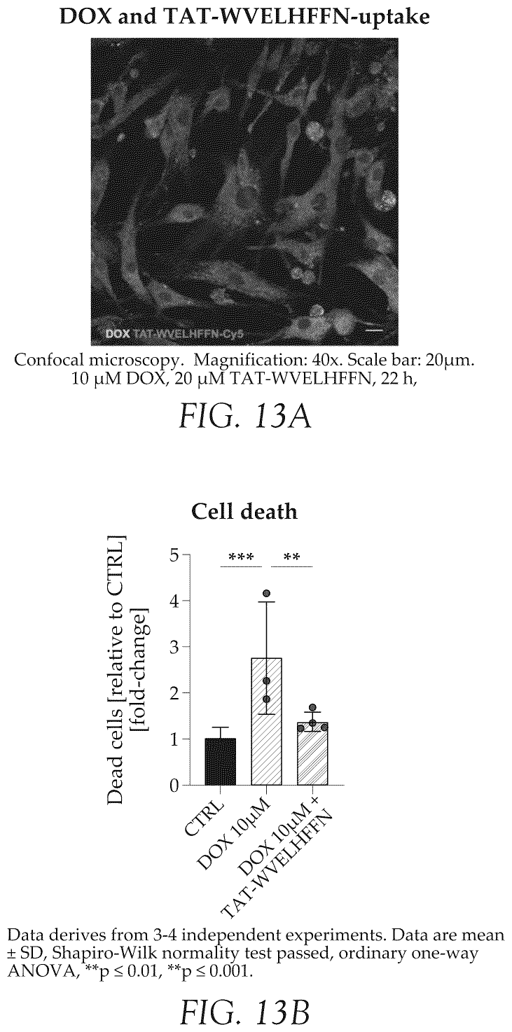

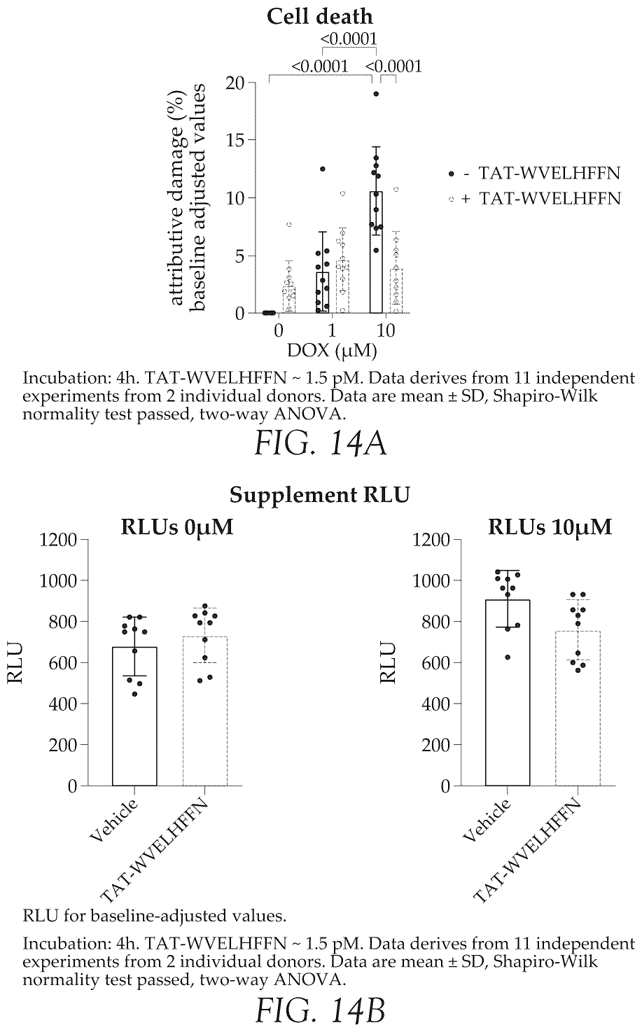

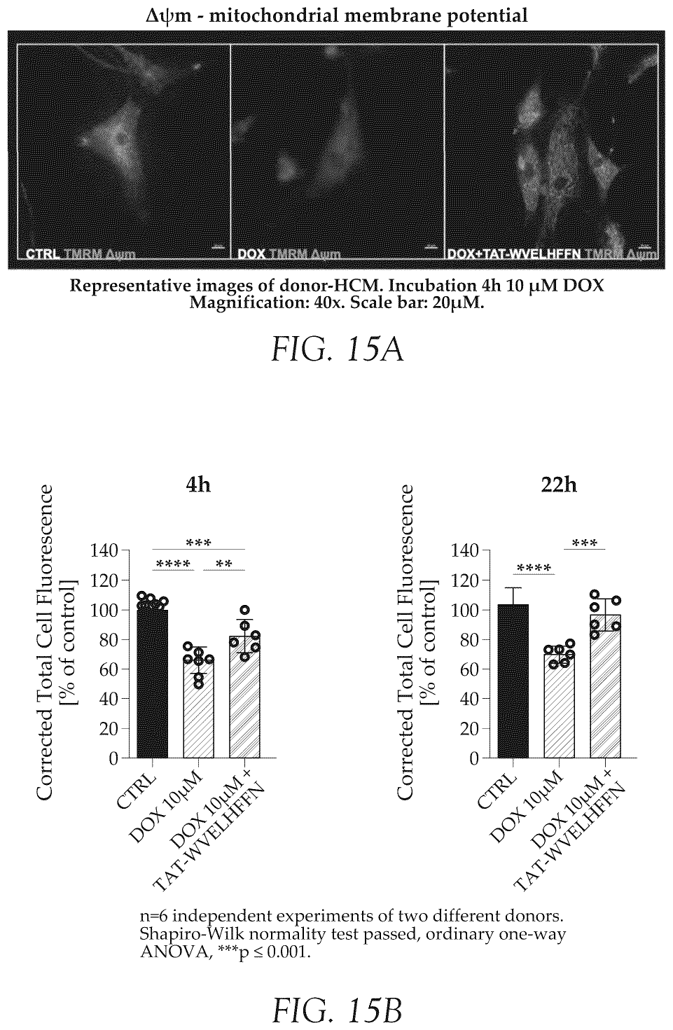

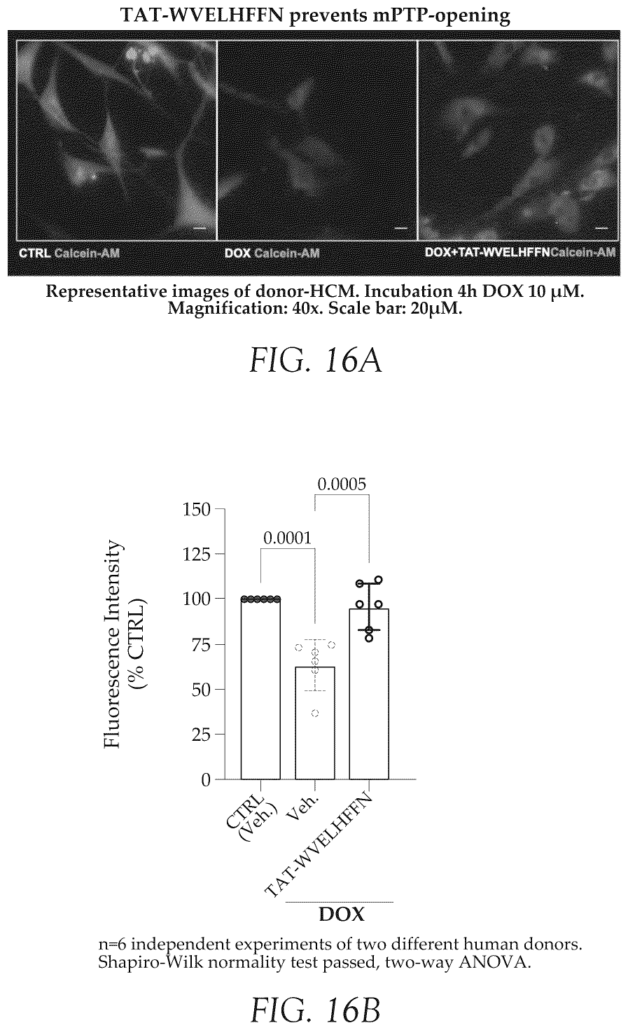

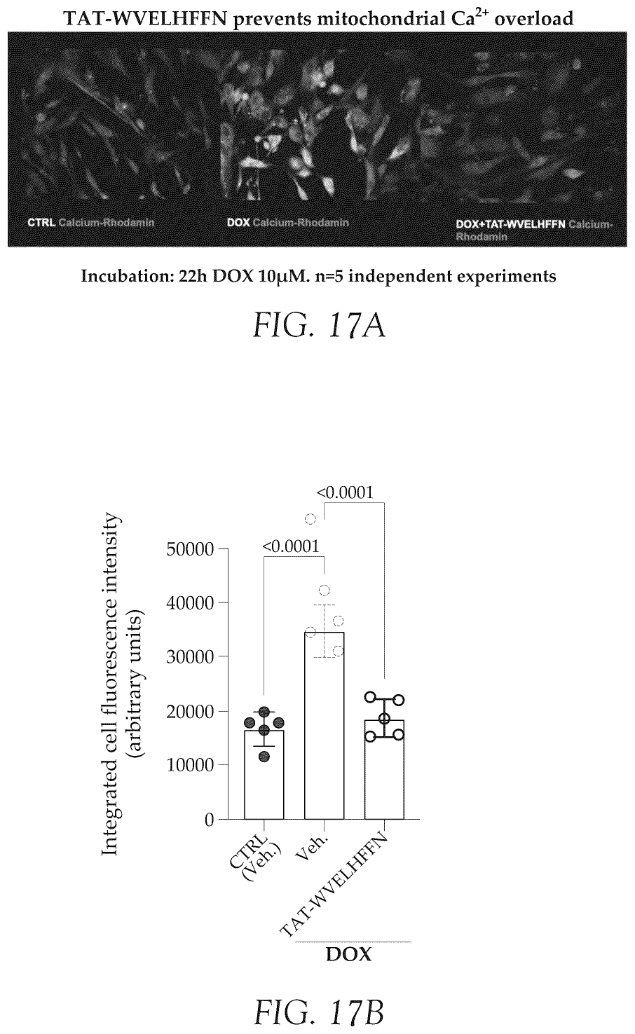

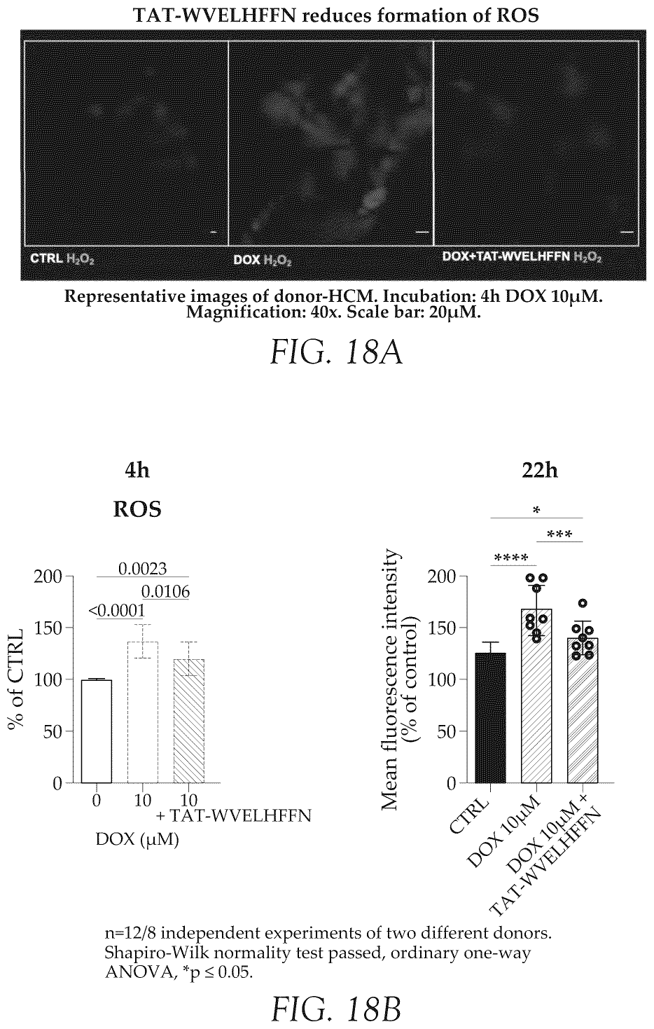

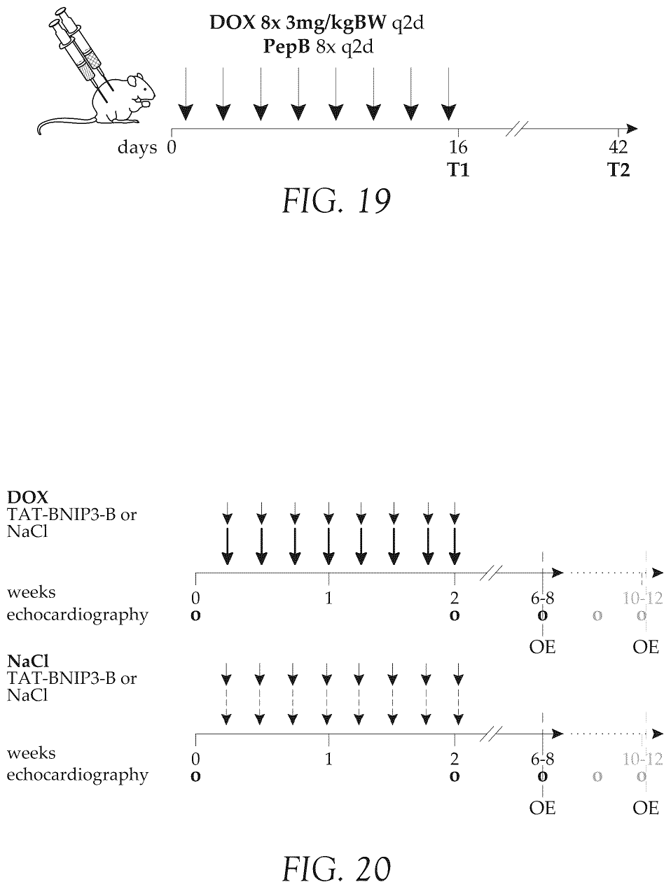

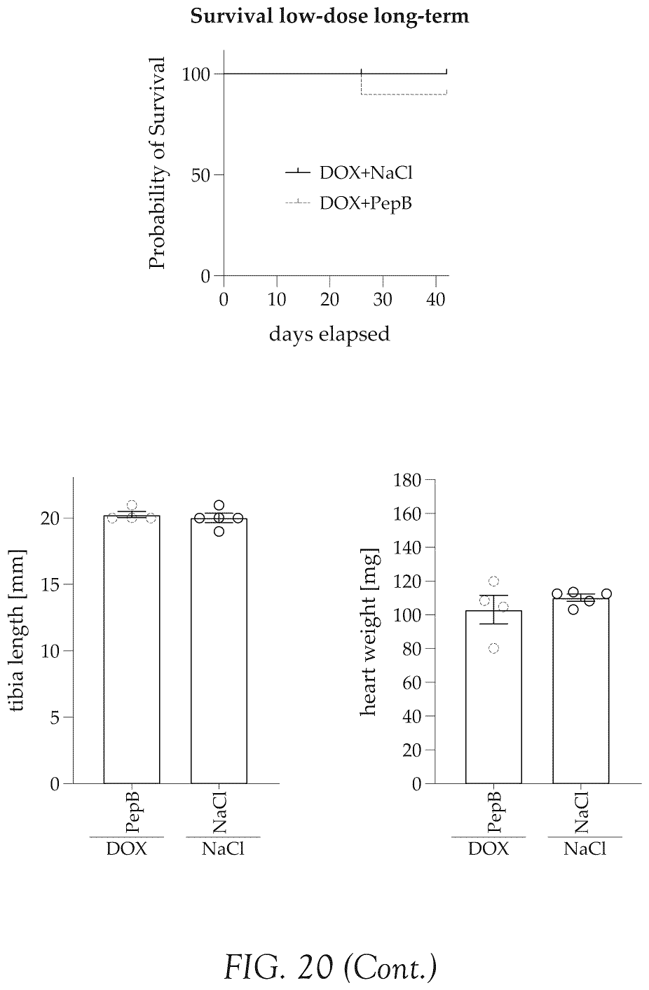

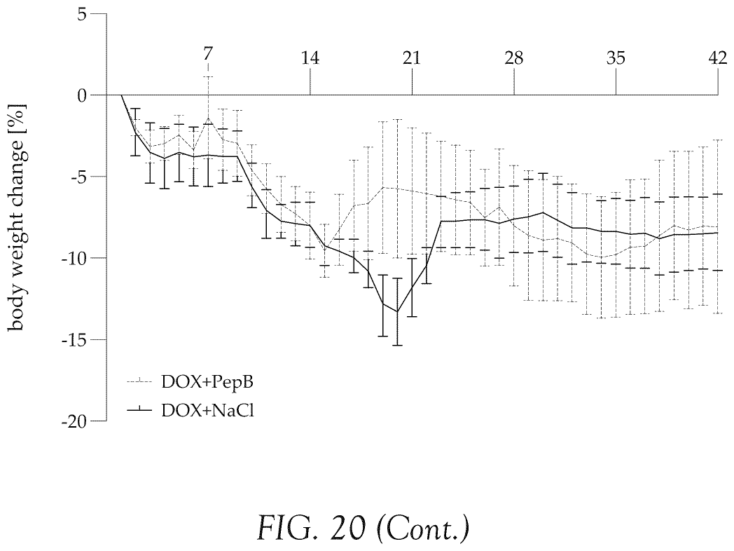

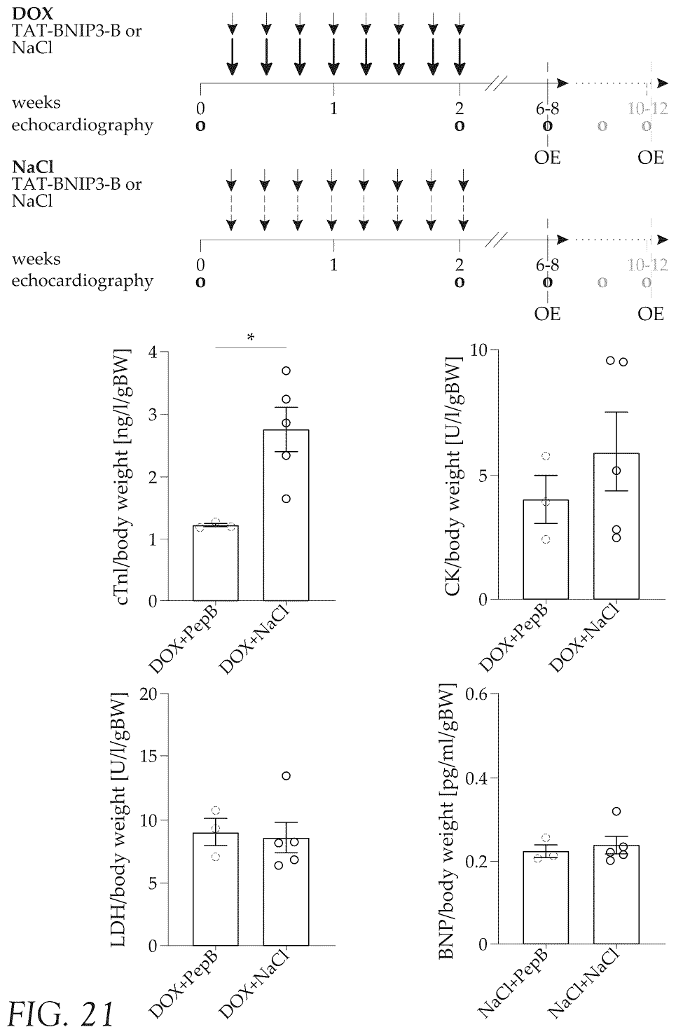

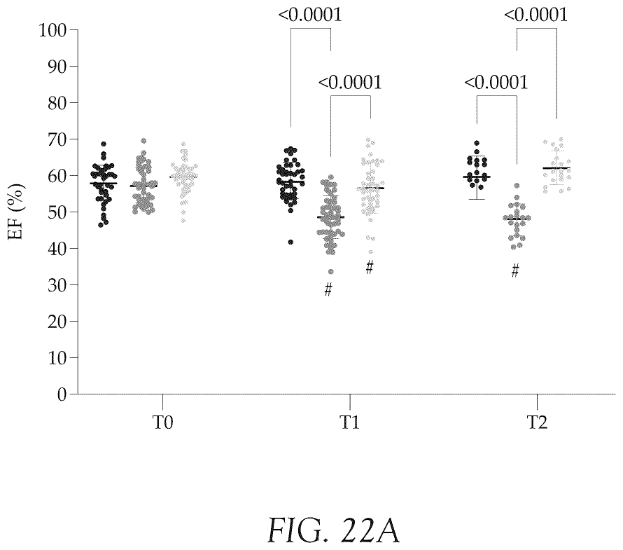

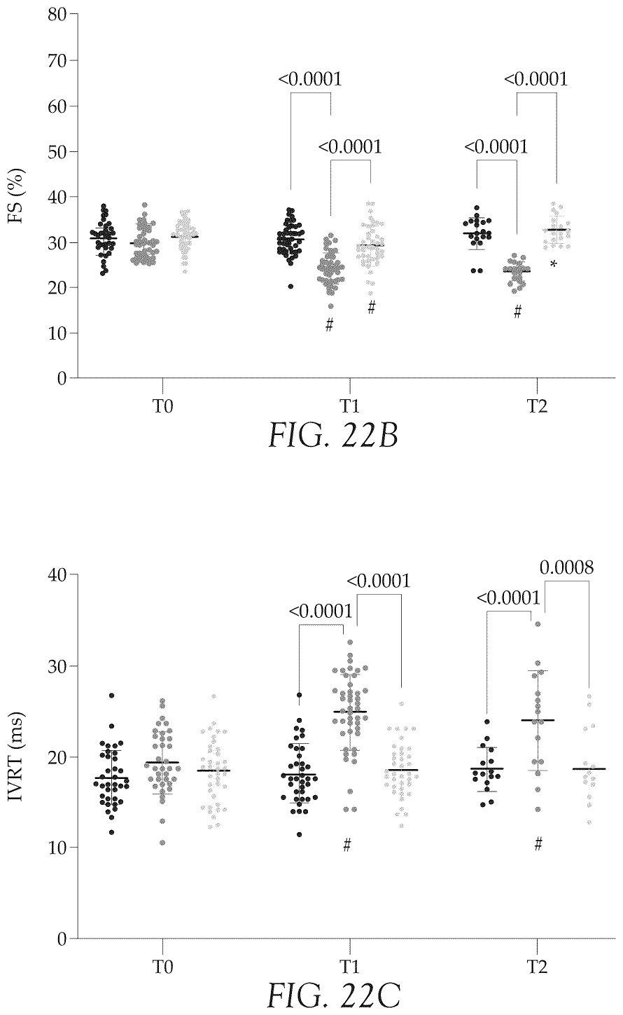

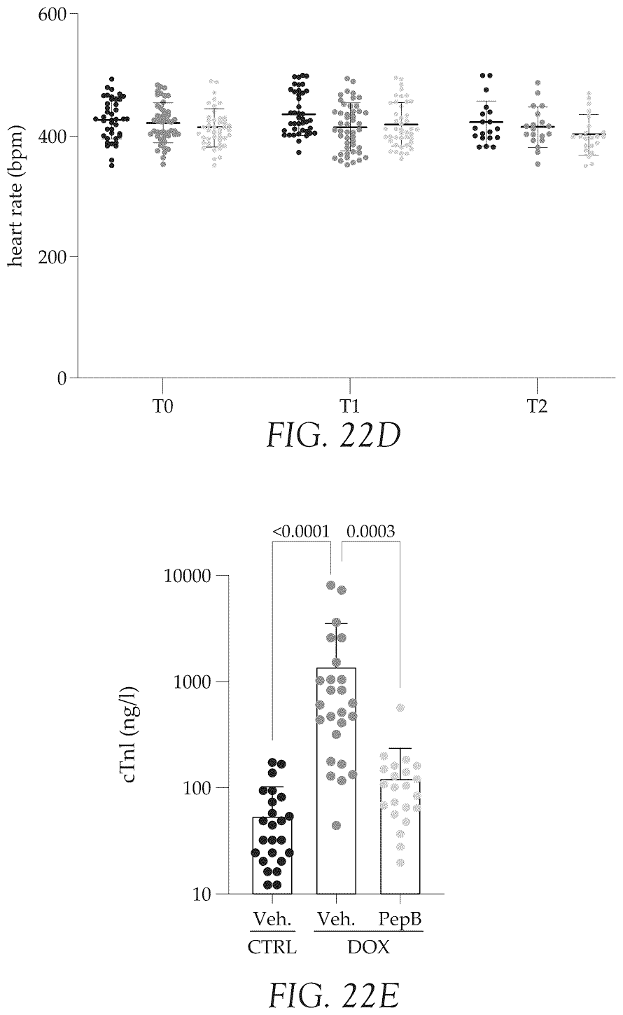

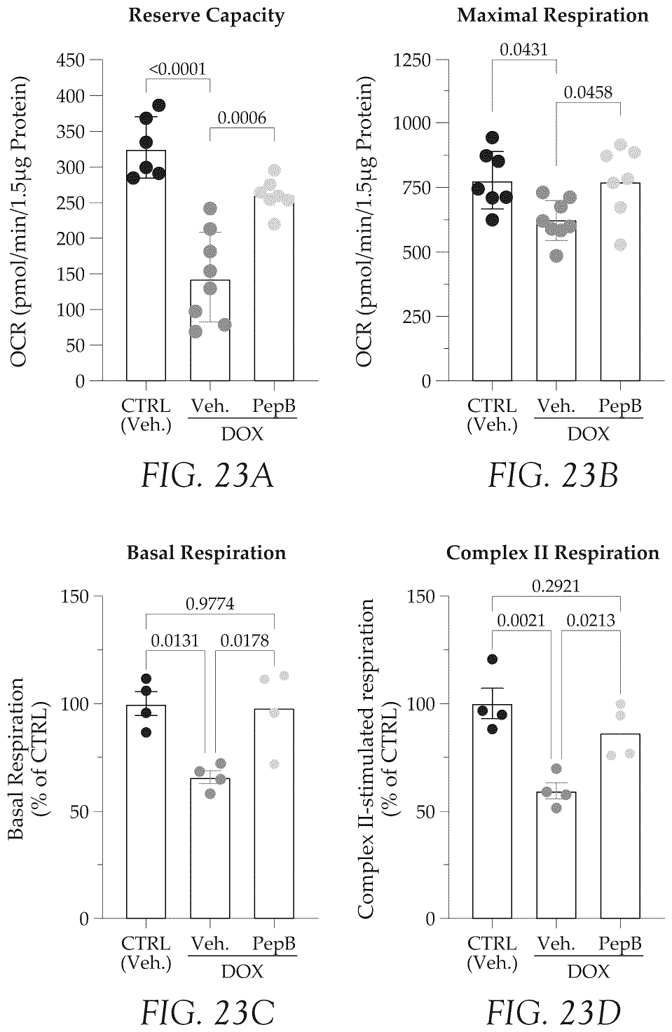

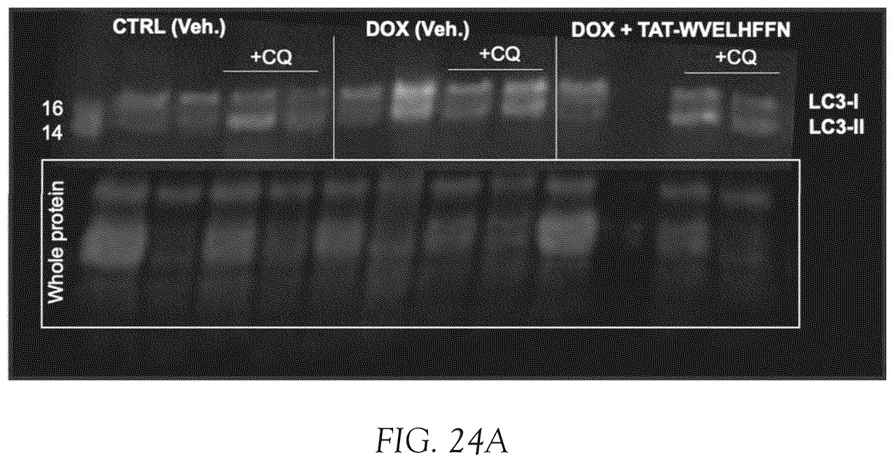

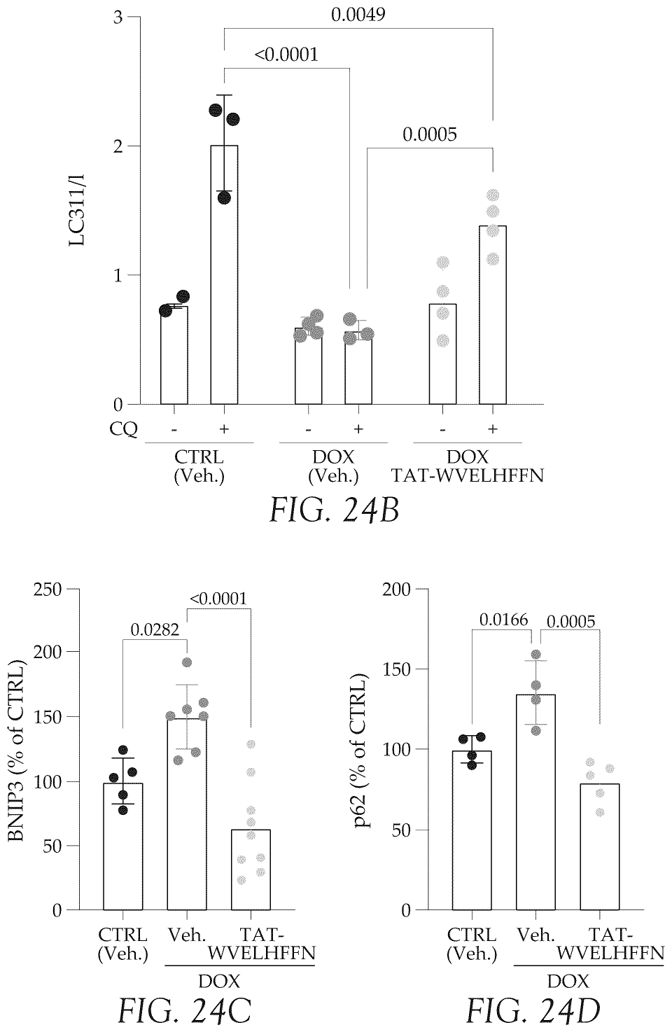

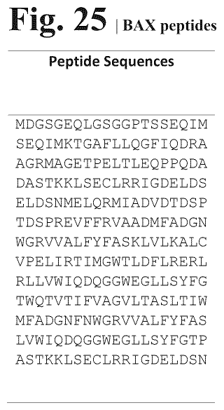

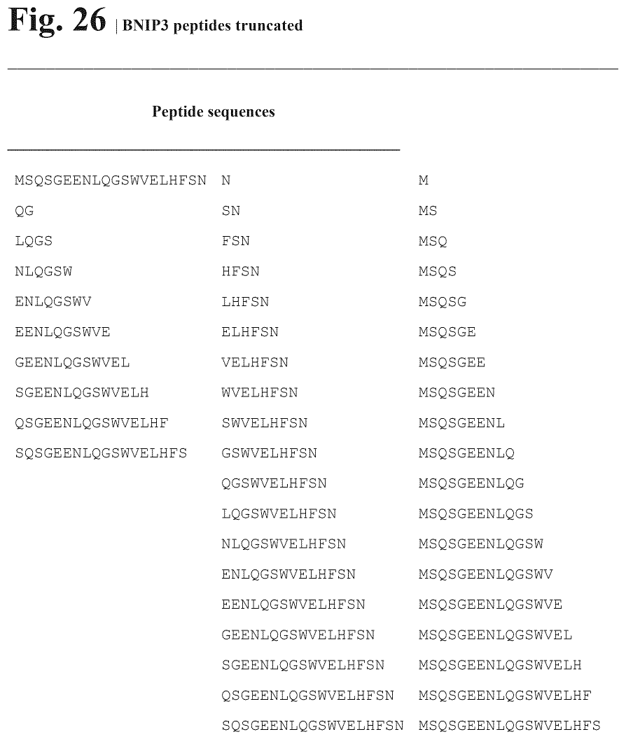

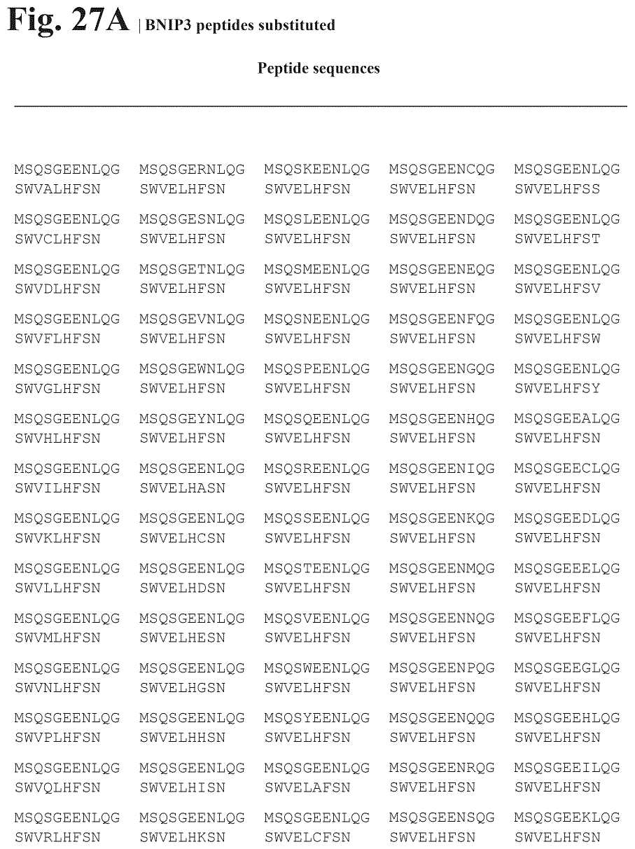

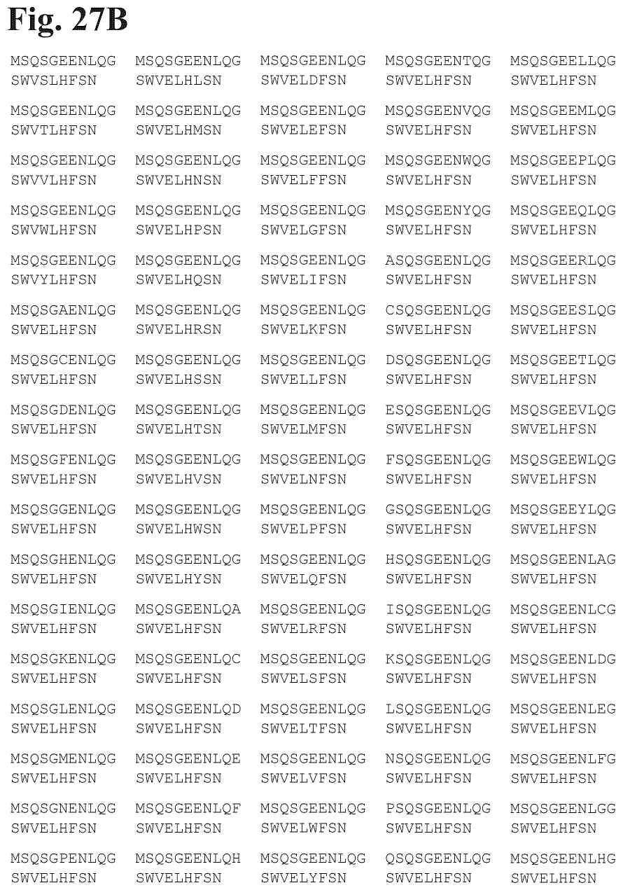

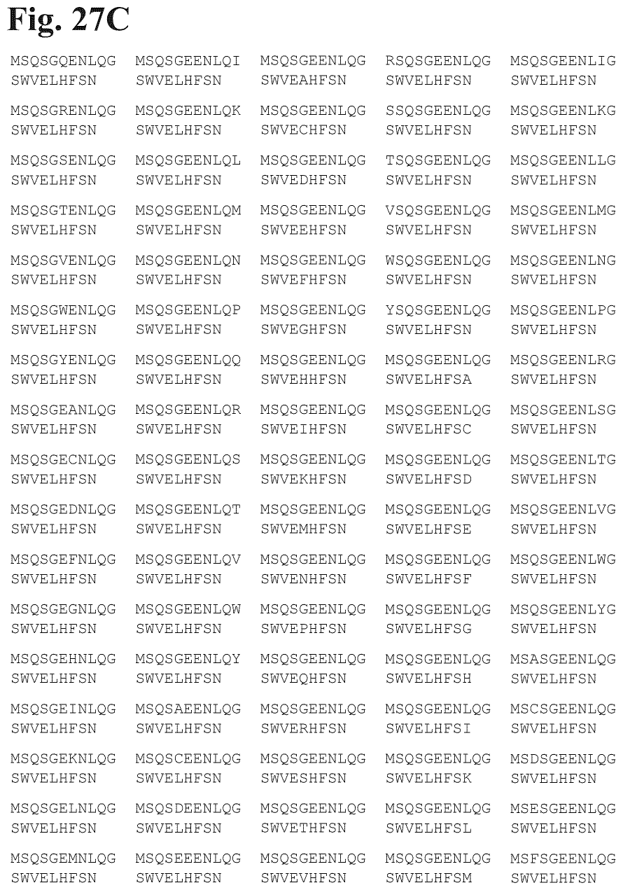

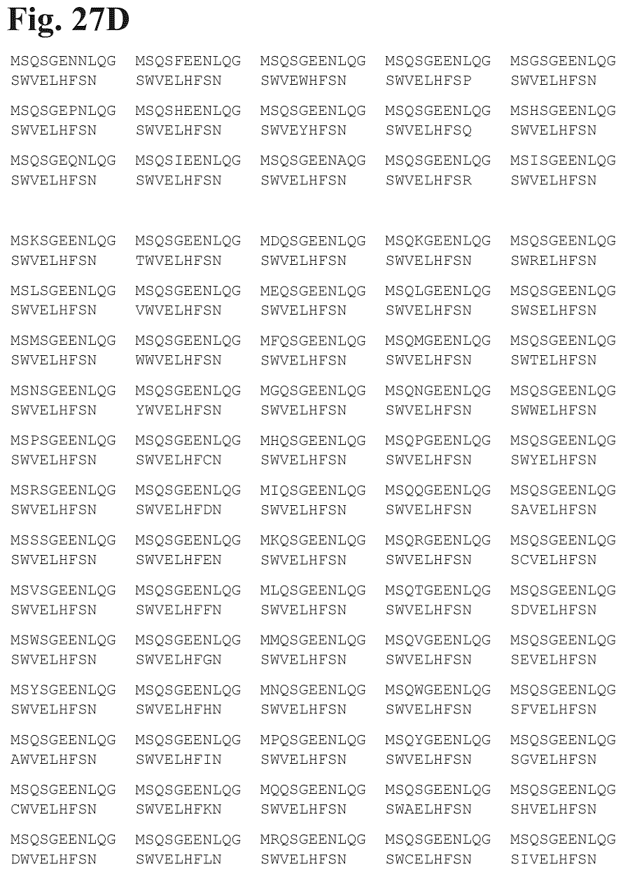

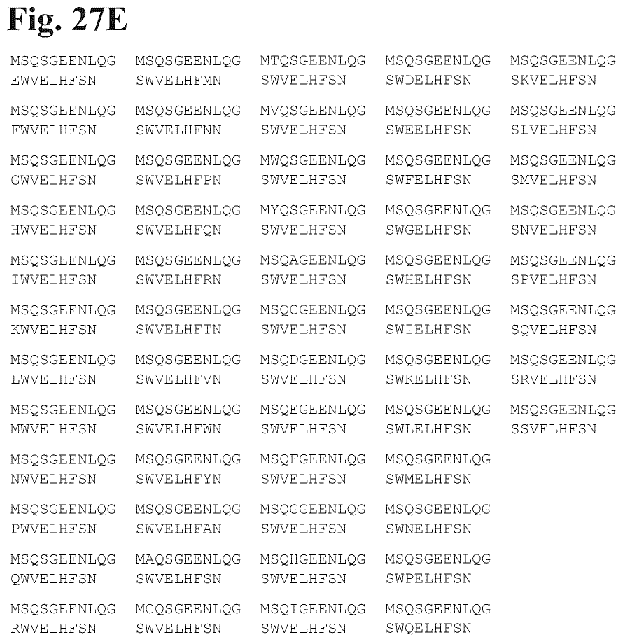

FIG. 11A. Proteinase K accumulation related to FIG. 4B. Figure discloses “WVELAASN” as SEQ ID NO: 7.

FIG. 11B WVELHFFN (SEQ ID NO. 3) impact on ejection fraction and fractional shortening related to FIG. 4B. Figure discloses “WVELHFFN” as SEQ ID NO: 3.

FIG. 11C. TAT-WVELAASN (“WVELAASN” disclosed as SEQ ID NO: 7) and TAT-WVELHFFN (“WVELHFFN” disclosed as SEQ ID NO: 3) have comparable effects on cell viability related to FIG. 4B. Figure discloses SEQ ID NOS 3 and 7, respectively, in order of appearance.

FIG. 12A. Confirmatory synthesis assay.

FIG. 12B. Confirmatory synthesis assay.

FIG. 12C. Confirmatory synthesis assay.

FIG. 12D. Confirmatory synthesis assay.

FIG. 13A. DOX and TAT-WVELHFFN (“WVELHFFN” disclosed as SEQ ID NO: 3) uptake. Figure discloses SEQ ID NOS 3, 10 and 3, respectively in order of appearance.

FIG. 13B. TAT-WVELHFFN (“WVELHFFN” disclosed as SEQ ID NO. 3) counters DOX induced cell death. Figure discloses SEQ ID NO: 3.

FIG. 14A. TAT-WVELHFFN (“WVELHFFN” disclosed as SEQ ID NO: 3) counters DOX induced cell death at high DOX concentrations. Figure discloses “WVELHFFN” as SEQ ID NO: 3.

FIG. 14B. TAT-WVELHFFN (“WVELHFFN” disclosed as SEQ ID NO: 3) reduces RLU count at 10 uM DOX administration. Figure discloses “WVELHFFN” as SEQ ID NO: 3.

FIG. 15A. TAT-WVELHFFN (“WVELHFFN” disclosed as SEQ ID NO. 3) counters DOX induced mitochondrial membrane potential loss. Figure discloses SEQ ID NO: 3.

FIG. 15B. TAT-WVELHFFN (“WVELHFFN” disclosed as SEQ ID NO: 3) counters DOX induced mitochondrial membrane potential loss. Figure discloses “WVELHFFN” as SEQ ID NO: 3.

FIG. 16A. TAT-WVELHFFN (“WVELHFFN” disclosed as SEQ ID NO: 3) counters DOX induced mPTP-opening. Figure discloses “WVELHFFN” as SEQ ID NO: 3.

FIG. 16B. TAT-WVELHFFN (“WVELHFFN” disclosed as SEQ ID NO: 3) counters DOX induced mPTP-opening. Figure discloses SEQ HD NO: 3.

FIG. 17A. TAT-WVELHFFN (“WVELHFFN” disclosed as SEQ ID NO: 3) counters DOX induced calcium ion accumulation. Figure discloses “WVELHFFN” as SEQ ID NO: 3.

FIG. 17B. TAT-WVELHFFN (“WVELHFFN” disclosed as SEQ ID NO: 3) counters DOX induced calcium ion accumulation. Figure discloses SEQ ID NO: 3.

FIG. 18A. TAT-WVELHFFN (“WVELHFFN” disclosed as SEQ ID NO: 3) counters DOX induced reactive oxygen species (ROS) accumulation. Figure discloses “WVELHFFN” as SEQ ID NO: 3.

FIG. 18B. TAT-WVELHFFN (“WVELHFFN” disclosed as SEQ ID NO. 3) counters DOX induced reactive oxygen species (ROS) accumulation. Figure discloses “WVELHFFN” as SEQ ID NO: 3.

FIG. 19. Treatment regimen schematic.

FIG. 20. Survival curves, body weight change measurements, tibial total length and heart weight measurements indicate that TAT-WVELHFFN (“WVELHFFN” disclosed as SEQ ID NO: 3) does not impact long term survival of healthy mice.

FIG. 21. TAT-WVELHFFN (“WVELHFFN” disclosed as SEQ ID NO: 3) influences cardiac troponin I/body weight and creatine kinase/body weight but not LDH/body weight or BNP/body weight for the indicated treatment regimen.

FIG. 22A. For non-zero time points, TAT-WVELHFFN (“WVELHFFN” disclosed as SEQ ID NO: 3) (right data points) counteracts the negative effect of DOX (center data points) to restores cardiac function to vehicle levels (left data points).

FIG. 22B. For non-zero time points, TAT-WVELHFFN (“WVELHFFN” disclosed as SEQ ID NO: 3) (right data points) counteracts the negative effect of DOX (center data points) to restores cardiac function to vehicle levels (left data points).

FIG. 22C. For non-zero time points, TAT-WVELHFFN (“WVELHFFN” disclosed as SEQ ID NO: 3) (right data points) counteracts the effect of DOX (center data points) to restores cardiac function to vehicle levels (left data points).

FIG. 22D. TAT-WVELHFFN (“WVELHFFN” disclosed as SEQ ID NO: 3) and DOX do not affect heart rate.

FIG. 22E. TAT-WVELHFFN (“WVELHFFN” disclosed as SEQ ID NO: 3) counteracts the effect of DOX to prevent cardiomyocyte death to vehicle levels.

FIG. 23A. TAT-WVELHFFN (“WVELHFFN” disclosed as SEQ ID NO: 3) counteracts the effect of DOX to maintain respiratory reserve capacity.

FIG. 23B. TAT-WVELHFFN (“WVELHFFN” disclosed as SEQ ID NO: 3) counteracts the effect of DOX to maintain maximal respiration capacity.

FIG. 23C. TAT-WVELHFFN (“WVELHFFN” disclosed as SEQ ID NO: 3) counteracts the effect of DOX to maintain basal respiration to vehicle levels.

FIG. 23D. TAT-WVELHFFN (“WVELHFFN” disclosed as SEQ ID NO: 3) counteracts the effect of DOX to maintain Complex II stimulated respiration to vehicle levels as a measure of Reserve Capacity.

FIG. 24A. Protein blots. Figure discloses SEQ ID NO: 3.

FIG. 24B. TAT-WVELHFFN (“WVELHFFN” disclosed as SEQ ID NO: 3) partially restores LC3II/I levels in response to cloroquine addition to DOX samples. Figure discloses SEQ ID NO: 3.

FIG. 24C. TAT-WVELHFFN (“WVELHFFN” disclosed as SEQ ID NO: 3) counteracts DOX induced BNIP3 accumulation. Figure discloses SEQ ID NO: 3.

FIG. 24D. TAT-WVELHFFN (“WVELHFFN” disclosed as SEQ ID NO. 3) counteracts DOX induced p62 accumulation.

FIG. 25. BAX peptide sequences. Figure discloses SEQ ID NOS 87, 494, 88-91, 26, 33-34, 29, 92, 31, and 93, respectively in order of appearance.

FIG. 26. BNIP3 peptides truncated. Figure discloses SEQ ID NOS 1, 94-101, 53, 52, 51, 8, 2, 50, 49, 48, 47, 46, 45, 44, 43, 42, 41, 40, and 102-117, respectively, in order of columns.

FIG. 27A-FIG. 27E. BNIP3 peptides substituted. Figures disclose discloses SEQ ID NOS 118-493, respectively, in order of appearance.

### DETAILED DESCRIPTION

To improve outcomes of individuals suffering from or at risk of infarction damage resulting from disruption in oxygenation at an affected area, we developed methods and compositions for the treatment of or amelioration of symptoms of disruption in oxygenation to an affected area. This work was then expanded to include a broad range of approaches for amelioration of secondary damage pursuant to an intervention, particularly by effecting mitochondrial stabilization, such as through the inhibition of mitochondrial interaction and import involved in mitochondrial destruction signaling.

Our research identified systems, methods and compositions for the amelioration, or treatment of ischemia/reperfusion (I/R) injury. In particular, we disclose herein compositions and methods for treatment of I/R injury conditions, for the prophylactic treatment of individuals at risk of I/R injury as well as for the additional post-treatment of individuals at risk of I/R injury consequences, so as to reduce the harm that may arise from a subsequent I/R event in an individual at risk thereof. We further disclose compositions and methods for the amelioration of a number of interventions associated with detrimental effects arising from mitochondrial destabilization.

We used unbiased biochemical approaches, computational modeling, and in vivo models of myocardial and brain I/R injury caused by a mixture of necrotic and apoptotic cell death as clinically relevant and elegant systems. Our results identify interaction and translocation across mitochondrial membranes as a mechanism to perturbate to reduce mitochondrial cell death signaling in response to reperfusion and to other sources of secondary deleterious effects. We further identify, as a particular example validating this mechanism, cytosolic BNIP3 as a key regulator of BAX in a heteromeric state for both mitochondrial necrotic and apoptotic signaling in mammalian cells including human cardiomyocytes.

As a specific example relating to the general idea of mitochondrial stabilization, we found that cytosolic oligomers comprising BNIP3/BAX heterodimers are critical mitochondrial membrane attack complexes with BNIP3-phosphorylation as the master switch for necrotic and apoptotic cell death-inducing activity. By combining in silico BNIP3 3D modelling with structural and binding behavioral interrogation of BNIP3 and BAX-derived peptides, we developed a BNIP3 octapeptide antagonist that prevents the identified mitochondrial membrane attack of oligomeric BNIP3/BAX heterodimers. This effectively reduces cell death in human cardiomyocytes, mouse and pig tissues in I/R injury by 40-60%. These results were found to generalizable to other sources of secondary deleterious effects, such those associated with chemotherapeutic intervention, where mitochondrial stabilization through import inhibition yielded similar improvements in outcomes in cell death in human cardiomyocytes, mouse and pig tissue

Our results have implications for understanding the molecular mechanism of mitochondrial membrane disintegration leading to necrosis and apoptosis and pinpoint the therapeutic potential of a BNIP3 antagonist for I/R injury conditions such as myocardial infarction and stroke in humans. Our results further have implications for methods of treatment and compositions for use in treatment, in that incorporation of a mitochondrial protecting and/or stabilizing agent such as a mitochondrial interaction and import inhibitor as disclosed herein may have a substantial positive effect in the reduction of secondary cell death.

Mitochondria are essential end effectors in physiological and pathological cell death induction (Kwong and Molkentin, 2015). When damaged, they translate their injury into lethal signaling pathways (Nunnari and Suomalainen, 2012). Previous studies postulated BNIP3 as a potential upstream modulator of BAX disturbing MOM and MIM integrity and inducing necrotic and apoptotic cell death (Hamacher-Brady et al., 2007; Kubli et al., 2007, 2008). In particular, BNIP3 is suggested to exert its pro-death activity as stable homodimers localized to the MOM by orchestrating BAX activation (Kubli et al., 2007). However, fundamental issues regarding the underlying mechanisms remained unresolved, in part because the complete structure of BNIP3 is lacking. Since spin-labeling followed by pulsed electron paramagnetic resonance spectroscopy has identified that BNIP3 and BAX are able to heterodimerize (Hendgen-Cotta et al., 2017), we explored the activity of BNIP3/BAX in a potential heterodimeric state, which may cause mitochondrial disintegration. We developed a general strategy for preventing mitochondrial-induced necrosis and apoptosis based on further insights into underlying mechanisms. We have discovered that BNIP3 and BAX form inert heterodimers in the cytosol, which are assembled into higher molecular weight complexes under natural and I/R conditions. Thereby, the first 20 amino acids including a conserved region represent the functional domain in binding to BAX. These oligomeric BNIP/BAX heterodimers are mandatory for the interaction with mitochondria inducing subsequently the depolarization of the MIM. Unexpectedly, BAX activation and pore formation did not occur contemporaneously. This is important as BAX activation is required for the apoptotic signaling cascade (Tang et al., 2019). These observations therefore render a post-translational modification of BNIP3 the potential stimulus to excite activation of BAX by conformational changes. Phosphorylation modifies proteins from hydrophobic apolar to hydrophilic polar, allowing the protein to change conformation when interacting with other molecules (Ardito et al., 2017). Our data represent the first report of phosphorylation of BNIP3 in regulating BAX activation and pore formation. During ischemia and the early phase of reperfusion ATP synthesis is highly limited (Chen and Zweier, 2014), which explains the chronological sequence of BNIP3/BAX interaction-induced necrosis and apoptosis in I/R. Taken together, our findings suggest that phosphorylation of BNIP3 may transduce BAX conformation to form MOMP. Protection of mitochondria to avoid mitochondrial-driven cell death limiting tissue injury is currently an unmet therapeutic goal (Nunnari and Suomalainen, 2012). The striking influence of BNIP3/BAX heterodimeric activity under the control of BNIP3 on cell death in response to e.g., reperfusion therapy, which is indispensable to rescue patients during ischemic events (Heusch and Rassaf, 2016; Kloner et al., 2017), signify BNIP3/BAX heterodimers an outstanding novel target for the development of a small peptide antagonist. Guided by the newly modelled 3D structural information of BNIP3 and the specific N-terminal sequence behavioral interrogations, we discovered the crucial amino acids and engineered the cell-permeant octapeptide TAT-WVELHFFN (“WVELHFFN” disclosed as SEQ ID NO: 3) that inhibits oligomeric mitochondrial membrane attack complexes containing BNIP3/BAX heterodimers in the cytosol as defense against mitochondrial damage and cell death. Treatment with the BNIP3 octapeptide antagonist TAT-WVELHFFN (“WVELHFFN” disclosed as SEQ ID NO. 3) shows superior tissue protection in myocardial infarction and in stroke. Highlighting the translational potential of this approach, the octapeptide TAT-WVELHFFN (“WVELHFFN” disclosed as SEQ ID NO: 3) prevents cell death in mice, pigs and human cardiomyocytes.

Accordingly, disclosed herein are compositions, compositions for use and methods relating to amelioration of I/R damage, for example through administration of a compositions comprising a mitochondrial membrane interaction and translocation inhibitor or other mitochondrial integrity-preserving agent. More generally, disclosed herein are compositions, compositions for use and methods for amelioration of secondary damage caused by an intervention regimen, such as a chemotherapeutic, radiotherapeutic, a surgical intervention, or chronic condition, among others.

A number of mitochondrial membrane interaction and translocation inhibitor or other mitochondrial integrity-preserving agents are consistent with the disclosure herein. Some mitochondrial membrane interaction and translocation inhibitors or other mitochondrial integrity-preserving agents disclosed or contemplated herein share an effect of protecting mitochondria from degradation upon reintroduction of oxygen following an ischemic event. Others share a property of ameliorating a measurement of mitochondrial status change as presented in FIG. 13A through FIG. 25H, such as ROS accumulation, calcium ion accumulation, mitochondrial mPTP opening, mitochondrial membrane integrity preservation, caspase-3 activation or cytochrome c release. Protection from degradation variously comprises at least one of maintaining mitochondrial membrane potential, preventing cytochrome c release into the cytoplasm, reducing mitochondrial swelling, preventing protein translocation such as BAX protein or BNIP3 interaction with and translocation into the outer membrane, or other mitochondrial degradation. An effect of this protection is that there is no subsequent initiation of a cell death pathway in the affected cell, such that reperfusion of oxygen or therapeutic compound or regimen application can be accomplished without concurrent mitochondrial degradation and induction of cell death.

Attention is paid to particular protein, polypeptide or peptide mitochondrial membrane interaction and translocation inhibitors or other mitochondrial integrity-preserving agents. However, a number of protein, hormonal and small molecule effectors are consistent with the methods of the disclosure herein.

Representative mitochondrial integrity-preserving agent are seen in the substantial number of mitochondrial membrane translocation inhibitors derived from the BNIP3 near N-terminal fragments disclosed herein and previously in PCT Publication No. WO2020/229362, published Nov. 19, 2020, and incorporated by reference herein in its entirety.

Exemplary segments described herein include BNIP3 segments, such as N-terminal segments of BNIP3, for example regions drawn from MSQSGEENLQGSWVELHFSN (SEQ ID NO: 1) of BNIP3, in particular WVELHFSN (SEQ ID NO: 2) of BNIP3. Some such composition present unmodified segments of BNIP3, while alternate compositions present modified BNIP3 segments, such as polypeptides or peptides comprising the sequence WVELHFFN (SEQ ID NO: 3). These proteins, polypeptides or peptides optionally comprise a cellular localization signal such as an HIV TAT localization signal at their N- or C-terminus.

A number of routes of administration are consistent with the methods and compositions herein. Exemplary routes of administration comprise direct administration of a mitochondrial interaction and translocation inhibitor or other mitochondrial integrity-preserving agent in a biocompatible solution to a mammalian subject such as a human patient. Methods often comprise intravenous administration via a peripheral or central vein, intra-arterial administration via a peripheral artery, intra-arterial administration via catheter directly into an impacted area such as an impacted ventricle, or intra-arterial administration via catheter into a corresponding coronary artery or arteries as well as intra-peritoneal. Routes of administration often correlate to the impacted region for which I/R damage is to be ameliorated, and may vary for target areas such as heart, brain, kidney, or other impacted area.

Consistent with these routes of administration, a number of carriers are contemplated as part of the disclosure herein. NaCl solutions, Ringer solution and most solutions consistent with human administration are compatible with the compositions and methods herein. Solutions are in some cases selected or engineered to have a desired molecular oxygen concentration. In some cases, solutions are selected to be administered prior to reperfusion or reoxygenation and are formulated to have an oxygen concentration that is at least as low as the local impacted area concentration. Alternately, some solutions are formulated to accomplish a ‘pre-oxygenation’ by harboring an oxygen concentration that is elevated but below that of a subsequently applied high oxygen reperfusion composition. Alternately, some compositions are delivered in solutions having a high, or reperfusion level of dissolved oxygen, so as in some cases to effect concurrent mitochondrial translocation inhibition and oxygenation.

In addition to a carrier, a interaction and mitochondrial translocation inhibitor or other mitochondrial integrity-preserving agent is optionally administered concurrently with or in a composition comprising one or more additional factors, such as anesthetics, sedatives, anticoagulants, antibiotics, or other factors conducive to amelioration of an infarction event, prevention of a further infarction event, patient recovery or toleration of a procedure or any other reason conducive to a beneficial outcome. Exemplary anticoagulant cofactors include heparins, GPIIb/IIIa antagonists, any kind of platelet inhibitors, among others. Pain relief factors consistent with coadministration include morphine and/or morphine derivates or any other drugs which inhibit/relief pain, for example, fentanyl, hydromorphone, morphine, oxycodone, oxymorphone, dilaudid, tramado, as well as butorphanol, an NSAID (nonsteroidal anti-inflammatory drug, such as ibuprofen, acetaminophen, Ketorolac or other pain medications consistent with liquid formulation. Compositions may alternately or additionally comprise a sedative, such as. Propofol or benzodiazepines. Similarly, compositions may alternately or additionally comprise an antibiotic.

Methods of administration optionally comprise concurrent administration through alternate routes of complementary medicaments, such as oral administration or inhalation. Examples include orally administered anticoagulants such as warfarin, orally administered painkillers such as opiates or NSAIS, among others, or inhaled or orally administered anesthetics, or antibiotics.

As disclosed herein, mitochondrial interaction and translocation inhibition has a beneficial effect in individuals suffering from an infarction event or undergoing a therapeutic intervention, and may be administered prior to, concurrent with or even subsequent to initiation of reoxygenation or application of the therapeutic. Compositions are variously administered no more than 48 hours, no more than 24 hours, no more than 12 hours, no more than 6 hours, no more than 5 hours, no more than 4 hours, no more than 3 hours, no more than 3 hours, no more than 1 hour, no more than 30 minutes, no more than 15 minutes, no more than 10 minutes, or no more than 5 minutes prior to reoxygenation. Administration is of a single dose, or alternately comprises an administration regimen of at least 2, 3, 4, 5, 6, 7, 8, 9, 10 or more than 10 doses. Doses comprise consecutive administration of comparable medicament amounts, or alternately comprise changes in dose volume or medicament concentration, such that in some cases effective composition may ‘ramp up’ from an initial low level prior to oxygenation.

Administration is often performed in a hospital setting, such as an operating room. Alternately, in some cases administration occurs before the patient enters the hospital or prior to surgery. For example, paramedics are in some cases able to administer the compositions herein as soon as they see a patient or as soon as an acute myocardial infarction is diagnosed or suspected. Early administration has the benefit of reducing time to infarction surgery, and extending the time that the mitochondrial interaction and translocation inhibitor or other mitochondrial integrity-preserving agent has to act prior to reoxygenation.

Administration is similarly performed prior to or concurrent with reperfusion of the ischemic myocardium. For example, using interventional catheter systems, a percutaneous coronary intervention is performed and the occluded coronary artery is recanalized using a wire, a balloon and/or a stent system. This procedure is often performed using anticoagulants like heparin to inhibit blood clotting, using platelets inhibitors like e.g. aspirin, clopidogrel, cangrelor, ticagrelor, prasugrel, GPIIB/IIIa antagonists, using drugs to inhibit/relief pain (like e.g. morphine or its derivates), using drugs to sedate and relax patients (like e.g. propofol, benzodiazepines etc.), oxygen in different concentration before, during and/or after the procedure. A mitochondrial interaction and translocation inhibitor is administered concurrently with, prior to subsequent to, or even additional over a certain period of time post such a procedure.

In some cases, myocardial infarction is treated using fibrinolysis. Fibrinolytic therapy is an important reperfusion strategy in settings where primary PCI cannot be offered in a timely manner, and prevents 30 early deaths per 1000 patients treated within 6 h after symptom onset. The largest absolute benefit is seen among patients at highest risk, including the elderly, and when treatment is offered is <2 h after symptom onset. A mitochondrial interaction and translocation inhibitor is administered concurrently with, prior to, subsequent to or even additional over a certain period of time post such a procedure.

In some cases, myocardial infarction is treated via CABG (coronary artery bypass graft) surgery in the operation room. Emergent CABG is considered for patients with a patent infarct related artery but with unsuitable anatomy for PCI, and either a large myocardial area at jeopardy or with cardiogenic shock. In patients with MI-related mechanical complications who require coronary revascularization, CABG is recommended at the time of repair. In STEMI patients with failed PCI or coronary occlusion not amenable to PCI, emergent CABG is infrequently performed because the benefits of surgical revascularization in this setting are uncertain. Again, a mitochondrial interaction and translocation inhibitor is administered concurrently with, prior to, subsequent to or even additional over a certain period of time post such a procedure.

Alternately, some methods disclosed herein comprise administration to a patient at risk of an infarction event. Infarction event such as strokes, for example, are a substantial risk to individuals undergoing surgery or suffering from a trauma such as a bruising trauma that may arise from a fall or accident. A number of surgeries carry higher infarction risk, such as cardiac surgery (valve surgery, heart failure surgery, coronary surgery or other cardiac or aortic surgery, as well as long lasting operations generally might affect the heart and lead to damage of the organ.

Individuals suffering from an acute infarction event such as a heart attack are in some cases at heightened risk of a second such event, such as a stroke, often due to disruption of blood clotting factors pursuant to treatment.

Additional acute events harboring an increased infarction event risk include, for example, myocarditis, SARS-Cov2 or other viral infection, such as infection leading to myocardial damage, particularly in ICU-patients. Similarly, trauma of the heart (e.g., accident with compression of the thorax), Hypoxia (e.g. as may be suffered by a mountaineer at high altitude, suffocation, ICU-treatment, for example), Anemia due to e.g. bleeding or hematologic diseases or cancer, or Hypertensive crisis. It is understood that additional acute conditions may lead to infarction or cardiac damage requiring reperfusion, each of which may be ameliorated by treatment with a composition or practice of a method herein.

Tissue or organ transplant often carries a heightened risk of infarction or myocardial damage. Explanting a heart and transporting this organ to a heart transplant recipient will often lead to damage and cell death, which may be ameliorated through practice of the disclosure herein. Compositions may be added to the preservation solution during transport or when given the patient before explanation of the heart or subsequent to the procedure. Aside from heart, the compositions disclosed herein in some cases protect cell and thus organ death in kidney, liver, lung or other transplants.

Similarly, some individuals exhibit higher infarction risk due to a chronic condition such as high blood pressure, high cholesterol, diabetes, malignant or benign cancer, obesity or even being moderate or mildly overweight, among other conditions raising infarction risk. In addition, prolonged treatment regimens such as cancer-therapy (radiation, chemotherapy, anti-cancer immunotherapies or other cancer therapy) or other treatment regimen comprising a course of repeated treatments may carry increased infarction risk and accordingly are suitable for concurrent practice of the methods or administration of compositions as disclosed or contemplated herein. A partial list of chemotherapeutics consistent with the mediation of mitochondrial degradation risk, or benefiting from mitochondrial stabilization as a concurrent step in therapy, include the radiotherapeutic or a chemotherapeutic, such as a chemotherapeutic that targets a cancerous cell, cell population, or tumor. Some such chemotherapeutics cause off-target or unintended cell death, such as cardiomyocyte death. Exemplary chemotherapeutics used to target cancer cells or tumors comprise, for example, a compound selected from the list consisting of an Alkylating agent, such as altretamine, busulfan, carboplatin, carmustine, cisplatin, cyclophosphamide, dacarbazine, ifosfamide, lomustine, melphalan, temozolomide, trabectedin; Antimetabolite, such as 5-fluorouracil, 6-mercaptopurine, azacitidine, capecitabine, clofarabine, cytarabine, floxuridine, fludarabine, gemcitabine, methotrexate, pemetrexed, pentostatin, pralatrexate, trifluridine and tipiracil; antibodies, such as HER2 inhibitors e.g. trastuzumab, pertuzumab, margetuximab, immune checkpoint inhibitors e.g. nivolumab (anti-PD-1), avelumab (anti-PD-L1), ipilimumab (anti-CTLA-4), relatlimab (anti-LAG-3); a Plant alkyloid, such as, vincristine, vinblastine, vinorelbine, paclitaxel, docetaxel, etoposide, teniposide, irinotecan, topotecan; an anti-tumor agent, such as, daunorubicin, doxorubicin, doxorubicin liposomal, epirubicin, idarubicin, and valrubicin. Exemplary chemotherapeutics include anthracyclines, such as daunorubicin, doxorubicin, doxorubicin liposomal, epirubicin, idarubicin, and valrubicin, for example doxorubicin.

Accordingly, some methods comprise prophylactic or concurrent administration of a mitochondrial translocation inhibitor or other mitochondrial integrity-preserving agent to a subject at risk of an infarction event or otherwise at risk of secondary effects due to mitochondrial destabilization. Such administration may serve to ameliorate the harm directly, or to facilitate reperfusion in response to an event by preparing the impacted tissue, or the subject as a whole, for reperfusion at the impacted area.

Administration is in some cases acute, such as prior to surgery or in response to a trauma or other isolated infarction risk event.

Compositions are variously administered no more than 48 hours, no more than 24 hours, no more than 12 hours, no more than 6 hours, no more than 5 hours, no more than 4 hours, no more than 3 hours, no more than 2 hours, no more than 1 hour, no more than 30 minutes, no more than 15 minutes, no more than 10 minutes, or no more than 5 minutes prior to an upcoming infarction risk event. Alternately, compositions are variously administered at least or no more than 96 hours, no more than 72 hours, no more than 48 hours, no more than 24 hours, no more than 12 hours, no more than 6 hours, no more than 5 hours, no more than 4 hours, no more than 3 hours, no more than 2 hours, no more than 1 hour, no more than 30 minutes, no more than 15 minutes, no more than 10 minutes, or no more than 5 minutes subsequent to an infarction risk event. Administration is of a single dose, or alternately comprises an administration regimen of at least 2, 3, 4, 5, 6, 7, 8, 9, 10 or more than 10 doses. Doses comprise consecutive administration of comparable medicament amounts, or alternately comprise changes in dose volume or medicament concentration, such that in some cases effective composition may ‘ramp up’ from an initial low level prior to a risk event such as a surgery.

Alternately, administration is regular or sporadic in response to a chronic condition carrying an elevated risk of an infarction event. Administration is variously monthly, weekly, daily, or at other regular or irregular interval. Administration is often of a steady dose, although irregular dosages or time intervals are also contemplated herein. Irregular administration, such as in response to a biometric output such as insulin level, blood sugar level, cholesterol level or other biometric readout is also contemplated herein. Administration may be alone or in combination with, for example a cholesterol lowering or regulating medicament.

Compositions and methods herein improve outcomes for mammals such as human patients who suffer an infarction event or who are subject to a treatment regime comprising a risk of secondary mitochondrial destabilization. Improvement is measured any number of ways, starting with increased survival and extending to more specific measurements of increased performance among surviving individuals. Amelioration is measured in some cases through MRI visualization or measurement of infarcted area or measurement of the salvaged myocardium using the area at risk and the infarcted area. Measurement is also readily accomplished through SPECT, PET, CT or other scanning technology. Measurements may also be accomplished though echocardiography: measuring infarcted area using contrast agent, function of left ventricle (regional and global), or relying upon strain analyses.

For assessments specific to heart infarction recovery, one may rely upon EKG: ST-segment resolution. Assays may also be accomplished using Ventriculography in the cath lab using contrast agent and determining function of the left ventricle

Lab analysis or blood-draw based assays include measurements of troponin, high sensitive troponin, creatine kinase, creatine kinase Mb.

Method or composition performance is often assessed as cell survival rate or infarction area. A higher cell survival rate correlates to lower size of infarction. Infarct size is of prognostic relevance and correlates with mortality. A higher cell survival rate is expected to correlate with smaller infarct size in MRI, higher myocardial salvage in MRI, lower infarct size in CT, PET, SPECT, with better and faster ST-segment resolution, with lower release of troponin, high sensitive troponin, creatine kinase, creatine kinase Mb, with lower infarct size in echocardiography, with better left ventricular function in echo, with normal strain/strain rate in echo, with better left ventricular function in ventriculography. Cell survival rate is improved relative to a control by in some cases at least 10%, at least 20%, at least 30%, at least 40%, at least 50%, at least 60%, at least 70% at least 80%, at least 90% or greater than 90%.

Method or composition performance may also be measured at the cellular level, by impact on mitochondrial membrane translocation, for example of BNIP3 or BAX, mitochondrial swelling, BAX mitochondrial active concentration, cytochrome c release, Inf/AAR proportion, or caspase activity. Reduction of any of these parameters is in some cases at least 10%, at least 20%, at least 30%, at least 40%, at least 50%, at least 60%, at least 70% at least 80%, at least 90% or greater than 90%.

Controls for these rates are variously selected from measurements of previous untreated individual or mammal outcomes, measurements of experimental controls, fixed values known in the field, values measured from control or untreated populations, or other reference parameters.

Also disclosed herein are methods and compositions for the identification of further candidate molecules for the prevention, amelioration or treatment of infarction or damage associated with infarction, or for the prevention, amelioration or treatment of secondary effects related to mitochondrial destabilization or degradation, such as mitochondrial import related degradation, and concomitant signaling. Without being bound to theory, some such methods relate to the discovery that BAX/BNIP3 complexes are oligomeric in the cytoplasm, and that binding to such oligomeric complexes or inhibition of BAX, BNIP3 or both BAX and BNIP3 translocation into the mitochondrion is indicative of candidate molecules for the prevention, amelioration or treatment of infarction or damage associated with infarction.

Methods are performed by contacting a composition comprising a candidate molecule to at least one cell, cell extract or tissue and assaying the interaction of the molecule or localization of the molecule, or impact of the molecule on localization of BAX, BNIP3, or BAX/BNIP3 heterodimers or oligomers.

Cells, cell extracts or tissue are subjected to oxygen starvation pursuant to induction of an infarction event or in simulation of the effect of an infarction event. A number of oxygen starvation regimes are consistent with the disclosure herein, such performing assays within 10 minutes of oxygen starvation, or alternately no more than any of 1, 2, 3, 4, 5, 6, 7, 8, 9, 10, 11, 12, 13, 14, 15, 20, 25, 30 or more than 30 minutes after oxygen starvation, or longer periods after oxygen starvation. Any of these assays may be performed at baseline, or after ischemia at 5 minutes, 10 minutes, 30 minutes, 1 hour, 2 hours, 4 hours, 8 hours 16 hours 24 hours or longer after reperfusion. In some cases oxygen starvation is absolute, while in alternatives oxygen levels are reduced relative to healthy conditions, or are in some cases unchanged relative to healthy conditions or even elevated. Exemplary extents of oxygen reduction include as much as 100%, 95%, 90%, 80%, 70%, 60%, 50%, 40%, 30%, 20%, 10%, 5%, or less than 5% reduction relative to healthy or pre-assay conditions.

Alternately, Cells, cell extracts or tissue are subjected to radiotherapeutic, chemotherapeutic or other treatment pursuant to induction of secondary mitochondrial destabilization. A number of treatment regimes are consistent with the disclosure herein, such performing assays within 10 minutes of treatment, or alternately no more than any of 1, 2, 3, 4, 5, 6, 7, 8, 9, 10, 11, 12, 13, 14, 15, 20, 25, 30 or more than 30 minutes after treatment, or longer periods after treatment. Any of these assays may be performed at baseline, or after treatment at 5 minutes, 10 minutes, 30 minutes, 1 hour, 2 hours, 4 hours, 8 hours 16 hours 24 hours or longer after treatment. A wide range of mitochondrial status outputs are available for assay, such as those used in FIGS. 1A through FIG. 25H.

Intact cells are preferred in some embodiments, though in alternate cases cell extracts are used, such as cell extracts having intact mitochondria or mitochondrial membranes, or cell extracts that are generated without substantial disruption of oligomeric complexes such as BAX/BNIP3 complexes.

Molecules are assayed for capacity to bind to BAX, BNIP3, BAX/BNIP3 dimers or, in exemplary cases, BAX/BNIP3 oligomers. Without being bound by theory, binding to BAX, BNIP3, BAX/BNIP3 dimers or, in exemplary cases, BAX/BNIP3 oligomers is indicative of efficacy or potential for efficacy of a candidate molecule for the prevention, amelioration or treatment of infarction or damage associated with infarction.

A number of binding assays are consistent with the disclosure herein. Assaying for binding variously comprises assaying for colocalization of a molecule and BAX/BNIP3 oligomeric complexes, for example as determined by fluorescence microscopy. Alternately or in combination, said assaying comprises assaying for comigration of the molecule and BAX/BNIP3 oligomeric complexes, often wherein said comigration is performed under conditions to maintain oligomeric BAX/BNIP3 oligomeric complex integrity, such as nondenaturing conditions. Comigration is determined, for example using any of gel electrophoresis, SDS-PAGE or Western Blot analysis. Assaying for binding may additionally or alternately comprise assaying for coprecipitation of the molecule and BAX/BNIP3 oligomeric complexes, for example using co-immunoprecipitation.

Alternately or in combination, molecules are assayed for an effect upon BAX/BNIP3 complex localization upon contacting to the molecule. Said contacting may occur, for example, in a cell subjected to oxygen starvation or an extract of a cell subjected to oxygen starvation, in particular an extract that comprises functional mitochondrial membranes or functional mitochondria. In some such assays, retention of BAX/BNIP3 outside of a mitochondrion of the cell indicates efficacy in infarction amelioration. Similarly, inhibition of BAX/BNIP3 translocation into a mitochondrion of the cell in some cases indicates efficacy in infarction amelioration. Inhibition of BAX translocation into a mitochondrion of the cell in some cases indicates efficacy in infarction amelioration. In some cases inhibition of BNIP3 translocation into a mitochondrion of the cell indicates efficacy in infarction amelioration. Assaying for BAX, BNIP3 or BAX/BNIP3 localization may comprise fluorescence assays, such as may be effected using fluorescently labeled antibodies or fluorescent-protein or epitope labeled BAX, BNIP3 or BAX and BNIP3 proteins. Alternately, or in combination, molecule localization may be determined using immunofluorescence or detection or a radiolabel and overlaying such signal against an image of the cell or mitochondrion in question. Assay for BAX, BNIP3 or BAX and BNIP3 in a mitochondrial fraction is an alternative approach toward determining localization.

Alternately or in combination, complex localization assays may be supported by or replaced by assaying for mitochondrial integrity, assaying for mitochondrial swelling, assaying for cytochrome c release, assaying for caspase-3 activity, or other assay for mitochondrial activity or necrosis or apoptosis related mitochondrial degradation.

Alternately or in combination, efficacy may be measured by an ability to ameliorate an impact of a treatment on mitochondrial integrity, stability, or cell death signaling.

Numbered Embodiments. The disclosure is further elucidated with reference to the following partial list of numbered embodiments. 1. A method of ameliorating infarction damage to a subject at risk of an infarction damage risk event comprising administering a mitochondrial membrane translocation inhibitor. 2. The method of any previous embodiment, wherein the mitochondrial membrane translocation inhibitor inhibits BAX translocation into a mitochondrial membrane. 3. The method of any previous embodiment, wherein the mitochondrial membrane translocation inhibitor inhibits BNIP3 translocation into a mitochondrial membrane. 4. The method of any previous embodiment, wherein the infarction damage risk event comprises a heart attack. 5. The method of any previous embodiment, wherein the infarction damage risk event comprises a stroke. 6. The method of any previous embodiment, wherein the infarction damage risk event comprises kidney failure. 7. The method of any previous embodiment, wherein the infarction damage risk event comprises an acute circulation failure. 8. The method of any previous embodiment, wherein the infarction damage risk event comprises an organ transplantation. 9. The method of any previous embodiment, wherein the infarction damage risk event comprises surgery. 10. The method of any previous embodiment, wherein the translocation inhibitor is administered intravenously. 11. The method of any previous embodiment, wherein the translocation inhibitor is administered prior to an infarction damage risk event. 12. The method of any previous embodiment, wherein the translocation inhibitor is administered prior to an infarction damage risk event. 13. The method of any previous embodiment, wherein the infarction damage risk event comprises surgery. 14. The method of any previous embodiment, wherein the translocation inhibitor is administered subsequent to an infarction damage risk event. 15. The method of any previous embodiment, wherein the translocation inhibitor is administered prior to reperfusion. 16. The method of any previous embodiment, wherein the infarction damage risk event comprises bruising. 17. The method of any previous embodiment, wherein the infarction damage risk event comprises heart failure. 18. The method of any previous embodiment, wherein the infarction damage risk event comprises a physiological response to stress. 19. The method of any previous embodiment, wherein the infarction damage risk event comprises a physiological response to diabetes. 20. The method of any previous embodiment, wherein the infarction damage risk event comprises a physiological response to hypertension. 21. The method of any previous embodiment, wherein the infarction damage risk event comprises a physiological response to hyperlipidemia. 22. The method of any previous embodiment, wherein the infarction damage risk event comprises a physiological response to obesity. 23. The method of any previous embodiment, wherein the infarction damage risk event comprises a physiological response to a genetic disorder. 24. The method of any previous embodiment, wherein the infarction damage risk event comprises a physiological response to a lung disorder. 25. The method of any previous embodiment, wherein the infarction damage risk event comprises an inflammatory response. 26. The method of any previous embodiment, wherein the infarction damage risk event comprises an autoinflammatory response. 27. The method of any previous embodiment, wherein the BAX translocation inhibitor is administered in multiple doses. 28. The method of any previous embodiment, wherein the multiple doses are administered at regular intervals. 29. The method of any previous embodiment, wherein the BAX translocation inhibitor comprises a chimeric peptide. 30. The method of any previous embodiment, wherein the chimeric peptide comprises a segment having at least 75% identity to at least 8 consecutive residues of BNIP3. 31. The method of any previous embodiment, wherein the at least 8 consecutive residues of BNIP3 comprise a phenylalanine residue at a 7th of the at least 8 consecutive residues of BNIP3. 32. The method of any previous embodiment, wherein the 8 consecutive residues comprise residues having at least 75% identity to residues corresponding to residues 13-20 of BNIP3. 33. The method of any previous embodiment, wherein the 8 consecutive residues comprise residues having at least 87.5% identity to residues corresponding to residues 13-20 of BNIP3. 34. The method of any previous embodiment, wherein the chimeric protein comprises a segment having 75% identity to no more than 50 residues of BNIP3. 35. The method of any previous embodiment, wherein the chimeric protein comprises a segment having 87.5% identity to no more than 50 residues of BNIP3. 36. The method of any previous embodiment, wherein the chimeric protein does not comprise a BH3 motif. 37. The method of any previous embodiment, wherein the chimeric protein does not comprise a PESTQ motif (SEQ ID NO: 4). 38. The method of any previous embodiment, wherein the chimeric protein comprises a BAX binding motif. 39. The method of any previous embodiment, wherein the translocation inhibitor reduces damage from a subsequent infarction event by at least 10%, 40. The method of any previous embodiment, wherein the translocation inhibitor reduces damage from a subsequent infarction event by at least 20%, 41. The method of any previous embodiment, wherein the translocation inhibitor reduces damage from a subsequent infarction event by at least 30%. 42. The method of any previous embodiment, wherein the translocation inhibitor reduces damage from a subsequent infarction event by at least 40%. 43. The method of any previous embodiment, wherein the translocation inhibitor reduces damage from a subsequent infarction event by at least 50%. 44. The method of any previous embodiment, wherein monitoring infarction damage recovery. 45. A method of reducing damage of an infarction event to a subject suffering from an infarction event, comprising administering a mitochondrial membrane translocation inhibitor. 46. The method of any previous embodiment, wherein the translocation inhibitor is administered prior to reoxygenation pursuant to treatment of the infarction event. 47. The method of any previous embodiment, wherein the peptide is administered at least 5 minutes prior to reperfusion. 48. The method of any previous embodiment, wherein the peptide is administered no more than 5 minutes prior to reperfusion. 49. The method of any previous embodiment, wherein the peptide is administered at least one day prior to an infarction risk event. 50. The method of any previous embodiment, wherein the peptide is administered at least two days prior to an infarction risk event. 51. The method of any previous embodiment, wherein the peptide is administered at least three days prior to an infarction risk event. 52. The method of any previous embodiment, wherein the peptide is administered at least seven days prior to an infarction risk event. 53. The method of any previous embodiment, wherein the translocation inhibitor is administered concurrent with reoxygenation pursuant to treatment of the infarction event. 54. The method of any previous embodiment, wherein the treatment reduces BAX mitochondrial concentration by at least 30% relative to an untreated reference. 55. The method of any previous embodiment, wherein the treatment reduces BNIP3 mitochondrial concentration by at least 30% relative to an untreated reference. 56. The method of any previous embodiment, wherein the treatment reduces mitochondrial swelling by at least 30% relative to an untreated reference. 57. The method of any previous embodiment, wherein the treatment reduces BAX active concentration by at least 75% relative to an untreated reference. 58. The method of any previous embodiment, wherein the treatment reduces cytochrome c release by at least 75% relative to an untreated reference. 59. The method of any previous embodiment, wherein the treatment improves Inf/AAR proportion by at least 50% relative to an untreated reference. 60. The method of any previous embodiment, wherein the treatment reduces caspase activity relative to an untreated reference. 61. The method of any previous embodiment, wherein the treatment reduces membrane depolarization relative to an untreated reference. 62. The method of any previous embodiment, wherein the infarction damage risk event comprises a heart attack. 63. The method of any previous embodiment, wherein the infarction damage risk event comprises a stroke. 64. The method of any previous embodiment, wherein the translocation inhibitor is administered intravenously. 65. The method of any previous embodiment, wherein the translocation inhibitor is administered via catheter. 66. The method of any previous embodiment, wherein the translocation inhibitor comprises a chimeric peptide. 67. The method of any previous embodiment, wherein the chimeric protein comprises a segment having at least 75% identity to at least 8 consecutive residues of BNIP3. 68. The method of any previous embodiment, wherein the at least 8 consecutive residues of BNIP3 comprise a phenylalanine residue at a 7th of the at least 8 consecutive residues of BNIP3. 69. The method of any previous embodiment, wherein the 8 consecutive residues comprise residues corresponding to residues 13-20 of BNIP3. 70. The method of any previous embodiment, wherein the chimeric protein comprises a segment having 75% identity to no more than 50 residues of BNIP3. 71. The method of any previous embodiment, wherein the chimeric protein does not comprise a BH3 motif. 72. The method of any previous embodiment, wherein the chimeric protein does not comprise a PESTQ motif (SEQ ID NO. 4). 73. The method of any previous embodiment, wherein the chimeric protein comprises a BAX binding motif. 74. The method of any previous embodiment, comprising monitoring infarction damage recovery. 75. A method of assessing a molecule for infarction amelioration, said method comprising assaying for binding of the molecule to BAX/BNIP3 oligomeric complexes. 76. The method of any previous embodiment, wherein binding of the molecule to the BAX/BNIP3 oligomeric complex indicates efficacy in infarction amelioration. 77. The method of any previous embodiment, wherein said assaying comprises assaying for colocalization of a molecule and BAX/BNIP3 oligomeric complexes. 78. The method of any previous embodiment, wherein said colocalization is assayed by fluorescence microscopy. 79. The method of any previous embodiment, wherein said assaying comprises assaying for comigration of the molecule and BAX/BNIP3 oligomeric complexes. 80. The method of any previous embodiment, wherein said comigration is performed under conditions to maintain oligomeric BAX/BNIP3 oligomeric complex integrity. 81. The method of any previous embodiment, wherein said comigration is assayed using gel electrophoresis. 82. The method of any previous embodiment, wherein said gel electrophoresis comprises SDS-PAGE. 83. The method of any previous embodiment, wherein said gel electrophoresis comprises western blot analysis. 84. The method of any previous embodiment, wherein said comigration is performed under nondenaturing conditions. 85. The method of any previous embodiment, wherein said assaying comprises assaying for coprecipitation of the molecule and BAX/BNIP3 oligomeric complexes. 86. The method of any previous embodiment, wherein said coprecipitation comprises immunoprecipitation. 87. The method of any previous embodiment, wherein said assay comprises contacting said molecule to a cell subjected to oxygen starvation. 88. The method of any previous embodiment, wherein said assay comprises contacting said molecule to a cell within 10 minutes of subjecting the cell to oxygen starvation. 89. A method of assessing a molecule for infarction amelioration, said method comprising contacting the molecule to a cell subjected to oxygen starvation and assaying for BAX/BNIP3 complex localization. 90. The method of any previous embodiment, wherein retention of BAX/BNIP3 outside of a mitochondrion of the cell indicates efficacy in infarction amelioration. 91. The method of any previous embodiment, wherein inhibition of BAX/BNIP3 translocation into a mitochondrion of the cell indicates efficacy in infarction amelioration. 92. The method of any previous embodiment, wherein inhibition of BAX translocation into a mitochondrion of the cell indicates efficacy in infarction amelioration. 93. The method of any previous embodiment, wherein inhibition of BNIP3 translocation into a mitochondrion of the cell indicates efficacy in infarction amelioration. 94. The method of any previous embodiment, wherein assaying for BAX/BNIP3 complex localization comprises immunofluorescence. 95. The method of any previous embodiment, wherein assaying for BAX/BNIP3 complex localization comprises assaying for mitochondrial integrity. 96. The method of any previous embodiment, wherein assaying for BAX/BNIP3 complex localization comprises assaying for mitochondrial swelling. 97. The method of any previous embodiment, wherein assaying for BAX/BNIP3 complex localization comprises assaying for cytochrome c release. 98. The method of any previous embodiment, wherein assaying for BAX/BNIP3 complex localization comprises assaying for caspase-3 activity. 99. The method of any previous embodiment, wherein said assay comprises contacting said molecule to a cell within 10 minutes of subjecting the cell to oxygen starvation. 100. A method of ameliorating a side effect of a treatment, comprising administering a mitochondrial Protecting and/or stabilizing agent. 101. The method of any previous embodiment, wherein the treatment comprises administering a chemotherapeutic. 102. The method of any previous embodiment, wherein the mitochondrial protecting and/or stabilizing agent comprises a mitochondrial interaction and import inhibitor. 103. The method of any previous embodiment, wherein the chemotherapeutic targets a cancerous cell. 104. The method of any previous embodiment, wherein the chemotherapeutic targets a tumor. 105. The method of any previous embodiment, wherein the chemotherapeutic causes cardiomyocyte cell death. 106. The method of any previous embodiment, wherein the chemotherapeutic comprises a compound selected from the list consisting of an Alkylating agent, such as altretamine, busulfan, carboplatin, carmustine, cisplatin, cyclophosphamide, dacarbazine, ifosfamide, lomustine, melphalan, temozolomide, trabectedin; Antimetabolite, such as 5-fluorouracil, 6-mercaptopurine, azacitidine, capecitabine, clofarabine, cytarabine, floxuridine, fludarabine, gemcitabine, methotrexate, pemetrexed, pentostatin, pralatrexate, trifluridine and tipiracil; antibodies, such as HER2 inhibitors e.g. trastuzumab, pertuzumab, margetuximab, immune checkpoint inhibitors e.g. nivolumab (anti-PD-1), avelumab (anti-PD-L1), ipilimumab (anti-CTLA-4), relatlimab (anti-LAG-3); a Plant alkyloid, such as, vincristine, vinblastine, vinorelbine, paclitaxel, docetaxel, etoposide, teniposide, irinotecan, topotecan; an anti-tumor agent, such as, daunorubicin, doxorubicin, doxorubicin liposomal, epirubicin, idarubicin, and valrubicin. 107. The method of any previous embodiment, wherein the chemotherapeutic comprises a compound selected from the list consisting of daunorubicin, doxorubicin, doxorubicin liposomal, epirubicin, idarubicin, and valrubicin. 108. The method of any previous embodiment, wherein the chemotherapeutic comprises an anthracycline. 109. The method of any previous embodiment, wherein the anthracycline comprises doxorubicin. 110. The method of any previous embodiment, wherein the inhibitor impacts BNIP3 activity. 111. The method of any previous embodiment, wherein the inhibitor impacts at least one indication selected from the list comprising DOX cell death induction, mitochondrial membrane potential stabilization, mitochondrial pore opening prevention, mitochondrial Calcium ion overload prevention, mitochondrial ROS or accumulation prevention. 112. The method of any previous embodiment, wherein the inhibitor attenuates cardiotoxicity. 113. The method of any previous embodiment, wherein the inhibitor attenuates cardiomyocyte death. 114. The method of any previous embodiment, wherein the inhibitor preserves cardiac function. 115. The method of any previous embodiment, wherein the inhibitor increases mitochondrial fitness. 116. The method of any previous embodiment, wherein the inhibitor preserves autophagic flux. 117. The method of any previous embodiment, wherein the inhibitor comprises a polypeptide. 118. The method of any previous embodiment, wherein the inhibitor comprises a BNIP3 fragment. 119. The method of any previous embodiment, wherein the inhibitor comprises a BAX fragment. 120. The method of any previous embodiment, wherein administering comprises injecting. 121. A composition for use in ameliorating a negative impact of a treatment, the composition comprising a chemotherapeutic and a mitochondrial interaction and import inhibitor. 122. The composition for use of any previous embodiment, wherein the chemotherapeutic targets a cancerous cell. 123. The composition for use of any previous embodiment, wherein the chemotherapeutic targets a tumor. 124. The composition for use of any previous embodiment, wherein the chemotherapeutic causes cardiomyocyte cell death. 125. The composition for use of any previous embodiment, wherein the chemotherapeutic comprises a compound selected from the list consisting of an Alkylating agent, such as altretamine, busulfan, carboplatin, carmustine, cisplatin, cyclophosphamide, dacarbazine, ifosfamide, lomustine, melphalan, temozolomide, trabectedin; Antimetabolite, such as 5-fluorouracil, 6-mercaptopurine, azacitidine, capecitabine, clofarabine, cytarabine, floxuridine, fludarabine, gemcitabine, methotrexate, pemetrexed, pentostatin, pralatrexate, trifluridine and tipiracil; antibodies, such as HER2 inhibitors e.g. trastuzumab, pertuzumab, margetuximab, immune checkpoint inhibitors e.g. nivolumab (anti-PD-1), avelumab (anti-PD-L1), ipilimumab (anti-CTLA-4), relatlimab (anti-LAG-3); a Plant alkyloid, such as, vincristine, vinblastine, vinorelbine, paclitaxel, docetaxel, etoposide, teniposide, irinotecan, topotecan; an anti-tumor agent, such as, daunorubicin, doxorubicin, doxorubicin liposomal, epirubicin, idarubicin, and valrubicin. 126. The composition for use of any previous embodiment, wherein the chemotherapeutic comprises a compound selected from the list consisting of daunorubicin, doxorubicin, doxorubicin liposomal, epirubicin, idarubicin, and valrubicin. 127. The composition for use of any previous embodiment, wherein the chemotherapeutic comprises an anthracycline. 128. The composition for use of any previous embodiment, wherein the anthracycline comprises doxorubicin. 129. The composition for use of any previous embodiment, wherein the inhibitor impacts BNIP3 activity. 130. The composition for use of any previous embodiment, wherein the inhibitor impacts at least one indication selected from the list comprising DOX cell death induction, mitochondrial membrane potential stabilization, mitochondrial pore opening prevention, mitochondrial Calcium ion overload prevention, mitochondrial ROS or accumulation prevention. 131. The composition for use of any previous embodiment, wherein the inhibitor attenuates cardiotoxicity. 132. The composition for use of any previous embodiment, wherein the inhibitor attenuates cardiomyocyte death. 133. The composition for use of any previous embodiment, wherein the inhibitor preserves cardiac function. 134. The composition for use of any previous embodiment, wherein the inhibitor increases mitochondrial fitness. 135. The composition for use of any previous embodiment, wherein the inhibitor preserves autophagic flux. 136. The composition for use of any previous embodiment, wherein the inhibitor comprises a polypeptide. 137. The composition for use of any previous embodiment, wherein the inhibitor comprises a BNIP3 fragment. 138. The composition for use of any previous embodiment, wherein the inhibitor comprises a BAX fragment. 139. The composition for use of any previous embodiment, wherein administering comprises injecting. 140. A method of ameliorating harm to cardiac function, comprising identifying a patient at risk of harm related to mitochondrial disruption, and administering a mitochondrial import inhibitor to the patient. 141. The method of any previous embodiment, wherein the harm related to mitochondrial disruption is a risk of harm to cardiac function. 142. The method of any previous embodiment, wherein a patient at risk of harm to cardiac function comprises a patient suffering from a cardiac infarction. 143. The method of any previous embodiment, wherein a patient at risk of harm to cardiac function comprises a patient suffering from cardiac arrest. 144. The method of any previous embodiment, wherein a patient at risk of harm to cardiac function comprises a patient expected to undergo surgery. 145. The method of any previous embodiment, wherein a patient at risk of harm to cardiac function comprises a patient undergoing surgery. 146. The method of any previous embodiment, wherein a patient at risk of harm to cardiac function comprises a patient having undergone surgery. 147. The method of any previous embodiment, wherein a patient at risk of harm to cardiac function comprises a patient receiving a chemotherapeutic. 148. The method of any previous embodiment, wherein the chemotherapeutic targets a cancer. 149. The method of any previous embodiment, wherein the chemotherapeutic targets a tumor. 150. The method of any previous embodiment, wherein the chemotherapeutic and the mitochondrial import inhibitor are administered concurrently. 151. The method of any previous embodiment, wherein the chemotherapeutic and the mitochondrial import inhibitor are administered in a common composition. 152. The method of any previous embodiment, wherein the chemotherapeutic and the mitochondrial import inhibitor are not administered concurrently. 153. The method of any previous embodiment, wherein the chemotherapeutic targets a cancerous cell. 154. The method of any previous embodiment, wherein the chemotherapeutic targets a tumor. 155. The method of any previous embodiment, wherein the chemotherapeutic causes cardiomyocyte cell death. 156. The method of any previous embodiment, wherein the chemotherapeutic comprises a compound selected from the list consisting of an Alkylating agent, such as altretamine, busulfan, carboplatin, carmustine, cisplatin, cyclophosphamide, dacarbazine, ifosfamide, lomustine, melphalan, temozolomide, trabectedin; Antimetabolite, such as 5-fluorouracil, 6-mercaptopurine, azacitidine, capecitabine, clofarabine, cytarabine, floxuridine, fludarabine, gemcitabine, methotrexate, pemetrexed, pentostatin, pralatrexate, trifluridine and tipiracil; antibodies, such as HER2 inhibitors e.g. trastuzumab, pertuzumab, margetuximab, immune checkpoint inhibitors e.g. nivolumab (anti-PD-1), avelumab (anti-PD-L1), ipilimumab (anti-CTLA-4), relatlimab (anti-LAG-3); a Plant alkyloid, such as, vincristine, vinblastine, vinorelbine, paclitaxel, docetaxel, etoposide, teniposide, irinotecan, topotecan; an anti-tumor agent, such as, daunorubicin, doxorubicin, doxorubicin liposomal, epirubicin, idarubicin, and valrubicin. 157. The method of any previous embodiment, wherein the chemotherapeutic comprises a compound selected from the list consisting of daunorubicin, doxorubicin, doxorubicin liposomal, epirubicin, idarubicin, and valrubicin. 158. The method of any previous embodiment, wherein the chemotherapeutic comprises an anthracycline. 159. The method of any previous embodiment, wherein the anthracycline comprises doxorubicin. 160. The method of any previous embodiment, wherein the inhibitor impacts BNIP3 activity. 161. The method of any previous embodiment, wherein the inhibitor impacts at least one indication selected from the list comprising DOX cell death induction, mitochondrial membrane potential stabilization, mitochondrial pore opening prevention, mitochondrial Calcium ion overload prevention, mitochondrial ROS or accumulation prevention. 162. The method of any previous embodiment, wherein the inhibitor attenuates cardiotoxicity. 163. The method of any previous embodiment, wherein the inhibitor attenuates cardiomyocyte death. 164. The method of any previous embodiment, wherein the inhibitor preserves cardiac function. 165. The method of any previous embodiment, wherein the inhibitor increases mitochondrial fitness. 166. The method of any previous embodiment, wherein the inhibitor preserves autophagic flux. 167. The method of any previous embodiment, wherein the inhibitor comprises a polypeptide. 168. The method of any previous embodiment, wherein the inhibitor comprises a BNIP3 fragment. 169. The method of any previous embodiment, wherein the inhibitor comprises a BAX fragment. 170. The method of any previous embodiment, wherein administering comprises injecting.

Turning to the Figures, one sees the following.

FIG. 1A-Q. Cytosolic BNIP3 determines necrotic and apoptotic cell death successively by regulating BAX in a heterodimeric state (A) Immunoblot for cytosolic BNIP3 following blue native-PAGE at baseline (n=5). (B) Immunoblot for cytosolic BNIP3 following size exclusion chromatography (n=3) (FIG. 7A). (C) In vivo I/R model timeline and schematic of a heart section depicting the non-ischemic area (remote; blue), ischemic area (area at risk, AAR; red), and infarct area (white, embedded in the AAR). (D) Infarct sizes per area at risk (Inf/AAR) in wild-type and Bnip3^{-/-} mice treated with TATBNIP3 as indicated (n=3-6). This indicates that increasing TAT-Bnip3 administration returns Inf/AAR percent ratios to wild-type levels. (See FIG. 7B) (E-L and N-Q) Wild-type mice were treated with vehicle or TAT-BNIP3-ΔTM. (E) Representative images of the necrotic area (blue), troponin I (red) and nuclei (blue) at 24 hours reperfusion. Scale bars, 1 mm and plasma troponin I after 24 hours of reperfusion (n=4-12). Sham operated mice served as controls. Delta-TM administered mice show a substantiual return to sham treated Troponin I levels (See FIG. 7C. (F) Representative images of apoptotic cells (red) and nuclei (blue) in the AAR 4 hours after I/R. Scale bars, 2,000 μm (left), 200 μm (right) and apoptotic events in the whole heart and plane sections from apical to basal after 4 hours of reperfusion (n=3-5). (G) Depolarized mitochondria at baseline and for the indicated reperfusion times (n=3-5) assessed by flow cytometry (see FIG. 8A). At I/R₁₀, delta-TM mice showed a return to basal levels of depolarized mitochondria. For 2D scatter plots, see FIG. 8C. (H) Swollen mitochondria in the AAR after 10 minutes of reperfusion (OD, optical density; n=3-4). Sham operated mice served as controls. At I/R₁₀, delta-TM mice showed a return to basal levels of swollen mitochondria (I) Representative electron micrographs depicting swollen mitochondria in the AAR at 10 minutes reperfusion. Scale bars, 2 μm (left in each pair), 1 μm (right) (n=3). For fragmented mitochondria, see FIGS. 8B and D. At I/R₁₀, delta-TM mice showed a return to basal levels of mitochondrial swelling (J and K) Quantification and representative immunoblots of the mitochondrial BAX concentration (J) and mitochondrial BNIP3 level (*p=0.0148 and *p=0.0185) (K) in the area at risk (AAR) at baseline and 10 minutes reperfusion (AAR) (n=5-7) (see FIG. 8E-I). Delta-TM mice showed a return to basal levels for both assays. (L) Quantification and representative immunoblot of active BAX at baseline and the indicated reperfusion time point (n=5). At I/R₃₀, delta-TM mice showed a return to basal levels of active BAX concentration (see FIG. 9A-C). (M) Quantification and representative immunoblot of mitochondrial BAX levels in the AAR of Bnip3−/− mice untreated and treated with TAT-BNIP3 (n=3; *p=0.0152). At I/R₁₀, delta-TM mice showed shift opposite that of untreated Bnip3−/− mice alone. (N) Western blot monitoring of cytosolic BAX co-immunoprecipitated with BNIP3 (left) and cytosolic BNIP3 co-immunoprecipitated with BAX (right) at baseline and after 10 and 30 minutes of reperfusion (n=3) (O) Cytoplasmic cytochrome c in the AAR at baseline and for the indicated reperfusion time points (in =5-6). At I/R₃₀, delta-TM mice maintained basal levels of cytochrome c. (P) ATP in the AAR at baseline and 10 minutes reperfusion (n=4; *p=0.0153 and *p=0.011). At I/R₁₀, delta-TM mice maintained basal levels of ATP. (Q) Serine-phosphorylated BNIP3 levels at baseline and the indicated reperfusion time points (n=5-8) (see FIG. 9D). Data represent the means±SEM; p values were determined by two-way ANOVA with Bonferroni's correction. At I/R₃₀, delta-TM mice maintained basal levels of BNIP3 phosphorylation.

FIG. 2A-G. N-terminus is the interacting domain of BNIP3 (A) Images of membranes spotted with BAX and BNIP3 and incubated with fluorescently labelled (Dy650) BNIP3 (left) and BAX (right). (B) Schematic of the protocol designed by JPT (Germany, Berlin). (C) Heat map depiction of BNIP3 incubation with the BAX peptide library displaying the helices α5, α6 and α7+α8 as interaction sites. Color coding ranges from white (low or no intensity) to yellow (middle intensity) and red (high intensity) and structure of mouse BAX (PDB 4S0OZit) in ribbon representation with BNIP3 interaction sites colored blue (α5), green (α6), yellow (α7) and orange (α8). (D and E) 3D structural model of BNIP3 predicted by Modeller 9.15 with marked helices. (F) Circular dichroism (CD) spectroscopic analysis of BNIP3. (G) Cartoon representations of BAX/BNIP3 interactions with the indicated binding sites resulting from in silico docking experiments using HADDOCK. BNIP3 is colored turquoise, and BAX is colored green. Interaction sites are in purple (BAX) and orange (BNIP3).

FIG. 3A-Q. N-terminus of BNIP3 is a critical functional domain in human cardiomyocytes protection (A) Structural formula of TAT-BNIP3-20A. (B) Structural formula of TAT-BNIP3-20C. (C-E) Mice were treated with TAT-BNIP3-20A (20A) or TAT-BNIP3-20C (20C). (C) Quantification and representative immunoblot of mitochondrial BNIP3 levels in the area at risk (AAR) at 10 minutes reperfusion (n=6). 20A mice showed a lower relative mitochondrial BNIP3 concentration. (D) Quantification of caspase-3 activity in the AAR at 1 hour reperfusion (n=7). 20A mice showed a lower relative Caspase 3 activity, closer to basal levels. (E) Infarct sizes per AAR (Inf/AAR) in wild-type mice treated with vehicle (NaCl), 20A or 20C at 24 hours reperfusion (n=5-6). 20A mice showed a lower INF/AAR percent than either vehicle or 20C (F) Immunoblot of BNIP3 in three human heart samples. (G) Alignment of the human and mouse BNIP3 sequences. The red box indicates the amino acids WVELHFSN (SEQ ID NO: 2), with the highest binding intensity representing a conserved region. (H and I) Peptide microarrays of BNIP3 with the N-terminal sequence of BNIP3 (1-20 aa). (H) N-terminal sequence truncation analysis elucidating BNIP3/peptide interactions. VELHFSN (SEQ ID NO: 8) showed a substantially higher luminescence (see FIG. 10A). (I) Single amino acid substitutions characterizing their binding capacities. (J) Docking experiments on BAX (PDB code 4S0O) with TAT-WVELHFFN (“WVELHFFN” disclosed as SEQ ID NO: 3) showing the interaction sites in BAX. (K) Docking experiments on BAX (PDB code 4S0O) with TAT-WVELHFFN (“WVELHFFN” disclosed as SEQ ID NO: 3) and TAT-WVELHFSN (“WVELHFSN” disclosed as SEQ ID NO: 2) and peptide microarrays of BAX with indicated peptide sequences. Addition of a third aromatic residue results in improved intrapeptide aromatic interactions, in particular for WVELHFFN (SEQ ID NO: 3). (L) Space-filling models and structure formula of TAT-WVELHFFN (“WVELHFFN” disclosed as SEQ ID NO: 3) (see FIG. 10B). (M) Circular dichroism (CD) spectroscopic analysis of TAT-WVELHFFN (“WVELHFFN” disclosed as SEQ ID NO: 3). (N) Images of membranes spotted with BNIP3 (upper) and BAX (bottom) and incubated with fluorescently labelled TAT-WVELHFFN (“WVELHFFN” disclosed as SEQ ID NO: 3) (TAT-WVELHFFN-fluo (“WVELHFFN-fluo” disclosed as SEQ ID NO: 9)). (O) Timeline of the in vitro hypoxia/reoxygenation (H/R) study design in human ventricular cardiomyocytes derived from human induced pluripotent stem cells (human CMs). (P) BNIP3/BAX interactions in human CMs after 1 h hypoxia/1 h of reperfusion (PLA, proximity ligation assay) (n=5) and representative staining confocal images. Cell structure (green), nucleus (blue), PLA spots (red; marked by white arrows), 40×. (Q) Depolarized mitochondria (green), healthy mitochondria (red), nucleus (blue) after 1 h hypoxia/1 h reoxygenation (upper panel). Apoptotic (green), necrotic (red) and healthy (blue) human CMs after 2 h hypoxia/2 h reoxygenation, treated with TAT-WVELAASN (“WVELAASN” disclosed as SEQ ID NO: 7) and TAT-WVELHFFN (“WVELHFFN” disclosed as SEQ ID NO: 3) (bottom panel). Scale bars, 200 m. Data represent the means±SEM; p values were determined by two-way ANOVA with Bonferroni's correction or two-tailed Student's t-test.

FIG. 4A-H. WVELHFFN peptide functions in necrotic and apoptotic signaling prevention in vivo (A) Timeline of the in vivo ischemia/reperfusion model. (B) Uptake of labelled TAT-WVELHFFN (“WVELHFFN” disclosed as SEQ ID NO: 3) in different organs at 10 minutes reperfusion. TAT-WVELHFFN (“WVELHFFN” disclosed as SEQ ID NO: 3) was administered 5 minutes before reperfusion (see FIG. 11A-C). (C) Representative 3D visualization of TAT-WVELHFFN-Cy5.5 (“WVELHFFN-Cy5.5” disclosed as SEQ ID NO: 10) (red) distribution across a whole murine heart after I/R (blue knot) with endogenous autofluorescence (green). (D-I) Mice were treated with TAT-WELVHFFN (“WELVHFFN” disclosed as SEQ ID NO: 11) or TAT-WELAASN (“WELAASN” disclosed as SEQ ID NO: 12). Sham operated mice served as controls. (D) Mitochondrial BNIP3 (*p=0.0161) and (E) mitochondrial BAX levels in the area at risk (AAR) at 10 minutes reperfusion. (n=5). (F) Swollen mitochondria from the AAR at 10 minutes reperfusion (OD, optical density; n=5). (G) Active BAX and (H) cytoplasmic cytochrome c levels in the AAR at 30 minutes reperfusion (n=5; *p=0.032). (I) Caspase-3 activity in the AAR at 1 hour reperfusion (n=8-12; *p=0.0164). Data represent the means±SEM; p values were determined by two-way ANOVA with Bonferroni's correction. In each of these assays, TAT-WELVHFFN (“WELVHFFN” disclosed as SEQ ID NO: 11) administration returned measurement outputs to near sham levels.

FIG. 5A-L WVELHFFN functions in inhibition of BNIP3/BAX heterodimers as components of the mitochondrial membrane attack oligomers (A and B) Representative immunoblot for BNIP3 (A) and BAX (B) following SDS-PAGE at baseline and 10 minutes reperfusion. Mice were treated with TAT-WELVHFFN (“WELVHFFN” disclosed as SEQ ID NO: 11) and TAT-WVELAASN (“WVELAASN” disclosed as SEQ ID NO: 7). Sham operated mice served as controls (n=3). (C) Representative BAX immunoblot following co-immunoprecipitation of BNIP3 as in (A) (n=3) (D) Fluorescent immunoblot at 5 minutes of reperfusion. Mice were treated with vehicle (NaCl) and fluorescence-labelled TAT-WELVHFFN (“WELVHFFN” disclosed as SEQ ID NO: 11). (E) BAX and BNIP3 immunoblot following co-immunoprecipitation of cytosolic BNIP3 and BAX and photometric fluorescence measurement of cytosolic fraction (n=3). (F) Immunoblot for cytosolic BNIP3 and BAX in the area at risk (AAR) following blue native-PAGE at baseline and 10 minutes reperfusion (n=3). (G) Immunoblot for cytosolic BNIP3 following size exclusion chromatography at 10 minutes reperfusion with and without TAT-WVELHFFN (“WVELHFFN” disclosed as SEQ ID NO: 3) treatment (n=3). (H) Immunoblot for cytosolic BAX following size exclusion chromatography at baseline and 10 minutes reperfusion with and without TAT-WVELHFFN (“WVELHFFN” disclosed as SEQ ID NO: 3) treatment (n=3). (I) Fluorescence measurement of TAT-WVELHFFN-fluo (“WVELHFFN-fluo” disclosed as SEQ ID NO: 9) following size exclusion chromatography at 5 min reperfusion (n=3).

FIG. 6A-N. TAT-WVELHFFN (“WVELHFFN” disclosed as SEQ ID NO: 3) improves clinical outcomes (A) Timeline of the in vivo ischemia/reperfusion (I/R) model. Animals were injected with vehicle (sodium chloride), β-Gal, TAT-WVELAASN (“WVELAASN” disclosed as SEQ ID NO: 7) or TAT-WVELHFFN (“WVELHFFN” disclosed as SEQ ID NO: 3). (B) Area at risk (AAR) per left ventricle (LV) and (C) infarct sizes per AAR (Inf/AAR) at 24 hours myocardial reperfusion in wild-type mice treated as indicated (n=6). TAT-WELVHFFN (“WELVHFFN” disclosed as SEQ ID NO: 11) administration substantially reduced infarct size. (D) Infarct sizes at 24 hours myocardial reperfusion in wild-type mice treated with the indicated TAT-WVELHFFN (“WVELHFFN” disclosed as SEQ ID NO: 3) doses (n=3-7; *p=0.0172). TAT-WELVHFFN (“WELVHFFN” disclosed as SEQ ID NO: 11) substantially reduced infarct size at suitable doses of administration. (E) Apoptotic events in heart plane sections at 4 hours reperfusion. At all plane sections tested, TAT-WELVHFFN (“WELVHFFN” disclosed as SEQ ID NO: 11) administration reduced the number of apoptosis events. (F) Representative images of apoptotic cells (red) and nuclei (blue) in the AAR at 4 hours reperfusion. (G) Inf/AAR at 24 hours myocardial reperfusion in wild-type mice treated with TAT-BNIP3-ATM (ATM) 5 minutes before ischemia and additional with TAT-WVELHFFN (“WVELHFFN” disclosed as SEQ ID NO: 3) 5 minutes before reperfusion (n=3-6 mice). (H) in vivo myocardial I/R in pigs subjected to 60 minutes of ischemia and 4 hours of reperfusion and treated with vehicle or TAT-WVELHFFN (“WVELHFFN” disclosed as SEQ ID NO: 3) (n=3). AAR/LV (H), Inf/AAR (I) and Inf/LV (*p=0.02) (J). Inf/AAR and Inf/LV ratios were significantly reduced upon administration of TAT-WELVHFFN (“WELVHFFN” disclosed as SEQ ID NO: 11). (I) Brain Inf/AAR at 24 hours reperfusion in wild-type mice treated with vehicle or TAT-WVELHFFN (“WVELHFFN” disclosed as SEQ ID NO: 3) (n=5-7). Inf/AAR ratios were significantly reduced upon administration of TAT-WELVHFFN (“WELVHFFN” disclosed as SEQ ID NO: 11). (J) Pre-treatment timeline. (K) Inf/AAR for treated vs control subjects. Inf/AAR ratios were significantly reduced upon administration of TAT-WVELHFFN (“WVELHFFN” disclosed as SEQ ID NO: 3) (PepB). (L) Ejection fraction (EF) % for control vs treated individuals, assayed at D−7, D−1, D0, D1, D3, and D5. Treated individuals exhibited significantly higher EF %, as indicated by the asterisks. (M) Post-treatment timeline. (N) Left ventricular ejection fraction (LVEF) % for control vs multiple treated individuals, assayed at d−1, d1, d3, d5, and d7. Treated individuals exhibited recovery of LV function at day 3, as indicated by the asterisk.

FIG. 7A-C. Apoptosis and necrosis analyses in vivo, Related to FIG. 1 (A) Workflow size exclusion chromatography. (B) Confocal microscopy images showing murine heart tissue from wild-type, Bnip3^{-/-} and Bnip3^{-/-} mice after injection of the TAT-BNIP3 fusion protein. Mitochondria were stained in red, nuclei in blue and BNIP3 in green. (C) Infarct sizes per area at risk (Inf/AAR) in wild-type mice treated as indicated (n=3-6; vehicle (NaCl), TAT-BNIP3ΔTM, ΔTM (left). Representative images of TTC-stained heart sections from mice treated with vehicle (NaCl) or ΔTM at 24 hours reperfusion (middle). AAR/left ventricle (LV) at 24 hours reperfusion in wild-type mice (n=3-6 mice). Data represent the means±SEM; p values were determined by one-way ANOVA with Bonferroni's correction.

FIG. 8A-8I. Characterization of mouse cardiac mitochondria and BNIP3, Related to FIG. 1(A) Quantification of depolarized mitochondria at baseline and for the indicated reperfusion times in mouse myocardial I/R injury by flow cytometry (n=5). (B) Quantification of intact, swollen and damaged mouse heart mitochondria at baseline and at 10 minutes reperfusion (n=3-5) and representative electron micrographs. Scale bars, 1 μm. (C) Representative flow cytometry 2D dot plots of mitochondrial membrane potential using JC-1 dye. (D) Representative electron micrographs depicting fragmentated mitochondria. Scale bar, 5 μm (left), scale bars, 1 μm (right). (E) Immunoblot of BNIP3 in a whole murine heart and isolated cardiomyocytes. (F and G) Time course of ischemia/reperfusion injury: BNIP3 mRNA transcript levels (n=4) (F) and protein concentrations (n=9) (G) at baseline and for the indicated reperfusion times. Representative immunoblot of BNIP3. (H) Western blot monitoring of the subcellular localization of BNIP3. (I) Representative images of mitochondria (red), cytosolic BNIP3 (green; denoted by yellow arrows), mitochondrial BNIP3 (yellow; denoted by white arrows) and nuclei (blue) in mouse heart slices. 64× magnification.

FIG. 9A-9D. Mouse mitochondria in cardiac health and disease, Related to FIG. 1 (A) Mitochondrial pore formation experiments in which isolated mitochondria were treated with cytoplasm and indicated inhibitors, demonstrating the need for BNIP3 in BAX pore formation, as shown by quantification of cytochrome c, AIF, and active BAX concentrations (n=3). (B and C) Schematics and representative traces of mitochondrial swelling experiments. assessed by measuring optical density (OD) in which mitochondria were treated with ΔTM and with either buffer inducing mitochondrial swelling (mPTP buffer) (B) or mitochondrial outer membrane rupture (non-mPTP inducing buffer) (C). (D) Quantification of BNIP3 serine phosphorylation and active BAX concentrations in HL-1 cells exposed to hypoxia (24 hours) and reoxygenation (1 hour) (H/R), treated or not with an AKT inhibitor (n=5 independent experiments). Data represent the means±SEM; p values were determined by two-way ANOVA with Bonferroni's correction.

FIG. 10A-10B. Alanine scan, Related to FIG. 3 (A) Heat map depiction of the alanine scan with the N-terminal sequence of BNIP3 (1-20 aa) incubated with recombinant BNIP3. Color coding ranges from white (low or no intensity) to yellow (middle intensity) and red (high intensity). (B) Space filling model and structural formula of WVELAASN (SEQ ID NO. 7).

FIG. 11A-C. Identification and in vitro/in vivo effects of the advanced TAT-WVELHFFN peptide (“WVELHFFN” disclosed as SEQ ID NO: 3), Related to FIG. 4 (A) Western blots of fluorescence-labelled TAT-WVELHFFN (“WVELHFFN” disclosed as SEQ ID NO: 3) level profiles in human serum, plasma, and whole blood after incubation at 37° C. for indicated times. Proteinase K treatment served as a control at 60 minutes. (B) Survival of isolated adult cardiomyocytes treated with TAT-WVELHFFN (“WVELHFFN” disclosed as SEQ ID NO: 3) and TAT-WVELAASN (“WVELAASN” disclosed as SEQ ID NO: 7) after 24 hours (n=2). (C) Echocardiographs, measured at baseline (pre) and 5 hours after injection of 8 nmol TAT-WVELHFFN (“WVELHFFN” disclosed as SEQ ID NO: 3) (n=3).

FIG. 12A-12D. TAT-peptides. HPLC (220 nm, C18, linear gradient) of newly synthesized chimera (A) MSQSGEENLQGSWVELHFSN (SEQ ID NO: 1); (B) LDAQHESGRSSSKSSHCDSP (SEQ ID NO: 6); (C) WVELHFFN (SEQ ID NO: 3) and (D) WVELAASN (SEQ ID NO: 7).

FIG. 13A-B. TAT-WVELHFFN (“WVELHFFN” disclosed as SEQ ID NO: 3) ameliorates DOX induced cell death in human cardiomyocytes. At FIG. 13(A), one sees an image indicating DOX and TAT-WVELHFFN (“WVELHFFN” disclosed as SEQ ID NO: 3) uptake. The image is generated using confocal microscopy. Magnification: 40×. Scale bar: 20 μm. 10 μM DOX, and 20 μM TAT-WVELHFFN (“WVELHFFN” disclosed as SEQ ID NO: 3) are administered, and the cells are visualized at 22 h. Dox is indicated in yellow, while TAT-WVELHFFN (“WVELHFFN” disclosed as SEQ ID NO: 3) is stained using Cy5. At FIG. 13(B) one sees that TAT-WVELHFFN (“WVELHFFN” disclosed as SEQ ID NO: 3) ameliorates DOX induced cell death in human cardiomyocytes. Dead cells are measured relative to the control, left, as compared to administration of DOX at 10 μM, center, and DOX at 10 μM plus TAT-WVELHFFN (“WVELHFFN” disclosed as SEQ ID NO: 3), right. Administration of the mitochondrial interaction and import inhibitor TAT-WVELHFFN (“WVELHFFN” disclosed as SEQ ID NO: 3) ameliorates the increased cell death induced by administration of the chemotherapeutic DOX, returning cell death to near control levels.

FIG. 14A-B. TAT-WVELHFFN (“WVELHFFN” disclosed as SEQ ID NO: 3) ameliorates DOX induced cell death in human cardiomyocytes. At FIG. 14(A) one sees that TAT-WVELHFFN (“WVELHFFN” disclosed as SEQ ID NO: 3) ameliorates DOX induced cell death in human cardiomyocytes. Attributive percent damage baseline adjusted values were measured for DOX concentrations of, from left to right, 0, 1, and 10 μM. For each concentration, results are shown without (left) and with (right) TAT-WVELHFFN (“WVELHFFN” disclosed as SEQ ID NO: 3). Cells were incubated for four hours, and TAT-WVELHFFN (“WVELHFFN” disclosed as SEQ ID NO: 3) was administered at 0 or 1.5 μM. The results show that the attributive damage caused by a 10 μM dose of the chemotherapeutic DOX is ameliorated by the mitochondrial interaction and import inhibitor TAT-WVELHFFN (“WVELHFFN” disclosed as SEQ ID NO: 3). At FIG. 14(B) one sees that TAT-WVELHFFN (“WVELHFFN” disclosed as SEQ ID NO: 3) ameliorates DOX induced cell death in human cardiomyocytes. RLUs, indicative of cell death, were measured for DOX concentrations of, from left to right, 1 and 10 μM. For each concentration, results are shown without (left) and with (right) TAT-WVELHFFN (“WVELHFFN” disclosed as SEQ ID NO: 3). Cells were incubated for four hours, and TAT-WVELHFFN (“WVELHFFN” disclosed as SEQ ID NO: 3) was administered at 0 or 1.5 μM. The results show that the attributive damage, indicated by an increase in RLU, caused by a 10 μM dose of the chemotherapeutic DOX is ameliorated by the mitochondrial interaction and import inhibitor TAT-WVELHFFN (“WVELHFFN” disclosed as SEQ ID NO: 3).

FIG. 15A-B. TAT-WVELHFFN (“WVELHFFN” disclosed as SEQ ID NO: 3) ameliorates mitochondrial membrane potential destabilization by DOX in human cardiomyocytes. At FIG. 15(A) one sees that WVELHFFN (SEQ ID NO: 3) ameliorates mitochondrial membrane potential destabilization by DOX. Representative images are of donor-human cardiomyocytes (HCM). Incubation is 4h 10 μM DOX Magnification: 40×. Scale bar: 20 μM. Fluorescence at left indicates that membrane potential is preserved in the control. At center, lack of fluorescence indicates that membrane potential is disrupted by administration of 10 μM DOX, while at right, one sees that co-administration of TAT-WVELHFFN (“WVELHFFN” disclosed as SEQ ID NO: 3) ameliorates the negative effect of DOX on mitochondrial membrane potential. At FIG. 15(B), one sees a quantification of the results of FIG. 15(A). Results are shown for corrected total cell fluorescence relative to control, for four- and 22 hour-incubation times. For each chart, control is shown at left, followed by DOX at 10 μM, center, and DOX plus TAT-WVELHFFN (“WVELHFFN” disclosed as SEQ ID NO: 3). In both time incubations, one sees a decrease relative to the control for the DOX administration, and an amelioration of that decrease upon co-administration of TAT-WVELHFFN (“WVELHFFN” disclosed as SEQ ID NO: 3). This indicates that the negative effect of the chemotherapeutic on mitochondrial membrane potential is ameliorated by co-administration of TAT-WVELHFFN (“WVELHFFN” disclosed as SEQ ID NO: 3).

FIG. 16A-B. TAT-WVELHFFN (“WVELHFFN” disclosed as SEQ ID NO: 3) prevents mPTP opening by DOX in human cardiomyocytes. At FIG. 16(A) one sees that TAT-WVELHFFN (“WVELHFFN” disclosed as SEQ ID NO: 3) prevents mPTP opening by DOX. Representative images are of donor-human cardiomyocyt4es (HCM). Incubation is 4h 10 μM DOX Magnification: 40×. Scale bar: 20 μM. Calcein-AM fluorescence at left indicates that mPTP opening is prevented in the control. At center, lack of fluorescence indicates that mPTP opening is activated by administration of 10 μM DOX, while at right, one sees that co-administration of TAT-WVELHFFN (“WVELHFFN” disclosed as SEQ ID NO: 3) prevents the negative effect of DOX on mPTP opening. At FIG. 16(B), one sees a quantification of the results of FIG. 16(A). Results are shown for corrected total cell fluorescence relative to control, for 22 hour-incubation time. For each chart, the effect of DOX at 0 and 10 μM is shown without (left) and with (right) TAT-WVELHFFN (“WVELHFFN” disclosed as SEQ ID NO: 3). One sees a decrease relative to the control for 10 μM DOX administration, and an prevention of that decrease to near control levels upon co-administration of TAT-WVELHFFN (“WVELHFFN” disclosed as SEQ ID NO: 3). This indicates that the negative effect of the chemotherapeutic on mPTP opening is prevented by co-administration of TAT-WVELHFFN (“WVELHFFN” disclosed as SEQ ID NO: 3).

FIG. 17. TAT-WVELHFFN (“WVELHFFN” disclosed as SEQ ID NO: 3) prevents mitochondrial calcium ion overload triggered by DOX in human cardiomyocytes. At Figure E one sees that TAT-WVELHFFN (“WVELHFFN” disclosed as SEQ ID NO: 3) prevents mitochondrial calcium ion overload triggered by DOX. Representative images are of donor-human cardiomyocytes (HCM). Incubation is 4h 10 μM DOX Magnification: 40×. Scale bar: 20 μM. Calcium-Rhodamin fluorescence at low levels, at left indicates that calcium overload does not occur in the control. At center, increase in fluorescence indicates that mitochondrial calcium ion overload is induced by administration of 10 uM DOX, while at right, one sees that co-administration of TAT-WVELHFFN (“WVELHFFN” disclosed as SEQ ID NO: 3) prevents the negative effect of DOX on calcium ion overload.

FIG. 18A-B. TAT-WVELHFFN (“WVELHFFN” disclosed as SEQ ID NO: 3) ameliorates mitochondrial reactive oxygen species (ROS) generation triggered by DOX in human cardiomyocytes. At FIG. 18(A) one sees that TAT-WVELHFFN (“WVELHFFN” disclosed as SEQ ID NO: 3) ameliorates mitochondrial ROS generation triggered by DOX. Representative images are of donor-human cardiomyocytes (HCM). Incubation is 4h 10 μM DOX Magnification: 40×. Scale bar: 20 μM. ROS fluorescence at low levels, at left indicates that ROS do not accumulate in the control. At center, increase in fluorescence indicates that ROS generation is induced by administration of 10 μM DOX, while at right, one sees that co-administration of TAT-WVELHFFN (“WVELHFFN” disclosed as SEQ ID NO: 3) ameliorates the negative effect of DOX on ROS generation. At FIG. 18(B), one sees a quantification of the results of FIG. 18(A). Results are shown for corrected ROS generation relative to control, for four- and 22 hour-incubation times. For each chart, control is shown at left, followed by DOX at 10 μM, center, and DOX plus TAT-WVELHFFN (“WVELHFFN” disclosed as SEQ ID NO: 3). In both time incubations, one sees an increase relative to the control for the DOX administration, and an amelioration of that increase upon co-administration of TAT-WVELHFFN (“WVELHFFN” disclosed as SEQ ID NO: 3). This indicates that the negative effect of the chemotherapeutic on ROS generation is ameliorated by co-administration of TAT-WVELHFFN (“WVELHFFN” disclosed as SEQ ID NO: 3).

FIGS. 15-18: Collectively, these results indicate that the conclusions made previously also generally apply to treatment of human cells.

FIG. 19. Administration time course of a chemotherapeutic and a mitochondrial import inhibitor.

FIG. 20. PepB (TAT-WVELHFFN (“WVELHFFN” disclosed as SEQ ID NO: 3)) does not have long term detrimental effects on mammalian recipients. Probability of survival, body weight change over time, tibia length, and heart weight were measured and in each case no significant difference was observed upon pepB administration relative to an NaCL carrier control.

FIG. 21. PepB (TAT-WVELHFFN (“WVELHFFN” disclosed as SEQ ID NO. 3)) prevents cardiomyocyte death. At FIG. 21, one sees that for the administration regimen at left, PepB prevents cardiomyocyte death. From center to right, one sees that cardiac troponin I (cTNI)/body weight is significantly lower for DOX coadministration with PepB relative to DOX administration with an NaCl control. Similarly, creantine kinase (CK)/body weight is significantly lower for DOX coadministration with PepB relative to DOX administration with an NaCl control. Further to the right, one sees that LDH/body weight center right, and BNP/body weight, right were unchanged by administration of PepB.

FIG. 22A-E. Inhibition of BNIP3 preserves cardiac function. At FIG. 22(A) one sees the effects on ejection fraction (EF) % at each of three time points T0, T1, and T2, of from left, control, DOX, and DOX plus PepB (TAT-WVELHFFN (“WVELHFFN” disclosed as SEQ ID NO: 3)) administration. At both T1 (16 days) and T2 (42 days), DOX negatively impacts EF %, and this impact is ameliorated by concurrent PepB administration. At FIG. 22(B) one sees the effects on fractional shortening (FS) % at each of three time points T0, T1, and T2, of from left, control, DOX, and DOX plus PepB administration. At both T1 and T2, DOX negatively impacts FS %, and this impact is ameliorated by concurrent PepB administration. At FIG. 22(C) one sees the effects on isovolumetric relaxation time (IVRT) (ms) at each of three time points TO, T1, and T2, of from left, control, DOX, and DOX plus PepB administration. At both T1 and T2, DOX slowed IVRT, and this impact is ameliorated by concurrent PepB administration. At FIG. 22(D) one sees the effects on heart rate at each of three time points TO, T1, and T2, of from left, control, DOX, and DOX plus PepB administration. Heart rate was unaffected by DOX administration, alone or in combination with PepB. At FIG. 22(E), one sees the effect of a control, DOX, and DOX plus PepB on cardiac troponin I (cTnl) (ng/l), presented on a logarithmic scale. One sees that DOX has a dramatic positive effect on cTnl, and that this effect is ameliorated by coadministration of PepB.

FIG. 23A-D. Inhibition of BNIP3 upon DOX administration increases mitochondrial fitness. At FIG. 23(A), one sees that DOX inhibits oxygen consumption rate (OCR) at reserve capacity (pmol/min/1.5 ug protein), and that PepB (TAT-WVELHFFN (“WVELHFFN” disclosed as SEQ ID NO: 3)) ameliorates this inhibition. From left, control DOX and DOX plus PepB. At FIG. 23(B), one sees that DOX inhibits OCR at maximal respiration (pmol/min/1.5 ug protein), and that PepB ameliorates this inhibition. From left, control DOX and DOX plus PepB. At FIG. 23(C), one sees that DOX inhibits basal respiration (as a percent of control), and that PepB ameliorates this inhibition. From left, control DOX and DOX plus PepB. At FIG. 23(D), one sees that DOX inhibits Complex II-stimulated respiration (as a percent of control), and that PepB ameliorates this inhibition. From left, control DOX and DOX plus PepB.

FIG. 24A-D. Inhibition of BNIP3 upon DOX administration restores autophagic flux. At FIG. 24(A), one sees a western blot of proteins extracted from, from left, Control, DOX treated and DOX plus PepB (TAT-WVELHFFN (“WVELHFFN” disclosed as SEQ ID NO: 3)) treated tissues. For each protein source, the left two lanes are untreated while the sample source of the right two lanes is additionally treated with cloroquine (CQ). Electrophoresed proteins were probed with LC3-I and LC3-II. At FIG. 24(B), one sees that DOX inhibits CQ mediated LC3II/I increase. From left, control DOX and DOX plus PepB, each presented without, left, or with, right CQ treatment. DOX abolishes the CQ-induced increase, while addition of PepB partially restores this increase. At FIG. 24(C), one sees that DOX triggers p62 accumulation, and that addition of PepB counteracts this increase. P62 levels are shown as a percent of control for control, DOX treated and DOX plus PepB treated samples. At FIG. 24(D), one sees that DOX triggers BNIP3 accumulation, and that addition of PepB counteracts this increase. BNIP3 levels are shown as a percent of control for control, DOX treated and DOX plus PepB treated samples.

FIG. 25A-H. Results presented herein are applicable to human cardiocyes as well as mouse tissues as presented previously. At FIG. 25(A), one sees that PepB reduces DOX mediated mitochondrial membrane depolarization. Left, control, center DOX, and right, DOX plus PepB treatment of human cardiocytes. DOX triggers membrane depolarization, indicated by a lack of fluorescence, while DOX plus PepB treated human cells retain control-level fluorescence. At FIG. 25(B) one sees a quantification of the results in Figure M(A). Fluorescence Intensity is presented as % of control for, from left, control, DOX treated, and DOX plus PepB treated human cardiocytes. The restoration of fluorescence upon PepB treatment indicates that PepB counteracts the negative effect of DOX on mitochondrial membrane depolarization. At FIG. 25(C), one sees that PepB reduces DOX mediated mPTP opening. Left, control, center DOX, and right, DOX plus PepB treatment of human cardiocytes. DOX triggers mPTP, indicated by a lack of fluorescence, while DOX plus PepB treated human cells retain control-level fluorescence. At FIG. 25(D) one sees a quantification of the results in Figure M(C). Fluorescence Intensity is presented as % of control for, from left, control, DOX treated, and DOX plus PepB treated human cardiocytes. The restoration of fluorescence upon PepB treatment indicates that PepB counteracts the negative effect of DOX on mPTP opening. At FIG. 25(E), one sees that PepB reduces DOX mediated ROS production. Left, control, center DOX, and right, DOX plus PepB treatment of human cardiocytes. DOX triggers ROS production, indicated by an increase of fluorescence, while DOX plus PepB treated human cells retain control-level fluorescence. At FIG. 25(F) one sees a quantification of the results in FIG. 25(E). Fluorescence Intensity is presented as % of control for, from left, control, DOX treated, and DOX plus PepB treated human cardiocytes. The restoration of control levels of fluorescence upon PepB treatment indicates that PepB counteracts the inductive effect of DOX on ROS generation. At FIG. 25(G), one sees that PepB reduces DOX mediated Calcium ion overload. Left, control, center DOX, and right, DOX plus PepB treatment of human cardiocytes. DOX triggers ROS production, indicated by an increase of fluorescence, while DOX plus PepB treated human cells retain control-level fluorescence. At FIG. 25(H) one sees a quantification of the results in FIG. 25(G). Fluorescence Intensity is presented as % of control for, from left, control, DOX treated, and DOX plus PepB treated human cardiocytes. The restoration of control levels of fluorescence upon PepB treatment indicates that PepB counteracts the inductive effect of DOX on calcium ion accumulation.

### Definitions

A partial list of definitions is as follows.

As used herein, “about” a number refers to a range spanning from 10% below to 10% above that number. “About” a range refers to an extended range spanning 10% below the listed lower limit of the range to 10% above the upper listed limit of the range.

As used herein the phrase “at least one of A, B, and C” refers to a set that may include only A, A and additional unlisted elements, A and B, A and B and additional unlisted elements, B, B and unlisted elements, A B and C, A B C and unlisted elements, B and C, B and C and unlisted elements, C alone, or C and unlisted elements.

As used herein, the terms peptide, polypeptide and protein are in some cases used interchangeably in reference to a molecule comprising a plurality of peptide bonds.

### EXAMPLES

### Example 1. Cytosolic BNIP3 Determines Necrotic and Apoptotic Cell Death Successively by Regulating BAX in a Heterodimeric State

We hypothesized that cytosolic BNIP3 directly acts on BAX in mitochondrial-driven cell death. To investigate this, we employed first blue native-PAGE (BN-PAGE) on cytosolic fractions of the myocardium followed by Western blotting. We detected BNIP3 in large-scale formation of oligomers with an apparent molecular mass between 272 kDa and 450 kDa by anti-BNIP3 antibody (FIG. 1A). Size exclusion chromatography followed by SDSPAGE/Western blotting (FIG. 7A) verified the apparent presence of BNIP3 in oligomers up to a molecular mass between 522 kDa and 921 kDa, but also showed oligomers down to a molecular mass of 54 kDa possessing BNIP3 (FIG. 1B).

We next evaluated the behavior of BNIP3 in necrosis and apoptosis using the well-established mouse myocardial in vivo I/R model as a clinically relevant system (Hendgen-Cotta et al. 2008; Luedike et al., 2012, Rassaf et al. 2014, Merz et al., 2019). Ischemia was induced by occlusion of the left coronary artery for 30 minutes, followed by 24 hours of reperfusion (FIG. 1C; see STAR Methods). By using Bnip3 deficient (Bnip3^{-/-}) mice, we found that Bnip3 deletion generally protected cardiac tissue from I/R injury, as evidenced by a reduction in infarct size by 51% compared to wild-type mice (FIG. 1D). BNIP3 restitution in Bnip3^{-/-} mice by injection with a TAT-BNIP3 fusion protein (FIG. 7B) restored infarct sizes up to levels comparable to those of wild-type mice dose-dependently (FIG. 1D). Even in mice with acutely affected BNIP3 activity by administration of the dominant-negative form of BNIP3, TAT-BNIP3ΔTM (Diwan et al., 2007; Hamacher-Brady et al., 2007), infarct sizes decreased dose-dependently up to a similar level (−55%) as in Bnip3^{-/-} mice (FIG. 7C). This cell death attenuation was accompanied by a decreased cardiac troponin I release, an indicator of cardiomyocyte necrosis (FIG. 1E). We also found a general decrease in apoptotic cells at 4 hours reperfusion (FIG. 1F). In addition, the spatial distribution of apoptotic cells was minimized throughout the LV in mice with affected BNIP3 activity compared to control mice (FIG. 1F).

On the subcellular level, we identified a time dependent dissipation of the electrical potential difference across the MIM during reperfusion, which culminated in mitochondrial depolarization and swelling at 10 minutes reperfusion (FIGS. 8A and 8B). When we affected BNIP3 activity by TAT-BNIP3ΔTM administration, only marginal MIM depolarization and minor mitochondrial swelling and fragmentation occurred (FIGS. 1G-II, FIGS. 8C and 8D).

Thus, our results raise the tantalizing idea that BNIP3 regulates necrotic signaling within the first 10 minutes of reperfusion. Transient opening of a pore in the MIM (mPTP) as crucial event for mitochondrial depolarization requires translocation of BAX at the MOM, but not its activation (Whelan et al., 2012, Karch et al. 2013). We first observed that BAX, but also BNIP3 translocated from the cytosol into the MOM (FIGS. 1J and 1K). Already at 10 minutes reperfusion, the time-point of loss of Δψm, significant elevated mitochondrial levels of BNIP3 and BAX dimers were present (FIGS. 1J and 1K), but no activated BAX was detectable (FIG. 1L). We observed the presence of activated BAX at 30 minutes of reperfusion. Notably, using TAT-BNIP3ΔTM we found that the large-scale BAX translocation was strongly diminished and, hence, dependent on BNIP3 (FIG. 1J). This impact also applied to BNIP3 (FIG. 1K). Its cytosolic presence and constitutive expression of BNIP3 at transcript and protein level in cardiomyocytes during I/R substantiated a translocation from cytosol to mitochondria (FIGS. 8E-8I).

To further strengthen the notion for BAX dependency on cytosolic BNIP3, we performed I/R surgery in Bnip3−/− mice and obtained similar findings at 10 minutes reperfusion (FIG. 1M). BNIP3 restitution by injection with a TAT-BNIP3 fusion protein was indispensable to achieve BAX translocation at the MOM in BNIP3 deficient mice (FIG. 1M), making it highly likely that in native cardiomyocytes cytosolic BNIP3 dictates BAX translocation.

Co-immunoprecipitation experiments revealed that BAX and BNIP3 were physically associated in the cytosol at baseline and 10 minutes reperfusion, and this association was DTT-resistant corroborating their non-covalent heterodimeric structure (FIG. 1N). Regarding the apoptotic signaling, we found no cytochrome c release within the first 10 minutes of reperfusion, but a significant increase in cytosolic cytochrome c content at 30 minutes reperfusion (FIG. 10). The release of this apoptogenic factor was prevented in mice with affected BNIP3 activity as demonstrated by attenuated cytosolic cytochrome c levels at 30 minutes reperfusion, which were similar to those at baseline and 10 min of reperfusion (FIG. 1O). However, MOMP induction requires the activation of BAX (Kalkavan and Green, 2018, Dewson and Kluck, 2009; Edlich et al., 2011). In accordance with this, the peak BAX activation occurred at 30 minutes reperfusion, and this activation was strongly inhibited by abrogation of BNIP3 activity (FIG. 1L).

We confirmed the dependency of MOMP formation on cytosolic BNIP3 using isolated mouse cardiac mitochondria incubated with cytoplasm by inhibiting BNIP3, BAX and the voltage dependent anion channel (Westphal et al., 2011) (FIG. 9A). BAX activation with release of cytochrome c and AIF was decreased by its inhibition, but also when BNIP3 activity was affected (FIG. 9A). BNIP3-associated MIM and MOM damage was further validated in isolated mouse mitochondria challenged with recombinant BNIP3 and BAX to simulate cytosolic proteins and TAT-BNIP3ΔTM. Both MIM perturbation and MOMP formation was diminished, as evidenced by limited cytochrome c release (FIGS. 9B and 9C). The data tempted us to speculate that a posttranslational modification of BNIP3 provokes BAX activation. To test this, we investigated the temporal phosphorylation of BNIP3 and a potential impact on BAX activation. In accordance with a demonstrated diminished cardiac capability of ATP synthesis within the first 10 minutes of reperfusion (FIG. 1P), the phosphorylation level of BNIP3 was similar to that at baseline, whereas BNIP3 was highly phosphorylated at 30 minutes reperfusion (FIG. 1Q), when BNIP3 drives BAX activation (FIG. 1N). We further confirmed BNIP3 phosphorylation and limited BAX activation by exposing HL-1 cells to hypoxia/reoxygenation and AKT inhibition (FIG. 9D).

Collectively, our findings identify that cytosolic BNIP3 activity governs necrotic and apoptotic cell death in a time-dependent manner by regulating BAX in a heterodimeric state. Thereby, phosphorylation of BNIP3 may activate BAX and MOMP formation constituting the master switch of BNIP3/BAX cell death activity from necrosis into apoptosis.

Example 2. N-terminus is the interacting domain of BNIP3. To map the interaction domain of BNIP3 and the possible binding sites in BAX, we first confirmed the BNIP3/BAX interaction seen in vivo by an in vitro protein-protein overlay devoid of cellular kinases (FIG. 2A). To identify possible binding sites for BNIP3 in BAX, we employed a protein-peptide interaction study with a library of 13 synthesized BAX peptides immobilized on microarrays (FIG. 2B). Fluorescence-labeled recombinant BNIP3 was incubated on peptide microarrays identifying the helices α5, α6, α7 and α8 of BAX as potential interaction sites with BNIP3 (FIG. 2C). Since the 3D structure of BNIP3 has not been fully solved with the exception of its transmembrane domain located at the C terminus using nuclear magnetic resonance spectroscopy (Bocharov et al., 2007; Sulistijo and Mackenzie, 2009), we predicted in silico the 3D structure of BNIP3 by homology modelling using Modeller 9.15 (Eswar et al., 2006) followed by energy minimization using NAMD 2.9 (Phillips et al., 2005) and the CHARMM36 force field (Vanommeslaeghe and MacKerell, 2015) (FIG. 2D). The template structures corresponded to PDB codes 2K7W (Gavathiotis et al., 2008) and 2KA1 (Sulistijo and Mackenzie, 2009). The 3D model features nine α-helices of variable length representing 64% of the secondary structure, followed by random coils (19%) and unidentified structures (17%) (FIG. 2E). We supported the predicted secondary structure of BNIP3 by circular dichroism (CD) spectroscopy (FIG. 2F). Computational docking simulations performed with Autodock Vina (Trott and Olson, 2010) and HADDOCK (Dominguez et al., 2003) also suggested the BAX helices α5, α6, α7 and α8 and BNIP3 sequence MSQSGEENLQGSWVELHFSN (SEQ ID NO: 1) (amino acids 1-20) as interaction sites while the first 10 amino acids alone failed to bind to BAX (FIG. 2G). Thus, BNIP3 amino acids 1-20 appear to be crucial for BNIP3 binding to BAX.

Example 3. N-terminus of BNIP3 is a critical functional domain in human cardiomyocyte protection. Based on our prediction of amino acids 1-20 of BNIP3 as the BAX interaction site, we hypothesized that this sequence is a functional domain of BNIP3 and may be sufficient to antagonize BNIP3/BAX activity. We initially designed a cell-permeable peptide, TAT-BNIP3-20A, composed of the HIV-1 Tat protein transduction domain48-59 (PTD; GRKKRRQRRRPQ (SEQ ID NO: 13)) (Shoji-Kawata et al., 2013) attached via a covalent bond to 20 amino acids derived from amino acids 1-20 of BNIP3 (FIG. 3A). The BNIP3 amino acids 42-61 were used to generate a control peptide, TAT-BNIP3-20C (FIG. 3B). To test first the efficacy of the peptide, we subjected wild-type mice to I/R with TAT-BNIP3-20A injection. Consistent with our hypothesis, TAT-BNIP3-20A prevented BNIP3 translocation and antagonized BNIP3 activity, demonstrated by a markedly reduced caspase-3 activity (FIGS. 3C and 3D). Notably, TAT-BNIP3-20A resulted in a reduction in infarct size by 41% relative to treatment with vehicle, but no difference was observed between TATBNIP3-20C and vehicle treatments (FIG. 3E). To address the translational aspect, since BNIP3 is expressed in the human myocardium (FIG. 3F) (Chaanine et al., 2013), we assessed mouse/human BNIP3 sequence alignment with focus on the N-terminus region at issue. The comparison revealed amino acids 9-20 as conserved residues (FIG. 3G). To identify the crucial amino acids required for activity, we next performed N-terminal truncation analysis of the first 20 amino acids of BNIP3 and identified the amino acids residues 13-20, WVELHFSN (SEQ ID NO: 2), within the evolutionarily conserved domain (FIG. 3H, FIG. 26). This truncated peptide led to the strongest signal with a 14-fold signal increase compared to the 20 residues of BNIP3-20A peptide in a BNIP3/peptide microarray (FIG. 2A). We next focused on substitution analyses (FIG. 27A-FIG. 27E). No peptides with double or triple alanine scan demonstrated stronger binding of the protein when compared to the wild-type sequence (FIG. 10A). Particular amino acid substitution by natural amino acids (A, C, D, E, F, G, H, I, K, L, M, N, P, Q, R, S, T, V, W, Y) within the truncated sequence identified that the exchanges of W13 and F18 by each of the canonical amino acids severely impaired BNIP3 binding (FIG. 3I). This assigns tryptophan and phenylalanine functional roles in the binding activity. Consistently, a S-to-F substitution at position 19 displayed substantially higher BNIP3 binding, underpinning a critical role for phenylalanine (FIG. 3I). Docking simulations of the discovered sequence with S-to-F substitution, WVELHFFN (SEQ ID NO: 3), and BAX showed that peptide binding to the BAX interface is dominated by conformations of the peptide located over the N terminus of helix α5 (FIGS. 3J and 3K). In addition, the obtained conformations suggest that the better binding of WVELHFFN (SEQ ID NO: 3) to BAX compared to the native sequence WVELHFSN (SEQ ID NO: 2) is related to improved aromatic intrapeptide interactions (Martinez and Iverson, 2012). This was confirmed by a BAX/microarray evidenced by a 4-fold increased signal intensity related the native sequence (FIG. 3K). Our findings indicate that these amino acids are necessary to antagonize BNIP3/BAX activity. We therefore designed a peptide in which the PTD was attached via a covalent bond to eight amino acids derived from amino acids 13-20 of BNIP3, but with the S-to-F-substitution (TAT-WVELHFFN (“WVELHFFN” disclosed as SEQ ID NO: 3); FIG. 3L). The CD spectrum of TAT-WVELHFFN (“WVELHFFN” disclosed as SEQ ID NO: 3) showed that the peptide exhibited a random coil conformation (FIG. 3M). To design a dysfunctional peptide as a control, we substituted potential interfacial residues, including the crucial phenylalanine residues F19 to S19 and F18 to A18 and H17 to A17, thereby generating the TAT-WVELAASN (“WVELAASN” disclosed as SEQ ID NO: 7) peptide (FIG. 10B). BNIP3-TAT-WVELHFFN (“WVELHFFN” disclosed as SEQ ID NO: 3) and BAX-TAT-WVELHFFN (“WVELHFFN” disclosed as SEQ ID NO. 3) fluorescence-based overlay assays then confirmed that TAT-WVELHFFN (“WVELHFFN” disclosed as SEQ ID NO: 3) binds to both BNIP3 and BAX (FIG. 3N). To scrutinize the translational aspect, we conducted hypoxia/reoxygenation experiments on human ventricular cardiomyocytes derived from human induced pluripotent stem cells (human CMs; FIG. 3O), which recapitulated BNIP3/BAX interactions assessed by the proximity ligation assay (FIG. 3P). Upon treatment with TAT-WVELHFFN (“WVELHFFN” disclosed as SEQ ID NO: 3), apoptotic and necrotic cell death and MIM depolarization were diminished relative to control peptide treatment (FIG. 3Q)

Example 4. WVELHFFN peptide functions in necrotic and apoptotic signaling prevention in vivo Our results regarding the binding capacity of WVELHFFN (SEQ ID NO: 3) to both BNIP3 and BAX and the protective effect of TAT-WVELHFFN (“WVELHFFN” disclosed as SEQ ID NO: 3) on necrotic and apoptotic death of cardiomyocytes predict an inhibitory effect on the BNIP3/BAX heterodimer induced lethal signaling pathways. To test this, we used the established in vivo myocardial I/R model with focus on the fundamental reperfusion times at 10 and 30 minutes and several complementary methods on the downstream signaling (FIG. 4A). As demonstrated, cell death signaling occurs during the first minutes of reperfusion (FIG. 1). Studies have shown that application of cardioprotective agents after the onset of reperfusion failed to reduce I/R injury (Davidson et al., 2019). Therefore, we decided to administer TAT-WVELHFFN (“WVELHFFN” disclosed as SEQ ID NO: 3) or the control peptide TAT-WVELAASN (“WVELAASN” disclosed as SEQ ID NO: 7) 5 minutes before the end of ischemia, which is a feasible time point for drug application in the clinical setting (FIG. 4A). To ascertain myocardial peptide up-take, distribution and temporal presence following in vivo intracardiac injection, we initially injected ischemic wild-type mice with fluorescence labeled TAT-WVELHFFN (“WVELHFFN” disclosed as SEQ ID NO: 3) 5 minutes before reperfusion. We found that TAT-WVELHFFN (“WVELHFFN” disclosed as SEQ ID NO: 3) was present in the heart at the time of BNIP3/BAX heterodimeric initialization of the cell death cascade (FIG. 4B). As with light sheet microscopic analysis of the whole heart (Merz et al., 2019), the distribution of the peptide occurred also within the area at risk (FIG. 4C). We further evaluated the pharmacokinetic profile of TAT-WVELHFFN (“WVELHFFN” disclosed as SEQ ID NO: 3) in human serum, plasma and whole blood, characterized its toxicity in murine cardiomyocytes and mice and the impact on heart function. The observed stability of TAT-WVELHFFN (“WVELHFFN” disclosed as SEQ ID NO. 3) in human whole blood, plasma and serum over 60 minutes provides in vivo inhibitory capacity (FIG. 11A). Of note, we could not detect signs of overt toxicity in either cardiomyocytes or mice (FIGS. 11B and 11C). Consistent with our hypothesis, upon treatment with TAT-WVELHFFN (“WVELHFFN” disclosed as SEQ ID NO: 3), BNIP3 and BAX translocation to the mitochondria at 10 minutes reperfusion was markedly diminished (FIGS. 4D and 4E). By consequence, necrotic and apoptotic signaling characterized by mitochondrial swelling at 10 minutes of reperfusion as well as BAX activation, cytochrome c release, and caspase-3 activity at 30 minutes reperfusion were strongly reduced (FIGS. 4F-41).

### Example 5. WVELHFFN Functions in Inhibition of BNIP3/BAX Heterodimers as Components of the Mitochondrial Membrane Attack Oligomers

Based on the knowledge of cytosolic non-covalent BNIP3/BAX heterodimers at baseline and 10 minutes at reperfusion obtained by co-immunoprecipitation (FIG. 1), we sought to determine the mode of action of WVELHFFN (SEQ ID NO: 3) on BNIP3/BAX heterodimers. Upon peptide treatment, neither BNIP3 nor BAX dimers were influenced at 10 minutes of reperfusion (FIGS. 5A and 5B). In co-immunoprecipitation experiments the unaffected heterodimeric state of BNIP3 and BAX was confirmed (FIG. 5C). To investigate whether TAT-WVELHFFN (“WVELHFFN” disclosed as SEQ ID NO: 3) interacts with the BNIP3/BAX heterodimer, we used fluorescence-labeled TAT-WVELHFFN (“WVELHFFN” disclosed as SEQ ID NO: 3) injected in wild-type mice, which were subjected to I/R. Western Blot analysis revealed the binding of TAT-WVELHFFN-fluo (“WVELHFFN-fluo” disclosed as SEQ ID NO: 9) to dimers (FIG. 5D). We further verified this by coimmunoprecipitation of cytosolic BNIP3 and BAX and photometric fluorescence measurement (FIG. 5E). As expected, 5 minutes post reperfusion, cytosolic BNIP3 and BAX coimmunoprecipitated with TAT-WVELHFFN-fluo (“WVELHFFN-fluo” disclosed as SEQ ID NO: 9) (FIG. 5E). BN-PAGE and size exclusion chromatography followed by SDS-PAGE/Western blotting confirmed the presence of BNIP3 and BAX in apparent oligomers up to a molecular mass between 95 kDa and 1627 kDa, but also showed oligomers with BNIP3 down to a molecular mass of 54 kDa in the cytosol at baseline and in I/R at 10 minutes reperfusion with and without TAT-WVELHFFN (“WVELHFFN” disclosed as SEQ ID NO: 3) treatment (FIG. 1B and FIG. 5F-5H). To discover which oligomeric complex with BNIP3/BAX subunits exerts the harmful activities on mitochondrial membranes, we treated the wild-type mice with the fluorescence-labeled TAT-WVELHFFN (“WVELHFFN” disclosed as SEQ ID NO: 3). Size exclusion chromatography followed by fluorescence measurement revealed that the higher oligomeric complexes led to the strongest signal with up to 250/6 signal increase compared to the fraction consisting of dimers/oligomers with a molecular mass between 54 and 167 kDa (FIG. 5I). Taken together, these data indicate that BNIP3/BAX heterodimers are components of higher oligomeric structures. As TAT-WVELHFFN (“WVELHFFN” disclosed as SEQ ID NO. 3) prevents BNIP3/BAX induced necrotic and apoptotic signaling, the binding of TAT-WVELHFFN (“WVELHFFN” disclosed as SEQ ID NO: 3) to oligomers outline their status as mitochondrial membrane attack complexes.

Example 6. TAT-WVELHFFN (“WVELHFFN” disclosed as SEQ ID NO: 3) improves clinical outcomes. The infarct size determines prognosis following myocardial infarction and stroke. Hence, we studied whether TAT-WVELHFFN (“WVELHFFN” disclosed as SEQ ID NO: 3) can alter the infarcted area when administered during ischemia, 5 minutes before reperfusion in wild-type mice subjected to myocardial I/R (FIG. 6A), a clinically relevant time-point. Notably, TAT-WVELHFFN (“WVELHFFN” disclosed as SEQ ID NO: 3) reduced myocardial infarct size in a dose-dependent manner by 40% relative to treatment with vehicle, but no difference was observed between TAT-fi-Gal, TAT-WVELAASN (“WVELAASN” disclosed as SEQ ID NO: 7) and vehicle treatments (FIGS. 6B-6D). We also observed a lower progression of apoptotic cell death throughout the LV myocardium (FIGS. 6E and 6F). Treating TAT-BNIP3ΔTM mice (with affected BNIP3 activity) with TAT-WVELHFFN (“WVELHFFN” disclosed as SEQ ID NO: 3) failed to induce further reduction in infarct size to TATBNIP3ΔTM treatment, indicating a TAT-WVELHFFN (“WVELHFFN” disclosed as SEQ ID NO: 3) effect via BNIP3 (FIG. 6G).

To explore the translational potential, we chose the catheter-based myocardial infarction model in pigs because of similarities of organ size, coronary anatomy, immunology, and physiology to humans (Lindsey et al., 2018; Milani-Nejad and Janssen, 2014). Pigs were exposed to 60 minutes of ischemia by balloon inflation in the left coronary artery followed by 4 hours of reperfusion. TAT-WVELHFFN (“WVELHFFN” disclosed as SEQ ID NO: 3) or sodium chloride was administered 5 minutes prior to reperfusion. Notably, one single intravenous bolus injection of 0.075 mg/kg of the peptide was sufficient to decrease infarct size by 60% in pigs related to controls (FIG. 6H). As BNIP3 might play a critical role in cerebral ischemia (Zhang et al., 2007), we also evaluated the effects of TAT-WVELHFFN (“WVELHFFN” disclosed as SEQ ID NO: 3) on focal cerebral ischemia. We subjected mice to a 30-minute transient middle cerebral artery occlusion and 24 hours of reperfusion. Here, TAT-WVELHFFN (“WVELHFFN” disclosed as SEQ ID NO: 3) treatment immediately before reperfusion markedly reduced the infarct size by 52% compared to controls (FIG. 6I). To explore the effect of a pre-treatment we administered TAT-WVELHFFN (“WVELHFFN” disclosed as SEQ ID NO: 3) on d−7, d−5, d−3, d−1, and 5 minutes before 24 h of reperfusion to wild-type mice subjected to a 30-minute ischemia (FIG. 6J). Here, multiple treatment reduced the infarct size by 40% (FIG. 6K). Treatment with TAT-WVELHFFN (“WVELHFFN” disclosed as SEQ ID NO: 3) on day −7, day −5, day −3, day −1 before the onset of I/R preserves cardiac function (FIG. 6L). To evaluate the effects of TAT-WVELHFN (“WVELHFN” disclosed as SEQ ID NO: 14) administered 5 minutes before reperfusion and on day 1, day 3, day 5, and day 7 after the start of reperfusion to wild-type mice exposed to 50 minutes of ischemia on cardiac function, echocardiographic measurements were performed (FIG. 6M). Notably, treated mice exhibited recovery of LV function at day 3 (FIG. 6N). Taken together, our findings demonstrate that this peptide improves clinical outcome in myocardial infarction and stroke.

Example 7: TAT-WVELHFFN (“WVELHFFN” disclosed as SEQ ID NO: 3) ameliorates DOX induced human cardiomyocyte death and preserves mitochondrial integrity. Anthracyclines remain the cornerstone of contemporary chemotherapeutic regimens for a variety of solid cancers and hematologic malignancies. Anthracycline-induced cardiotoxicity (AIC) is a feared side effect limiting their clinical use. The risk of AI heart failure increases with the administered cumulative dose, its incidence reaches nearly 10%. Here we studied whether TAT-WVELHFFN (“WVELHFFN” disclosed as SEQ ID NO: 3) can reduce the chemotherapeutic DOX induced cell death in human cardiomyocytes. Therefore, we administered DOX at 0, 1, and 10 μM to human cardiomyocytes. Cells were incubated for four and 22 hours, and TAT-WVELHFFN (“WVELHFFN” disclosed as SEQ ID NO: 3) was administered at 0 or 1.5 pM. The results show that the attributive damage caused by a 10 μM dose of the chemotherapeutic DOX was ameliorated by the mitochondrial interaction and import inhibitor TAT-WVELHFFN (“WVELHFFN” disclosed as SEQ ID NO: 3) (FIGS. 14A and B). We next explored the effects of TAT-WVELHFFN (“WVELHFFN” disclosed as SEQ ID NO: 3) on DOX-induced mitochondrial damage related to mitochondrial membrane potential destabilization, mPTP opening in the MIM, mitochondrial Ca2+ overload, and generation of mitochondrial ROS. Human cardiomyocytes were incubated with 10 μM DOX for four and 22 hours, respectively. TAT-WVELHFFN (“WVELHFFN” disclosed as SEQ ID NO: 3) was co-administrated. The mitochondrial membrane potential was disrupted and the mPTP opening was activated by administration of 10 μM DOX, while co-administration of TAT-WVELHFFN (“WVELHFFN” disclosed as SEQ ID NO: 3) ameliorated these negative effects of DOX. This indicates that the negative effects of the chemotherapeutic on mitochondrial membrane potential and mPTP opening are ameliorated by co-administration of TAT-WVELHFFN (“WVELHFFN” disclosed as SEQ ID NO: 3) (FIGS. 15 and 16). Furthermore, mitochondrial calcium ion overload was induced by administration of 10 μM DOX for four hours-incubation time and ROS generation was triggered by administration of 10 μM DOX for four- and 22 hours-incubation time. Co-administration of TAT-WVELHFFN (“WVELHFFN” disclosed as SEQ ID NO: 3) prevented the negative effect of DOX on calcium ion overload and ameliorated the negative effect of DOX on ROS generation. This indicates that the negative effects of the chemotherapeutic on mitochondrial Ca2+ overload and mitochondrial ROS generation are ameliorated by co-administration of TAT-WVELHFFN (“WVELHFFN” disclosed as SEQ ID NO: 3).

Example 8 TAT-WVELHFFN (“WVELHFFN” disclosed as SEQ ID NO: 3) preserves cardiac function by preventing cardiomyocyte death, protecting mitochondrial fitness and restoring autophagic flux. In a first attempt, we investigated whether DOX with concomitant administration of TAT-WVELHFFN (“WVELHFFN” disclosed as SEQ ID NO: 3) (PepB) over 42 days has long term detrimental effects on mouse recipients (FIG. 19). Upon Dox treatment with PepB co-administration relative to a sodium chloride carrier control in wild-type mice, we observed no significant differences related to survival, body weight change over time, tibia length, and heart weight (FIG. 20). Cardiac troponin I (cTNI)/body weight was significantly lower for DOX coadministration with PepB relative to DOX administration with an NaCl control. Similarly, creantine kinase (CK)/body weight was significantly lower for DOX coadministration with PepB relative to DOX administration with an NaCl control. LDH/body weight and BNP/body weight were unchanged by administration of PepB (FIG. 21). Next, we explored the effect of DOX treatment on cardiac function assessed with echocardiographic measurements at three time points (T0, T1 16 days, T2 42 days) (FIG. 19).

At both T1 (16 days) and T2 (42 days), DOX negatively impaired ejection fraction (EF %), fractional shortening (FS %), and the isovolumetric relaxation time (IVRT) and this impact was ameliorated by concurrent PepB administration. Heart rate was unaffected by DOX administration, alone or in combination with PepB. Notably, DOX had a dramatic positive effect on cardiomyocyte death, evidenced by high cardiac troponin I (cTnl) levels, and this effect was ameliorated by coadministration of PepB. In a next step, we investigated the impact of PepB on DOX-induced mitochondrial dysfunction. DOX treatment inhibited the respiratory reserve capacity and PepB ameliorated this inhibition. In addition, DOX also affected the maximal respiration and basal respiration, and PepB ameliorated these impairments. The inhibition of Complex II-stimulated respiration was also reduced by PepB co-treatment. To elucidate whether PepB co-administration is able to restore the DOX-induced disturbance of the autophagic flux, we treated the mice with DOX and PepB, and additionally with chloroquine (CQ). As autophagosome marker we measured the expression of LC3-I and LC3-II. DOX abolished the CQ mediated LC3II/I increase, while addition of PepB partially restores this increase. DOX also triggered p62 and BNIP3 accumulation, and addition of PepB counteracted these increases.

Additional methods and materials were drawn from the following sources: REAGENT or RESOURCE SOURCE IDENTIFIER Antibodies Anti-BNIP3 polyclonal antibody, Rabbit Abcam Cat #ab38621, RRID:AB_725737 Anti-BNIP3 monoclonal antibody, Mouse Abcam Cat #ab10433 RRID:AB_2066656 Anti-BAX monoclonal antibody [E63], Rabbit Abcam Cat #ab32503, RRID:AB_725631 Cat #ab7977 RRID:AB_306191 Anti-ANT 1 monoclonal antibody [5F51BB5AG7], Mouse Abcam Cat #ab110322, RRID:AB_10862212 Anti-Troponin I monoclonal Antibody (1H11L19), ABfinity™, Rabbit ThermoFisher Scientific Cat #701585, RRID:AB_2532494 Anti-Phosphoserine monoclonal antibody, clone 4A4, Mouse Merck Millipore Cat #05-1000, RRID:AB_11210897 Anti-TOMM22 Abcam Cat #ab57523 RRID:AB_945897 Anti-BAX (active monomer) monoclonal antibody (6A7) Enzo Life Sciences Cat #ALX-804-224-C100, RRID:AB_2050800 Anti-Tubulin antibody Abcam Cat #ab15246 RRID:AB_302787 Anti-AIF antibody Santa Cruz Cat #Sc9416 RRID:AB_2224665 Anti-Cytochrome C monoclonal antibody, Unconjugated, Clone 7H8.2C12, M Mouse s Abcam Cat #ab13575, RRID:AB_300470 AlexaFluor 488-conjugated goat anti-rabbit secondary antibody Invitrogen Cat #A11029 RRID:AB_138404 Goat Anti-rabbit-HRP secondary antibody Invitrogen Cat #32260 RRID:AB_1965959 Goat Anti-mouse-HRP secondary antibody Abcam Cat #AB 6789 RRID:AB_955439 AlexaFluor 594-conjugated goat anti-rabbit secondary antibody Invitrogen Cat #A-11080 RRID:AB_2534124 Bacterial and Virus Strains Tuner DE3pLys Competent Cells Novagen/Merck Millipore 70624 Biological Samples human endomyocardial biopsy Ethics Approval of the Ethics Committee, University Duisburg-Essen, No. 17-7392-BO N/A Chemicals, Peptides, and Recombinant Proteins Bax Mouse Recombinant (Bax Mouse) Novateinbio PT_39984 Recombinant Mouse BCL2/adenovirus EIB 19 kDa protein interacting protein 3(Bnip3) Cusabio CSB-CF002766Mo-50 TAT-BNIP3-20A JPT this paper TAT-BNIP3-20C JPT This paper TAT-WVELAASN (“WVELAASN” disclosed as SEQ ID NO: 7) JPT this paper TAT-WVELHFFN (“WVELHFFN” disclosed as SEQ ID NO: 3) JPT this paper TAT-WVELHFFN (“WVELHFFN” disclosed as SEQ ID NO: 3)-Fam JPT this paper TAT-WVELHFFN-Cy5.5 (“WVELHFFN-Cy5.5” disclosed as SEQ ID NO: 10) JPT this paper TAT-BNIP3 full length this paper TAT-BNIP3ΔTM this paper Tat-βGal this paper Critical Commercial Assays this paper Duo Link in Situ Starter Kit-Proximity Ligation Assay Sigma DUO92101-1KT Caspase 3 Assay Kit Colorimetric Abcam Ab39401 Apoptag red in Situ Detection Millipore S7165 ATP Detection Kit Abcam Ab83359 Apoptose/Necrose Assay Kit Abcam ab176749 Troponin I Ultra-sensitive Elisa Life Diagnostic CTNI-1-US DyLight Antibody Labeling Kits ThermoFisher Scientific 84535 Ni-NTA Fast Start Kit Qiagen 30600 Peptide microarrays JPT N/A JC-1 Kit ThermoFisher Scientific M34152 RNeasy Mini Kit Qiagen 74104 High Capacity RNA-to-cDNA Kit Life Technologies 4388950 Experimental Models: Cell Lines human IPSC-derived vCMs Axol Bioscience Ax 2505 HL-1 cell line W. C. Claycomb Louisiana State University Medical Center, New Orleans, LA, USA W. C. Claycomb Experimental Models: Organisms/Strains C57BL/6J-TgH (Bnip3−/−) Gerald W. Dorn Gerald Dorn C57BL/6J wild-type Jackson Laboratory 000664 Oligonucleotides 18S rRNA ThermoFisher Scientific Mm03928990_g1 Bnip3 ThermoFisher Scientific Mm00833810 Recombinant DNA TAT-Bnip33/pTriEx-1.1-HT Labor Ruhruniversitut Bochum R. Stoll TAT-Bnip3 dTM/pTriEx-1.1-HT Labor Ruhruniversitat Bochum, R. Stoll pTAT-HA-β-Gal vector S. F. Dowdy (University of California, San Diego, CA, USA) Science 3 Sep. 1999: Vol. 285, Issue 5433, pp. 1569-1572 DOI: 10.1126/science.285.5 433.1569 Software and Algorithms ImageJ Schneider et al., 2012 https://imagej.nih.gov/ij/Inkscape https://inkscape.org/Vevo 2100 Imaging System FUJIFILM, VisualSonics, Netherland Imaris Software (BitPlane) BitPlane www.bitplane.com GraphPad Prism 7 GraphPad Other Superdex 200 Increase 10/300 GL GE Healthcare 28-9909-44

EXPERIMENTAL MODEL AND SUBJECT DETAILS Mice All animal procedures were performed in accordance with institutional guidelines and approval from the local ethics committee in compliance with the European Convention for the Protection of Vertebrate Animals Used for Experimental and other Scientific Purposes (Directive 2010/63/EU). Male mice aged 12±3 weeks with an average body weight of 30 g were used. C57BL/6J wild-type mice were obtained from the Jackson Laboratory (Bar Harbor, ME, USA) and kept for 1 week in the local animal house for acclimatization. C57BL/6J-TgH (Bnip3−/−) mice were bred and maintained in the local animal house of the University Hospital Essen. All mice were housed on a 12-hour light/dark cycle. Cell lines The HL-1 cell line was provided by W. C. Claycomb (Louisiana State University Medical Center, New Orleans, LA, USA) and cultured as previously described (Claycomb et al., 1998). Human CMs were obtained from Axol Bioscience, UK, and cultured according to the manufacturer's instructions. METHOD DETAILS Recombinant protein expression The TAT-BNIP3 and TAT-BNIP3ΔTM plasmids were provided by R. Stoll (Ruhr-University Bochum, Germany). The pTAT-HA-β-Gal vector was provided by S. F. Dowdy (University of California, San Diego, CA, USA). His-tagged BNIP3 and BNIP3ΔTM were cloned into the pTriEx-1.1 vector. BNIP3 (full length), BNIP3ΔTM (amino acids 1-163) and β-galactosidase, all fused to the HIV-1 TAT protein transduction domain (GRKKRRQRRRPQ (SEQ ID NO: 13)), were grown in Escherichia coli (BL21) and expressed with 100 mM IPTG for 48 hours at 37° C. The bacteria were resuspended in PBS (pH 8.0), followed by incubation with 1 mg/mL lysozyme for 1 hour at 4° C. and sonication on ice. After centrifugation at 20,000×g for 50 minutes at 4° C., the supernatants were added to columns containing Ni-NTA. The proteins were eluted with 250 mM imidazole in phosphate buffer. Recombinant BAX (rBAX, PT_39984) and recombinant BNIP3 (rBNIP3, CSB-CF002766) were obtained from Hölzel Diagnostika (Cologne, Germany). In vivo I/R models In vivo murine myocardial IR For in vivo I/R, the open-chest model was used. Briefly, wild-type and Bnip3−/− mice were anesthetized with ketamine (100 mg kg-1 intraperitoneally, i.p.) and xylazine (10 mg kg-1 i.p.), intubated and ventilated. Deep anesthesia was maintained with 1.2-2 Vol % isoflurane in conjunction with a gas mixture of 0.2 l/minutes O2 and 0.2 l/minutes compressed air. A lateral thoracotomy was performed and left coronary artery (LCA) occlusion was achieved by tightening and tying a 6-0 prolene suture. After 30 minutes, the LCA was re-occluded for the desired reperfusion times. The thoracic cavity was closed using a 4-0 prolene suture. Buprenorphine was injected for analgesia every 8 hours. Infarct size was assessed after 30 minutes of ischemia followed by 24 hours of reperfusion using Evans blue staining for delineation of the ischemic area at risk (AAR) from the non-ischemic zone (remote area) and 1% 2,3,5-triphenyl tetrazolium chloride (TTC) staining for demarcation of the viable and nonviable myocardium within the AAR, as described (Hendgen-Cotta et al., 2007). The infarct area, AAR, and non-ischemic left ventricle were assessed with computer-assisted planimetry by an observer blinded to sample identity. The size of the myocardial infarction is expressed as a percentage of the AAR. Treatment in in vivo murine myocardial IR To evaluate the (patho)physiological relevance of BNIP3 in vivo, mice were injected with sodium chloride (NaCl) as vehicle, TAT-BNIP3 (67 nmol kg-1, 200 nmol kg-1, or 300 nmol kg-1 in 50 μL 0.9% NaCl) and TAT-BNIP3ΔTM (33 nmol kg-1, 67 nmol kg-1, or 133 nmol kg-1 in 50 μL 0.9% NaCl). The TAT-fusion proteins were injected into the left ventricular (LV) cavity 5 minutes before ischemia. To assess the in vivo inhibitory effects and efficacy of TAT-BNIP3-20A and TAT-WVELHFFN (“WVELHFFN” disclosed as SEQ ID NO. 3), mice were injected with NaCl, TAT-β-Gal, TAT-BNIP3-20A (67 nmol kg-1 in 50 μL 0.9% NaCl), TAT-BNIP3-20C (67 nmol kg-1 in 50 μL 0.9% NaCl), TAT-WVELHFFN (“WVELHFFN” disclosed as SEQ ID NO: 3), TAT-WVELHFFN-Lys (5(6)Carboxyfluorescein) (“WVELHFFN-Lys(5(6)-Carboxyfluorescein” disclosed as SEQ ID NO: 15), and TAT-WVELHFFN-Cy5.5 (“WVELHFFN-Cy5.5” disclosed as SEQ ID NO: 10) (67 nmol kg-1, 133 nmol kg-1, 267 nmol kg-1, or 667 nmol kg-1 in 50 μL 0.9% NaCl) or TAT-WVELAASN (“WVELAASN” disclosed as SEQ ID NO: 7) (267 nmol kg-1 in 50 μL 0.9% NaCl) 5 minutes before reperfusion. In vivo murine brain I/R Focal cerebral ischemia was induced by a 30-minute transient middle cerebral artery occlusion (tMCAO). Briefly, mice were anesthetized with 2% isoflurane in O2. A servo-controlled heating blanket was used to maintain a core body temperature close to 37° C. throughout surgery. After a midline neck incision, a standardized silicon rubber-coated No. 6.0 nylon monofilament (6023910PK10; Doccol, Sharon, MA, USA) was inserted into the right common carotid artery and advanced via the internal carotid artery to occlude the origin of the MCA. After 30 minutes, the mice were re-anesthetized, and the occluding filament was removed to allow reperfusion. Stroke volumes were assessed 24 hours after tMCAO, based on TTC staining. The mice were then randomly assigned to the operation by an independent researcher who was not involved in the data analysis. Investigators involved in the surgery and evaluation of all readout parameters were blinded to the experimental groups. Treatment in in vivo murine brain I/R The mice were injected with sodium chloride or TAT-WVELHFFN (“WVELHFFN” disclosed as SEQ ID NO. 3) (267 nmol kg-1 in 50 μL 0.9% NaCl) immediately before reperfusion. In vivo porcine myocardial I/R The experiments were performed by Charles River Laboratories, Inc. (Mattawan, MI, USA, Testing facility study No. 2865-001) in accordance with The Animal Welfare Act (9 CFR Parts 1, 2, and 3) of the US Department of Agriculture (USDA), and the Guide for the Care and Use of Laboratory Animals, Institute of Laboratory Animal Resources, National Academy Press, Washington, DC, 2011. For in vivo IR, the closed-chest model was used. Naïve Domestic Yorkshire crossbred swines (farm pigs, male, body weight ranging from 37.5 to 40.5 kg) were placed in a dorsal recumbency, and the surgical sites were prepared with alternating wipes of chlorhexidine scrub and solution. The animals were connected to a defibrillator and ECG machine and monitored thoroughly throughout the procedure. Following the induction of anesthesia, a small incision was made over the femoral artery and vein; the vessels were isolated. A small opening was made in the artery and a sheath was introduced. In addition, a sheath was placed in the femoral vein to allow for emergency drug administration, if necessary. An appropriate guide catheter was advanced into the ostium of the left anterior descending artery (LAD) using visual guidance followed by fluoroscopy. Non-ionic contrast was used for all procedures. A balloon catheter was introduced by advancing it through the guide catheter to the LAD coronary artery. The balloon was advanced into the coronary arteries through the guide catheter to a suitable place above the first diagonal branch of the LAD. The balloon was then inflated to a pressure sufficient to ensure complete occlusion of the artery, and verified via fluoroscopy. Once verified, the balloon was left inflated in the artery for 60 minutes. At the end of the ischemic period, the balloon was deflated and the ischemic area was allowed to reperfuse for 4 hours. Complete balloon deflation was confirmed via fluoroscopy. At the end of the procedure, all catheters were removed, the artery and vein were ligated, and the incision was closed in a standard fashion. For infarct size assessment, the hearts were removed and flushed with heparinized lactated ringer solution until clear of blood. The LAD was tied off at the location of the balloon occlusion during the myocardial infarction procedure. Once tied off, the LAD, LCX, and RCA were cannulated. Evans blue dye was injected. The hearts were cut from base to apex into approximately 1 cm serial sections. Each section was weighed and photographed. The sections of the heart were stained with TTC for 30 minutes at 37° C. and photographed a second time. The infarct size was calculated as a percent of the AAR. The images (including a ruler) were analyzed and the ratios of infarct/AAR, infarct/LV area, and AAR/LV were calculated. The heart sections were stored frozen in 10%/o neutral buffered formalin (NBF) at −60° C. to −90° C. for possible future analysis. Treatment in in vivo porcine myocardial I/R Following the 60-minute occlusion, TAT-WVELHFFN (“WVELHFFN” disclosed as SEQ ID NO: 3) (0.075 mg kg-1 BW) or sodium chloride was administered via intravenous bolus injection 5 minutes prior to reperfusion. Transthoracic echocardiography was used to measure LV systolic function in wild-type mice in response to TAT-WVELHFFN (“WVELHFFN” disclosed as SEQ ID NO: 3) treatment. Echocardiography was performed before and 5 hours after injection of TAT-WVELHFFN (“WVELHFFN” disclosed as SEQ ID NO: 3) (267 nmol kg-1) as well as before and after I/R at desired time point using a Vevo 2100 and Vevo 3100 Imaging System (FUJIFILM VisualSonics, The Netherlands). The investigator was blinded with respect to the untreated and treated groups. After sedation with 2 Vol % isoflurane and hair removal, the mice were placed on a heated plate with constant heart rate, respiratory rate and body temperature monitoring via a rectal probe. Fractional shortening was determined via M-mode in the midventricular short parasternal axis. The LV ejection fraction was calculated using Simpson's method. Isolation of cytosol and mitochondria Fractionation The hearts were washed in ice-cold homogenization buffer (250 mM sucrose, 10 mM HEPES, and 1 mM EGTA, pH 7.4), homogenized in 4 ml buffer containing 0.5% bovine serum albumin and centrifuged at 700×g for 10 minutes at 4° C. to remove unbroken tissue and nuclei. For general fractionation, the supernatant was centrifuged at 15,000×g for 10 minutes at 4° C. to obtain the cytosolic fraction in the supernatant and the mitochondria in the pellet. The mitochondrial pellet was further cleared of contaminations by washing twice in isolation buffer. The cytosolic and mitochondrial fractions were further processed for western blotting. Subfractionation of MIM and MOM The hearts were washed in ice-cold homogenization buffer (250 mM sucrose, 10 mM HEPES, and 1 mM EGTA, pH 7.4), homogenized in 4 ml buffer containing 0.5% bovine serum albumin and centrifuged at 700×g for 10 minutes at 4° C. to remove unbroken tissue and nuclei. The supernatant was centrifuged at 15,000×g for 10 minutes at 4° C. to pellet the mitochondria. Then, the supernatant was centrifuged at 105,000×g for 30 minutes at 4° C. to obtain the cytoplasm. The pellet containing the mitochondria was carefully washed two times in homogenization buffer. Mitochondrial protein concentrations were determined by Bradford method. For inner and outer membrane preparation, the isolated mitochondria were suspended in 0.25 M sucrose solution, incubated in 0.3% digitonin for 20 minutes on ice and centrifuged at 9,500-g for 15 minutes. The supernatant was centrifuged at 105,000×g for 1 hour at 4° C. to obtain the MIM in the supernatant. Pellet II was suspended in buffer A (0.25 M sucrose, 10 mM Tris-HCl, pH 7.4) and incubated overnight at 4° C. Pellet I was suspended in 0.1 M Na2CO3 solution, incubated for 20 minutes on ice and centrifuged at 105,000×g for 30 minutes. Pellet III was suspended in buffer A and incubated overnight at 4 C. The next day, pellets II and III were loaded on a discontinuous sucrose gradient (0.5 ml 51.3%, 0.5 ml 44.3%, 1 ml 37.4% and 1 ml 23.2% sucrose in 0.1 M phosphate buffer, pH 7.4) and centrifuged at 105,000×g for 2 hours at 4° C. Fractions (200 μL) were collected and analyzed by western blotting with the indicated antibodies. Histopathological analyses Infarct area Mouse hearts were perfused with PBS before collection, fixation and preparation of paraffin embedded sections for histopathological analyses. Standard Gomori Trichrome staining was performed to visualize the infarct area. Slices were incubated in Weigert's iron haematoxylin working solution (Merck, Darmstadt, Germany) for 10 minutes at room temperature and then washed 10 times in H2O. Next, the slices were incubated in Gomori's Trichrome solution (Morphisto, Cologne, Germany) for 15 minutes at RT and washed 10 times in H2O, two times in 0.5% acetic acid and 10 times in H2O. Apoptosis To detect apoptotic nuclei, TUNEL staining was performed using an ApopTag Red In situ Apoptosis Detection Kit (Millipore, S7165, Darmstadt, Germany) according to the manufacturer's instructions. The total numbers of TUNEL-positive nuclei were counted in five plane sections of the heart to calculate the number of TUNEL-positive nuclei per heart and per section for spatial distribution analyses. Images were captured under an inverted epifluorescence microscope (Zeiss Axio Observer Z1, Oberkochen, Germany) and processed in ImageJ software 1.52a (NIH). Necrosis Troponin staining was performed by deparaffinization of mouse heart sections (I/R), washing twice in H2O and twice in TBS-T (0.1% Tween 20), and blocking with 5% normal goat serum in PBS for 1 hour. Sections were incubated with anti-troponin I antibody (Invitrogen, 1:100 dilution) overnight at 4° C. and stained with the secondary goat anti-rabbit Alexa Fluor 594 antibody (1:200 dilution). After washing with TBS-T and staining with DAPI (Invitrogen, Carlsbad, CA, USA; 1:5,000 dilution), sections were mounted in ProLong Gold Antifade (ThermoFisher Scientific, Waltham, MA, USA). Images were captured under an inverted epifluorescence microscope (Zeiss Axio Observer Z1, Oberkochen, Germany). Troponin in plasma To examine necrotic cell death, blood was collected from the abdominal aorta after 24 hours of reperfusion following 30 minutes of ischemia; the blood was centrifuged at 3,000×g for 10 minutes at 4C, and plasma was collected. Troponin I levels were measured using a Troponin I ELISA (Life Diagnostics, CTNI-1-US, Westchester, PA, USA). Real-time quantitative RT-PCR (real-time qRT-PCR). Total RNA was isolated from hearts using an RNeasy Mini Kit (Qiagen, Hilden, Germany, 74104) according to the manufacturer's instructions. Purified RNA was quantified using a NanoDrop instrument (ThermoFisher Scientific, Waltham, MA, USA). cDNA was generated using a High Capacity RNA-to-cDNA Kit (Life Technologies, Carlsbad, CA, USA). Real-time qRT-PCR was performed using TaqMan Gene Expression Assays (ThermoFisher Scientific, Waltham, MA, USA, Mm00833810) in a 7900HT Fast Real-Time PCR System (ABI PRISM® 7900HT, Applied Biosystems, Foster City, CA, USA). Reactions were normalized to 18S rRNA levels, and relative transcript abundance was calculated using the comparative Ct method. Size Exclusion Chromatography One mg of cardiac cytosolic lysate was applied to a Superdex® 200 Increase 10/300 GL (GE Healthcare, Chicago, Illinois, USA) equilibrated in SEC buffer (10 mM Tris (pH 7.5), 1 mM EGTA, 200 mM Sucrose, Protease Phosphatase Inhibitors (Halt, Thermo Fisher Scientific, Waltham, Massachusetts, USA) and run at 4° C. Fractions of 1000 μl were collected of which 500 l were concentrated to 40 μl using Amicon® Ultra 0.5 ml Centrifugal Filters (Merck Millipore, Burlington, Massachusetts, USA). The recovered sample was directly processed for subsequent SDS-PAGE and Western Blot. To estimate the molecular weight of the BNIP3 oligomer, a gel filtration molecular weight marker (#1511901, BioRad, Hercules, California, USA) was applied to the columns to obtain a standard curve. Fluorescence measurements of Cy5.5 labeled TAT-WVELHFFN (“WVELHFFN” disclosed as SEQ ID NO: 3) were performed in triplicates in an Infinite® 200 PRO microplate reader (Tecan Group Ltd., Switzerland) with a black flat bottom 96-well plate using unprocessed 150 μl SEC-fraction per well. Fluorescence intensity was measured at 630 nm (±9 nm) excitation/680 nm (±20 nm) emission. Western blotting SDS-PAGE Frozen tissues, HL-1 cells and human CMs were lysed in ice-cold RIPA buffer (50 mM Tris-HCl, 150 mM NaCl, 0.5 mM EDTA, 1% NP-40 and protease and phosphatase inhibitor, pH 7.4). Isolated mitochondria were lysed in a Mito-lysis buffer (200 mM sucrose, 10 mM HEPES, 1 mM EGTA, 1% Triton X-100, protease and phosphatase inhibitor, pH 7.4) and lysates were cleared by centrifugation at 20,000-g for 15 minutes at 4° C. Protein concentrations in supernatants were measured using a DC Protein Assay (Bio-Rad Laboratories Inc., Hercules, CA, USA). Then, samples were diluted in 4×LDS sample buffer and 1Ox reducing agent (Invitrogen, Carlsbad, CA, USA) and boiled at 95° C. for 5 minutes and loaded onto NuPAGE™ 4-12% Bis-Tris Protein Gels (Invitrogen, Carlsbad, CA, USA). Western blot analyses were performed on nitrocellulose membranes after blocking in TBS-T containing 5% milk using the following antibodies incubated over night at 4° C.: anti-ANT1 (Abcam, Cambridge, UK, ab110322, 1:1,000 dilution), anti-AIF (Santa Cruz Biotechnology, Dallas, TX, USA, sc9416, 1:200 dilution), anti-BNIP3 (Abcam, Cambridge, UK, ab38621, 1:1,000 dilution), anti-BAX (Abcam, Cambridge, UK, ab7977, ab32503, 1:500 dilution), anti-cytochrome c (Abcam, Cambridge, UK, ab13575, 1:1,000 dilution), anti-TOMM22 (Abcam, Cambridge, UK, ab57523, 1:500 dilution), anti-tubulin (Abcam, Cambridge, UK, ab15246, 1:5,000 dilution), and anti-active BAX (Enzo Life Sciences, Lorrach, Germany, 6A7, 1:1,000 dilution). The membrane was incubated for 1 hour at room temperature with HRP-conjugated secondary from the appropriate species and exposed to SuperSignal West Pico Plus Substrate (ThermoFisher Scientific, Waltham, MA, USA) as instructed by the manufacturer with subsequent detection. BN-PAGE The protein concentration of the isolated cytosolic fractions was measured using a DC Protein Assay (Bio-Rad Laboratories Inc., Hercules, CA, USA). Then, the samples were diluted in 4×NativePAGE™ Sample Buffer (Invitrogen, Carlsbad, CA, USA) and loaded onto NativePAGE™ 4-16% Bis-Tris Protein Gels (Invitrogen, Carlsbad, CA, USA). The gel-run was performed at 4° C. utilizing the NativePAGE™ Running Buffer Kit (Invitrogen, Carlsbad, CA, USA). Western blot analyses were performed on PVDF membranes using the iBind™ Flex system (Invitrogen, Carlsbad, CA, USA) with anti-BNIP3 (Abcam, Cambridge, UK, ab38621, 1:1,000 dilution) and anti-BAX (Abcam, Cambridge, UK, ab32503, 1:500 dilution). Co-immunoprecipitation experiments Cytosol lysates were cleared by centrifugation at 20,000×g for 15 minutes at 4° C. Protein concentrations in the cytosolic fraction were measured using a DC Protein Assay (Bio-Rad Laboratories Inc., Hercules, CA, USA). Co-immunoprecipitation was performed using protein G-coupled Dynabeads (Invitrogen, Carlsbad, CA, USA) covalently bound to anti-BNIP3 (Abcam, Cambridge, UK, 10 μg/sample) or anti-BAX (Abcam, Cambridge, UK, 0.011 μg/sample). Unbound antibodies were washed of before subjecting the coupled bead antibodies to 500 pg protein-lysate over night at 4° C. with rotation. The precipitate bound to the bead-antibodies was cleared from unbound lysate and resuspended in 4×LDS sample buffer (Invitrogen, Carlsbad, CA, USA), 10× reducing agent (Invitrogen, Carlsbad, CA, USA) and boiled for 10 minutes at 70° C. to separate the precipitate from the bead-antibodies. The precipitate was then analyzed by western blot on nitrocellulose membranes using anti-BNIP3 (Abcam, Cambridge, UK, ab38621, 1:1,000) and anti-BAX (Abcam, Cambridge, UK, ab32503, 1:500). For fluorescent measurements, the precipitate was resuspended in 10× reducing agent and boiled for 10 minutes at 70° C., excited at 485 nm and emitted light measured at 520 nm. Subsequently 4×LDS was added and western blotting performed as described. Immunostaining of BNIP3 in tissue Wild-type and Bnip3−/− mouse hearts were washed free of blood with NaCl in vivo, and then removed and incubated in 40% sucrose solution overnight at 4° C. After fixation using 4% paraformaldehyde for 15 minutes, 8 μm slices were cut and washed three times for 5 minutes with PBS containing 0.5% Triton X-100 for cell permeabilization. Slices were stained with Mito-Tracker orange (Invitrogen, Carlsbad, CA, USA) and incubated with the anti-BNIP3 antibody overnight at 4° C. Staining was performed with an AlexaFluor 488-conjugated goat anti-rabbit secondary antibody and DAPI (Invitrogen, Carlsbad, CA, USA, 1:5,000 dilution). Confocal sections were visualized under a confocal laser scanning microscope using the 63×/1.4 Oil DIC M27 objective (Zeiss Elyra PS; Zeiss, Oberkochen, Germany). Caspase-3 activity Caspase-3 activity was measured in the AAR after 1 hour of reperfusion using a Caspase-3 Assay Kit (Abcam, Cambridge, UK, ab39401), according to the manufacturer's instructions. Bioenergetics For bioenergetics studies, ATP concentrations in the AAR were analyzed after 30 minutes of reperfusion using an ATP Bioluminescent Assay Kit (Sigma-Aldrich, St. Louis, MO, USA, FLAA). Tissue Clearing Hearts were fixed for 4 h in 4% paraformaldehyde (w/v PFA) at 4° C. Samples need to be protected from light in all following steps. Next, hearts were dehydrated using an ascending ethanol series (50%, 70%, 100% (v/v) EtOH in ddH2O) while shaking. overnight in 50% EtOH at 4° C., 30 minutes in 70% EtOH at room temperature, 30 minutes in 70% EtOH at room temperature. In order to enhance sample clarity, samples were bleached for 4 hours at 4° C. while shaking, using freshly prepared bleaching solution (5% (v/v) hydrogen peroxide, 5% (v/v) dimethyl sulfoxide in 100% ethanol). Afterwards samples were washed 3 times in 100% ethanol for 30 minutes at 4° C. while shaking. For the final clearing step, hearts were warmed to room temperature for 5 minutes and subsequently transferred to a glass vial containing pure ethyl cinnamate (ECi, Sigma Aldrich). Samples were cleared for at least 4 hours before imaging and were kept at room temperature and protected from light until and after imaging. Light sheet fluorescence microscopy and image processing Samples were imaged using the Ultramicroscope II and ImSpector software (both LaVision BioTec). Cleared hearts were immersed in pure ECi in a quartz cuvette. Since TAT-WVELHFFN (“WVELHFFN” disclosed as SEQ ID NO: 3) was coupled with Cy 5.5 fluorophores, the excitation wavelengths of the light sheets were set to 639 nm and the corresponding 680/30 band-pass emission filter was used. Additionally, autofluorescence was imaged in the FITC channel (488 nm excitation, 525/50 band-pass emission filter). Samples were clamped in a sample holder, apex and aorta horizontally aligned, between two blocks of ECi-cleared phytagel (1% phytagel in H2O) to prevent deformation. The knot of the ligation, which is used to induce the myocardial I/R, remains in situ and needs to face downwards during imaging to reduce blockage of excitation or emission light. Data sets of whole hearts were obtained with ×1.2 total magnification with 10 μm z spacing between optical planes. The sheet width was set to 4200 and numeric aperture to 0.148. The longest wavelength was imaged first, to prevent photobleaching. 16 bit OME.TIF stacks were converted (ImarisFileConverterx64, BitPlane) into Imaris files (.ims). 3D reconstruction and subsequent analysis was done using Imaris software (BitPlane). Mitochondrial experiments MIM permeahilization To analyze the MIM potential, 5′,6,6′-tetrachloro-1,1′,3,3′-tetraethylbenzimidazolylcarbocyanin iodide (JC-1) was used by performing the MitoProbe™ JC-1 Assay Kit for Flow Cytometry (ThermoFisher Scientific, Waltham, MA, USA, 34152). Each probe contained 250 μg of isolated mitochondria incubated with 1.5 pg JC-1/mg. Changes in mitochondrial MIM potential were analyzed by flow cytometry using FACS Verse (Becton Dickinson, Franklin Lakes, NJ, USA) with an excitation wavelength of 514 nm and emission wavelengths of 529 nm and 590 nm. To assess MIM permeabilization by mPTP opening, mitochondria were isolated from the AAR after 10 minutes of reperfusion. Mitochondrial swelling was measured by changes in light scattering at 540 nm. The final buffer volume was 200 μl (250 mM sucrose, 10 mM HEPES, and 1 mM EGTA, pH 7.4) containing 0.5 mg/mL mitochondria. To distinguish cytochrome c release induced by mPTP opening or MOMP formation, isolated mitochondria (1 mg/mL) were incubated in buffer (150 mM KCl, 5 mM Tris-HCl, pH 7.4) and energized with 5 mM succinate and 2 μM rotenone. rBNIP3 (1 μg), rBAX (1 μg) and 150 mM Ca2+ per mg of protein were added. To induce mPTP opening and swelling, 5 mM Pi was used. The optical density at 540 nm was measured over a 40 minutes period. Cytochrome c contents in the supernatants were analyzed by western blotting. MOM permeabilization To demonstrate the need for BNIP3 on BAX pore formation, isolated mitochondria were incubated with cytoplasm. To inhibit VDAC, BAX and BNIP3, the inhibitors DIDS (1 μg), anti-BAX antibody (1 μg) and BNIP3ΔTM (1 μg) were added. The cytosolic concentrations of cytochrome c, AIF, and active BAX were quantified by western blotting. Cell culture Isolated adult cardiomyocytes To isolate adult cardiomyocytes, wild-type mice were anesthetized with ketamine (100 mg kg-1 i.p.) and xylazine (10 mg kg-1 i.p.), and the hearts were quickly excised. Explanted hearts were perfused in a retrograde manner and digested with HEPES buffer (110 mM NaCl, 2.5 mM KCl, 1.2 mM KH2PO4, 2.5 mM MgSO4, 25 mM HEPES, 10 mM glucose-monohydrate, 4 μM collagenase type II and 28 μM Ca2+, pH 7.4) for 25 minutes at 37° C. The cell suspension was filtered through a 250-μm mesh collector and centrifuged at 18×g for 1 minute. The cell pellet was resuspended in 100 μM HEPES buffer with stepwise increases in the Ca2+ concentration. The cells were plated on cell culture dishes pre-coated with laminin and washed after 4 hours to remove dead and non-adherent cells. The cells were cultured in modified medium 199 including Earl's salts, 2 mM carnitine, 5 mM creatine and 5 mM taurine supplemented with 100 IU/mL penicillin and 100 μg/ml streptomycin. Cytosine-D-arabinofuranoside (10 μM) was added to inhibit the growth of non-myocytes. Hypoxia/reoxygenation (H/R) For hypoxia experiments, cells were incubated in HEPES buffer (113 mM NaCl, 4.7 mM KCl, 12 mM HEPES, 1.2 mM MgSO4, 30 mM taurine, and 1.3 mM CaCl2), pH 7.4) under 1% 02 at 37° C. for 2 h. For reoxygenation, the cells were incubated in HEPES buffer supplemented with 5.5 mM glucose under 21% O2 at 37° C. for 2 hours. Phosphorylation experiments To evaluate BNIP3 serine phosphorylation under AKT inhibition, HL-1 cells were treated with 10 μM AKT1/2 inhibitor (Sigma-Aldrich, St. Louis, MO, USA, A6730). The levels of BNIP3 serine phosphorylation and active BAX were analyzed by western blotting. Peptide effects in human CMs To assess the in vitro effects of TAT-WVELHFFN (“WVELHFFN” disclosed as SEQ ID NO: 3), human CMs were treated with TAT-WVELHFFN (“WVELHFFN” disclosed as SEQ ID NO: 3) (2 nmol in 500 μl HEPES-buffer) and TAT-WVELAASN (“WVELAASN” disclosed as SEQ ID NO: 7) (2 nmol in 500 μl HEPES) during H/R. To visualize apoptotic and necrotic human CMs after H/R, the cells were stained with 7-AAD (red), Apoxin Green (green) and CytoCalcein (blue) using an Apoptosis/Necrosis Detection Kit (Abcam, Cambridge, UK, ab176749), according to the manufacturer's instructions. Images were captured under an EVOS FL microscope (ThermoFisher Scientific, Waltham, MA, USA). To analyze the MIM potential, human CMs were stained with JC-1. The cells were incubated with 6 μM JC-1 in medium at 37° C. for 30 min, washed with PBS buffer at 37° C., and fixed with 4% paraformaldehyde for 15 minutes at RT. DAPI staining was performed, and the cells were analyzed using an EVOS FL microscope (ThermoFisher Scientific, Waltham, MA, USA). Transmission electron microscopy (TEM) Electron microscopic analyses of I/R injury in cardiac tissue specimens and isolated mitochondria were performed as previously described with minor modifications (Hendgen-Cotta et al., 2018). Specifically, cacodylate buffer was used for the fixation and washing steps. The staining procedure was performed by using potassium ferrocyanide (II), thiocarbohydrazide and lead nitrate asparagic acid solution. Samples were infiltrated and embedded in epoxy resin (Durcupan, Sigma-Aldrich, St. Louis, MO, USA). Peptide synthesis L-amino acid peptides were synthesized by JPT Peptide Technologies (Berlin, Germany) using solid-phase resin-based methodology and purified to >90% by HPLC (confirmed by mass spectrometry; Figure S6). The N-terminus of the peptides was capped with an acetyl group, the C-terminus with an amide group. The TAT-BNIP3-20A sequence, GRKKRRQRRRPQMSQSGEENLQGSWVELHFSN (SEQ ID NO: 16), consisted of 12 amino acids from the HIV-1 PTD at the N-terminus and 20 amino acids derived from BNIP3 1-20 at the C-terminus. The TAT-BNIP3-20C sequence, GRKKRRQRRRPQLDAQHESGRSSSKSSHCDSP (SEQ ID NO: 17), consisted of 12 amino acids from the HIV-1 PTD at the N-terminus and 20 amino acids derived from BNIP3 42-61 at the C-terminus. The TAT-octapeptide sequence, GRKKRRQRRRPQWVELHFFN (SEQ ID NO: 18), consisted of 12 amino acids from the HIV-1 PTD at the N terminus and eight amino acids derived from BNIP3 13-20 containing the substitution S19F at the C-terminus. The control TAT-octapeptide sequence, GRKKRRQRRRPQWVELAASN (SEQ ID NO: 19), consisted of 12 amino acids from the HIV-1 PTD at the N-terminus and eight amino acids derived from BNIP3 13-20 containing two substitutions, H17A and F18A, at the C-terminus. For in vivo and cell culture experiments, peptides were dissolved in H2O and diluted with 0.9% NaCl. For protein-peptide interaction studies, peptide libraries were synthesized and immobilized on microarray slides. Peptides were synthesized and immobilized for: (i) the BNIP3/BAX interaction study (provided in FIG. 25 (FIG. 25 discloses SEQ ID NOS 87, 494, 88-91, 26, 33-34, 29, 92, 31, and 93, respectively in order of appearance.)); (ii) the BNIP3/BNIP3 interaction study, in which C-, N- or C/N-terminal truncations of the wild-type sequence of BNIP3 1-20 were performed (provided in FIG. 26 (FIG. 26 discloses SEQ ID NOS 1, 94-101, 53, 52, 51, 8, 2, 50, 49, 48, 47, 46, 45, 44, 43, 42, 41, 40, and 102-117, respectively, in order of columns.)); and (iii) the BNIP3/BNIP3 interaction study, in which single residues of the wild-type sequence of BNIP3 1-20 were exchanged for 18 neutral amino acids (provided in FIG. 27A-FIG. 27E (FIG. 27A-27E disclose SEQ ID NOS 118-493, respectively, in order of appearance.)). Interaction studies with peptide microarrays Peptide microarrays For protein-peptide binding studies, including the rBNIP3-BAX peptide interaction analysis, rBNIP3-BNIP3 peptide alanine scan, truncation analysis, and substitution analysis, rBNIP3 was used at concentrations of 5 μg/mL and 1 μg/mL. The studies were performed by JPT Peptide Technologies (Berlin, Germany). For fluorescence-labeling, the DyLight Microscale Antibody Labeling Kit (ThermoFisher Scientific, Waltham, MA, USA) with label DyLight 650 was used. The assay was performed using the automated Tecan HS 4800 microarray processing station. Microarrays were incubated with customer-provided samples diluted in blocking buffer for 2 hours at 30° C. Before each step, the microarrays were washed with washing buffer. Microarrays were scanned using a high-resolution fluorescence scanner. The laser settings and applied resolution were identical for all performed measurements. The resulting images were analyzed and quantified using GenePix spot-recognition software (Molecular Devices, Sant Jose, CA, USA). The mean signal intensity was extracted (between 0 and 65535 arbitrary units) for each spot. For further data evaluation, the so-called MMC2 values were determined. The MMC2 equals the mean value of all three instances on the microarray except for when the coefficient of variation (CV)—the standard deviation divided by the mean value—is >0.5. In this case, the mean of the two closest values (MC2) was assigned to MMC2. Overlay assays For protein-protein/peptide overlay assays, 300 ng rBNIP3 or rBAX were spotted on a nitrocellulose membrane. After 5 minutes of incubation followed by 5 minutes of washing with TBS-T, the membranes were incubated for 1.5 hours with 2.5 μg rBNIP3 or rBAX protein labeled with Dy650 in 200 μl TBS-T or with 50 μg TAT-WVELHFFN (“WVELHFFN” disclosed as SEQ ID NO: 3) labeled with Lys(5(6))-Fam. After washing two times for 5 minutes with TBS-T, fluorescence was detected using the ImageQuant system (Amersham, Little Chalfont, UK). Docking simulation experiments Docking simulation experiments were performed using Autodock Vina and HADDOCK. The structure of BAX (pdb-ID: 4S0O) was taken from the Protein Data Bank, whereas the structure of BNIP3 and the peptide TAT-WVELHFFN (“WVELHFFN” disclosed as SEQ ID NO: 3) was modelled using Modeller 9.15. The template structures corresponded to PDB codes 2K7W and 2KA1. The created model was energy minimized using NAMD2.9 and the CHARMM36 force field. Proximity ligation assay To assess the BNIP3-BAX interaction, proximity ligation assays were performed in human CM after 1 hour hypoxia/1 hour reoxygenation using Duolink In Situ Red Starter Kit Mouse according to the manufacturer's protocol (Sigma-Aldrich, St. Louis, MO, USA, DU092101-1KT) using antibodies against BNIP3 (ab10433, 1:1,000 dilution) and BAX (ab7977, 1:1,000), followed by visualization by confocal laser microscopy (Zeiss Elyra PS; Zeiss, Oberkochen, Germany). Circular dichroism spectroscopy. The circular dichroism spectra of the BNIP3 protein and the TAT-WVELHFFN (“WVELHFFN” disclosed as SEQ ID NO. 3) peptide were recorded on a Jasco J-715 spectropolarimeter (Jasco, Pfungstadt, Germany) at 37° C. in 1×PBS, pH 7.4. Peptide uptake, distribution and stability For uptake and distribution analyses, mice were injected with TAT-WVELHFFN-Lys(5(6)-FAM (“WVELHFFN-Lys(5(6)-FAM” disclosed as SEQ ID NO: 15)) (267 nmol kg-1 in 50 μL 0.9% NaCl). Sham-operated mice and baseline values served as controls. For the pharmacokinetic studies, TAT-WVELHFFN (“WVELHFFN” disclosed as SEQ ID NO: 3) was incubated in human whole blood, serum and plasma for 0, 10, 20, 30, and 60 minutes at 37° C. Treatment with proteinase K (0.3 mg/ml) served as a control. Human samples Left ventricular tissue samples were obtained from 3 patients with suspected myocarditis who underwent endomyocardial biopsy (Ethics Approval of the Ethics Committee, University Duisburg-Essen, No. 17-7392-BO). The diagnosis was not confirmed histologically. The three donors had a median age of 47.

Human cardiomyocytes (HCM)

HCMs were obtained from PromoCell and cultured according to the manufacturer's instructions. The cells were incubated with 0, 1 and 10 μM Doxorubicin and treated simultaneously with 0 and 1.5 pM TAT-WVELHFFN (“WVELHFFN” disclosed as SEQ ID NO: 3) for four and 22 hours, respectively. Mitochondrial membrane potential was measured using TMRM-Kit (Abcam ab228569), calcium overload using Rhod-2AM, cell viability using Cytotoxicity Kit (Molecular Probes L3224), all according to the manufacturer's instructions.

### DOX Treatment In Vivo

Wild-type mice aged 12±3 weeks received eight times, every two days, an i.p. injection of 0.9% physiological saline (vehicle control) or DOX (3 mg/kg body weight). TAT-WVELHFFN (“WVELHFFN” disclosed as SEQ ID NO. 3) was administered i.p. simultaneously. Serial echocardiography was performed on all mice at baseline and on day 16 and day 42. Left ventricular ejection fraction, fractional shortening and isovolumetric relaxation time were evaluated. Cardiac troponin I (cTNI-ELISA life Diagnostic CTNI-1-US), BNP (BNP-ELISA EIAM-BNP), and LDH (activity assay Sigma MAK066) were measured according to the manufacturer's instructions. Mitochondrial OCR was assessed with a Seahorse XF24 Analyzer using the mitochondrial stress test (Agilent)

QUANTIFICATION AND STATISTICAL ANALYSIS GraphPad Prism 7 software (GraphPad, San Diego, CA, USA) was used to perform statistical tests and to generate graphs. Data are presented as the means±SEM, and p values were calculated as detailed in the corresponding legends. Sample sizes were determined on the basis of previous experimental experience or based on general practices in the field. All replicates constitute biological replicates. For the in vivo studies, mice were randomly allocated to groups. For all histopathological and immunostaining analyses, the experimenters were blinded to the experimental conditions. Comparisons between characteristics of subject groups were analyzed with two-tailed Student's t-test. For comparisons among more than two groups, one-way or two-way ANOVA with Bonferroni's post hoc test was used. DATA AND SOFTWARE AVAILABILITY The ImageJ software 1.52a and Fiji/ImageJ 1.52e are available at the website //imagej.net/Fiji.

We claim:

1.-170. (canceled)

171. A method of assessing a drug candidate, the method comprising: contacting the drug candidate to a sample comprising a mitochondrion, and assaying for localization of a mitochondrial modulator relative to the mitochondrion.

172. The method of claim 171, wherein the drug candidate comprises a protein segment.

173. The method of claim 172, wherein the drug candidate comprises a protein segment arising from a protein implicated in mitochondrial regulation.

174. The method of claim 172, wherein the drug candidate comprises a cellular translocation signal.

175. The method of claim 174, wherein the cellular translocation signal is selected from the list consisting of SEQ ID NO: 31 to SEQ ID NO: 50.

176. The method of claim 171, wherein the sample comprising a mitochondria comprises an intact cell.

177. The method of claim 171, wherein the sample comprising a mitochondria is an infarcted sample.

178. The method of claim 171, wherein the sample comprising a mitochondria is a cellular extract.

179. The method of claim 171, wherein the sample comprising a mitochondria is a human sample.

180. The method of claim 171, wherein the sample comprising a mitochondria is a mammalian sample.

181. The method of claim 171, wherein assaying for localization of a mitochondrial modulator comprises fractionating the sample comprising a mitochondrion into subcellular fractions, and performing immunoblotting on the subcellular fractions.

182. The method of claim 171, wherein assaying for localization of a mitochondrial modulator comprises in situ fluorescence detection.

183. The method of claim 171, wherein the mitochondrial modulator exhibits oxidative stress mediated localization to the mitochondrion.

184. The method of claim 171, wherein the mitochondrial modulator localizes outside of the mitochondrion in response to low oxygen conditions.

185. The method of claim 171, wherein the mitochondrial modulator localizes to the mitochondrion in response to reperfusion.

186. The method of claim 171, wherein the mitochondrial modulator is BNIP3.

187. The method of claim 171, wherein the mitochondrial modulator is BAX.

188. The method of claim 171, wherein the mitochondrial modulator is a BNIP3/BAX complex.

189. The method of claim 171, wherein the mitochondrial modulator is a BNIP3/BAX oligomer.

190. The method of claim 171, wherein blocking mitochondrial modulator localization to the mitochondria in response to oxygenation indicates a positive drug candidate assessment.
